(19) United States
(12) Patent Application Publication
(10) Pub. No.: US 2024/0148733 A1
(43) Pub. Date: May 9, 2024

(54) COMBINATION THERAPY

(71) Applicant: Genentech, Inc., South San Francisco, CA (US)

(72) Inventors: Anwesha DEY, Cupertino, CA (US); Marie EVANGELISTA, San Francisco, CA (US)

(21) Appl. No.: 18/510,227

(22) Filed: Nov. 15, 2023

Related U.S. Application Data
(63) Continuation of application No. PCT/US2022/072452, filed on May 19, 2022.
(60) Provisional application No. 63/190,766, filed on May 19, 2021.

(51) Int. Cl. A61K 31/519; A61K 31/343; A61K 31/416; A61K 31/4162; A61K 31/44; A61K 31/4402; A61K 31/4412; A61K 31/517; A61K 31/635; A61K 47/54; A61K 47/55; A61P 35/00

(52) U.S. Cl. CPC A61K 31/519; A61K 31/343; A61K 31/416; A61K 31/4162; A61K 31/44; A61K 31/4402; A61K 31/4412; A61K 31/517; A61K 31/635; A61K 47/545; A61K 47/55; A61P 35/00

(57) ABSTRACT

Provided herein is a combination, comprising: (i) one or more YAP/TAZ-TEAD inhibitors; and (ii) one or more KRAS inhibitors, such as the specific YAP/TAZ-TEAD inhibitors and KRAS inhibitors described herein.

Also provided herein are methods of modulating or inhibiting KRAS activity in a cell, comprising administering to the cell an effective amount of such combinations.

Also provided herein are methods of treating cancer in a subject in need thereof, comprising administering to the subject an effective amount of such combinations.

### CROSS-REFERENCE TO RELATED APPLICATIONS

This application is a continuation of International Application No. PCT/US2022/072452, filed May 19, 2022, which claims priority to and benefit of U.S. Provisional Patent Application No. 63/190,766, filed May 19, 2021, the disclosures of which are hereby incorporated herein by reference in their entireties.

### BACKGROUND

Ras is a small GTP-binding protein that functions as a nucleotide-dependent switch for central growth signaling pathways. In response to extracellular signals, Ras is converted from a GDP-bound (Ras^{GDP}) to a GTP-bound (Ras^{GTP}) state, as catalyzed by guanine nucleotide exchange factors (GEFs), notably the SOS1 protein. Active Ras^{GTP} mediates its diverse growth-stimulating functions through its direct interactions with effectors including Raf, PI3K, and Ral guanine nucleotide dissociation stimulator. The intrinsic GTPase activity of Ras then hydrolyzes GTP to GDP to terminate Ras signaling. The Ras GTPase activity can be further accelerated by its interactions with GTPase-activating proteins (GAPs), including the neurofibromin 1 tumor suppressor.

Mutant Ras has a reduced GTPase activity, which prolongs its activated conformation, thereby promoting Ras-dependent signaling and cancer cell survival or growth. Mutation in Ras which affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. Mutations in any one of the three main isoforms of RAS (H-Ras, N-Ras, or K-Ras) genes are common events in human tumorigenesis. Among the three Ras isoforms (K, N, and H), K-Ras is most frequently mutated.

The most common K-Ras (or KRAS) mutations are found at residue G12 and G13 in the P-loop and at residue Q61. G12C is a frequent mutation of K-Ras gene (glycine-12 to cysteine). G12C is a single point mutation with a glycine-to-cysteine substitution at codon 12. This substitution favors the activated state of KRAS, amplifying signaling pathways that lead to oncogenesis (see, e.g., Hallin et al. (Cancer Discov, 2020, 10(1): 54-71), Skoulidis et al. (N. Engl. J. Med., 2021, 384(25): 2371-2381), and Hong et al. (N. Engl. J. Med., 2020, 383(13): 1207-1217)). G12D, G12V, and G13D are other frequent mutations. Mutations of Ras in cancer are associated with poor prognosis.

Inactivation of oncogenic Ras in mice results in tumor shrinkage. Thus, Ras is widely considered an oncology target of exceptional importance. However, treatment with inhibitors of Ras (for example, KRAS) can lead to resistance through bypass of KRAS/MAPK pathway dependence, and activation of the Hippo pathway.

The Hippo pathway is a signaling pathway that regulates cell proliferation and cell death and determines organ size. The pathway is believed to play a role as a tumor suppressor in mammals, and disorders of the pathway are often detected in human cancers. The pathway is involved in and/or may regulate the self-renewal and differentiation of stem cells and progenitor cells. In addition, the Hippo pathway may be involved in wound healing and tissue regeneration. Furthermore, it is believed that, as the Hippo pathway cross-talks with other signaling pathways such as Wnt, Notch, Hedgehog, and MAPK/ERK, it may influence a wide variety of biological events, and that its dysfunction could be involved in many human diseases in addition to cancer.

The Hippo signaling pathway core consists of a cascade of kinases (Hippo-MST1-2 being upstream of Lats 1-2 and NDRI-2) leading to the phosphorylation of two transcriptional co-activators, YAP (Yes-Associated Protein) and TAZ (Transcription co-activator with PDZ binding motif or tafazzin). Non-phosphorylated, activated YAP is translocated into the cell nucleus where its major target transcription factors are the four proteins of the TEAD-domain-containing family (TEAD1-TEAD4, collectively “TEAD”). YAP together with TEAD (or other transcription factors such as Smad1, RUNX, ErbB4 and p73) has been shown to induce the expression of a variety of genes, including connective tissue growth factor (CTGF), Gli2, Birc5, Birc2, fibroblast growth factor 1 (FGF1), and amphiregulin (AREG). Like YAP, non-phosphorylated TAZ is translocated into the cell nucleus where it interacts with multiple DNA-binding transcription factors, such as peroxisome proliferator-activated receptor γ (PPARγ), thyroid transcription factor-1 (TTF-1), Pax3, TBX5, RUNX, TEAD1 and Smad2/3/4. Many of the genes activated by YAP/TAZ-transcription factor complexes mediate cell survival and proliferation. Therefore, under some conditions YAP and/or TAZ acts as an oncogene and the Hippo pathway acts as a tumor suppressor.

Because the Hippo signaling pathway is a regulator of animal development, organ size control, and stem cell regulation, it has been implicated in cancer development. In vitro, the overexpression of YAP or TAZ in mammary epithelial cells induces cell transformation, through interaction of both proteins with the TEAD family of transcription factors. Increased YAP/TAZ transcriptional activity induces oncogenic properties such as epithelial-mesenchymal transition and was also shown to confer stem cells properties to breast cancer cells. In vivo, in mouse liver, the overexpression of YAP or the genetic knockout of its upstream regulators MST1-2 triggers the development of hepatocellular carcinomas. Furthermore, when the tumor suppressor NF2 is inactivated in the mouse liver, the development of hepatocellular carcinomas can be blocked completely by the co-inactivation of YAP.

It is believed that deregulation of the Hippo tumor suppressor pathway is a major event in the development of a wide range of cancer types and malignancies.

Hence, pharmacological targeting of the Hippo cascade through inhibition of YAP, TAZ, TEAD, and/or the YAP:TEAD protein-protein interaction would be a valuable approach for the treatment of cancers that harbor functional alterations of this pathway. It has also been found that autopalmitoylation of TEAD proteins regulates transcriptional output of Hippo pathway, an the palmitoylation of TEAD transcription factors is required for their stability and function in Hippo pathways signaling, making the lipid binding pocket an attractive target (see, e.g., Chan et al. (Nat. Chem. Biol. 2016, 12(4): 282-289), Noland et al. (Structure, 2016, 24(1): 179-186), and Kim et al. (Biological Sciences, 2019, 116(20): 9877-9882)). Covalent TEAD inhibitors are described in, for example, Karats et al. (J. Med. Chem. 2020, 63, 11972-11989), Lu et al. (Acta Pharmaceutica Sinica B, 2021, 11(10): 3206-3219), Fan et al. (Biorxiv, 2022, DOI:10.1101/2022.05.10.491316), and Kaneda et al. (Am. J. Cancer Res., 2020, 10(12): 4399-4415).

There is, therefore, a need for therapies that improve the ability of inhibitors of Ras (for example, KRAS) and inhibitors of YAP, TAZ, TEAD, and/or the YAP:TEAD protein-protein interaction to treat a range of diseases, disorders, and conditions, including cancer.

### SUMMARY

In one aspect, the present disclosure is directed to methods of modulating YAP/TAZ-TEAD activity, or KRAS activity, or both, in a cell, comprising administering to the cell an effective amount of a combination comprising: (i) one or more YAP/TAZ-TEAD inhibitors; and (ii) one or more KRAS inhibitors.

In another aspect, the present disclosure is further directed to methods of inhibiting YAP/TAZ-TEAD activity, or KRAS activity, or both, in a cell, comprising administering to the cell an effective amount of a combination comprising: (i) one or more YAP/TAZ-TEAD inhibitors; and (ii) one or more KRAS inhibitors.

In another aspect, the present disclosure is directed to methods of treating cancer in a subject in need thereof, comprising administering to the subject an effective amount of a combination comprising: (i) one or more YAP/TAZ-TEAD inhibitors; and (ii) one or more KRAS inhibitors.

In one aspect, the present disclosure is directed to compositions, comprising: (i) one or more YAP/TAZ-TEAD inhibitors; and (ii) one or more KRAS inhibitors.

In one aspect, the present disclosure is directed to kits, comprising (i) an effective amount of a combination comprising one or more YAP/TAZ-TEAD inhibitors and one or more KRAS inhibitors; and (ii) instructions for administering the combination to treat cancer in a subject in need thereof.

In one aspect, the present disclosure is directed to the use of a combination comprising one or more YAP/TAZ-TEAD inhibitors and one or more KRAS inhibitors in the manufacture of a medicament for use in the treatment of cancer in a subject in need thereof.

In one aspect, the present disclosure is directed to compositions comprising: (i) one or more YAP/TAZ-TEAD inhibitors; and (ii) one or more KRAS inhibitors for use in the treatment of cancer.

In a further aspect, the present disclosure is directed to processes for preparing a composition, comprising: (i) one or more YAP/TAZ-TEAD inhibitors; and (ii) one or more KRAS inhibitors.

### BRIEF DESCRIPTION OF THE DRAWINGS

The following embodiments are representative of some aspects of the disclosure.

FIG. 1 depicts % inhibition of H2122 cells (KRAS G12C) following administration of a combination comprising Compound T1 (a YAP/TAZ-TEAD inhibitor) and Compound K1 (a KRAS inhibitor), demonstrating that administration of Compound T1 sensitizes cells resistant to Compound K1.

FIG. 2 depicts % inhibition of H2122 cells (KRAS G12C) following administration of a combination comprising Compound T2 (a YAP/TAZ-TEAD inhibitor) and Compound K1 (a KRAS inhibitor), demonstrating that administration of Compound T2 sensitizes cells resistant to Compound K1.

FIG. 3 depicts % inhibition of H2122 cells (KRAS G12C) following administration of a combination comprising Compound T3 (a YAP/TAZ-TEAD inhibitor) and Compound K1 (a KRAS inhibitor), demonstrating that administration of Compound T3 sensitizes cells resistant to Compound K1.

FIG. 4 depicts % inhibition of H2122 cells (KRAS G12C) following administration of a combination comprising Compound T4 (a YAP/TAZ-TEAD inhibitor) and Compound K1 (a KRAS inhibitor), demonstrating that administration of Compound T4 sensitizes cells resistant to Compound K1.

FIG. 5 depicts % inhibition of H2122 cells (KRAS G12C) following administration of a combination comprising Compound T1 (a YAP/TAZ-TEAD inhibitor) and Compound K2 (a KRAS inhibitor), demonstrating that administration of Compound T1 sensitizes cells resistant to Compound K2.

FIG. 6 depicts % inhibition of H2122 cells (KRAS G12C) following administration of a combination comprising Compound T2 (a YAP/TAZ-TEAD inhibitor) and Compound K2 (a KRAS inhibitor), demonstrating that administration of Compound T2 sensitizes cells resistant to Compound K2.

FIG. 7 depicts % inhibition of H2122 cells (KRAS G12C) following administration of a combination comprising Compound T3 (a YAP/TAZ-TEAD inhibitor) and Compound K2 (a KRAS inhibitor), demonstrating that administration of Compound T3 sensitizes cells resistant to Compound K2.

FIG. 8 depicts % inhibition of H2122 cells (KRAS G12C) following administration of a combination comprising Compound T4 (a YAP/TAZ-TEAD inhibitor) and Compound K2 (a KRAS inhibitor), demonstrating that administration of Compound T4 sensitizes cells resistant to Compound K2.

FIG. 9 depicts soft agar 3D culture growth following administration of a combination comprising Compound T2 (a YAP/TAZ-TEAD inhibitor) and Compound K2 (a KRAS inhibitor), demonstrating that administration of Compound T2 sensitizes cells resistant to Compound K2.

FIG. 10 depicts % inhibition of H2030 cells (KRAS G12C) following administration of a combination comprising Compound T1 (a YAP/TAZ-TEAD inhibitor) and Compound K1 (a KRAS inhibitor), demonstrating that administration of Compound T1 sensitizes cells resistant to Compound K1.

FIG. 11 depicts % inhibition of H2030 cells (KRAS G12C) following administration of a combination comprising Compound T3 (a YAP/TAZ-TEAD inhibitor) and Compound K1 (a KRAS inhibitor), demonstrating that administration of Compound T3 sensitizes cells resistant to Compound K1.

FIG. 12 depicts % inhibition of H2030 cells (KRAS G12C) following administration of a combination comprising Compound T5 (a YAP/TAZ-TEAD inhibitor) and Compound K1 (a KRAS inhibitor), demonstrating that administration of Compound T5 sensitizes cells resistant to Compound K1.

FIG. 13 depicts % inhibition of H2030 cells (KRAS G12C) following administration of a combination comprising Compound T6 (a YAP/TAZ-TEAD inhibitor) and Compound K1 (a KRAS inhibitor), demonstrating that administration of Compound T6 sensitizes cells resistant to Compound K1.

FIG. 14 depicts % inhibition of H2030 cells (KRAS G12C) following administration of a combination comprising Compound T7 (a YAP/TAZ-TEAD inhibitor) and Compound K1 (a KRAS inhibitor), demonstrating that administration of Compound T7 sensitizes cells resistant to Compound K1.

FIG. 15 depicts % inhibition of H2030 cells (KRAS G12C) following administration of a combination comprising Compound T8 (a YAP/TAZ-TEAD inhibitor) and Compound K1 (a KRAS inhibitor), demonstrating that administration of Compound T8 sensitizes cells resistant to Compound K1.

FIG. 16 depicts % inhibition of H2030 cells (KRAS G12C) following administration of a combination comprising Compound T9 (a YAP/TAZ-TEAD inhibitor) and Compound K1 (a KRAS inhibitor), demonstrating that administration of Compound T9 sensitizes cells resistant to Compound K1.

FIG. 17 depicts % inhibition of H2030 cells (KRAS G12C) following administration of a combination comprising Compound T10 (a YAP/TAZ-TEAD inhibitor) and Compound K1 (a KRAS inhibitor), demonstrating that administration of Compound T10 sensitizes cells resistant to Compound K1.

FIG. 18 depicts % inhibition of H2030 cells (KRAS G12C) following administration of a combination comprising Compound T1 (a YAP/TAZ-TEAD inhibitor) and Compound K2 (a KRAS inhibitor), demonstrating that administration of Compound T1 sensitizes cells resistant to Compound K2.

FIG. 19 depicts % inhibition of H2030 cells (KRAS G12C) following administration of a combination comprising Compound T3 (a YAP/TAZ-TEAD inhibitor) and Compound K2 (a KRAS inhibitor), demonstrating that administration of Compound T3 sensitizes cells resistant to Compound K2.

FIG. 20 depicts % inhibition of H2030 cells (KRAS G12C) following administration of a combination comprising Compound T5 (a YAP/TAZ-TEAD inhibitor) and Compound K2 (a KRAS inhibitor), demonstrating that administration of Compound T5 sensitizes cells resistant to Compound K2.

FIG. 21 depicts % inhibition of H2030 cells (KRAS G12C) following administration of a combination comprising Compound T6 (a YAP/TAZ-TEAD inhibitor) and Compound K2 (a KRAS inhibitor), demonstrating that administration of Compound T6 sensitizes cells resistant to Compound K2.

FIG. 22 depicts % inhibition of H2030 cells (KRAS G12C) following administration of a combination comprising Compound T7 (a YAP/TAZ-TEAD inhibitor) and Compound K2 (a KRAS inhibitor), demonstrating that administration of Compound T7 sensitizes cells resistant to Compound K2.

FIG. 23 depicts % inhibition of H2030 cells (KRAS G12C) following administration of a combination comprising Compound T8 (a YAP/TAZ-TEAD inhibitor) and Compound K2 (a KRAS inhibitor), demonstrating that administration of Compound T8 sensitizes cells resistant to Compound K2.

FIG. 24 depicts % inhibition of H2030 cells (KRAS G12C) following administration of a combination comprising Compound T9 (a YAP/TAZ-TEAD inhibitor) and Compound K2 (a KRAS inhibitor), demonstrating that administration of Compound T9 sensitizes cells resistant to Compound K2.

FIG. 25 depicts % inhibition of H2030 cells (KRAS G12C) following administration of a combination comprising Compound T10 (a YAP/TAZ-TEAD inhibitor) and Compound K2 (a KRAS inhibitor), demonstrating that administration of Compound T10 sensitizes cells resistant to Compound K2.

FIG. 26 depicts % inhibition of H2030 cells (KRAS G12C) following administration of a combination comprising Compound T1 (a YAP/TAZ-TEAD inhibitor) and Compound K3 (a KRAS inhibitor), demonstrating that administration of Compound T1 sensitizes cells resistant to Compound K3.

FIG. 27 depicts % inhibition of H2030 cells (KRAS G12C) following administration of a combination comprising Compound T3 (a YAP/TAZ-TEAD inhibitor) and Compound K3 (a KRAS inhibitor), demonstrating that administration of Compound T3 sensitizes cells resistant to Compound K3.

FIG. 28 depicts % inhibition of H2030 cells (KRAS G12C) following administration of a combination comprising Compound T5 (a YAP/TAZ-TEAD inhibitor) and Compound K3 (a KRAS inhibitor), demonstrating that administration of Compound T5 sensitizes cells resistant to Compound K3.

FIG. 29 depicts % inhibition of H2030 cells (KRAS G12C) following administration of a combination comprising Compound T6 (a YAP/TAZ-TEAD inhibitor) and Compound K3 (a KRAS inhibitor), demonstrating that administration of Compound T6 sensitizes cells resistant to Compound K3.

FIG. 30 depicts % inhibition of H2030 cells (KRAS G12C) following administration of a combination comprising Compound T7 (a YAP/TAZ-TEAD inhibitor) and Compound K3 (a KRAS inhibitor), demonstrating that administration of Compound T7 sensitizes cells resistant to Compound K3.

FIG. 31 depicts % inhibition of H2030 cells (KRAS G12C) following administration of a combination comprising Compound T8 (a YAP/TAZ-TEAD inhibitor) and Compound K3 (a KRAS inhibitor), demonstrating that administration of Compound T8 sensitizes cells resistant to Compound K3.

FIG. 32 depicts % inhibition of H2030 cells (KRAS G12C) following administration of a combination comprising Compound T9 (a YAP/TAZ-TEAD inhibitor) and Compound K3 (a KRAS inhibitor), demonstrating that administration of Compound T9 sensitizes cells resistant to Compound K3.

FIG. 33 depicts % inhibition of H2030 cells (KRAS G12C) following administration of a combination comprising Compound T10 (a YAP/TAZ-TEAD inhibitor) and Compound K3 (a KRAS inhibitor), demonstrating that administration of Compound T10 sensitizes cells resistant to Compound K3.

FIG. 34 depicts % inhibition of H2030 cells (KRAS G12C) following administration of a combination comprising Compound T1 (a YAP/TAZ-TEAD inhibitor) and Compound K4 (a KRAS inhibitor), demonstrating that administration of Compound T1 sensitizes cells resistant to Compound K4.

FIG. 35 depicts % inhibition of H2030 cells (KRAS G12C) following administration of a combination comprising Compound T3 (a YAP/TAZ-TEAD inhibitor) and Compound K4 (a KRAS inhibitor), demonstrating that administration of Compound T3 sensitizes cells resistant to Compound K4.

FIG. 36 depicts % inhibition of H2030 cells (KRAS G12C) following administration of a combination comprising Compound T5 (a YAP/TAZ-TEAD inhibitor) and Compound K4 (a KRAS inhibitor), demonstrating that administration of Compound T5 sensitizes cells resistant to Compound K4.

FIG. 37 depicts % inhibition of H2030 cells (KRAS G12C) following administration of a combination comprising Compound T6 (a YAP/TAZ-TEAD inhibitor) and Compound K4 (a KRAS inhibitor), demonstrating that administration of Compound T6 sensitizes cells resistant to Compound K4.

FIG. 38 depicts % inhibition of H2030 cells (KRAS G12C) following administration of a combination comprising Compound T7 (a YAP/TAZ-TEAD inhibitor) and Compound K4 (a KRAS inhibitor), demonstrating that administration of Compound T7 sensitizes cells resistant to Compound K4.

FIG. 39 depicts % inhibition of H2030 cells (KRAS G12C) following administration of a combination comprising Compound T8 (a YAP/TAZ-TEAD inhibitor) and Compound K4 (a KRAS inhibitor), demonstrating that administration of Compound T8 sensitizes cells resistant to Compound K4.

FIG. 40 depicts % inhibition of H2030 cells (KRAS G12C) following administration of a combination comprising Compound T9 (a YAP/TAZ-TEAD inhibitor) and Compound K4 (a KRAS inhibitor), demonstrating that administration of Compound T9 sensitizes cells resistant to Compound K4.

FIG. 41 depicts % inhibition of H2030 cells (KRAS G12C) following administration of a combination comprising Compound T10 (a YAP/TAZ-TEAD inhibitor) and Compound K4 (a KRAS inhibitor), demonstrating that administration of Compound T10 sensitizes cells resistant to Compound K4.

FIG. 42 depicts % inhibition of H358 parental cells (KRAS G12C) following administration of a combination comprising Compound T1 (a YAP/TAZ-TEAD inhibitor) and Compound K1 (a KRAS inhibitor), demonstrating that administration of Compound T1 sensitizes cells resistant to Compound K1.

FIG. 43 depicts % inhibition of H358 parental cells (KRAS G12C) following administration of a combination comprising Compound T3 (a YAP/TAZ-TEAD inhibitor) and Compound K1 (a KRAS inhibitor), demonstrating that administration of Compound T3 sensitizes cells resistant to Compound K1.

FIG. 44 depicts % inhibition of H358 parental cells (KRAS G12C) following administration of a combination comprising Compound T5 (a YAP/TAZ-TEAD inhibitor) and Compound K1 (a KRAS inhibitor), demonstrating that administration of Compound T5 sensitizes cells resistant to Compound K1.

FIG. 45 depicts % inhibition of H358 parental cells (KRAS G12C) following administration of a combination comprising Compound T6 (a YAP/TAZ-TEAD inhibitor) and Compound K1 (a KRAS inhibitor), demonstrating that administration of Compound T6 sensitizes cells resistant to Compound K1.

FIG. 46 depicts % inhibition of H358 parental cells (KRAS G12C) following administration of a combination comprising Compound T7 (a YAP/TAZ-TEAD inhibitor) and Compound K1 (a KRAS inhibitor), demonstrating that administration of Compound T7 sensitizes cells resistant to Compound K1.

FIG. 47 depicts % inhibition of H358 parental cells (KRAS G12C) following administration of a combination comprising Compound T8 (a YAP/TAZ-TEAD inhibitor) and Compound K1 (a KRAS inhibitor), demonstrating that administration of Compound T8 sensitizes cells resistant to Compound K1.

FIG. 48 depicts % inhibition of H358 parental cells (KRAS G12C) following administration of a combination comprising Compound T9 (a YAP/TAZ-TEAD inhibitor) and Compound K1 (a KRAS inhibitor), demonstrating that administration of Compound T9 sensitizes cells resistant to Compound K1.

FIG. 49 depicts % inhibition of H358 parental cells (KRAS G12C) following administration of a combination comprising Compound T10 (a YAP/TAZ-TEAD inhibitor) and Compound K1 (a KRAS inhibitor), demonstrating that administration of Compound T10 sensitizes cells resistant to Compound K1.

FIG. 50 depicts % inhibition of H358 parental cells (KRAS G12C) following administration of a combination comprising Compound T1 (a YAP/TAZ-TEAD inhibitor) and Compound K2 (a KRAS inhibitor), demonstrating that administration of Compound T1 sensitizes cells resistant to Compound K2.

FIG. 51 depicts % inhibition of H358 parental cells (KRAS G12C) following administration of a combination comprising Compound T3 (a YAP/TAZ-TEAD inhibitor) and Compound K2 (a KRAS inhibitor), demonstrating that administration of Compound T3 sensitizes cells resistant to Compound K2.

FIG. 52 depicts % inhibition of H358 parental cells (KRAS G12C) following administration of a combination comprising Compound T5 (a YAP/TAZ-TEAD inhibitor) and Compound K2 (a KRAS inhibitor), demonstrating that administration of Compound T5 sensitizes cells resistant to Compound K2.

FIG. 53 depicts % inhibition of H358 parental cells (KRAS G12C) following administration of a combination comprising Compound T6 (a YAP/TAZ-TEAD inhibitor) and Compound K2 (a KRAS inhibitor), demonstrating that administration of Compound T6 sensitizes cells resistant to Compound K2.

FIG. 54 depicts % inhibition of H358 parental cells (KRAS G12C) following administration of a combination comprising Compound T7 (a YAP/TAZ-TEAD inhibitor) and Compound K2 (a KRAS inhibitor), demonstrating that administration of Compound T7 sensitizes cells resistant to Compound K2.

FIG. 55 depicts % inhibition of H358 parental cells (KRAS G12C) following administration of a combination comprising Compound T8 (a YAP/TAZ-TEAD inhibitor) and Compound K2 (a KRAS inhibitor), demonstrating that administration of Compound T8 sensitizes cells resistant to Compound K2.

FIG. 56 depicts % inhibition of H358 parental cells (KRAS G12C) following administration of a combination comprising Compound T9 (a YAP/TAZ-TEAD inhibitor) and Compound K2 (a KRAS inhibitor), demonstrating that administration of Compound T9 sensitizes cells resistant to Compound K2.

FIG. 57 depicts % inhibition of H358 parental cells (KRAS G12C) following administration of a combination comprising Compound T10 (a YAP/TAZ-TEAD inhibitor) and Compound K2 (a KRAS inhibitor), demonstrating that administration of Compound T10 sensitizes cells resistant to Compound K2.

FIG. 58 depicts % inhibition of H358 parental cells (KRAS G12C) following administration of a combination comprising Compound T1 (a YAP/TAZ-TEAD inhibitor) and Compound K3 (a KRAS inhibitor), demonstrating that administration of Compound T1 sensitizes cells resistant to Compound K3.

FIG. 59 depicts % inhibition of H358 parental cells (KRAS G12C) following administration of a combination comprising Compound T3 (a YAP/TAZ-TEAD inhibitor) and Compound K3 (a KRAS inhibitor), demonstrating that administration of Compound T3 sensitizes cells resistant to Compound K3.

FIG. 60 depicts % inhibition of H358 parental cells (KRAS G12C) following administration of a combination comprising Compound T5 (a YAP/TAZ-TEAD inhibitor) and Compound K3 (a KRAS inhibitor), demonstrating that administration of Compound T5 sensitizes cells resistant to Compound K3.

FIG. 61 depicts % inhibition of H358 parental cells (KRAS G12C) following administration of a combination comprising Compound T6 (a YAP/TAZ-TEAD inhibitor) and Compound K3 (a KRAS inhibitor), demonstrating that administration of Compound T6 sensitizes cells resistant to Compound K3.

FIG. 62 depicts % inhibition of H358 parental cells (KRAS G12C) following administration of a combination comprising Compound T7 (a YAP/TAZ-TEAD inhibitor) and Compound K3 (a KRAS inhibitor), demonstrating that administration of Compound T7 sensitizes cells resistant to Compound K3.

FIG. 63 depicts % inhibition of H358 parental cells (KRAS G12C) following administration of a combination comprising Compound T8 (a YAP/TAZ-TEAD inhibitor) and Compound K3 (a KRAS inhibitor), demonstrating that administration of Compound T8 sensitizes cells resistant to Compound K3.

FIG. 64 depicts % inhibition of H358 parental cells (KRAS G12C) following administration of a combination comprising Compound T9 (a YAP/TAZ-TEAD inhibitor) and Compound K3 (a KRAS inhibitor), demonstrating that administration of Compound T9 sensitizes cells resistant to Compound K3.

FIG. 65 depicts % inhibition of H358 parental cells (KRAS G12C) following administration of a combination comprising Compound T10 (a YAP/TAZ-TEAD inhibitor) and Compound K3 (a KRAS inhibitor), demonstrating that administration of Compound T10 sensitizes cells resistant to Compound K3.

FIG. 66 depicts % inhibition of H358 parental cells (KRAS G12C) following administration of a combination comprising Compound T1 (a YAP/TAZ-TEAD inhibitor) and Compound K4 (a KRAS inhibitor), demonstrating that administration of Compound T1 sensitizes cells resistant to Compound K4.

FIG. 67 depicts % inhibition of H358 parental cells (KRAS G12C) following administration of a combination comprising Compound T3 (a YAP/TAZ-TEAD inhibitor) and Compound K4 (a KRAS inhibitor), demonstrating that administration of Compound T3 sensitizes cells resistant to Compound K4.

FIG. 68 depicts % inhibition of H358 parental cells (KRAS G12C) following administration of a combination comprising Compound T5 (a YAP/TAZ-TEAD inhibitor) and Compound K4 (a KRAS inhibitor), demonstrating that administration of Compound T5 sensitizes cells resistant to Compound K4.

FIG. 69 depicts % inhibition of H358 parental cells (KRAS G12C) following administration of a combination comprising Compound T6 (a YAP/TAZ-TEAD inhibitor) and Compound K4 (a KRAS inhibitor), demonstrating that administration of Compound T6 sensitizes cells resistant to Compound K4.

FIG. 70 depicts % inhibition of H358 parental cells (KRAS G12C) following administration of a combination comprising Compound T7 (a YAP/TAZ-TEAD inhibitor) and Compound K4 (a KRAS inhibitor), demonstrating that administration of Compound T7 sensitizes cells resistant to Compound K4.

FIG. 71 depicts % inhibition of H358 parental cells (KRAS G12C) following administration of a combination comprising Compound T8 (a YAP/TAZ-TEAD inhibitor) and Compound K4 (a KRAS inhibitor), demonstrating that administration of Compound T8 sensitizes cells resistant to Compound K4.

FIG. 72 depicts % inhibition of H358 parental cells (KRAS G12C) following administration of a combination comprising Compound T9 (a YAP/TAZ-TEAD inhibitor) and Compound K4 (a KRAS inhibitor), demonstrating that administration of Compound T9 sensitizes cells resistant to Compound K4.

FIG. 73 depicts % inhibition of H358 parental cells (KRAS G12C) following administration of a combination comprising Compound T10 (a YAP/TAZ-TEAD inhibitor) and Compound K4 (a KRAS inhibitor), demonstrating that administration of Compound T10 sensitizes cells resistant to Compound K4.

FIG. 74 depicts % inhibition of H358 resistant cells (KRAS G12C) following administration of a combination comprising Compound T1 (a YAP/TAZ-TEAD inhibitor) and Compound K1 (a KRAS inhibitor), demonstrating that administration of Compound T1 sensitizes cells resistant to Compound K1.

FIG. 75 depicts % inhibition of H358 resistant cells (KRAS G12C) following administration of a combination comprising Compound T3 (a YAP/TAZ-TEAD inhibitor) and Compound K1 (a KRAS inhibitor), demonstrating that administration of Compound T3 sensitizes cells resistant to Compound K1.

FIG. 76 depicts % inhibition of H358 resistant cells (KRAS G12C) following administration of a combination comprising Compound T5 (a YAP/TAZ-TEAD inhibitor) and Compound K1 (a KRAS inhibitor), demonstrating that administration of Compound T5 sensitizes cells resistant to Compound K1.

FIG. 77 depicts % inhibition of H358 resistant cells (KRAS G12C) following administration of a combination comprising Compound T6 (a YAP/TAZ-TEAD inhibitor) and Compound K1 (a KRAS inhibitor), demonstrating that administration of Compound T6 sensitizes cells resistant to Compound K1.

FIG. 78 depicts % inhibition of H358 resistant cells (KRAS G12C) following administration of a combination comprising Compound T7 (a YAP/TAZ-TEAD inhibitor) and Compound K1 (a KRAS inhibitor), demonstrating that administration of Compound T7 sensitizes cells resistant to Compound K1.

FIG. 79 depicts % inhibition of H358 resistant cells (KRAS G12C) following administration of a combination comprising Compound T8 (a YAP/TAZ-TEAD inhibitor) and Compound K1 (a KRAS inhibitor), demonstrating that administration of Compound T8 sensitizes cells resistant to Compound K1.

FIG. 80 depicts % inhibition of H358 resistant cells (KRAS G12C) following administration of a combination comprising Compound T9 (a YAP/TAZ-TEAD inhibitor) and Compound K1 (a KRAS inhibitor), demonstrating that administration of Compound T9 sensitizes cells resistant to Compound K1.

FIG. 81 depicts % inhibition of H358 resistant cells (KRAS G12C) following administration of a combination comprising Compound T10 (a YAP/TAZ-TEAD inhibitor) and Compound K1 (a KRAS inhibitor), demonstrating that administration of Compound T10 sensitizes cells resistant to Compound K1.

FIG. 82 depicts % inhibition of H358 resistant cells (KRAS G12C) following administration of a combination comprising Compound T1 (a YAP/TAZ-TEAD inhibitor) and Compound K2 (a KRAS inhibitor), demonstrating that administration of Compound T1 sensitizes cells resistant to Compound K2.

FIG. 83 depicts % inhibition of H358 resistant cells (KRAS G12C) following administration of a combination comprising Compound T3 (a YAP/TAZ-TEAD inhibitor) and Compound K2 (a KRAS inhibitor), demonstrating that administration of Compound T3 sensitizes cells resistant to Compound K2.

FIG. 84 depicts % inhibition of H358 resistant cells (KRAS G12C) following administration of a combination comprising Compound T5 (a YAP/TAZ-TEAD inhibitor) and Compound K2 (a KRAS inhibitor), demonstrating that administration of Compound T5 sensitizes cells resistant to Compound K2.

FIG. 85 depicts % inhibition of H358 resistant cells (KRAS G12C) following administration of a combination comprising Compound T6 (a YAP/TAZ-TEAD inhibitor) and Compound K2 (a KRAS inhibitor), demonstrating that administration of Compound T6 sensitizes cells resistant to Compound K2.

FIG. 86 depicts % inhibition of H358 resistant cells (KRAS G12C) following administration of a combination comprising Compound T7 (a YAP/TAZ-TEAD inhibitor) and Compound K2 (a KRAS inhibitor), demonstrating that administration of Compound T7 sensitizes cells resistant to Compound K2.

FIG. 87 depicts % inhibition of H358 resistant cells (KRAS G12C) following administration of a combination comprising Compound T8 (a YAP/TAZ-TEAD inhibitor) and Compound K2 (a KRAS inhibitor), demonstrating that administration of Compound T8 sensitizes cells resistant to Compound K2.

FIG. 88 depicts % inhibition of H358 resistant cells (KRAS G12C) following administration of a combination comprising Compound T9 (a YAP/TAZ-TEAD inhibitor) and Compound K2 (a KRAS inhibitor), demonstrating that administration of Compound T9 sensitizes cells resistant to Compound K2.

FIG. 89 depicts % inhibition of H358 resistant cells (KRAS G12C) following administration of a combination comprising Compound T10 (a YAP/TAZ-TEAD inhibitor) and Compound K2 (a KRAS inhibitor), demonstrating that administration of Compound T10 sensitizes cells resistant to Compound K2.

FIG. 90 depicts % inhibition of H358 resistant cells (KRAS G12C) following administration of a combination comprising Compound T1 (a YAP/TAZ-TEAD inhibitor) and Compound K3 (a KRAS inhibitor), demonstrating that administration of Compound T1 sensitizes cells resistant to Compound K3.

FIG. 91 depicts % inhibition of H358 resistant cells (KRAS G12C) following administration of a combination comprising Compound T3 (a YAP/TAZ-TEAD inhibitor) and Compound K3 (a KRAS inhibitor), demonstrating that administration of Compound T3 sensitizes cells resistant to Compound K3.

FIG. 92 depicts % inhibition of H358 resistant cells (KRAS G12C) following administration of a combination comprising Compound T5 (a YAP/TAZ-TEAD inhibitor) and Compound K3 (a KRAS inhibitor), demonstrating that administration of Compound T5 sensitizes cells resistant to Compound K3.

FIG. 93 depicts % inhibition of H358 resistant cells (KRAS G12C) following administration of a combination comprising Compound T6 (a YAP/TAZ-TEAD inhibitor) and Compound K3 (a KRAS inhibitor), demonstrating that administration of Compound T6 sensitizes cells resistant to Compound K3.

FIG. 94 depicts % inhibition of H358 resistant cells (KRAS G12C) following administration of a combination comprising Compound T7 (a YAP/TAZ-TEAD inhibitor) and Compound K3 (a KRAS inhibitor), demonstrating that administration of Compound T7 sensitizes cells resistant to Compound K3.

FIG. 95 depicts % inhibition of H358 resistant cells (KRAS G12C) following administration of a combination comprising Compound T8 (a YAP/TAZ-TEAD inhibitor) and Compound K3 (a KRAS inhibitor), demonstrating that administration of Compound T8 sensitizes cells resistant to Compound K3.

FIG. 96 depicts % inhibition of H358 resistant cells (KRAS G12C) following administration of a combination comprising Compound T9 (a YAP/TAZ-TEAD inhibitor) and Compound K3 (a KRAS inhibitor), demonstrating that administration of Compound T9 sensitizes cells resistant to Compound K3.

FIG. 97 depicts % inhibition of H358 resistant cells (KRAS G12C) following administration of a combination comprising Compound T10 (a YAP/TAZ-TEAD inhibitor) and Compound K3 (a KRAS inhibitor), demonstrating that administration of Compound T10 sensitizes cells resistant to Compound K3.

FIG. 98 depicts % inhibition of H358 resistant cells (KRAS G12C) following administration of a combination comprising Compound T1 (a YAP/TAZ-TEAD inhibitor) and Compound K4 (a KRAS inhibitor), demonstrating that administration of Compound T1 sensitizes cells resistant to Compound K4.

FIG. 99 depicts % inhibition of H358 resistant cells (KRAS G12C) following administration of a combination comprising Compound T3 (a YAP/TAZ-TEAD inhibitor) and Compound K4 (a KRAS inhibitor), demonstrating that administration of Compound T3 sensitizes cells resistant to Compound K4.

FIG. 100 depicts % inhibition of H358 resistant cells (KRAS G12C) following administration of a combination comprising Compound T5 (a YAP/TAZ-TEAD inhibitor) and Compound K4 (a KRAS inhibitor), demonstrating that administration of Compound T5 sensitizes cells resistant to Compound K4.

FIG. 101 depicts % inhibition of H358 resistant cells (KRAS G12C) following administration of a combination comprising Compound T6 (a YAP/TAZ-TEAD inhibitor) and Compound K4 (a KRAS inhibitor), demonstrating that administration of Compound T6 sensitizes cells resistant to Compound K4.

FIG. 102 depicts % inhibition of H358 resistant cells (KRAS G12C) following administration of a combination comprising Compound T7 (a YAP/TAZ-TEAD inhibitor) and Compound K4 (a KRAS inhibitor), demonstrating that administration of Compound T7 sensitizes cells resistant to Compound K4.

FIG. 103 depicts % inhibition of H358 resistant cells (KRAS G12C) following administration of a combination comprising Compound T8 (a YAP/TAZ-TEAD inhibitor) and Compound K4 (a KRAS inhibitor), demonstrating that administration of Compound T8 sensitizes cells resistant to Compound K4.

FIG. 104 depicts % inhibition of H358 resistant cells (KRAS G12C) following administration of a combination comprising Compound T9 (a YAP/TAZ-TEAD inhibitor) and Compound K4 (a KRAS inhibitor), demonstrating that administration of Compound T9 sensitizes cells resistant to Compound K4.

FIG. 105 depicts % inhibition of H358 resistant cells (KRAS G12C) following administration of a combination comprising Compound T10 (a YAP/TAZ-TEAD inhibitor) and Compound K4 (a KRAS inhibitor), demonstrating that administration of Compound T10 sensitizes cells resistant to Compound K4.

### DETAILED DESCRIPTION

### I. Definitions

Unless otherwise indicated, the following specific terms and phrases used in the description and claims are defined as follows:

The terms “moiety” and “substituent” refer to an atom or group of chemically bonded atoms that is attached to another atom or molecule by one or more chemical bonds thereby forming part of a molecule.

The term “substituted” refers to the replacement of at least one of hydrogen atom of a compound or moiety with another substituent or moiety. Examples of such substituents include, without limitation, halogen, —OH, —CN, oxo, alkoxy, alkyl, aryl, heteroaryl, haloalkyl, haloalkoxy, cycloalkyl and heterocycle. For example, the term “alkyl substituted by halogen” refers to the fact that one or more hydrogen atoms of a alkyl (as defined below) is replaced by one or more halogen atoms (e.g., trifluoromethyl, difluoromethyl, fluoromethyl, chloromethyl, etc.).

The term “alkyl” refers to an aliphatic straight-chain or branched-chain saturated hydrocarbon moiety having 1 to 20 carbon atoms unless provided otherwise. For example, in particular embodiments, the alkyl has 1 to 10 carbon atoms. In particular embodiments the alkyl has 1 to 6 carbon atoms. Alkyl groups may be optionally substituted independently with one or more substituents described herein.

The term “alkoxy” denotes a group of the formula —O—R′, wherein R′ is an alkyl group. Alkoxy groups may be optionally substituted independently with one or more substituents described herein. Examples of alkoxy moieties include methoxy, ethoxy, isopropoxy, and tert-butoxy.

“Aryl” means a cyclic aromatic hydrocarbon moiety having a mono-, bi- or tricyclic aromatic ring of 6 to 20 carbon ring atoms unless provided otherwise. For example, in particular embodiments, the aryl has 6 to 10 carbon atoms. Bicyclic aryl ring systems include fused bicyclics having two fused five-membered aryl rings (denoted as 5-5), having a five-membered aryl ring and a fused six-membered aryl ring (denoted as 5-6 and as 6-5), and having two fused six-membered aryl rings (denoted as 6-6). The aryl group can be optionally substituted as defined herein. Examples of aryl moieties include, but are not limited to, phenyl, naphthyl, phenanthryl, fluorenyl, indenyl, pentalenyl, azulenyl, and the like. The term “aryl” also includes partially hydrogenated derivatives of the cyclic aromatic hydrocarbon moiety provided that at least one ring of the cyclic aromatic hydrocarbon moiety is aromatic, each being optionally substituted.

The term “heteroaryl” denotes an aromatic heterocyclic mono-, bi- or tricyclic ring system of 5 to 20 ring atoms unless provided otherwise, comprising 1, 2, 3 or 4 heteroatoms selected from N, O and S, the remaining ring atoms being carbon. For example, in some aspects, monocyclic heteroaryl rings may be 5-6 membered. In some aspects, heteroaryl rings may contain 5 to 10 carbon atoms. Bicyclic heteroaryl ring systems include fused bicyclics having two fused five-membered heteroaryl rings (denoted as 5-5), having a five-membered heteroaryl ring and a fused six-membered heteroaryl ring (denoted as 5-6 and 6-5), and having two fused six-membered heteroaryl rings (denoted as 6-6). The heteroaryl group can be optionally substituted as defined herein. Examples of heteroaryl moieties include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, or quinoxalinyl.

The terms “halo”, “halogen” and “halide”, which may be used interchangeably, refer to a substituent fluoro, chloro, bromo, or iodo.

The term “haloalkyl” denotes an alkyl group wherein one or more of the hydrogen atoms of the alkyl group has been replaced by the same or different halogen atoms, particularly fluoro atoms. Examples of haloalkyl include monofluoro-, difluoro- or trifluoromethyl, -ethyl or -propyl, for example 3,3,3-trifluoropropyl, 2-fluoroethyl, 2,2,2-trifluoroethyl, fluoromethyl, difluoromethyl or trifluoromethyl.

“Cycloalkyl” means a saturated or partially unsaturated carbocyclic moiety having mono-, bi- (including bridged bicyclic) or tricyclic rings and 3 to 10 carbon atoms in the ring unless provided otherwise. For example, in particular embodiments cycloalkyl contains from 3 to 8 carbon atoms (i.e., (C₃-C₈)cycloalkyl). In other particular embodiments cycloalkyl contains from 3 to 6 carbon atoms (i.e., (C₃-C₆)cycloalkyl). Examples of cycloalkyl moieties include, but are not limited to, cyclopropyl, cyclobutyl, cyclopentyl, cyclohexyl, cycloheptyl, and partially unsaturated (cycloalkenyl) derivatives thereof (e.g. cyclopentenyl, cyclohexenyl, and cycloheptenyl), bicyclo[3.1.0]hexanyl, bicyclo[3.1.0]hexenyl, bicyclo[3.1.1]heptanyl, and bicyclo[3.1.1]heptenyl. The cycloalkyl moiety can be attached in a “spirocycloakyl” fashion such as “spirocyclopropyl”:

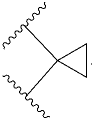 [SMILES: *C1(*)CC1]

The cycloalkyl moiety can optionally be substituted with one or more substituents.

“Heterocycle” or “heterocyclyl” refers to a 3, 4, 5, 6 and 7-membered monocyclic, 7, 8, 9 and 10-membered bicyclic (including bridged bicyclic) or 10, 11, 12, 13, 14 and 15-membered bicyclic heterocyclic moiety, unless provided otherwise, that is saturated or partially unsaturated, and has one or more (e.g., 1, 2, 3 or 4 heteroatoms selected from oxygen, nitrogen and sulfur in the ring with the remaining ring atoms being carbon. For example, in particular embodiments heterocycle or heterocyclyl refers to a 4, 5, 6 or 7-membered heterocycle. In some aspects, the heterocycle is a heterocycloalkyl. When used in reference to a ring atom of a heterocycle, a nitrogen or sulfur may also be in an oxidized form, and a nitrogen may be substituted with one or more groups such as C₁-C₆alkyl. The heterocycle can be attached to its pendant group at any heteroatom or carbon atom that results in a stable structure. Any of the heterocycle ring atoms can be optionally substituted with one or more substituents described herein. Examples of such saturated or partially unsaturated heterocycles include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The term the term heterocycle also includes groups in which a heterocycle is fused to one or more aryl, heteroaryl, or cycloalkyl rings, such as indolinyl, 3H-indolyl, chromanyl, azabicyclo[2.2.1]heptanyl, azabicyclo[3.1.0]hexanyl, azabicyclo[3.1.1]heptanyl, octahydroindolyl, or tetrahydroquinolinyl.

The term “fused bicyclic” denotes a ring system including two fused rings, including bridged cycloalkyl and bridged heterocycloalkyl as defined elsewhere herein. The rings are each independently, aryl, heteroaryl, cycloalkyl, and heterocycle. In some aspects, the rings are each independently, C₅₋₆ aryl, 5-6 membered heteroaryl, C₃₋₆ cycloalkyl, and 4-6 membered heterocycle. Non-limiting examples of fused bicyclic ring systems include C₅₋₆ aryl-C₅₋₆ aryl, C₅₋₆ aryl-4-6 membered heteroaryl, and C₅₋₆ aryl-C₅₋₆ cycloalkyl.

Unless otherwise indicated, the term “hydrogen” or “hydro” refers to the moiety of a hydrogen atom (—H) and not H₂.

In the description herein, if the stereochemistry of a structure or a portion of a structure is not indicated with, for example, bold wedged, or dashed lines, the structure or portion of the structure is to be interpreted as encompassing all stereoisomers of it. In some cases, however, where more than one chiral center exists, the structures and names may be represented as single enantiomers to help describe the relative stereochemistry.

Unless otherwise indicated, the term “a compound of the formula” or “a compound of formula” or “compounds of the formula” or “compounds of formula” refers to any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. As used herein, “pharmaceutically acceptable” refers to a carrier, diluent or excipient that is compatible with the other ingredients of the formulation and not deleterious to the recipient thereof. Salts may be formed with inorganic acids such as hydrochloric acid, hydrobromic acid, sulfuric acid, nitric acid, phosphoric acid and the like, preferably hydrochloric acid, and organic acids such as acetic acid, propionic acid, glycolic acid, pyruvic acid, oxalic acid, maleic acid, malonic acid, salicylic acid, succinic acid, fumaric acid, tartaric acid, citric acid, benzoic acid, cinnamic acid, mandelic acid, methanesulfonic acid, ethanesulfonic acid, p-toluenesulfonic acid, N-acetylcystein and the like. In addition, salts may be prepared by the addition of an inorganic base or an organic base to the free acid. Salts derived from an inorganic base include, but are not limited to, the sodium, potassium, lithium, ammonium, calcium, and magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present disclosure can be present in the form of pharmaceutically acceptable salts. Another embodiment provides non-pharmaceutically acceptable salts of the compounds provided herein, which can be useful as an intermediate for isolating or purifying such compounds. The compounds of the present disclosure can also be present in the form of pharmaceutically acceptable esters (e.g., methyl and ethyl esters to be used as prodrugs). The compounds of the present disclosure can also be solvated, e.g. hydrated. The solvation can be effected in the course of the manufacturing process or can take place, e.g., as a consequence of hygroscopic properties of an initially anhydrous compound provided herein.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−) isomers respectively). A chiral compound can exist as either individual enantiomer or as a mixture thereof. A mixture containing equal proportions of the enantiomers is called a “racemic mixture”. In certain embodiments the compound is enriched by at least about 90% by weight with a single diastereomer or enantiomer. In other embodiments the compound is enriched by at least about 95%, 98%, or 99% by weight with a single diastereomer or enantiomer.

Certain compounds of the present disclosure possess asymmetric carbon atoms (optical centers) or double bonds; the racemates, diastereomers, regioisomers and individual isomers (e.g., separate enantiomers) are all intended to be encompassed within the scope of the present disclosure.

The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. In some instances, the stereochemistry has not been determined or has been provisionally assigned. Many organic compounds exist in optically active forms, i.e., they have the ability to rotate the plane of plane polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. Enantiomers may be separated from a racemic mixture by a chiral separation method, such as supercritical fluid chromatography (SFC). Assignment of configuration at chiral centers in separated enantiomers may be tentative, while stereochemistry is definitively established, such as from X-ray crystallographic data.

The terms “an effective amount” and “a therapeutically effective amount” of a compound/combination/composition mean an amount of compound/combination/composition that is effective to achieve the desired outcome. For example, in some embodiments, an effective amount/therapeutically effective amount prevents, alleviates, or ameliorates symptoms of a disease or prolongs the survival of the subject being treated. Determination of a (therapeutically) effective amount is within the skill in the art. The (therapeutically) effective amount or dosage of a compound, combination, and/or composition according to this disclosure can vary within wide limits and may be determined in a manner known in the art. Such dosage will be adjusted to the individual requirements in each particular case including the specific compound(s), combination(s), and/or composition(s) being administered, the route of administration, the condition being treated, as well as the patient being treated. In general, in the case of oral or parenteral administration to adult humans weighing approximately 70 kg, a daily dosage of about 0.1 mg to about 5,000 mg, 1 mg to about 1,000 mg, or 1 mg to 100 mg may be appropriate, although the lower and upper limits may be exceeded when indicated. The daily dosage can be administered as a single dose or in divided doses, or for parenteral administration, it may be given as continuous infusion.

The terms “pharmaceutically acceptable carrier”, “pharmaceutically acceptable carrier, adjuvant, or vehicle”, or “therapeutically inert carrier” may be used interchangeably throughout and are intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound(s), use thereof in the compositions of the disclosure is contemplated. Supplementary active compounds can also be incorporated into the compositions.

Useful pharmaceutically acceptable carriers for the preparation of the compositions hereof, can be solids, liquids or gases; thus, the compositions can take the form of tablets, pills, capsules, suppositories, powders, enterically coated or other protected formulations (e.g., binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The term “subject”, “patient”, or “individual” as used herein, refers to an animal, such as a human or a non-human mammal. In one embodiment, subject, patient, or individual refers to a human.

In the practice of the method of the present disclosure, a (therapeutically) effective amount of any one of the compounds of this disclosure or a combination of any of the compounds, combinations, and/or compositions of this disclosure is administered via any of the usual and acceptable methods known in the art. The compounds, combinations, and/or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

### II. Compositions

In one aspect, provided herein is a composition, comprising: (i) one or more YAP/TAZ-TEAD inhibitors; and (ii) one or more KRAS inhibitors.

### YAP/TAZ-TEAD Inhibitors

In some embodiments, the one or more YAP/TAZ-TEAD inhibitors comprise a compound of formula (I):

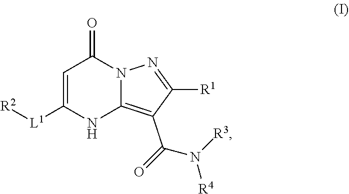

or a stereoisomer or tautomer thereof, or a pharmaceutically acceptable salt of any of the foregoing, wherein:
R¹ is selected from the group consisting of —C(O)—N(R^{a})(R^{b}), C₆₋₂₀aryl, 5-20 membered heteroaryl, 5-20 membered heterocyclyl, and C₁₋₆alkyl, wherein the C₆₋₂₀aryl, 5-20 membered heteroaryl and 5-20 membered heterocyclyl of R¹ are independently optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl, and wherein the C₁₋₆alkyl of R¹ is optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl;
R^{a} and R^{b} are each independently H or C₁₋₆alkyl, wherein the C₁₋₆alkyl of R^{a} or R^{b} is optionally substituted with one or more substituents selected from the group consisting of halo, —OH, —CN, C₁₋₆alkyl, C₁₋₆haloalkyl, and C₁₋₆alkoxy,
or R^{a} and R^{b} are taken, together with the atoms to which they attached, to form a 3-10 membered heterocyclyl, wherein the 3-10 membered heterocyclyl is optionally substituted with one or more substituents selected from the group consisting of halo, —OH, —CN, C₁₋₆alkyl, C₁₋₆haloalkyl, and C₁₋₆alkoxy;
L¹ is absent or is *—O—CH₂—**, *—CH₂—O—**, or —O—, wherein ** denotes the point of attachment to the R² moiety and * denotes the point of attachment to the remainder of the molecule;
R² is C₂₋₁₂alkyl, C₂₋₁₂alkenyl, or C₆₋₁₀aryl, wherein the C₂₋₁₂alkyl, C₂₋₁₂alkenyl, and C₆₋₁₀aryl of R² are independently optionally substituted with one or more substituents selected from the group consisting of halo, —OH, —CN, C₁₋₆alkyl, C₁₋₆haloalkyl, C₁₋₆alkoxy, C₆₋₁₀aryl, and C₃₋₁₀cycloalkyl, wherein the C₁₋₆alkyl, C₁₋₆alkoxy, and C₃₋₁₀cycloalkyl are independently optionally substituted with one or more halo, C₁₋₆haloalkyl, C₆₋₁₀aryl, or C₃₋₁₀cycloalkyl; and
R³ and R⁴ are each independently H or C₁₋₆alkyl, wherein the C₁₋₆alkyl of R³ or R⁴ is optionally substituted with one or more substituents selected from the group consisting of halo, —OH, —CN, C₁₋₆alkyl, C₁₋₆haloalkyl, and C₁₋₆alkoxy,
or R³ and R⁴, together with the atoms to which they are attached, form a 3-10 membered heterocyclyl, wherein the 3-10 membered heterocyclyl is optionally substituted with one or more substituents selected from the group consisting of halo, —OH, —CN, and C₁₋₆alkyl, wherein the C₁₋₆alkyl is optionally substituted with one or more substituents selected from the group consisting of halo, —OH, —CN, C₁₋₆alkyl, C₁₋₆haloalkyl, and C₁₋₆alkoxy.

In some embodiments of formula (I), the compound is of formula (I-A):

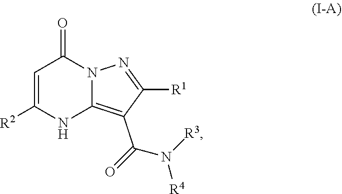

or a stereoisomer or tautomer thereof, or a pharmaceutically acceptable salt of any of the foregoing.

In some embodiments of formula (I), the compound is of formula (I-B):

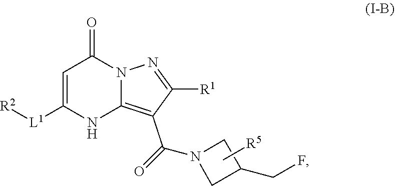

or a stereoisomer or tautomer thereof, or a pharmaceutically acceptable salt of any of the foregoing, wherein R⁵ is selected from the group consisting of H, halo, OH, cyano, and C₁₋₆alkyl, wherein the C₁₋₆alkyl is optionally substituted with one or more substituents selected from the group consisting of halo, OH, cyano, C₁₋₆alkyl, C₁₋₆haloalkyl, and C₁₋₆alkoxy.

In some embodiments of formula (I-B), the compound is of formula (I-B1):

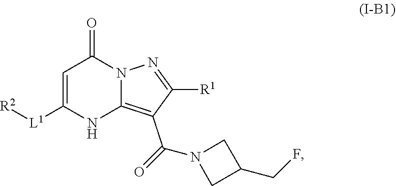

or a stereoisomer or tautomer thereof, or a pharmaceutically acceptable salt of any of the foregoing.

In some embodiments of formula (I), such as a compound of formula (I-B) or formula (I-B1), L¹ is absent. In some embodiments of formula (I), such as a compound of formula (I-A), formula (I-B), or formula (I-B1), R² is C₆₋₁₀aryl, wherein the C₆₋₁₀aryl of R² is optionally substituted with one or more C₃₋₁₀cycloalkyl, wherein the C₃₋₁₀cycloalkyl is optionally substituted with one or more halo, C₁₋₆haloalkyl, C₆₋₁₀aryl, or C₃₋₁₀cycloalkyl. In some embodiments, R² is

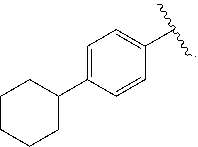 [SMILES: *c1ccc(C2CCCCC2)cc1]

In some embodiments of formula (I), such as a compound of formula (I-A), formula (I-B), or formula (I-B1), R¹ is 5-20 membered heteroaryl, wherein the 5-20 membered heteroaryl of R¹ is optionally substituted with one or more C₁₋₆alkyl. In some embodiments, R¹ is 5-6 membered heteroaryl, wherein the 5-6 membered heteroaryl of R¹ is optionally substituted with one or more C₁₋₆alkyl. In some embodiments, R¹ is pyrazinyl, wherein the pyrazinyl of R¹ is optionally substituted with one or more C₁₋₆alkyl. In some embodiments, R¹ is

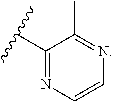 [SMILES: *c1nccnc1C]

In some embodiments of formula (I), the compound is

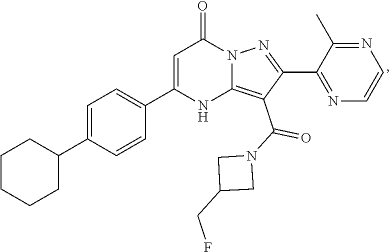 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing.

In some embodiments of formula (I), the compound is of formula (I-C):

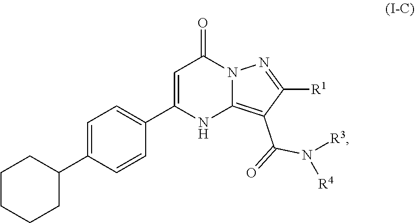

or a stereoisomer or tautomer thereof, or a pharmaceutically acceptable salt of any of the foregoing, wherein R¹, R³, and R⁴ are as defined in formula (I).

In some embodiments, in conjunction with embodiments above or below, the one or more YAP/TAZ-TEAD inhibitors comprise a compound of formula (I), (I-A), (I-B), or (I-C), or a stereoisomer or tautomer thereof, or a pharmaceutically acceptable salt of any of the foregoing. Description of formulae (I), (I-A), (I-B), and (I-C) can be found in WO2021/108483A1, the entirety of which is incorporated herein by reference. Formulae (I), (I-A), (I-B), and (I-C) are described as formulae (I), (IA), (IB), and (IC), respectively in WO2021/108483A1 (see, e.g., paragraphs [0046]-[00124]), which paragraphs and description of formula (I), (IA), (IB), or (IC), and methods of making compounds of formula (I), (IA), (IB), or (IC), are hereby incorporated herein by reference. Moieties of formula (I), (I-A), (I-B), or (I-C), such as R¹, R², R³, R⁴, R⁵, and L¹ are as defined in WO2021/108483A1, including any variations or embodiments thereof. R¹, R², R³, R⁴, R⁵, and L¹ of formulae (I), (I-A), (I-B), and (I-C) correspond to the moieties R¹, R², R³, R⁴, R⁵, and L, respectively, in WO2021/108483A1.

In some embodiments, in conjunction with embodiments above or below, a compound of formula (I), (I-A), (I-B), or (I-C) is Compound T1, or a stereoisomer or tautomer thereof, or a pharmaceutically acceptable salt of any of the foregoing. Compound T1 is chemically described as 5-(4-cyclohexylphenyl)-3-(3-(fluoromethyl)azetidine-1-carbonyl)-2-(3-methylpyrazin-2-yl)pyrazolo[1,5-a]pyrimidin-7(4H)-one, having the structure below:

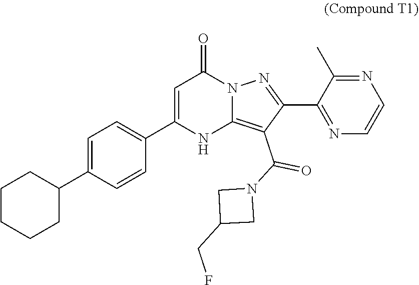 [SMILES: Cc1nccnc1-c1nn2c(=O)cc(-c3ccc(C4CCCCC4)cc3)[nH]c2c1C(=O)N1CC(CF)C1]

Description of Compound T1 and methods of making Compound T1 can be found in, e.g., Compound 27 on page 31 and Example 27 on pages 140-142 of WO2021/108483A1.

In some embodiments, in conjunction with embodiments above or below, a compound of formula (I), (I-A), (I-B), or (I-C) is Compound T9, or a stereoisomer or tautomer thereof, or a pharmaceutically acceptable salt of any of the foregoing. Compound T9 is chemically described as 5-(4-cyclohexylphenyl)-2-(3-methylpyrazin-2-yl)-3-[rac-(2S,3S)-3-(fluoromethyl)-2-methyl-azetidine-1-carbonyl]-4H-pyrazolo[1,5-a]pyrimidin-7-one, having the structure below:

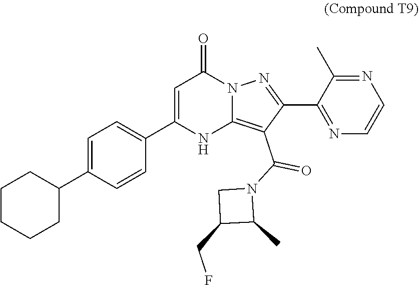 [SMILES: Cc1nccnc1-c1nn2c(=O)cc(-c3ccc(C4CCCCC4)cc3)[nH]c2c1C(=O)N1C[C@H](CF)[C@@H]1C]

Description of Compound T9 and methods of making Compound T9 can be found in, e.g., Compound 44 on page 35 and Example 41 on pages 156-158 of WO2021/108483A1.

In some embodiments, the one or more YAP/TAZ-TEAD inhibitors comprise a compound of formula (II):

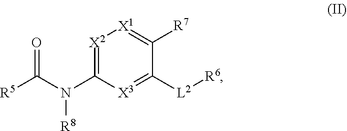

or a stereoisomer or tautomer thereof, or a pharmaceutically acceptable salt of any of the foregoing, wherein:
X¹ is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h}), or
the R⁹ of X¹ is taken together with R⁷, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH;
X² is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h});
X³ is N or C—H,
   provided that, when X³ is N, and R⁵ is

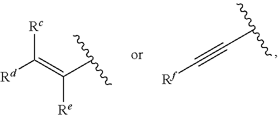

then at least one of X¹ and X² is N;
R⁵ is:
   (i) oxiranyl or oxetanyl, wherein the oxiranyl or oxetanyl of R⁵ is optionally substituted with one or more C₁₋₆alkyl, wherein the C₁₋₆alkyl is independently optionally substituted with one or more —C(O)—NH₂, and
      L² is absent or is selected from the group consisting of —O—, *—CH₂—O—**,
      *—O—CH₂—**, —CH═CH—, and —C≡C—, wherein ** indicates the attachment point to the R⁶ moiety and * indicates the attachment point to the remainder of the molecule, or
   (ii) —N(R^{g})(CN), and
      L² is absent or is selected from the group consisting of —O—, *—CH₂—O—**,
      *—O—CH₂—**, —CH═CH—, and —C≡C—, wherein ** indicates the attachment point to the R⁶ moiety and * indicates the attachment point to the remainder of the molecule, or
   (iii)

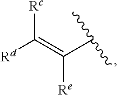

wherein R^{c}, R^{d}, and R^{e} are each independently selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{c}, R^{d}, or R^{e} is optionally substituted with one or more —OH, provided that at least two of R^{c}, R^{d}, and R^{e} are H, and
   L² is absent or is selected from the group consisting of *—CH₂—O—**,
   *—O—CH₂—**, —CH═CH—, and —C≡C—, wherein ** indicates the attachment point to the R⁶ moiety and * indicates the attachment point to the remainder of the molecule, or
(iv)

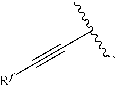

wherein R^{f} is selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{f} is optionally substituted with —OH, and
   L² is selected from the group consisting of —O—, *—CH₂—O—**, *—O—CH₂—**,
   —CH═CH—, and —C≡C—, wherein ** indicates the attachment point to the R⁶ moiety and * indicates the attachment point to the remainder of the molecule;
R⁶ is C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl, wherein:
   the C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂,
   —N(R^{g})(R^{h}), —O(R^{g}), and —SF₅,
   provided that, when R⁶ is C₁₋₁₂alkyl, wherein the C₁₋₁₂alkyl is independently optionally substituted with one or two substituents selected from the group consisting of —CN, halo, C₁₋₆alkyl, C₁₋₆haloalkyl, C₃₋₁₀cycloalkyl, —NO₂, —N(R^{g})(R^{h}), and —O(R^{g}), then L² is —CH═CH— or —C≡C—;
R⁷ is —CN, C₁₋₆alkyl, C₁₋₄alkoxy, or C₂₋₄alkenyl, wherein the C₂₋₄alkenyl of R⁷ is optionally substituted with —N(R^{g})(R^{h}), or
R⁷ is taken together with R⁹ of X¹, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH, or
R⁷ is taken together with the carbon atom of *—CH₂—O—** of L², and the atoms to which they are attached, to form a C₆aryl or a 6-membered heteroaryl,
   provided that:
(i) when R⁷ is —CN, C₁₋₆alkyl, C₁₋₄alkoxy, or C₂₋₄alkenyl, wherein the C₂₋₄alkenyl is optionally substituted with —N(R^{g})(R^{h}), and
   R⁵ is

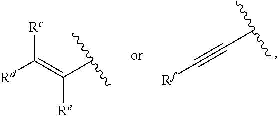

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is *—CH₂—O—**, —CH═CH—, or —C≡C—, wherein ** indicates the attachment point to the R⁶ moiety and * indicates the attachment point to the remainder of the molecule, and
(ii) when R⁷ is taken together with R⁹ of X¹, and the atoms to which they are attached, to form a 5-membered heterocyclyl or a 5-membered heteroaryl, provided that X³ is CH, and
   R⁵ is

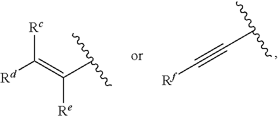

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is absent or is *—CH₂—O—**, —CH═CH—, or —C≡C—, wherein ** indicates the attachment point to the R⁶ moiety and * indicates the attachment point to the remainder of the molecule, and
(iii) when R⁷ is taken together with the carbon atom of *—CH₂—O—** of L², and the atoms to which they are attached, to form a C₆aryl or a 6-membered heteroaryl, and
   R⁵ is

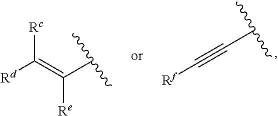

then R⁶ is 3-10 membered saturated heterocyclyl or 5-20 membered heteroaryl, wherein the 3-10 membered saturated heterocyclyl or 5-20 membered heteroaryl is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}) and —O(R^{g});
R⁸ is H or C₁₋₆alkyl, wherein the C₁₋₆alkyl is optionally substituted with one or more —OH; and
R^{g} and R^{h} are, independently of each other and independently at each occurrence, selected from the group consisting of H, —CN, —OH, C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl of R^{g} and R^{h} are each independently optionally substituted with one or more substituents independently selected from the group consisting of C₁₋₆alkyl, C₁₋₆haloalkyl, C₁₋₆alkoxy, oxo, —CN, halo, —NO₂, and —OH.

In some embodiments of formula (II), the R⁹ of X¹ is taken together with R⁷, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH.

In some embodiments of formula (II), the compound is of formula (II-A):

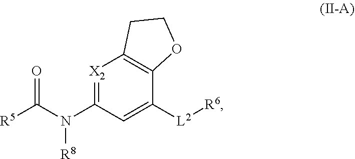

or a stereoisomer or tautomer thereof, or a pharmaceutically acceptable salt of any of the foregoing.

In some embodiments of formula (II), the compound is of formula (II-A1);

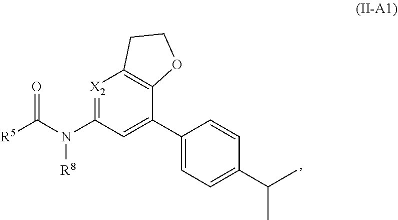

or a stereoisomer or tautomer thereof, or a pharmaceutically acceptable salt of any of the foregoing.

In some embodiments of formula (II), such as formula (II-A) and formula (II-A1), R⁵ is

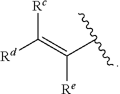

In some embodiments of the foregoing, each of R^{c}, R^{d}, and R^{e} is H.

In some embodiments of formula (II), the compound is

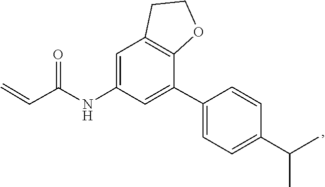 [SMILES: C=CC(=O)Nc1cc2c(c(-c3ccc(C(C)C)cc3)c1)OCC2]

or a stereoisomer or tautomer thereof, or a pharmaceutically acceptable salt of any of the foregoing.

In some embodiments of formula (II), the compound is of formula (II-B):

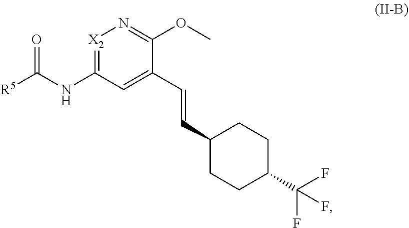

or a stereoisomer or tautomer thereof, or a pharmaceutically acceptable salt of any of the foregoing.

In some embodiments of formula (II-B), R⁵ is

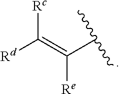

In some embodiments of the foregoing, each of R^{c}, R^{d}, and R^{e} is H.

In some embodiments of formula (II), the compound is

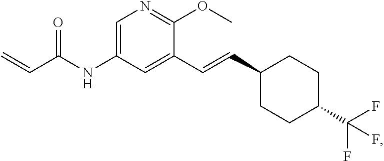 [SMILES: C=CC(=O)Nc1cnc(OC)c(/C=C/[C@H]2CC[C@H](C(F)(F)F)CC2)c1]

or a stereoisomer or tautomer thereof, or a pharmaceutically acceptable salt of any of the foregoing.

In some embodiments, in conjunction with embodiments above or below, the one or more YAP/TAZ-TEAD inhibitors comprise a compound of formula (II), (II-A), or (II-B), or a stereoisomer or tautomer thereof, or a pharmaceutically acceptable salt of any of the foregoing. Description of formulae (II), (II-A), and (II-B) can be found in WO2021/097110A1, the entirety of which is incorporated herein by reference. Formulae (II), (II-A), and (II-B) are described as formulae (B-1), (IA), and (IF), respectively, in WO2021/097110A1 (see, e.g., paragraphs [0054], [0067], and [0087]), which paragraphs and description of formula (B-1), (IA), or (IF), and methods of making compounds of formula (B-1), (IA), or (IF), are hereby incorporated herein by reference. Moieties of formula (II), (II-A), or (II-B), such as R⁵, R⁶, R⁷, R⁸, X¹, X², X³, and L² are as defined in WO2021/097110A1, including any variations or embodiments thereof. X¹, X², X³, R⁵, R⁶, R⁷, R⁸, R⁹, L², R^{c}, R^{d}, R^{e}, R^{f}, R^{g}, and R^{h} of formulae (II), (II-A), and (II-B) correspond to the moieties X₁, X₂, X₃, R₁, R₂, R₃, R₄, R₅, L, R^{a}, R^{b}, R^{c}, R^{d}, R^{e}, and R^{f}, respectively, in WO2021/097110A1.

In some embodiments, in conjunction with embodiments above or below, a compound of formula (II), (II-A), or (II-B) is Compound T2, or a stereoisomer or tautomer thereof, or a pharmaceutically acceptable salt of any of the foregoing. Compound T2 is chemically described N-(7-(4-isopropylphenyl)-2,3-dihydrobenzofuran-5-yl)acrylamide, having the structure below:

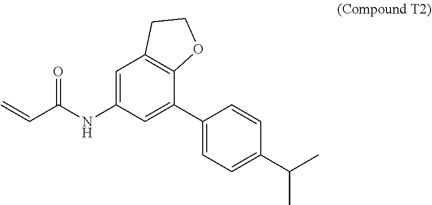 [SMILES: C=CC(=O)Nc1cc2c(c(-c3ccc(C(C)C)cc3)c1)OCC2]

Description of Compound T2 and methods of making Compound T2 can be found in, e.g., Compound 33 on page 73 and Example 33 on pages 245-246 of WO2021/097110A1.

In some embodiments, in conjunction with embodiments above or below, a compound of formula (II), (II-A), or (II-B) is Compound T3, or a stereoisomer or tautomer thereof, or a pharmaceutically acceptable salt of any of the foregoing. Compound T3 is chemically described N-(6-methoxy-5-((E)-2-((1r,4r)-4-(trifluoromethyl)cyclohexyl)vinyl)pyridin-3-yl)acrylamide, having the structure below:

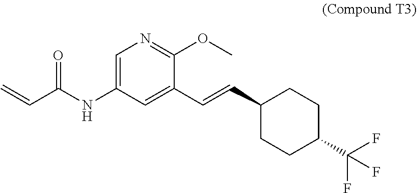 [SMILES: C=CC(=O)Nc1cnc(OC)c(/C=C/[C@H]2CC[C@H](C(F)(F)F)CC2)c1]

Description of Compound T3 and methods of making Compound T3 can be found in, e.g., Compound 2 on page 68 and Example 2 on page 192 of WO2021/097110A1.

In some embodiments, the one or more YAP/TAZ-TEAD inhibitors comprise a compound of formula (III):

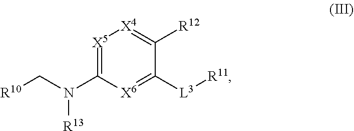

or a stereoisomer or tautomer thereof, or a pharmaceutically acceptable salt of any of the foregoing, wherein:
X⁴ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ), or
the R¹⁴ of X⁴ is taken together with R¹², and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl;
X⁵ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ);
X₆ is N or C—H;
R¹⁰ is:
   (i) a 3-5 membered saturated heterocyclyl comprising at least one annular oxygen atom, wherein the 3-5 membered saturated heterocyclyl is optionally substituted with one or more C₁₋₆alkyl, or
   (ii) —N(R^{m})(Rⁿ), or
   (iii)

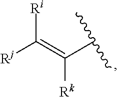

wherein Rⁱ, R^{j}, and R^{k} are each independently selected from the group consisting of H, halo, —CN, —OH, —B(OH)₂, —C(O)—OH, —C(O)—N(R^{m})(Rⁿ), —C(O)—C₁₋₆alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆alkyl of Rⁱ, R^{j}, or R^{k} is optionally substituted with one or more —OH, or
(iv)

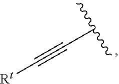

wherein R^{t} is selected from the group consisting of H, halo, —CN, —OH, —B(OH)₂, —C(O)—OH, —C(O)—N(R^{m})(Rⁿ), —C(O)—C₁₋₆alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆alkyl is optionally substituted with one or more —OH;
L³ is absent or is selected from the group consisting of —O—, *—CH₂—O—**, *—O—CH₂—**,
—CH═CH—, and —C≡C—, wherein ** indicates the attachment point to the R¹¹ moiety and * indicates the attachment point to the remainder of the molecule;
R¹¹ is C₁₋₁₂alkyl, C₃₋₁₀cycloalkyl, 3-10 membered heterocyclyl, C₅₋₁₃spirocyclyl, C₆₋₂₀aryl, or 5-20 membered heteroaryl,
   wherein the C₁₋₁₂alkyl, C₃₋₁₀cycloalkyl, 3-10 membered saturated heterocyclyl, C₅₋₁₃spirocyclyl, C₆₋₂₀aryl, or 5-20 membered heteroaryl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}),
   provided that, when R¹¹ is C₁₋₁₂alkyl, wherein the C₁₋₁₂alkyl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}), then L³ is —CH═CH— or —C≡C—;
R¹² is —CN, C₁₋₆alkyl, C₁₋₄alkoxy, or C₂₋₄alkenyl, wherein the C₂₋₄alkenyl is optionally substituted with —N(R^{m})(Rⁿ), or
R¹² is taken together with R¹⁴ of X⁴, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, or
R¹² is taken together with the carbon atom of *—CH₂—O—** of L³, and the atoms to which they are attached, to form a C₆aryl or a 6-membered heteroaryl;
R¹³ is H or C₁₋₆alkyl, wherein the C₁₋₆alkyl of R¹³ is optionally substituted with one or more —OH; and
R^{m} and Rⁿ are, independently of each other and independently at each occurrence, selected from the group consisting of H, —CN, —OH, C₁₋₆alkyl, C₂₋₆alkenyl, C₂₋₆alkynyl, C₃₋₁₀cycloalkyl, —C₁₋₆alkyl-C₃₋₁₀cycloalkyl, 3-10 membered heterocyclyl, C₅₋₁₃spirocyclyl, C₆₋₂₀aryl, and 5-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₅₋₁₃spirocyclyl, C₆₋₂₀ aryl, and 5-20 membered heteroaryl of R^{m} and Rⁿ are each independently optionally substituted with one or more substituents independently selected from the group consisting of C₁₋₆alkyl, C₁₋₆haloalkyl, C₁₋₆alkoxy, oxo, —CN, halo, —NO₂, and —OH.

In some embodiments of formula (III), the R¹⁴ of X⁴ is taken together with R¹², and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl.

In some embodiments of formula (III), the compound is of formula (III-A):

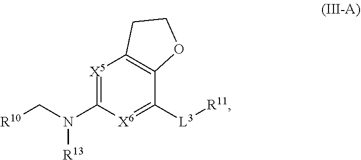

or a stereoisomer or tautomer thereof, or a pharmaceutically acceptable salt of any of the foregoing.

In some embodiments of the foregoing, L³ is absent. In some embodiments of formula (III-A), the compound is of formula (III-A1):

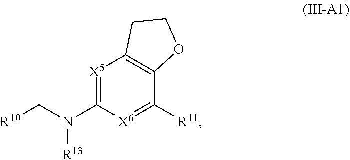

or a stereoisomer or tautomer thereof, or a pharmaceutically acceptable salt of any of the foregoing.

In some embodiments of formula (III), such as a compound of formula (III-A1) or formula (III-A2), R¹⁰ is

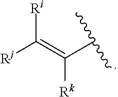

In some embodiments of the foregoing, Rⁱ and R^{j} are both H, and R^{k} is —C(O)OH.

In some embodiments of formula (III), such as a compound of formula (III-A1) or formula (III-A2), R¹¹ is C₆₋₂₀aryl, wherein the C₆₋₂₀aryl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}). In some embodiments, R¹¹ is C₆₋₂₀aryl, wherein the C₆₋₂₀aryl of R¹¹ is independently optionally substituted with one or more C₁₋₆alkyl. In some embodiments, R¹¹ is

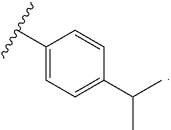 [SMILES: *c1ccc(C(C)C)cc1]

In some embodiments of formula (III), the compound is

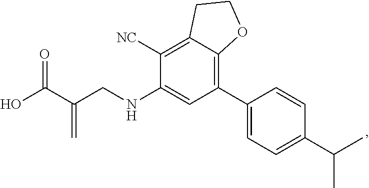 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing.

In some embodiments, in conjunction with embodiments above or below, a compound of formula (III), (III-A), or (III-A1) is Compound T4, or a stereoisomer or tautomer thereof, or a pharmaceutically acceptable salt of any of the foregoing. Compound T4 is chemically described as 2-(((4-cyano-7-(4-isopropylphenyl)-2,3-dihydrobenzofuran-5-yl)amino)methyl)acrylic acid, having the structure below:

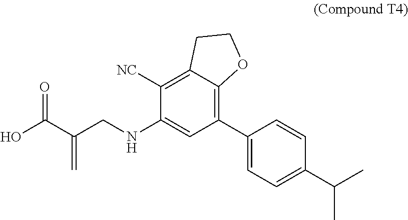 [SMILES: C=C(CNc1cc(-c2ccc(C(C)C)cc2)c2c(c1C#N)CCO2)C(=O)O]

Description of Compound T4 and methods of making Compound T4 can be found in, e.g., Compound 3 on page 48 and Example 3 on pages 164-165 of WO2022/020716.

In some embodiments, in conjunction with embodiments above or below, the one or more YAP/TAZ-TEAD inhibitors comprise a compound of formula (IV):

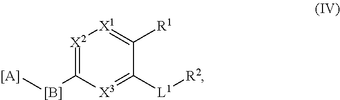

wherein:
[A] is a ligase ligand;
[B] is a linker moiety;
X¹, X², and X³ are each independently N or C—R⁵, wherein each R⁵ is independently selected from the group consisting of H, halo, cyano, C₁₋₁₂alkyl, O—C₁₋₁₂alkyl, and C₁₋₁₂haloalkyl;
L¹ is a bond or is —C≡C—, —CH═CH—, or —(CH₂)ₘ—, wherein m is 1-6;
R¹ is H, C₁₋₁₂alkyl, C₃₋₁₀cycloalkyl, C₁₋₁₂alkyl-C₃₋₁₀cycloalkyl, C₁₋₁₂haloalkyl, O—C₁₋₁₂alkyl, O—C₃₋₁₀cycloalkyl, O—C₁₋₁₂alkyl-C₃₋₁₀cycloalkyl, or O—C₁₋₁₂haloalkyl; and
R² is H, C₃₋₁₀cycloalkyl, C₁₋₁₂alkyl-C₃₋₁₀cycloalkyl, 3-10 membered heterocyclyl, C₆₋₂₀aryl, 5-20 membered heteroaryl, or C₅₋₁₃spirocyclyl, wherein:
the C₃₋₁₀cycloalkyl, C₁₋₁₂alkyl-C₃₋₁₀cycloalkyl, 3-10 membered heterocyclyl, C₆₋₂₀aryl, 5-20 membered heteroaryl, or C₅₋₁₃spirocyclyl of R² is independently optionally substituted with one or more oxo, cyano, halo, C₁₋₁₂alkyl, C₁₋₁₂haloalkyl, C₃₋₁₀cycloalkyl, C₆₋₂₀aryl, NO₂, N(R^{x})(R^{y}), and O(R^{x}), wherein:
each R^{x} and R^{y} is independently selected from the group consisting of H, C₁₋₁₂alkyl, C₂₋₁₂alkenyl, C₂₋₁₂alkylenyl, C₃₋₁₀cycloalkyl, C₁₋₁₂alkyl-C₃₋₁₀cycloalkyl, 3-10 membered heterocyclyl, C₆₋₂₀aryl and 5-20 membered heteroaryl, wherein:
each C₁₋₁₂ alkyl, C₂₋₁₂ alkenyl, C₂₋₁₂ alkylnyl, C₃₋₁₀cycloalkyl, C₁₋₁₂alkyl-C₃₋₁₀cycloalkyl, 3-10 membered heterocyclyl, C₆₋₂₀aryl and 5-20 membered heteroaryl of R^{x} and R^{y} is independently optionally substituted with one or more oxo, cyano, halo, NO₂, NH₂, hydroxy, C₁₋₁₂ alkyl, C₁₋₁₂haloalkyl, or O—C₁₋₁₂alkyl; or a stereoisomer or tautomer thereof, or a pharmaceutically acceptable salt of any of the foregoing.

Description of formula (IV) can be found in WO2021/178339A1, the entirety of which is incorporated herein by reference. Formula (IV) is described as Formula (I) in WO2021/178339A1 (see, e.g., paragraphs [0057]-[0058]), which paragraphs and description of Formula (I), and methods of making compounds of Formula (I) are hereby incorporated herein by reference. Moieties of formula (IV), such as A, B, R¹, R², X¹, X², X³, and L¹ are as defined in WO2021/178339A1, including any variations or embodiments thereof.

In some embodiments, in conjunction with embodiments above or below, the one or more YAP/TAZ-TEAD inhibitors comprise a compound of formula (IV-A), (IV-B), (IV-C), (IV-D), (IV-E), or (IV-F):

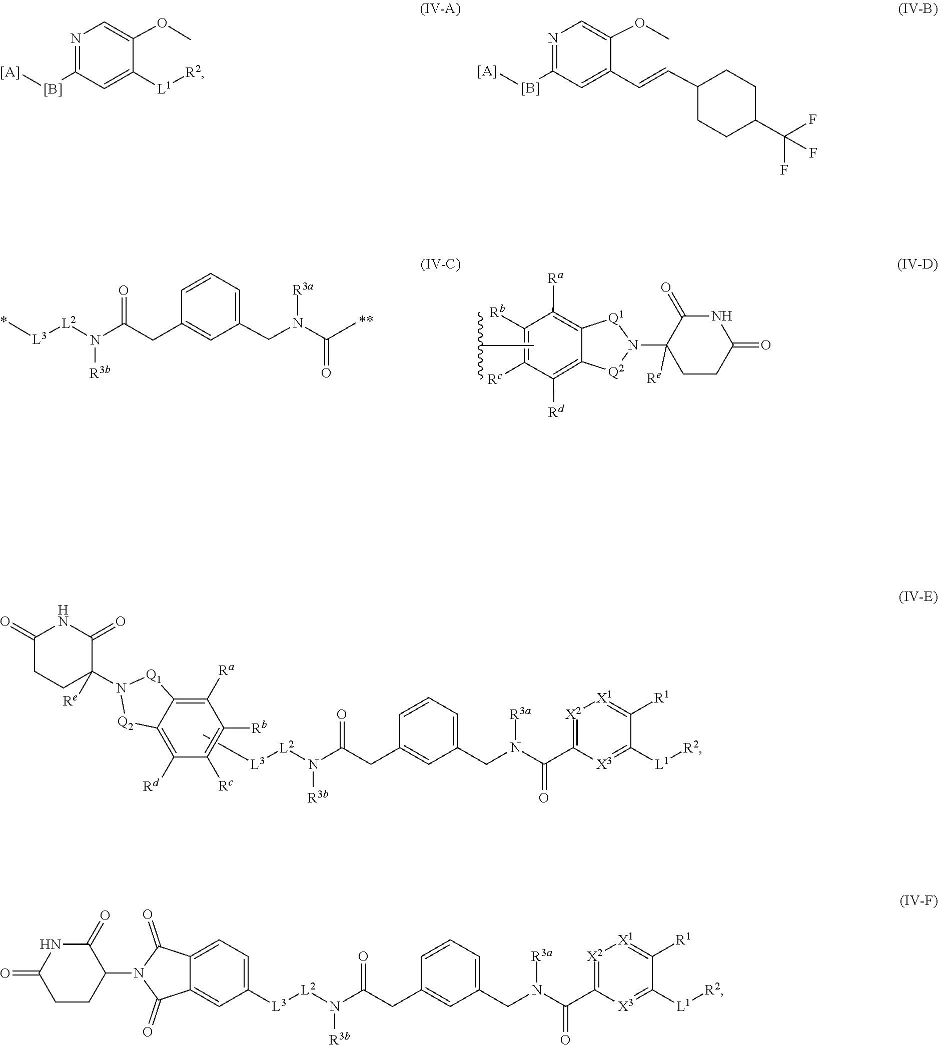

wherein:
* of the linker moiety denotes the point of attachment to the ligase ligand and ** of the linker moiety denotes the point of attachment to the remainder of the molecule;
L² is —(CH₂)ₙ— or —(CH₂CH₂O)ₙ—, wherein n is 1-12;
L³ is a bond or is —C≡C—, —CH═CH—, —(CH₂)ₘ—, —O—, —NH—, or

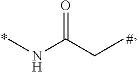

wherein # of L³ denotes the point of attachment to L² and * of L³ denotes the point of attachment to the ligase ligand; and
each of R^{3a} and R^{3b} is independently H, C₁₋₁₂alkyl, C₂₋₁₂ alkenyl, C₂₋₁₂ alkynyl, C₃₋₁₀cycloalkyl, C₁₋₁₂alkyl-C₃₋₁₀cycloalkyl, C₁₋₁₂alkyl-C₆₋₂₀aryl, 3-10 membered heterocyclyl, C₆₋₂₀ aryl, or 5-20 membered heteroaryl, wherein:
each C₁₋₁₂alkyl, C₂₋₁₂ alkenyl, C₂₋₁₂ alkynyl, C₃₋₁₀cycloalkyl, C₁₋₁₂alkyl-C₃₋₁₀cycloalkyl, C₁₋₁₂alkyl-C₆₋₂₀aryl, 3-10 membered heterocyclyl, C₆₋₂₀aryl, or 5-20 membered heteroaryl is independently optionally substituted with at least one of oxo, CN, C₁₋₁₂alkyl, C₁₋₁₂haloalkyl, halo, NO₂, N(R^{e})(R^{f}), C₁₋₁₂alkyl-C(O)—N(R^{e})(R^{f}), and OR^{e}, wherein: each R^{e} and R^{f} is independently selected from the group consisting of H, C₁₋₁₂alkyl, C₂₋₁₂alkenyl, C₂₋₁₂alkynyl, C₃₋₁₀cycloalkyl, C₁₋₁₂alkyl-C₃₋₁₀cycloalkyl, 3-10 membered heterocyclyl, C₆₋₂₀aryl, and 5-20 membered heteroaryl, wherein each C₁₋₁₂alkyl, C₂₋₁₂alkenyl, C₂₋₁₂alkynyl, C₃₋₁₀cycloalkyl, C₁₋₁₂alkyl-C₃₋₁₀cycloalkyl, 3-10 membered heterocyclyl, C₆₋₂₀aryl, or 5-20 membered heteroaryl is independently optionally substituted with one or more oxo, CN, C₁₋₁₂alkyl, C₁₋₁₂haloalkyl, halo, NO₂, O—C₁₋₁₂alkyl, or OH;
one of Q¹ and Q² is C═O, and the other of Q¹ and Q² is C═O or CH₂;
one of R^{a}, R^{b}, R^{c}, and R^{d} is the

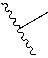

bond to L³ of the linker moiety, and the others of R^{a}, R^{b}, R^{c}, and R^{d} are each independently H, halo, C₁₋₁₂alkyl, C₁₋₁₂haloalkyl, or O—C₁₋₁₂alkyl; and
R^{e} is H, halo, C₁₋₁₂alkyl, C₁₋₁₂haloalkyl, O—C₁₋₁₂alkyl, or phenyl, or a stereoisomer or tautomer thereof, or a pharmaceutically acceptable salt of any of the foregoing, wherein [A], [B], X¹, X², X³, L¹, R¹, and R² are as defined in Formula (IV). It is understood that [A], [B], X¹, X², X³, L¹, R¹, and R² of such embodiments of compound of Formulae (IV-A), (IV-B), (IV-C), (IV-D), (IV-E), (IV-F) may include [A], [B], X¹, X², X³, L¹, R¹, and R² as descried for Formula (IV).

Formulae (IV-A), (IV-B), (IV-C), (IV-D), (IV-E), and (IV-F) are described as formulae (XII), (XIII), (II), (III), (IV), and (V), respectively, in WO2021/178339A1 (see, e.g., paragraphs [0059]-[0064], and [0119]-[0121]), which paragraphs and description of formula (XII), (XIII), (II), (III), (IV), or (V), and methods of making compounds of formula (XII), (XIII), (II), (III), (IV), or (V), are hereby incorporated herein by reference. Moieties of formula (IV-A), (IV-B), (IV-C), (IV-D), (IV-E), or (IV-F), such as A, B, R^{a}, R^{b}, R^{c}, R^{d}, R^{e}, R^{3a}, R^{3b}, R¹, R², X¹, X², X³, Q¹, Q², L¹, L², and L³ are as defined in WO2021/178339A1, including any variations or embodiments thereof.

In some embodiments, in conjunction with embodiments above or below, a compound of formula (IV), (IV-A), (IV-B), (IV-C), (IV-D), (IV-E), or (IV-F) is Compound T5, or a stereoisomer or tautomer thereof, or a pharmaceutically acceptable salt of any of the foregoing. Compound T5 is chemically described as N-[[3-[2-[2-[2-[2-[2-[2-[2-(2,6-dioxo-3-piperidyl)-1,3-dioxo-isoindolin-5-yl]oxyethoxy]ethoxy]ethoxy]ethoxy]ethyl-methyl-amino]-2-oxo-ethyl]phenyl]methyl]-5-methoxy-4-[rac-(E)-2-[4-(trifluoromethyl)cyclohexyl]vinyl]pyridine-2-carboxamide, having the structure below:

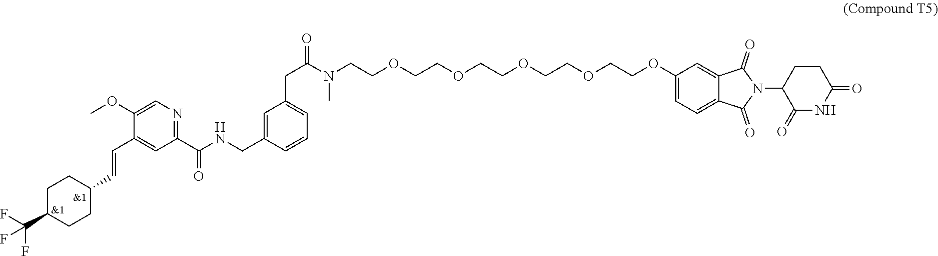 [SMILES: COc1cnc(C(=O)NCc2cccc(CC(=O)N(C)CCOCCOCCOCCOCCOc3ccc4c(c3)C(=O)N(C3CCC(=O)NC3=O)C4=O)c2)cc1/C=C/[C@H]1CC[C@H](C(F)(F)F)CC1]

Description of Compound T5 and methods of making Compound T5 can be found in, e.g., Table 1 on page 51 and Example 4 on pages 123-126 of WO2021/178339A1.

In some embodiments, in conjunction with embodiments above or below, the one or more YAP/TAZ-TEAD inhibitors comprise a compound of formula (V):

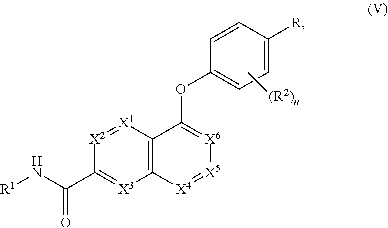

wherein,
each X¹, X⁴, X⁵, and X⁶, is independently N or CR^{X};
each X² and X³ is independently N or CR^{Y};
each R^{X} is independently hydrogen, halogen, nitro, —OR³, —SR³, —CN, —C(═O)R³, —C(═O)N(R³)₂, —C(═O)OR³, —S(═O)R³, S(═O)₂R³, —N(R³)₂, —NR³S(═O)₂R³, —NR³C(═O)R³, —NR³C(═O)OR³, substituted or unsubstituted C₁-C₆ alkyl, substituted or unsubstituted C₁-C₆fluoroalkyl, substituted or unsubstituted C₂-C₄alkenyl, substituted or unsubstituted C₂-C₄alkylnyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R^{Y} is independently hydrogen, halogen, nitro, —CN, —C(═O)R³, —C(═O)N(R³)₂, —C(═O)OR³, —S(═O)R³, —S(═O)₂R³, —N(R³)₂, —NR³S(═O)₂R³, —NR³C(═O)R³, —NR³C(═O)OR³, substituted or unsubstituted C₁-C₆ alkyl, substituted or unsubstituted C₁-C₆fluoroalkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
R is halogen, nitro, —CN, —OR³, —SR³, —C(═O)R³, —C(═O)N(R³)₂, —C(═O)OR³, —S(═O)R³, —S(═O)₂R³, —N(R³)₂, —NR³S(═O)₂R³, —NR³C(═O)R³, —NR³C(═O)OR³, or substituted or unsubstituted C₁-C₆fluoroalkyl;
R¹ is substituted or unsubstituted C₁-C₆alkyl, substituted or unsubstituted C₁-C₆fluoroalkyl, substituted or unsubstituted C₂-C₆alkenyl, substituted or unsubstituted C₂-C₆alkynyl, substituted or unsubstituted C₃-C₁₀cycloalkyl, substituted or unsubstituted C₂-C₁₀heterocycloalkyl, substituted or unsubstituted C₁-C₆heteroalkyl, —CN, or —S(═O)₂R⁴;
each R² is independently halogen, nitro, —N₃, —CN, —OR³, —SR³, —S(═O)₂R³, —N(R³)₂, —C(═O)OR³, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ is independently hydrogen, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or if two R³ are on the same nitrogen atom, then two R³ are taken together with the nitrogen atom to which they are attached to form a substituted or unsubstituted C₃-C₇ heterocycloalkyl;
R⁴ is substituted or unsubstituted C₁-C₆alkyl, substituted or unsubstituted C₁-C₆fluoroalkyl, substituted or unsubstituted C₃-C₁₀cycloalkyl, or —NH₂; and
n is 0, 1, 2, 3, or 4; or a stereoisomer or tautomer thereof, or a pharmaceutically acceptable salt of any of the foregoing.

Description of formula (V) can be found in US2020/0347009A1, the entirety of which is incorporated herein by reference. Formula (V) is described as Formula (I) in US2020/0347009A1 (see, e.g., paragraphs [0099]-[0110]), which paragraphs and description of Formula (I), and methods of making compounds of Formula (I) are hereby incorporated herein by reference. Moieties of formula (V), such as R¹, R², X¹, X², X³, X⁴, X⁵, X⁶, and n are as defined in US2020/0347009A1, including any variations or embodiments thereof.

In some embodiments, in conjunction with embodiments above or below, a compound of formula (V) is Compound T6, or a stereoisomer or tautomer thereof, or a pharmaceutically acceptable salt of any of the foregoing. Compound T6 is chemically described as N-[(1R)-1-(6-amino-2-pyridyl)ethyl]-5-[4-(trifluoromethyl)phenoxy]naphthalene-2-carboxamide, having the structure below:

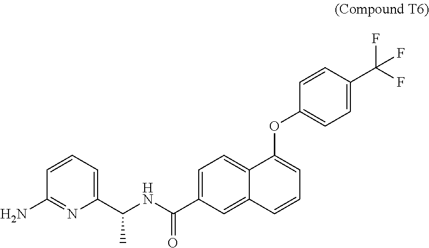 [SMILES: C[C@@H](NC(=O)c1ccc2c(Oc3ccc(C(F)(F)F)cc3)cccc2c1)c1cccc(N)n1]

Description of Compound T6 and methods of making Compound T6 can be found in, e.g., Compound 66 on page 46 and Example 55 on pages 112-115 of US2020/0347009A1.

In some embodiments, in conjunction with embodiments above or below, the one or more YAP/TAZ-TEAD inhibitors comprise a compound of formula (VI):

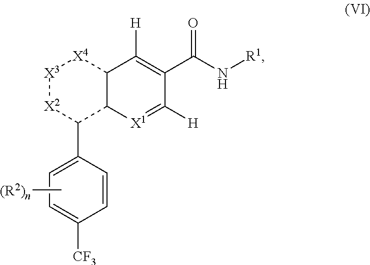

wherein,
each X¹ and X² is independently N, NR^{X}, C(═O), or CR^{X};
each X³ and X⁴ is independently N, NR^{X}, C(═O), or CR^{X}; or if both X³ and X⁴ are each independently NR^{X} or CR^{X}, then the two R^{X} are taken together with the intervening atoms to which they are attached to form a 5-membered heterocyclic ring;
each R^{X} is independently hydrogen, halogen, nitro, oxo, thioxo, imino, oximo, —OR³, —SR³, —CN, —C(═O)R², —S(═O)R³, —S(═O)₂R³, —N(R³)₂, —NR³S(═O)₂R³, —NR³C(═O)R³, —NR³C(═O)OR³, substituted or unsubstituted C₁-C₆ alkyl, substituted or unsubstituted C₁-C₆haloalkyl, substituted or unsubstituted C₂-C₄alkenyl, substituted or unsubstituted C₂-C₄alkynyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
R¹ is substituted or unsubstituted C₁-C₆alkyl, substituted or unsubstituted C₁-C₆haloalkyl, substituted or unsubstituted C₂-C₄alkenyl, substituted or unsubstituted C₂-C₄alkynyl, substituted or unsubstituted C₃-C₁₀cycloalkyl, substituted or unsubstituted C₂-C₁₀heterocycloalkyl, substituted or unsubstituted C₁-C₆heteroalkyl, or —S(═O)₂R⁴;
each R² is independently —N₃, —CN, —OR³, —SR³, —S(═O)₂R³, —N(R³)₂, —C(═O)OR³, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
each R³ is independently hydrogen, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;
R⁴ is substituted or unsubstituted C₁-C₆alkyl, substituted or unsubstituted C₁-C₆haloalkyl, substituted or unsubstituted C₃-C₁₀cycloalkyl, or —NH₂;
each - - - is independently a single or double bond; and
n is 0, 1, 2, 3, or 4; or a stereoisomer or tautomer thereof, or a pharmaceutically acceptable salt of any of the foregoing. Description of formula (VI) can be found in WO2020/097389A1, the entirety of which is incorporated herein by reference. Formula (VI) is described as Formula (I) in WO2020/097389A1 (see, e.g., paragraphs [0070]-[0082]), which paragraphs and description of Formula (I), and methods of making compounds of Formula (I) are hereby incorporated herein by reference. Moieties of formula (VI), such as R¹, R², X¹, X², X³, X₄, and n are as defined in WO2020/097389A1, including any variations or embodiments thereof.

In some embodiments, in conjunction with embodiments above or below, the one or more YAP/TAZ-TEAD inhibitors comprise a compound of formula (VI-A):

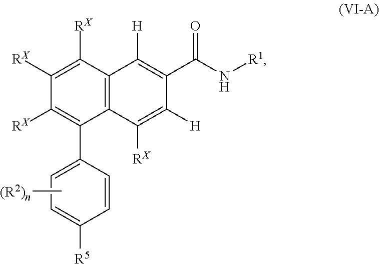

wherein,
each R^{X} is independently hydrogen, halogen, —OH, —NH₂, —CH₃, —CH₂CH₃, cyclopropyl, —CF₃, —OCH₃, —OCH₂CH₃, cyclopropyloxy, or —OCF₃;
R¹ is substituted or unsubstituted C₁-C₆ alkyl, wherein if C₁-C₆alkyl is substituted, then it is substituted with 1 or 2 substituents each independently selected from —OH, —NH₂, azetidinyl, pyridyl, and aminopyridyl;
R² is F;
R⁵ is —CF₃; and
n is 0 or 1; or a stereoisomer or tautomer thereof, or a pharmaceutically acceptable salt of any of the foregoing. Formula (VI-A) is described as Formula (Ia) in (see, e.g., paragraphs [0082]-[0089] of WO2020/097389A1, which paragraphs and description of Formula (Ia), and methods of making compounds of Formula (Ia) are hereby incorporated herein by reference. Moieties of formula (VI-A), such as R¹, R², R⁵, R^{X}, and n are as defined in WO2020/097389A1, including any variations or embodiments thereof.

In some embodiments, in conjunction with embodiments above or below, a compound of formula (VI) or (VI-A) is Compound T7, or a stereoisomer or tautomer thereof, or a pharmaceutically acceptable salt of any of the foregoing. Compound T7 is chemically described as N-[(1S)-1-(2-pyridyl)ethyl]-5-[4-(trifluoromethyl)phenyl]naphthalene-2-carboxamide, having the structure below:

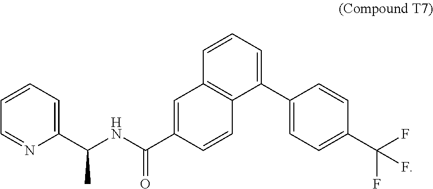 [SMILES: C[C@H](NC(=O)c1ccc2c(-c3ccc(C(F)(F)F)cc3)cccc2c1)c1ccccn1]

Description of Compound T7 and methods of making Compound T7 can be found in, e.g., Compound 90 on page 65 and Example 84 on pages 195-196 of WO2020/097389A1.

In some embodiments, in conjunction with embodiments above or below, the one or more YAP/TAZ-TEAD inhibitors comprise a compound of formula (VII):

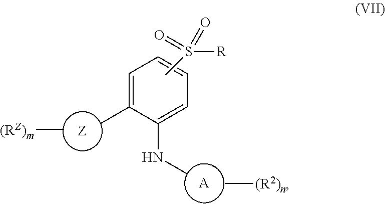

wherein,

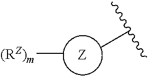

is a substituted or unsubstituted monocyclic 5-membered heterocyclic ring containing at least one N atom or a substituted or unsubstituted monocyclic 6-membered heteroaryl ring containing at least one N atom;
each R^{Z} is independently H, halogen, —CN, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, -L¹-Y¹ or -L²-L³-Y²;
m is 0, 1, 2, 3, 4, or 5;
L¹ is substituted or unsubstituted C₁-C₆alkylene, substituted or unsubstituted C₂-C₁₀cycloalkylene, or
substituted or unsubstituted C₂-C₁₀heterocycloalkylene;
Y¹ is substituted or unsubstituted C₃-C₁₀cycloalkyl, substituted or unsubstituted C₂-C₁₀ heterocycloalkyl, substituted or unsubstituted aryl, or substituted or unsubstituted heteroaryl;
L² is absent, substituted or unsubstituted C₁-C₆alkylene, substituted or unsubstituted C₂-C₁₀cycloalkylene, or substituted or unsubstituted C₂-C₁₀heterocycloalkylene;
L³ is —O—, —S—, —(S═O)—, —(SO₂)—, —NR³—, —(C═O)—, —(C═O)O—, —O(C═O)—, —(C═O)NR³—, —(C═O)NR³—O—, —O—NR³(C═O)—, —NR³(C═O)—, —NR³(C═O)NR³—, —O(C═O)NR³—, —NR³(C═O)O—, —NR₃(SO₂)NR³—, —NR₃(SO₂)—, —(SO₂)NR³—, —(SO₂)NR³—(C═O)—, —(C═O)—NR³(SO₂)—, —(SO₂)NR³—(C═O)O—, —O(C═O)—NR³(SO₂)—, —NR³(SO₂)NR³—(C═O)—, —(C═O)—NR₃(SO₂)NR³—, —O(C═O)—NR³(SO₂)—NR³—, or —NR³(SO₂)NR³—(C═O)O—;
each R³ is independently H or substituted or unsubstituted C₁-C₆alkyl;
Y² is H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aryl, or substituted or unsubstituted heteroaryl;
or R³ and Y² on the same N atom are taken together with the N atom to which they are attached to form a substituted or unsubstituted N-containing heterocycle;
R is NHR¹ or R¹;
R¹ is substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl;

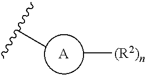

is substituted or unsubstituted phenyl or substituted or unsubstituted cyclohexyl;
each R² is independently H, halogen, —N₃, —CN, —OR⁴, —SR⁴, —(SO₂)R⁴, —N(R⁴)₂, —CO₂R⁴, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, substituted or unsubstituted heteroaryl, or

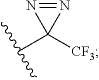 [SMILES: *C1(C(F)(F)F)N=N1]

n is 0, 1, 2, 3, 4, or 5; and
each R⁴ is independently H, substituted or unsubstituted C₁-C₆alkyl, substituted or unsubstituted C₁-C₆haloalkyl, substituted or unsubstituted C₁-C₆heteroalkyl, substituted or unsubstituted C₃-C₁₀cycloalkyl, substituted or unsubstituted C₂-C₁₀heterocycloalkyl, substituted or unsubstituted aralkyl, substituted or unsubstituted aryl, or substituted or unsubstituted heteroaryl; or a stereoisomer or tautomer thereof, or a pharmaceutically acceptable salt of any of the foregoing. Description of formula (VII) can be found in US2020/0354325A1, the entirety of which is incorporated herein by reference. Formula (VII) is described as Formula (I) in US2020/03543525A1 (see, e.g., paragraphs [0129]-[0149]), which paragraphs and description of Formula (I), and methods of making compounds of Formula (I) are hereby incorporated herein by reference. Moieties of formula (VII), such as A, Z, R, R^{z}, R², m, and n are as defined in US2020/03543525A1, including any variations or embodiments thereof.

In some embodiments, in conjunction with embodiments above or below, the one or more YAP/TAZ-TEAD inhibitors comprise a compound of formula (VII-A) or (VII-B):

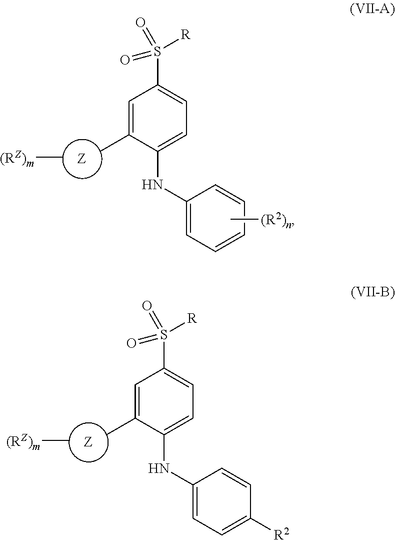

or a stereoisomer or tautomer thereof, or a pharmaceutically acceptable salt of any of the foregoing, wherein Z, R, R^{Z}, R², n, and m are as defined in Formula (VII). It is understood that Z, R, R^{Z}, R², n, and m of such embodiments of compounds of Formulae (VII-A) and (VII-B) may include Z, R, R^{Z}, R², n, and m as described for Formula (VII). Formulae (VII-A) and (VII-B) are described as Formulae (Id) and (Ie), respectively, in, e.g., paragraphs [0194] and [0195] of US2020/0354325A1, which paragraphs and description of Formula (Id) or (Ie), and methods of making compounds of Formula (Id) or (Ie) are hereby incorporated herein by reference. Moieties of formula (VII-A) or (VII-B), such as Z, R, R^{z}, R², m, and n are as defined in US2020/0354325A1, including any variations or embodiments thereof.

In some embodiments, in conjunction with embodiments above or below, a compound of formula (VII), (VII-A), or (VII-B) is Compound T8, or a stereoisomer or tautomer thereof, or a pharmaceutically acceptable salt of any of the foregoing. Compound T8 is chemically described as N-methyl-3-(1-methylimidazol-4-yl)-4-[4-(trifluoromethyl)anilino]benzenesulfonamide, having the structure below:

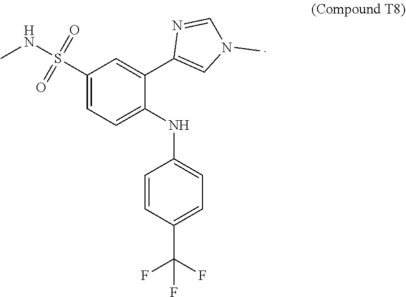

Description of Compound T8 and methods of making Compound T8 can be found in, e.g., Compound 121 on page 55 and Example 113 on pages 157-158 of US2020/0354325A1.

In some embodiments, in conjunction with embodiments above or below, the one or more YAP/TAZ-TEAD inhibitors comprise a compound of formula (VIII):

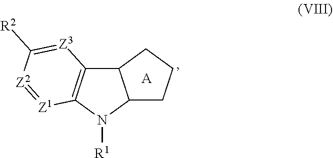

wherein,
ring A represents a five-membered heteroaromatic ring selected from the group consisting of the following ring moieties:

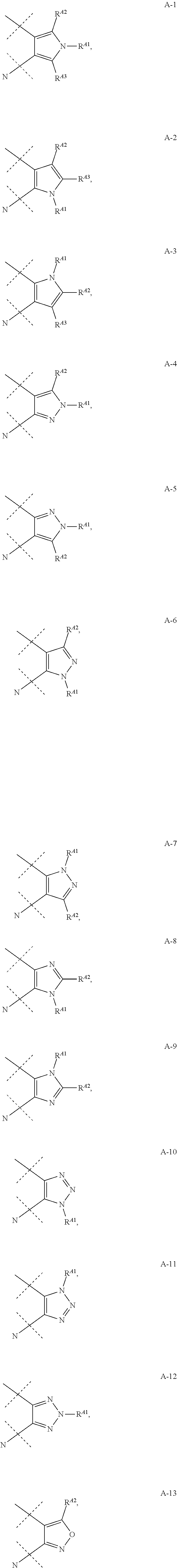

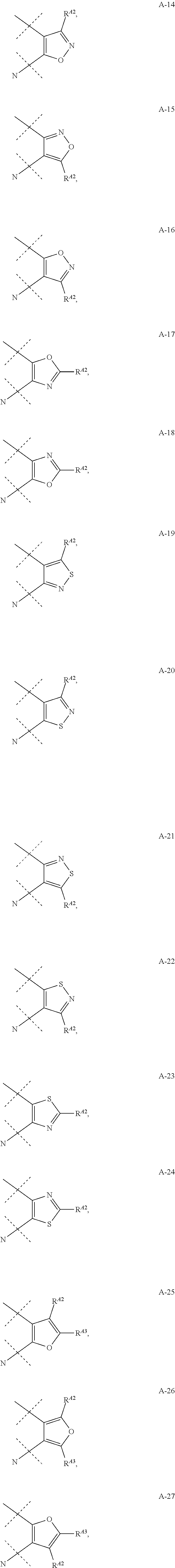

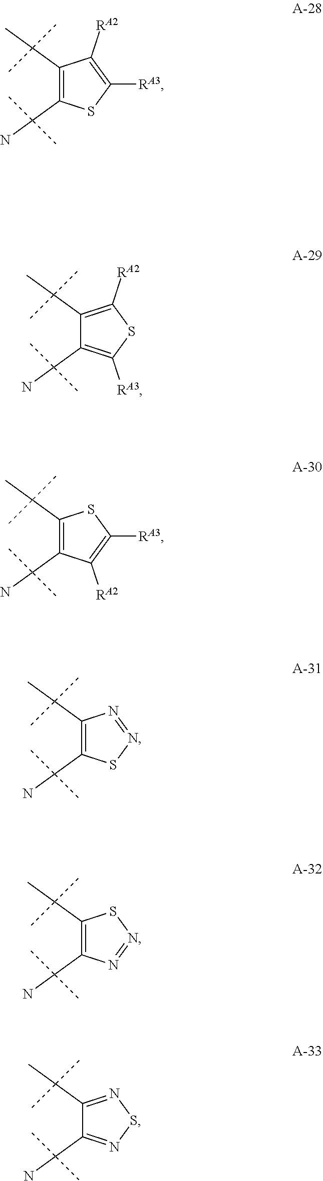

wherein
R^{A1} represents H, D, C₁₋₆aliphatic, —CH₂—Ar^{A1} or —CH₂—CH₂—Ar^{A1};
R^{A2} represents H, D, halogen, C₁₋₆aliphatic, —CH₂—Ar^{A2} or —CH₂—CH₂—Ar^{A2};
R^{A3} represents H, D, C₁₋₆aliphatic, —CH₂—Ar^{A3} or —CH₂—CH₂—Ar^{A3};
Z¹ is CR^{Z1} or N;
Z² is CR^{Z2} or N;
Z³ is CR^{Z3} or N;
wherein at least two of Z¹, Z² and Z³ are not N;
R¹ represents Ar¹, Hetar¹, Cyc¹, Hetcyc¹, L¹-Ar¹, L¹-Hetar¹, L²-Cyc¹, L²-Hetcyc¹, unsubstituted or substituted, straight-chain or branched C₁₋₈aliphatic;
R² represents —C(═O)—OR^{2a}, —C(═O)—NR^{2b}R^{2c}, —(CH₂)_{w}—C(═O)—NR^{2b}R^{2c}, —(CH₂)ₓ—NR^{2d}—C(═O)—R^{2e}, —S—R^{2f}, —S(═O)—R^{2f}, —S(═O)₂—R^{2g}, —S(═O)₂—NR^{2h}R²ⁱ, —S(═O)₂—OH, —S(═O)(═NR^{2j})—OH, —S(═O)(═NR^{2j})—R^{2g}, S(═O)(═NR^{2k})—NR^{2l}R^{2m}, F, Cl, Br, I, —CN, —(CH₂)ᵥ—CN, —P(═O)(OR^{2o})(OR^{2p}), —(CH₂)_{y}—NR^{2q}R^{2r}, —(CH₂)_{z}—NR^{2d}—S(═O)₂—R^{2g}, —C(═O)—N═S(═O)—R^{2s}R^{2t}, —C(═O)—N═S(═N—R^{2u})—R^{2s}R^{2t}, —B(OH)₂ or Hetcyc^{X};
Ar^{A1}, Ar^{A2}, Ar^{A3} represent independently from each other phenyl which may be unsubstituted or mono- or di-substituted with independently from each other R^{A11} and/or R^{A12};
R^{Z1} represents H or halogen;
R^{Z2} represents H or halogen; or forms together with R² a divalent radical —S(═O)₂—N(H)—C(═O)—;
R^{Z3} represents H or halogen;
R^{2a} represents H, un-substituted or substituted C₁₋₈aliphatic, aryl, heteroaryl, saturated or partially unsaturated heterocyclyl, or carbohydrate derived radical, or Cat;
Cat represents a monovalent cation;
R^{2b}, R^{2c}, R^{2q}, R^{2r} represent independently from each other H, unsubstituted or substituted C₁₋₈aliphatic including C₃₋₇cycloaliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms; wherein said heterocycle may optionally be fused with Hetar^{Z}; or one of R^{2b} and R^{2c} represents —CN, —NH₂, —OH, —O—C₁₋₆alkyl, —S(═O)₂—R^{2g}, Ar², Hetar², Cyc² or Hetcyc², while the other represents H or unsubstituted or substituted C₁₋₈aliphatic;
R^{2d}, R^{2j}, R^{2k}, R^{2o}, R^{2p} represent independently from each other H, unsubstituted or substituted C₁₋₈aliphatic;
R^{2e} represents H, halogen, unsubstituted or substituted C₁₋₈aliphatic, heteroaryl;
R^{2f}, R^{2g} represent independently from each other unsubstituted of substituted C₁₋₈aliphatic;
R^{2h}, R²ⁱ represent independently from each other H, un-substituted or substituted C₁₋₈aliphatic, aryl, heterocyclyl, heteroaryl; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms;
R^{2l}, R^{2m} represent independently from each other H, unsubstituted or substituted C₁₋₈aliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms;
R^{2s}, R^{2t} represent independently from each other unsubstituted or substituted C₁₋₈aliphatic; or form together an unsubstituted or substituted divalent C₃₋₆alkylene radical;
R^{2u} represents hydrogen or unsubstituted or substituted C₁₋₆aliphatic;
Ar¹ is a mono-, bi- or tricyclic aryl with 5, 6, 7, 8, 9, 10, 11, 12, 13, 14 ring carbon atoms, wherein that aryl may be unsubstituted or substituted with substituents R^{B1}, R^{B2}, R^{B3}, R^{B4}, R^{B5}, R^{B6} and/or R^{B7} which may be the same or different;
Hetar¹ is a mono-, bi- or tricyclic heteroaryl with 5, 6, 7, 8, 9, 10, 11, 12, 13, 14 ring atoms wherein 1, 2, 3, 4, 5 of said ring atoms is/are a hetero atom(s) selected from N, O, and/or S and the remaining are carbon atoms, wherein that heteroaryl may be unsubstituted or substituted with substituents R^{B1}, R^{B2}, R^{B3}, R^{B4}, R^{B5}, R^{B6} and/or R^{B7} which may be the same or different;
Cyc¹ is a saturated or partially unsaturated, mono-, bi- or tricyclic carbocycle with 3, 4, 5, 6, 7, 8, 9, 10, 11, 12, 13, 14, 15 ring carbon atoms, wherein that carbocycle may be unsubstituted or substituted with R^{B8}, R^{B9}, R^{B10}, R^{B11}R^{B12} and/or R^{B13} which may be the same or different; and wherein that carbocycle may optionally be fused to Ar^{X} via 2 adjacent ring atoms of said Ar^{X} and wherein that fused carbocycle may further be unsubstituted or substituted with R^{C1}, R^{C2}, R^{C3}, R^{C4}, R^{C5}, R^{C6} which may be the same or different;
Hetcyc¹ is a saturated or partially unsaturated, mono-, bi- or tricyclic heterocycle with 3, 4, 5, 6, 7, 8, 9, 10, 11, 12, 13, 14, 15 ring atoms wherein 1, 2, 3, 4, 5 of said ring atoms is/are a hetero atom(s) selected from N, O, and/or S and the remaining are carbon atoms, wherein that heterocycle may be unsubstituted or substituted with R^{B8}, R^{B9}, R^{B10}, R^{B11}, R^{B12} and/or R^{B13} which may be the same or different;
L¹ is a divalent radical selected from the group consisting of —S(═O)₂—, —C(═O)—, un-substituted or substituted, straight-chain or branched C₁₋₆alkylene or C₂₋₆alkenylene, in both of which one of the carbon units of the alkylene or alkenylene chain may be replaced by —O—;
L² is a divalent radical selected from the group consisting of unsubstituted or substituted, straight-chain or branched C₁₋₆alkylene or C₂₋₆alkenylene, in both of which one of the carbon units of the alkylene or alkenylene chain may be replaced by —O—;
R^{A11}, R^{A12} represent independently from each other halogen or unsubstituted or substituted, straight-chain or branched C₁₋₆aliphatic;
R^{B1}, R^{B2}, R^{B3}, R^{B4}, R^{B5}, R^{B6}, R^{B7} represent independently from each other un-substituted or substituted, straight-chain or branched C₁₋₆aliphatic, C₁₋₆aliphatoxy, —S—C₁₋₆aliphatic; halogen, —CN, —S(═O)—R^{b1}, S(═O)₂—R^{b1}, —NR^{b2}NR^{b3}, Ar², —CH₂—Ar², Hetar², Cyc², Hetcyc²; and/or two adjacent R^{B1}, R^{B2}, R^{B3}, R^{B4}, R^{B5}, R^{B6} and/or R^{B7} form together a divalent —C₂₋₄alkylene radical in which one of the alkylene carbon units may be replaced by a carbonyl unit (—C(═O)—), or a divalent —O—C₁₋₃alkylene radical or a divalent —O—C₁₋₃alkylene-O-radical;
R^{b1} represents unsubstituted or substituted C₁₋₈aliphatic;
R^{b2}, R^{b3} represent independently from each other H, unsubstituted or substituted C₁₋₈aliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms;
R^{B8}, R^{B9}, R^{B10}, R^{B11}, R^{B12}, R^{B13} represent independently from each other halogen, un-substituted or substituted C₁₋₆aliphatic, C₁₋₆aliphatoxy, Ar^{Y}; and/or two of R^{B8}, R^{B9}, R^{B10}R^{B11}, R^{B12}, R^{B13} which are attached to the same carbon atom of said carbocycle or said heterocycle form a divalent oxo (═O) group; and/or two of R^{B8}, R^{B9}, R^{B10}, R^{B11}, R^{B12}, R^{B13} or four of R^{B8}, R^{B9}, R^{B10}, R^{B11}, R^{B12}, R^{B13} which are attached to the same sulfur atom of said heterocycle form a divalent oxo (═O) group thereby forming either an —S(═O)— or an —S(═O)₂— moiety;
Ar² is a mono- or bicyclic aryl with 5, 6, 7, 8, 9, 10 ring carbon atoms, wherein that aryl may be unsubstituted or substituted with substituents R^{D1}, R^{D2}, R^{D3}, R^{D4} and/or R^{D5} which may be the same or different;
Hetar² is a mono- or bicyclic heteroaryl with 5, 6, 7, 8, 9, 10 ring atoms wherein 1, 2, 3, 4, 5 of said ring atoms is/are a hetero atom(s) selected from N, O, and/or S and the remaining are carbon atoms, wherein that heteroaryl may be unsubstituted or substituted with substituents R^{D1}, R^{D2}, R^{D3}, R^{D4} and/or R^{D5} which may be the same or different;
Cyc² is a saturated or partially unsaturated monocyclic carbocycle with 3, 4, 5, 6 or 7 ring carbon atoms, wherein that carbocycle may be unsubstituted or substituted with R^{D6}, R^{D7}, R^{D8}, R^{D9} and/or R^{D10} which may be the same or different; wherein that carbocycle may optionally be fused to Ar^{Z} or Hetar^{Z} via 2 adjacent ring atoms of said Ar^{Z} or Hetar^{Z} and wherein that fused carbocycle may further be unsubstituted or substituted with R^{C1}, R^{C2}, R^{C3}, R^{C4}, R^{C5}, R^{C6} which may be the same or different;
Hetcyc² is a saturated or partially unsaturated, monocyclic heterocycle with 3, 4, 5, 6, 7 ring atoms wherein 1 or 2 of said ring atoms is/are a hetero atom(s) selected from N, O, and/or S and the remaining are carbon atoms, wherein that heterocycle may be unsubstituted or substituted with R^{D6}, R^{D7}, R^{D8}, R^{D9} and/or R^{D10} which may be the same or different; wherein that heterocycle may optionally be fused to Ar^{Z} or Hetar^{Z} via 2 adjacent ring atoms of said Ar^{Z} or Hetar^{Z} and wherein that fused heterocycle may further be unsubstituted or substituted with R^{C1}, R^{C2}, R^{C3}, R^{C4}, R^{C5}, R^{C6} which may be the same or different;
Ar^{X}, Ar^{Z} independently from each other an unsubstituted or substituted benzo ring;
Ar^{Y} is an unsubstituted or mono- or di-substituted phenyl;
Hetar^{Y1} is a 5 or 6 membered monocyclic heteroaryl wherein 1, 2, 3, 4 ring atoms are hetero atoms selected from N, O, and/or S and the remaining are carbon atoms, wherein that heteroaryl may be unsubstituted or substituted with halogen, C₁₋₄alkyl which may optionally be substituted with OH;
Hetar^{Z} is an unsubstituted or substituted 5 or 6 membered heteroaryl ring selected from the group consisting of pyrrole, furan, thiophene, pyrazole, imidazole, oxaole, isoxazole, thiazole, oxadiazole, triazole, tetrazole, pyridine, pyrimidine, pyrazine, pyrane;
Cyc^{Y1} is a saturated monocyclic carbocycle with 3, 4, 5, 6 or 7 ring carbon atoms, wherein that carbocycle may be unsubstituted or substituted with halogen, OH, C₁₋₄alkyl;
Hetcyc^{X} is a saturated, partially unsaturated or aromatic, monocyclic heterocycle with 3, 4, 5, 6, 7 ring atoms wherein 1, 2, 3, 4 of said ring atoms is/are a hetero atom(s) selected from N, O, and/or S and the remaining are carbon atoms, wherein said heterocycle may be unsubstituted or substituted with R^{X1}, R^{X2}, R^{X3}, R^{X4}, R^{X5}, R^{X6}, R^{X7} and/or R^{X8} which may be the same or different, and wherein that heterocycle is optionally a carboxylic acid bioisostere;
Hetcyc^{Y} is a saturated, partially unsaturated or aromatic, monocyclic heterocycle with 3, 4, 5, 6, 7 ring atoms wherein 1, 2, 3, 4 of said ring atoms is/are a hetero atom(s) selected from N, O, and/or S and the remaining are carbon atoms;
Hetcyc^{Y1} is a saturated or partially unsaturated monocyclic heterocycle with 5 or 6 ring atoms wherein 1 or 2 of said ring atoms are heteroatoms selected from N, O, and/or S and the remaining are carbon atoms;
R^{C1}, R^{C2}, R^{C3}, R^{C4}, R^{C5}, R^{C6} represent independently from each other unsubstituted or substituted C₁₋₆aliphatic;
R^{D1}, R^{D2}, R^{D3}, R^{D4}, R^{D5} represent independently from each other unsubstituted or substituted C₁₋₆aliphatic;
R^{D6}, R^{D7}, R^{D8}, R^{D9}, R^{D10} represent independently from each other unsubstituted or substituted C₁₋₆aliphatic, unsubstituted or substituted C₁₋₆aliphatoxy, halogen, hydroxy; Hetar^{Y1}, CH₂—Hetar^{Y1}, Cyc^{Y1}, Hetcyc^{Y1}, —CH₂-Hetcyc^{Y1}; and/or two of R^{D6}, R^{D7}, R^{D8}, R^{D9}, R^{D10} which are attached to the same ring atom of said carbocycle or heterocycle may form a divalent C₂₋₆alkylene radical, wherein one or two non-adjacent carbon units of said alkylene radical may optionally be replaced by independently from each other O, N—H, or N—C₁₋₄alkyl, and wherein that alkylene radical may optionally be substituted with OH, C₁₋₄alkyl or —O—C₁₋₄alkyl; and/or two of R^{D6}, R^{D7}, R^{D8}, R^{D9}, R^{D10} which are attached to different ring atoms of said carbocycle or heterocycle may form a divalent C₁₋₆alkylene radical, wherein one or two non-adjacent carbon units of said alkylene radical may optionally be replaced by independently from each other O, N—H, or N—C₁₋₄alkyl;
R^{X1}, R^{X2}, R^{X3}, R^{X4}, R^{X5}, R^{X6}, R^{X7}, R^{X8} represent independently from each other unsubstituted or substituted C₁₋₆aliphatic, C₁₋₆aliphatoxy, halogen, —OH, —NR^{2d}—S(═O)₂—R^{2g}, Hetcyc^{Y}, O-Hetcyc^{Y}; and/or two of R^{X1}, R^{X2}, R^{X3}, R^{X4}, R^{X5}, R^{X6}, R^{X7}, R^{X8} which are attached to the same carbon atom of said heterocycle form a divalent oxo (═O) group; and/or two of R^{X1}, R^{X2}, R^{X3}, R^{X4}, R^{X5}, R^{X6}, R^{X7}, R^{X8} or four of R^{X1}, R^{X2}, R^{X3}, R^{X4}, R^{X5}, R^{X6}, R^{X7}, R^{X8} which are attached to the same sulfur atom of said heterocycle form a divalent oxo (═O) group thereby forming either an —S(═O)— or an —S(═O)₂— moiety;
halogen is F, Cl, Br, I;
v is 1 or 2;
w is 1 or 2;
x is 0, 1, or 2;
y is 0, 1, or 2;
z is 0, 1, or 2; or a stereoisomer or tautomer thereof, or a pharmaceutically acceptable salt of any of the foregoing.

Description of formula (VIII) can be found in WO2021/224291A1, the entirety of which is incorporated herein by reference. Formula (VIII) is described as formula (I-A) in WO2021/224291A1 (see, e.g., pages 3-12), which paragraphs and description of formula (I-A), and methods of making compounds of formula (I-A) are hereby incorporated herein by reference. Moieties of formula (VIII), such as A, Z¹, Z², Z³, R¹, and R² are as defined in WO2021/224291A1, including any variations or embodiments thereof.

In some embodiments, in conjunction with embodiments above or below, the one or more YAP/TAZ-TEAD inhibitors comprise a compound of formula (VIII-A):

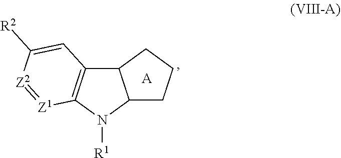

wherein,
ring A represents a five-membered heteroaromatic ring selected from the group consisting of the following ring moieties:

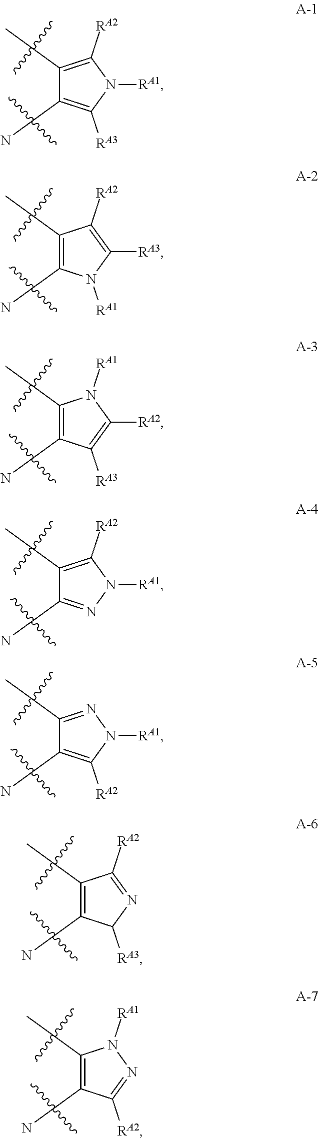

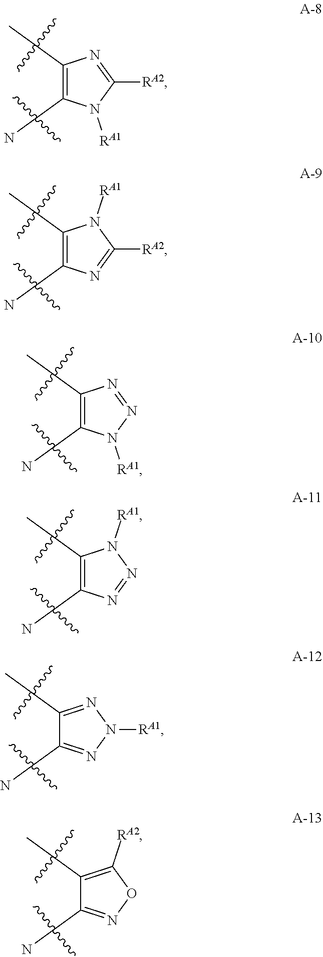

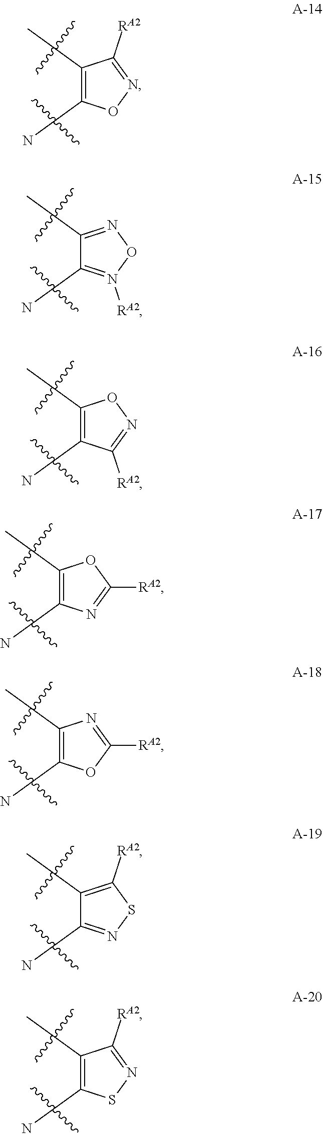

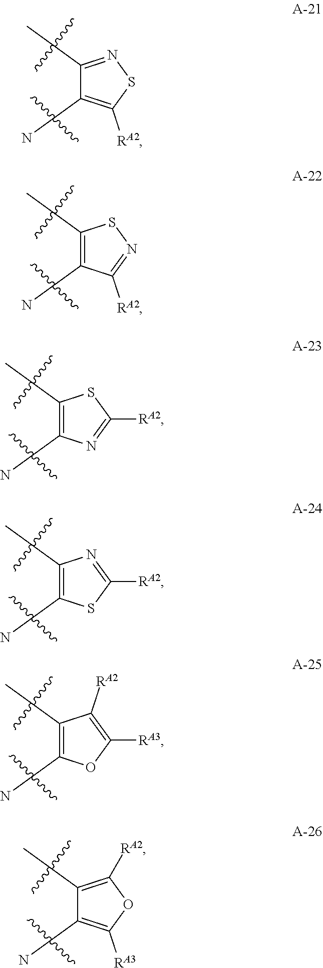

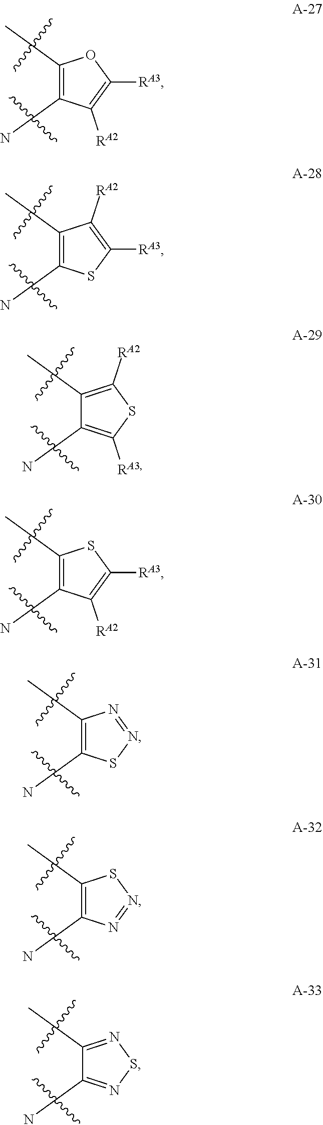

wherein,
R^{A1} represents H, C₁₋₆aliphatic, —CH₂—Ar^{A1} or —CH₂—CH₂—Ar^{A1};
R^{A2} represents H, halogen, C₁₋₆aliphatic, —CH₂—Ar^{A2} or —CH₂—CH₂—Ar^{A2};
R^{A3} represents H, C₁₋₆aliphatic, —CH₂—Ar^{A3} or —CH₂—CH₂—Ar^{A3};
Z¹ is CR^{Z1} or N;
Z² is CR^{Z2} or N;
wherein at least one of Z¹ and Z² is not N;
R¹ represents Ar¹, Hetar¹, Cyc¹, Hetcyc¹, L¹-Ar¹, L¹-Hetar¹, L²-Cyc¹, L²-Hetcyc¹, unsubstituted or substituted, straight-chain or branched C₁₋₈aliphatic;
R² represents —C(═O)—OR^{2a}, —C(═O)—NR^{2b}R^{2c}, —(CH₂)_{w}—C(═O)—NR^{2b}R^{2c}, —(CH₂)ₓ—NR^{2a}—C(═O)—R^{2e}, —S—R^{2f}, —S(═O)—R^{2f}, —S(═O)₂—R^{2g}, —S(═O)₂—NR^{2h}R²ⁱ, —S(═O)₂—OH, —S(═O)(═NR^{2j})—OH, —S(═O)(═NR^{2j})—R^{2g}, S(═O)(═NR^{2k})—NR^{2l}R^{2m}, F, Cl, Br, I, —CN, —(CH₂)ᵥ—CN, —P(═O)(OR^{2o})(OR^{2p}), —(CH₂)_{y}—NR^{2q}R^{2r}, —(CH₂)_{z}—NR^{2l}—S(═O)₂—R^{2g}, —C(═O)—N═S(═O)—R^{2s}R^{2t}, —C(═O)—N═S(═N—R^{2u})—R^{2s}R^{2t}, —B(OH)₂ or Hetcyc^{X};
Ar^{A1}, Ar^{A2}, Ar^{A3} represent independently from each other phenyl which may be unsubstituted or mono- or di-substituted with independently from each other R^{A11} and/or R^{A12};
R^{Z1} represents H or halogen;
R^{Z2} represents H or halogen; or forms together with R² a divalent radical —S(═O)₂—N(H)—C(═O)—;
R^{2a} represents H, unsubstituted or substituted C₁₋₈aliphatic, aryl, heteroaryl, saturated or partially unsaturated heterocyclyl, or carbohydrate derived radical, or Cat;
Cat represents a monovalent cation;
R^{2b}, R^{2c}, R^{2q}, R^{2r} represent independently from each other H, unsubstituted or substituted C₁₋₈aliphatic including C₃₋₇cycloaliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms; wherein said heterocycle may optionally be fused with Hetar^{Z}; or one of R^{2b} and R^{2c} represents —CN, —NH₂, —OH, —O—C₁₋₆alkyl, —S(═O)₂—R^{2g}, Ar², Hetar², Cyc² or Hetcyc², while the other of R^{2b} and R^{2c} represents H or unsubstituted or substituted C₁₋₈aliphatic;
R^{2d}, R^{2j}, R^{2k}, R^{2o}, R^{2p} represent independently from each other H, unsubstituted or substituted C₁₋₈aliphatic;
R^{2c} represents H, halogen, unsubstituted or substituted C₁₋₈aliphatic, heteroaryl;
R^{2f}, R^{2g} represent independently from each other unsubstituted of substituted C₁₋₈aliphatic;
R^{2h}, R²ⁱ represent independently from each other H, un-substituted or substituted C₁₋₈aliphatic, aryl, heterocyclyl, heteroaryl; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms;
R^{2l}, R^{2m} represent independently from each other H, unsubstituted or substituted C₁₋₈aliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms;
R^{2s}, R^{2t} represent independently from each other unsubstituted or substituted C₁₋₈aliphatic; or form together an unsubstituted or substituted divalent C₃₋₆alkylene radical;
R^{2u} represents hydrogen or unsubstituted or substituted C₁₋₆aliphatic;
Ar¹ is a mono-, bi- or tricyclic aryl with 5, 6, 7, 8, 9, 10, 11, 12, 13, 14 ring carbon atoms, wherein that aryl may be unsubstituted or substituted with substituents R^{B1}, R^{B2}, R^{B3}, R^{B4}, R^{B5}, R^{B6} and/or R^{B7} which may be the same or different;
Hetar¹ is a mono-, bi- or tricyclic heteroaryl with 5, 6, 7, 8, 9, 10, 11, 12, 13, 14 ring atoms wherein 1, 2, 3, 4, 5 of said ring atoms is/are a hetero atom(s) selected from N, O, and/or S and the remaining are carbon atoms, wherein that heteroaryl may be unsubstituted or substituted with substituents R^{B}1, R^{B2}, R^{B3}, R^{B4}, R^{B5}, R^{B6} and/or R^{B7} which may be the same or different;
Cyc¹ is a saturated or partially unsaturated, mono-, bi- or tricyclic carbocycle with 3, 4, 5, 6, 7, 8, 9, 10, 11, 12, 13, 14, 15 ring carbon atoms, wherein that carbocycle may be unsubstituted or substituted with R^{B8}, R^{B9}, R^{B10}, R^{B11} R^{B12} and/or R^{B13} which may be the same or different; and wherein that carbocycle may optionally be fused to Ar^{X} via 2 adjacent ring atoms of said Ar^{X} and wherein that fused carbocycle may further be unsubstituted or substituted with R^{C1}, R^{C2}, R^{C3}, R^{C4}, R^{C5}, R^{C6} which may be the same or different;
Hetcyc¹ is a saturated or partially unsaturated, mono-, bi- or tricyclic heterocycle with 3, 4, 5, 6, 7, 8, 9, 10, 11, 12, 13, 14, 15 ring atoms wherein 1, 2, 3, 4, 5 of said ring atoms is/are a hetero atom(s) selected from N, O, and/or S and the remaining are carbon atoms, wherein that heterocycle may be unsubstituted or substituted with R^{B8}, R^{B9}, R^{B10}, R^{B11}R^{B12} and/or R^{B13} which may be the same or different;
L¹ is a divalent radical selected from the group consisting of —S(═O)₂—, —C(═O)—, un-substituted or substituted, straight-chain or branched C₁₋₆alkylene or C₂₋₆alkenylene, in both of which one of the carbon units of the alkylene or alkenylene chain may be replaced by —O—;
L² is a divalent radical selected from the group consisting of unsubstituted or substituted, straight-chain or branched C₁₋₆alkylene or C₂₋₆alkenylene, in both of which one of the carbon units of the alkylene or alkenylene chain may be replaced by —O—;
R^{A11}, R^{A12} represent independently from each other halogen or unsubstituted or substituted, straight-chain or branched C₁₋₆aliphatic;
R^{B1}, R^{B2}, R^{B3}, R^{B4}, R^{B5}, R^{B6}, R^{B7} represent independently from each other un-substituted or substituted, straight-chain or branched C₁₋₆aliphatic, C₁₋₆aliphatoxy, —S—C₁₋₆aliphatic; halogen, —CN, —S(═O)—R^{b1}, S(═O)₂—R^{b1}, —NR^{b2}NR^{b3}, Ar², —CH₂—Ar², Hetar², Cyc², Hetcyc²; and/or two adjacent R^{B1}, R^{B2}, R^{B3}, R^{B4}, R^{B5}, R^{B6} and/or R^{B7} form together a divalent —C₂₋₄alkylene radical in which one of the alkylene carbon units may be replaced by a carbonyl unit (—C(═O)—), or a divalent —O—C₁₋₃alkylene radical or a divalent —O—C₁₋₃alkylene-O-radical;
R^{b1} represents unsubstituted or substituted C₁₋₈aliphatic;
R^{b2}, R^{b3} represent independently from each other H, unsubstituted or substituted C₁₋₈ aliphatic; or form together with the nitrogen atom to which they are attached to an unsubstituted or substituted saturated, partially unsaturated or aromatic heterocycle with 3, 4, 5, 6, 7 ring atoms wherein 1 of said ring atoms is said nitrogen atom and no or one further ring atom is a hetero atom selected from N, O, or S and the remaining are carbon atoms;
R^{B8}, R^{B9}, R^{B10}, R^{B11}, R^{B12}, R^{B13} represent independently from each other halogen, un-substituted or substituted C₁₋₆aliphatic, C₁₋₆aliphatoxy, Ar^{Y}; and/or two of R^{B8}, R^{B9}, R^{B10}, R^{B11}, R^{B12}, R^{B13} which are attached to the same carbon atom of said carbocycle or said heterocycle form a divalent oxo (═O) group; and/or two of R^{B8}, R^{B9}, R^{B10}, R^{B11}, R^{B12}, R^{B13} or four of R^{B8}, R^{B9}, R^{B10}, R^{B11}, R^{B12}, R^{B13} which are attached to the same sulfur atom of said heterocycle form a divalent oxo (═O) group thereby forming either an —S(═O)— or an —S(═O)₂— moiety;
Ar² is a mono- or bicyclic aryl with 5, 6, 7, 8, 9, 10 ring carbon atoms, wherein that aryl may be unsubstituted or substituted with substituents R^{D1}, R^{D2}, R^{D3}, R^{D4} and/or R^{D5} which may be the same or different;
Hetar² is a mono- or bicyclic heteroaryl with 5, 6, 7, 8, 9, 10 ring atoms wherein 1, 2, 3, 4, 5 of said ring atoms is/are a hetero atom(s) selected from N, O, and/or S and the remaining are carbon atoms, wherein that heteroaryl may be unsubstituted or substituted with substituents R^{D1}, R^{D2}, R^{D3}, R^{D4} and/or R^{D5} which may be the same or different;
Cyc² is a saturated or partially unsaturated monocyclic carbocycle with 3, 4, 5, 6 or 7 ring carbon atoms, wherein that carbocycle may be unsubstituted or substituted with R^{D6}, R^{D7}, R^{D8}, R^{D9} and/or R^{D10} which may be the same or different; wherein that carbocycle may optionally be fused to Ar^{Z} or Hetar^{Z} via 2 adjacent ring atoms of said Ar^{Z} or Hetar^{Z} and wherein that fused carbocycle may further be unsubstituted or substituted with R^{C1}, R^{C2}, R^{C3}, R^{C4}, R^{C5}, R^{C6} which may be the same or different;
Hetcyc² is a saturated or partially unsaturated, monocyclic heterocycle with 3, 4, 5, 6, 7 ring atoms wherein 1 or 2 of said ring atoms is/are a hetero atom(s) selected from N, O, and/or S and the remaining are carbon atoms, wherein that heterocycle may be unsubstituted or substituted with R^{D6}, R^{D7}, R^{D8}, R^{D9} and/or R^{D10} which may be the same or different; wherein that heterocycle may optionally be fused to Ar^{Z} or Hetar^{Z} via 2 adjacent ring atoms of said Ar^{Z} or Hetar^{Z} and wherein that fused heterocycle may further be unsubstituted or substituted with R^{C1}, R^{C2}, R^{C3}, R^{C4}, R^{C5}, R^{C6} which may be the same or different;
Ar^{X}, Ar^{Z} independently from each other an unsubstituted or substituted benzo ring;
Ar^{Y} is an unsubstituted or mono- or di-substituted phenyl;
Hetar^{Y1} is a 5 or 6 membered monocyclic heteroaryl wherein 1, 2, 3, 4 ring atoms are hetero atoms selected from N, O, and/or S and the remaining are carbon atoms, wherein that heteroaryl may be unsubstituted or substituted with halogen, C₁₋₄alkyl which may optionally be substituted with OH;
Hetar^{Z} is an unsubstituted or substituted 5 or 6 membered heteroaryl ring selected from the group consisting of pyrrole, furan, thiophene, pyrazole, imidazole, oxaole, isoxazole, thiazole, oxadiazole, triazole, tetrazole, pyridine, pyrimidine, pyrazine, pyrane;
Cyc^{Y1} is a saturated monocyclic carbocycle with 3, 4, 5, 6 or 7 ring carbon atoms, wherein that carbocycle may be unsubstituted or substituted with halogen, OH, C₁₋₄alkyl;
Hetcyc^{X} is a saturated, partially unsaturated or aromatic, monocyclic heterocycle with 3, 4, 5, 6, 7 ring atoms wherein 1, 2, 3, 4 of said ring atoms is/are a hetero atom(s) selected from N, O, and/or S and the remaining are carbon atoms, wherein said heterocycle may be unsubstituted or substituted with R^{X1}, R^{X2}, R^{X3}, R^{X4}, R^{X5}, R^{X6}, R^{X7} and/or R^{X8} which may be the same or different, and wherein that heterocycle is optionally a carboxylic acid bioisostere;
Hetcyc^{Y} is a saturated, partially unsaturated or aromatic, monocyclic heterocycle with 3, 4, 5, 6, 7 ring atoms wherein 1, 2, 3, 4 of said ring atoms is/are a hetero atom(s) selected from N, O, and/or S and the remaining are carbon atoms;
Hetcyc^{Y1} is a saturated or partially unsaturated monocyclic heterocycle with 5 or 6 ring atoms wherein 1 or 2 of said ring atoms are heteroatoms selected from N, O, and/or S and the remaining are carbon atoms;
R^{C1}, R^{C2}, R^{C3}, R^{C4}, R^{C5}, R^{C6} represent independently from each other unsubstituted or substituted C₁₋₆aliphatic;
R^{D1}, R^{D2}, R^{D3}, R^{D4}, R^{D5} represent independently from each other unsubstituted or substituted C₁₋₆aliphatic;
R^{D6}, R^{D7}, R^{D8}, R^{D9}, R^{D10} represent independently from each other unsubstituted or substituted C₁₋₆aliphatic, unsubstituted or substituted C₁₋₆aliphatoxy, halogen, hydroxy; Hetar^{Y1}, CH₂—Hetar^{Y1}, Cyc^{Y1}, Hetcyc^{Y1}, —CH₂-Hetcyc^{Y1}; and/or two of R^{D6}, R^{D7}, R^{D8}, R^{D9}, R^{D10} which are attached to the same ring atom of said carbocycle or heterocycle may form a divalent C₂₋₆alkylene radical, wherein one or two non-adjacent carbon units of said alkylene radical may optionally be replaced by independently from each other O, N—H, or N—C₁₋₄alkyl, and wherein that alkylene radical may optionally be substituted with OH, C₁₋₄alkyl or —O—C₁₋₄alkyl; and/or two of R^{D6}, R^{D7}, R^{D8}, R^{D9}, R^{D10} which are attached to different ring atoms of said carbocycle or heterocycle may form a divalent C₁₋₆alkylene radical, wherein one or two non-adjacent carbon units of said alkylene radical may optionally be replaced by independently from each other O, N—H, or N—C₁₋₄alkyl;
R^{X1}, R^{X2}, R^{X3}, R^{X4}, R^{X5}, R^{X6}, R^{X7}, R^{X8} represent independently from each other unsubstituted or substituted C₁₋₆aliphatic, C₁₋₆aliphatoxy, halogen, —OH, —NR^{2d}—S(═O)₂—R^{2g}, Hetcyc^{Y}, O-Hetcyc^{Y}; and/or two of R^{X1}, R^{X2}, R^{X3}, R^{X4}, R^{X5}, R^{X6}, R^{X7}, R^{X8} which are attached to the same carbon atom of said heterocycle form a divalent oxo (═O) group; and/or two of R^{X1}, R^{X2}, R^{X3}, R^{X4}, R^{X5}, R^{X6}, R^{X7}, R^{X8} or four of R^{X1}, R^{X2}, R^{X3}, R^{X4}, R^{X5}, R^{X6}, R^{X7}, R^{X8} which are attached to the same sulfur atom of said heterocycle form a divalent oxo (═O) group thereby forming either an —S(═O)— or an —S(═O)₂— moiety; halogen is F, Cl, Br, I;
v is 1 or 2;
w is 1 or 2;
x is 0, 1, or 2;
y is 0, 1, or 2;
z is 0, 1, or 2; or a stereoisomer or tautomer thereof, or a pharmaceutically acceptable salt of any of the foregoing.

Formula (VIII-A) is described as formula (I) in, e.g., pages 12-94, of WO2021/224291A1, which paragraphs and description of formula (I), and methods of making compounds of formula (I) are hereby incorporated herein by reference. Moieties of formula (VIII-A), such as A, Z¹, Z², R¹, and R² are as defined in WO2021/224291A1, including any variations or embodiments thereof.

In some embodiments, in conjunction with embodiments above or below, a compound of formula (VIII) or (VIII-A) is Compound T10, or a stereoisomer or tautomer thereof, or a pharmaceutically acceptable salt of any of the foregoing. Compound T10 is chemically described as 2-methyl-8-[4(trifluoromethyl)phenyl]-2H,8H-pyrazolo[3,4-blindole-5-carboxylic acid, having the structure below:

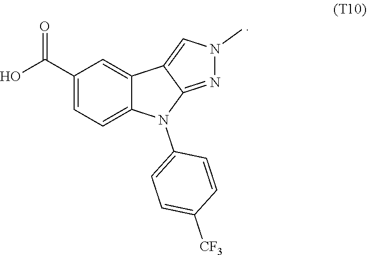 [SMILES: Cn1cc2c3cc(C(=O)O)ccc3n(-c3ccc(C(F)(F)F)cc3)c2n1]

Description of Compound T10 and methods of making Compound T10 can be found in, e.g., Compound 2 on page 198 and Example 2-4 on page 152 of WO2021/224291A1.

In some embodiments, in conjunction with embodiments above or below, the one or more TEAD inhibitors comprise

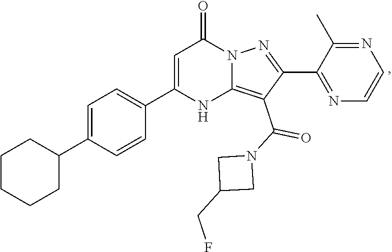 [SMILES: Cc1nccnc1-c1nn2c(=O)cc(-c3ccc(C4CCCCC4)cc3)[nH]c2c1C(=O)N1CC(CF)C1]

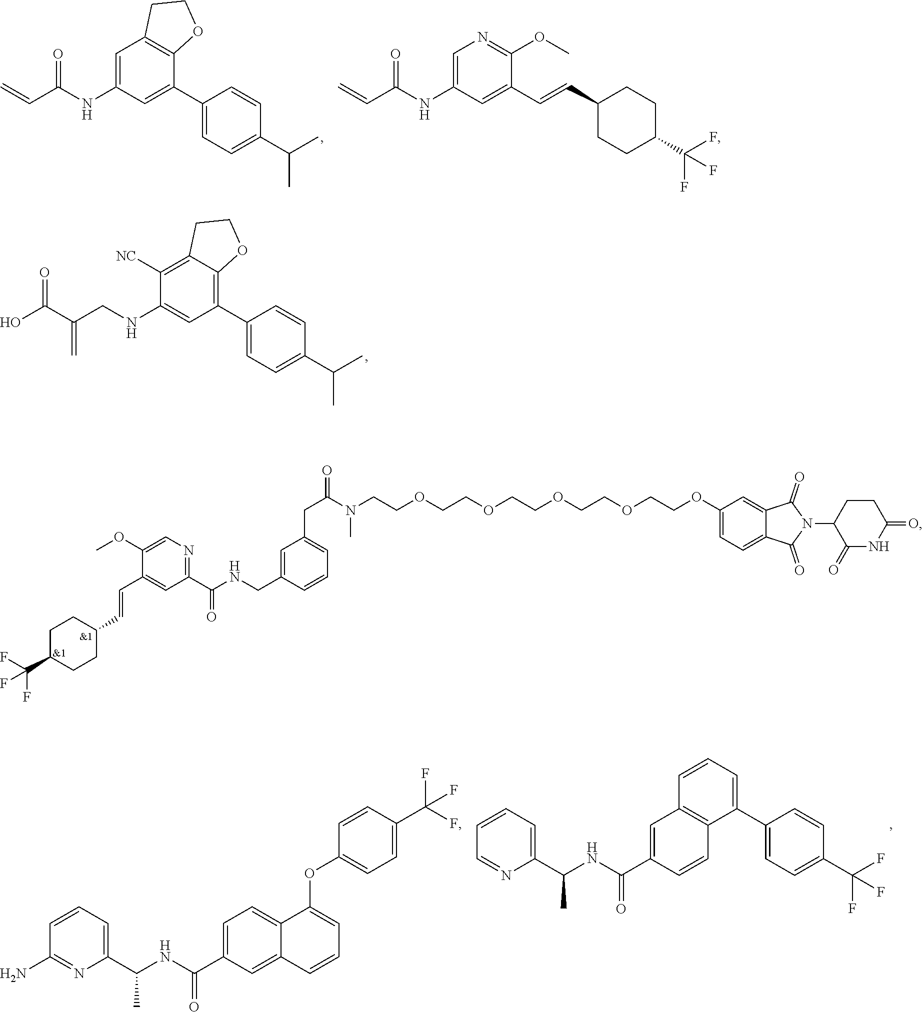

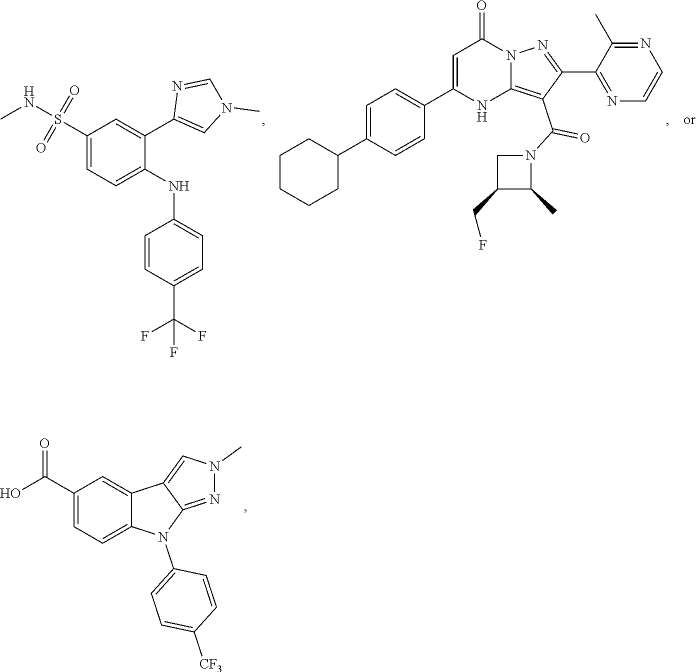

or any combination thereof, or a stereoisomer or tautomer thereof, or a pharmaceutically acceptable salt of any of the foregoing.

In some embodiments, in conjunction with embodiments above or below, the one or more TEAD inhibitors comprise a TEAD palmitate pocket binding inhibitor (e.g., any one of Compound T1, Compound T2, Compound T3, Compound T4, Compound T5, Compound T6, Compound T7, Compound T8, Compound T9, or Compound T10). TEAD palmitate pocket binding inhibitors are described in, for example, Chan et al. (Nat. Chem. Biol. 2016, 12(4): 282-289), Noland et al. (Structure, 2016, 24(1): 179-186), and Kim et al. (Biological Sciences, 2019, 116(20): 9877-9882), each of which is incorporated herein by reference in its entirety and specifically with respect to TEAD palmitate pocket binding inhibitors described therein.

In some embodiments, in conjunction with embodiments above or below, the one or more TEAD inhibitors comprise a covalent TEAD inhibitor (e.g., any one of Compound T2, Compound T3, or Compound T4). Covalent TEAD inhibitors are described in, for example, Karats et al. (J. Med. Chem. 2020, 63, 11972-11989), Lu et al. (Acta Pharmaceutica Sinica B, 2021, 11(10): 3206-3219), Fan et al. (Biorxiv, 2022, DOI:10.1101/2022.05.10.491316), and Kaneda et al. (Am. J. Cancer Res., 2020, 10(12): 4399-4415), each of which is incorporated herein by reference in its entirety and specifically with respect to covalent TEAD inhibitors described therein.

### KRAS Inhibitors

In some embodiments, in conjunction with embodiments above or below, the one or more KRAS inhibitors comprise a compound of formula (K-I):

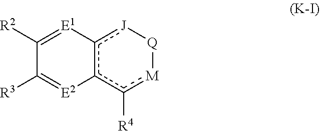

wherein E1 and E2 are each independently N or CR¹; J is N, NR¹⁰, or CR¹⁰; M is N, NR¹³, or CR¹³; [symbol] is a single or double bond as necessary to give every atom its normal valence; R¹ is independently H, hydroxy, C₁₋₄alkyl, C₁₋₄haloalkyl, C₁₋₄alkoxy, NH—C₁₋₄alkyl, N(C₁₋₄alkyl)₂, cyano, or halo; R² is halo, C₁₋₆alkyl, C₁₋₆haloalkyl, OR′, N(R′)₂, C₂₋₃alkenyl, C₂₋₃alkynyl, C₀₋₃alkylene-C₃₋₈cycloalkyl, C₀₋₃alkylene-C₂₋₇heterocycloalkyl, C₀₋₃alkylenearyl, or C₀₋₃alkyleneheteroaryl, and each R′ is independently H, C₁₋₆alkyl, C₁₋₆haloalkyl, C₃₋₄cycloalkyl, C₂₋₃alkenyl, C₂₋₃alkynyl, aryl, or heteroaryl, or two R′ substituents, together with the nitrogen atom to which they are attached, form a 3-7-membered ring; R³ is halo, C₁₋₃alkyl, C₁₋₂haloalkyl, C₁₋₃alkoxy, C₃₋₄cycloalkyl, C₂₋₃alkenyl, C₂₋₃alkynyl, aryl, or heteroaryl; R⁴ is

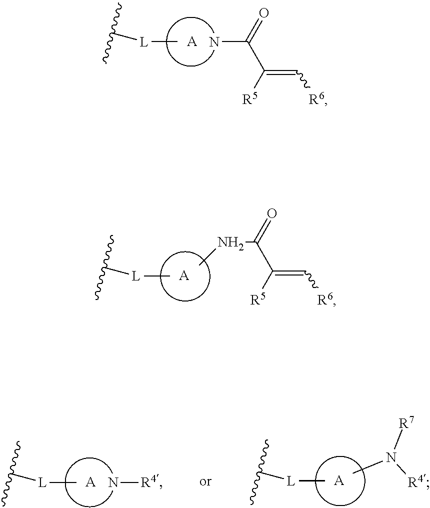

ring A is a monocyclic 4-7 membered ring or a bicyclic, bridged, fused, or spiro 6-11 membered ring; L is a bond, C₁₋₆alkylene, —O—C₀₋₅alkylene, —S—C₀₋₅alkylene, or —NH—C₀₋₅alkylene, and for C₂₋₆alkylene, —O—C₂₋₅alkylene, —S—C₂₋₅alkylene, and NH—C₂₋₅alkylene, one carbon atom of the alkylene group can optionally be replaced with O, S, or NH; R⁴, is H, C₁₋₈alkyl, C₂₋₈alkynyl, C₁₋₆alkylene-O—C₁₋₄alkyl, C₁₋₆alkylene-OH, C₁₋₆haloalkyl, C₀₋₃alkylene-C₃₋₈cycloalkyl, C₀₋₃alkylene-C₂₋₇heterocycloalkyl, C₀₋₃alkylenearyl, or selected from

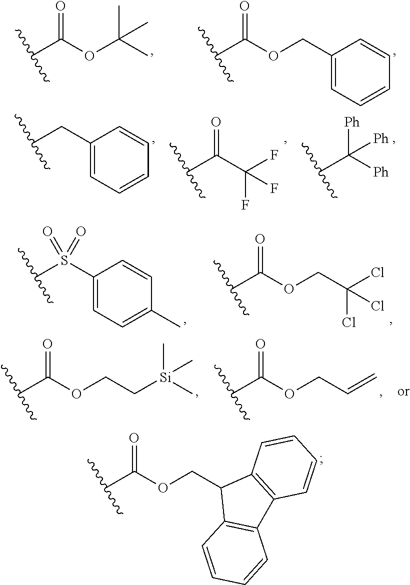

R⁵ and R⁶ are each independently H, halo, C₁₋₈alkyl, C₂₋₈alkynyl, C₁₋₆alkylene-O—C₁₋₄alkyl, C₁₋₆alkylene-OH, C₁₋₆haloalkyl, C₁₋₆alkyleneamine, C₀₋₆alkyleneamide, C₀₋₃alkylene-C(O)OH, C₀₋₃alkylene-C(O)OC₁₋₄alkyl, C₁₋₆alkylene-O-aryl, C₀₋₃alkylene-C(O)C₁₋₄alkylene-OH, C₀₋₃alkylene-C₃₋₈cycloalkyl, C₀₋₃alkylene-C₂₋₇heterocycloalkyl, C₀₋₃alkylenearyl, or cyano, or R⁵ and R⁶, together with the atoms to which they are attached, form a 4-6 membered ring; R⁷ is H or C₁₋₃alkyl, or R⁷ and R⁵, together with the atoms to which they are attached, form a 4-6 membered ring; Q is CR⁸R⁹, C═CR⁸R⁹, C═O, C═S, or C═NR⁸; R⁸ and R⁹ are each independently H, C₁₋₃alkyl, hydroxy, C₁₋₃alkoxy, cyano, nitro, or C₃₋₆cycloalkyl, or R⁸ and R⁹, taken together with the carbon atom to which they are attached, can form a 3-6 membered ring; R¹⁰ is C₁₋₈alkyl, C₀₋₃alkylenearyl, C₀₋₃alkyleneheteroaryl, C₀₋₃alkylene-C₃₋₈ cycloalkyl, C₀₋₃alkylene-C₂₋₇heterocycloalkyl, C₁₋₆alkoxy, O—C₀₋₃alkylenearyl, O—C₀₋₃alkyleneheteroaryl, O—C₀₋₃alkylene-C₃₋₈cycloalkyl, O—C₀₋₃alkylenearyl, O—C₀₋₃alkylene-C₂₋₇heterocycloalkyl, NH—C₁₋₈alkyl, N(C₁₋₈alkyl)₂, NH—C₀₋₃alkylenearyl, NH—C₀₋₃alkyleneheteroaryl, NH—C₀₋₃alkylene-C₃₋₈cycloalkyl, NH—C₀₋₃alkylene-C₂₋₇heterocycloalkyl, halo, cyano, or C₁₋₆alkyleneamine; and R¹³ is C₁₋₄alkyl, C₁₋₃haloalkyl, C₁₋₃alkyleneamine, and C₃₋₅cycloalkyl, or a stereoisomer or tautomer thereof, or a pharmaceutically acceptable salt or any of the foregoing, with the proviso that (1) when J is NR¹⁰, M is N or CR¹³; (2) when M is NR¹³, J is N or CR¹⁰; (3) when J is CR¹⁰, M is N or NR¹³; and (4) when M is CR¹³, J is N or NR¹⁰.

Description of formula (K-I) can be found in US2018/0334454A1, the entirety of which is incorporated herein by reference. Formula (K-I) is described as formula (II) in US2018/0334454A1 (see, e.g., paragraphs [0033]-[0053]), which paragraphs and description of formula (II) and methods of making compounds of formula (II) are hereby incorporated herein by reference. Moieties of formula (K-I), such as J, Q, M, E¹, E², R², R³, and R⁴ are as defined in US2018/0334454A1, including any variations or embodiments thereof.

In some embodiments, in conjunction with embodiments above or below, the one or more KRAS inhibitors comprise a compound of formula (K-I-A):

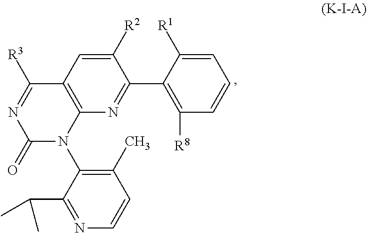

wherein
R¹ is H, halo, or —CH₃;
R² is H, halo, or —CH₃;
R³ is

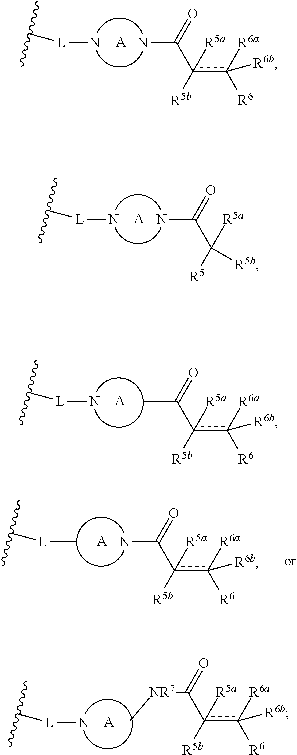

b is optionally a single or a double bond;
ring A is a monocyclic 4-7 membered ring or a bicyclic, bridged, fused, or spiro 6-11 membered ring;
L is a bond or NR⁴;
R⁴ is H, —C₁₋₆alkyl, —C₂₋₆alkynyl, C₁₋₆alkylene-O—C₁₋₄alkyl, C₁₋₆alkylene-OH, C₁₋₆haloalkyl, —C₁₋₆alkyleneamine, —C₀₋₆alkylene-amide, —C(O)OH, —C(O)OC₁₋₄alkyl, —C₁₋₆alkylene-O-aryl, —N═N, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R⁵ is H, halo, an —C₁₋₆alkyl, —C₂₋₆alkynyl, —C₀₋₆alkylene-O—C₁₋₆alkyl, —C₁₋₆alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₀₋₆ alkylene-O—C₆₋₁₄aryl, —C₀₋₃alkylene-C(O)C₁₋₄alkylene-OH, cyclocalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃-alkylene-C₆₋₁₄aryl, —C₀₋₃alkylene-C₂₋₁₄heteroaryl, or cyano;
R^{5a} is selected from H, —C₁₋₆alkyl, —C₂₋₆alkynyl, —C₁₋₆ alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R^{5b} is selected from H, —C₁₋₆alkyl, —C₂₋₆alkynyl, —C₁₋₆ alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
or R^{5a} and R^{5b} together, may represent an ═O or ═N═N;
R⁶ is H, halo, —C₁₋₆alkyl, —C₂₋₆alkynyl, —C₁₋₆alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₀₋₆alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl;
R^{5a} and R^{6a}, together with the atoms to which they are attached, may form a 3-6 membered ring that optionally includes one or two heteroatoms selected from O, S or N; or
R^{5a} and R^{6a} are absent when b is a double bond;
R^{6a} is H, or —C₁₋₆alkyl;
R^{6b} is H, —C₁₋₆alkyl, —C₂₋₆alkynyl, —C₁₋₆ alkylene-O—C₁₋₄alkyl, —C₁₋₆alkylene-OH, —C₁₋₆haloalkyl, —C₁₋₆alkyleneamine, —C₀₋₆ alkylene-amide, —C(O)OH, —C(O)OC₁₋₄alkyl, —C₀₋₆ alkylene-O—C₆₋₁₄aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, —C₀₋₃alkylene-C₂₋₁₄heteroaryl, or cyano;
or R^{6a} and R^{6b} together, may represent an ═O;
R⁷ is H or C₁₋₈alkyl;
R⁸ is H, OH, NR^{a}R^{b};
wherein R^{a} and R^{b} are each independently H, halo, —C₁₋₆alkyl, —C₂₋₆alkynyl;
wherein the ring A or the —C₁₋₆alkyl, —C₂₋₆alkynyl, —C₁₋₆ alkylene-O—C₁₋₄alkyl, —C₁₋₆alkylene-OH, —C₁₋₆haloalkyl, —C₁₋₆alkyleneamine, —C₀₋₆ alkylene-amide, —C(O)OC₁₋₄alkyl, —C₁₋₆ alkylene-O-aryl, —C₀₋₃alkylene-C(O)C₁₋₄alkylene-OH, cycloalkyl, heterocycloalkyl, aryl, heteroaryl, —C₀₋₃alkylene-C₃₋₁₄cycloalkyl, —C₀₋₃alkylene-C₂₋₁₄heterocycloalkyl, —C₀₋₃alkylene-C₆₋₁₄aryl, or —C₀₋₃alkylene-C₂₋₁₄heteroaryl groups of any of the R⁴, R⁵, R^{5a}, R^{5b}, R⁶, R^{6a}, R^{6b}, R⁷ and R⁸ may be unsubstituted or substituted with 1, 2, 3, or 4 substituents, as allowed, independently selected from halo, —C₁₋₆alkyl, —O—C₁₋₆alkyl, —OH, or —C₁₋₆alkyl-CN; or a stereoisomer or tautomer thereof, or a pharmaceutically acceptable salt of any of the foregoing.

Description of formula (K-I-A) can be found in WO2021/081212A1, the entirety of which is incorporated herein by reference. Formula (K-I) is described as formula (I) in WO2021/081212A1 (see, e.g., Embodiment 1, paragraph [0037]), which paragraphs and description of formula (I) and methods of making compounds of formula (I) are hereby incorporated herein by reference. Moieties of formula (K-I-A), such as R¹, R², R³, and R⁸ are as defined in WO2021/081212A1, including any variations or embodiments thereof.

In some embodiments, in conjunction with embodiments above or below, a compound of formula (K-I) or (K-I-A) is sotorasib (Compound K1), or a stereoisomer or tautomer thereof, or a pharmaceutically acceptable salt of any of the foregoing. Sotorasib is chemically described as 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one, having the structure below:

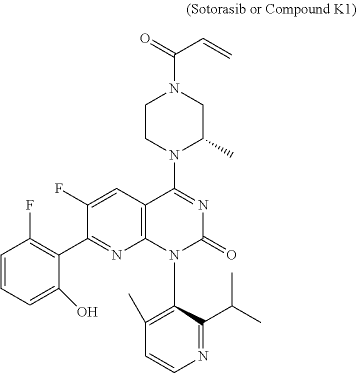

Description of sotorasib (Compound K1) and methods of making sotorasib can be found in US2018/0334454A1, the entirety of which is incorporated herein by reference. Description of sotorasib (Compound K1) and methods of making sotorasib can be found in, e.g., Example 41, pages 210-212 of US2018/0334454A1.

In some embodiments, in conjunction with embodiments above or below, the one or more KRAS inhibitors comprise a compound of formula (K-II):

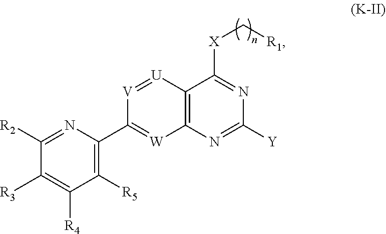

wherein,
R₁ is an electrophilic moiety capable of forming a covalent bond with a cysteine residue at position 12 of a K-Ras G12C mutant protein;
R₂ is selected from a group consisting of H, OH, NH₂, halo, C₁₋₆ alkyl, C₁₋₆ haloalkyl, cyclopropyl, and —NHR, wherein R is selected from the group consisting of C₁₋₆ alkyl, C₁₋₆ alkoxy, C₁₋₆ alkanoyl, C₁₋₆ hydroxyalkanoyl, C₁₋₆ cyanoalkyl, C₁₋₆ alkylamino, —(C₁₋₆ alkenyl)NH(CH₃)—(C₁₋₆ alkylenyl)N(CH₃)₂, and —(C₁₋₃ alkylenyl)(3-7 membered-heterocyclyl);
R₃ and R₄ are each independently selected from the group consisting of H, NH₂, halo, C₁₋₆ alkyl, C₁₋₆ haloalkyl, C₁₋₆ alkoxy, C₁₋₆ haloalkoxy, C₁₋₆ alkylthio, C₁₋₆ haloalkylthio, C₁₋₆ alkylamino, and cyclopropyl;
R₅ is selected from the group consisting of H, NH₂, halo, C₁₋₆ alkyl, C₁₋₆ haloalkyl, C₁₋₆ alkoxy, C₁₋₆ haloalkoxy, C₁₋₆ alkylthio, C₁₋₆ haloalkylthio, C₁₋₆ alkylamino, and C₃₋₇ cycloalkyl,
Wherein at least one of R₂, R₃, R₄, and R₅ is other than H; or
R₂ and R₃, R₃ and R₄, or R₄ and R₅, together with the atoms to which they are each bonded, form a C₃₋₇ cycloalkyl, 3 to 7 membered heterocycloalkyl, C₆₋₁₄ aryl, or 5- to 10-membered heteroaryl; each of which is optionally substituted with 1 to 4 substituents, wherein each substituent is independently selected from the group consisting of OH, NH₂, halo, C₁₋₃ alkyl, C₁₋₃ haloalkyl, C₁₋₃ alkoxy, and C₁₋₃ haloalkoxy;
X is selected from the group consisting of NH₂, C₁₋₆ alkoxy, C₁₋₆ alkyl, C₁₋₆ alkylamino, C₁₋₆ alkylsulfanyl, C₁₋₆ alkylthio, C₃₋₇ cycloalkyl, 4- to 7-membered heterocyclyl, and 4- to 7-membered heterocyclylamino; each of which is optionally substituted with 1 to 4 substituents, wherein each substituent is independently selected from the group consisting of OH, NH₂, halo, cyano, carboxy, carbamoyl, C₁₋₆ alkyl, C₁₋₆ aminoalkyl, C₁₋₆ carbamoylalkyl, C₁₋₆ carboxyalkyl, C₁₋₆ cyanoalkyl, C₁₋₆ haloalkyl, C₁₋₆ hydroxyalkyl, and 4- to 7-membered heterocyclyl; wherein two geminal substituents may be taken together to form C₃₋₇ spirocycloalkyl or 4- to 7-membered spiroheterocyclyl;
Y is selected from the group consisting of -L-Y₁ or Y₁;
Y₁ is selected from the group consisting of H, NH₂, halo, cyano, carbamoyl, C₂₋₆ alkenyl, C₁₋₆ alkoxy, C₁₋₆ alkyl, C₁₋₆ alkyl substituted with a 4- to 10-membered heterocyclyl that is optionally substituted with 1-4 Y₁ₐ substituents, C₁₋₆ alkyl substituted with a C₁₋₆ dialkylamino substituent, C₁₋₆ alkyl substituted with a C₁₋₆ dialkylamino cyclopropyl, C₁₋₆ alkylsulfanyl, C₁₋₆ alkylsulfonyl, C₁₋₆ alkylthio, C₂₋₆ alkynyl, C₁₋₆ alkylamino, C₆₋₁₄ aryl, C₆₋₁₄ aryl substituted with a C₁₋₆ alkyl, C₁₋₆ aminoalkyl, C₁₋₆ carbamoylalkyl, C₁₋₆ carboxyalkyl, C₁₋₆ haloalkoxy, C₁₋₆ haloalkyl, 5- to 10-membered heteroaryl, 4- to 10-membered heterocyclyl, a 4- to 10-membered heterocyclyl substituted with methyl, hydroxyl, and oxo;
each Y₁ₐ is independently selected from the group consisting of halo, C₁₋₆ alkyl, C₁₋₆ alkoxy, 3- to 7-membered heterocyclyl, C₁₋₆alkoxy C₁₋₆alkyl, C₁₋₆ haloalkyl, oxo, hydroxyl, NH₂, cyano, C₁₋₆ carboxyalkyl, C₁₋₆ cyanoalkyl, C₁₋₆ hydroxyalkyl, and C₁₋₆ haloalkoxy;
L is selected from the group consisting of a bond, O, S, and N(L^{a});
L^{a} is selected from the group consisting of hydrogen and C₁₋₃ alkyl;
U is C(R₆ₐ);
V is C(R_{6b});
W is C(R_{6c}) or N;
each of R₆ₐ, R_{6b}, and R_{6c} are independently selected from the group consisting of H, OH, NH₂, halo, cyano, carbamoyl, C₂₋₆ alkenyl, C₁₋₆ alkoxy, C₁₋₆ alkyl, C₁₋₆ alkyl substituted with a 4- to 10-membered heterocyclyl substituent, C₁₋₆ alkylsulfanyl, C₁₋₆ alkylsulfonyl, C₁₋₆ alkylthio, C₁₋₆ haloalkylthio, C₂₋₆ alkynyl, C₁₋₆ alkylamino, C₆₋₁₄ aryl, C₁₋₆ aminoalkyl, C₁₋₆ carbamoylalkyl, C₁₋₆ carboxyalkyl, C₁₋₆ cyanoalkyl, C₃₋₇ cycloalkyl, C₁₋₆ haloalkoxy, C₁₋₆ haloalkyl, 5- to 10-membered heteroaryl, and 4- to 10-membered heterocyclyl; and
n is selected from the group consisting of 0, 1, and 2;
or a stereoisomer or tautomer thereof, or a pharmaceutically acceptable salt of any of the foregoing.

Description of formula (K-II) can be found in US2021/0230142A9, the entirety of which is incorporated herein by reference. Formula (K-II) is described as Formula (I) in US2021/0230142A9 (see, e.g., paragraphs [0113]-[0132]), which paragraphs and description of Formula (I) and methods of making compounds of Formula (I) are hereby incorporated herein by reference. Moieties of formula (K-II), such as U, V, W, X, Y, R¹, R², R³, R⁴ and R⁵ are as defined in US2021/0230142A9, including any variations or embodiments thereof.

In some embodiments, in conjunction with embodiments above or below, the one or more KRAS inhibitors comprise a compound of formula (K-II-A):

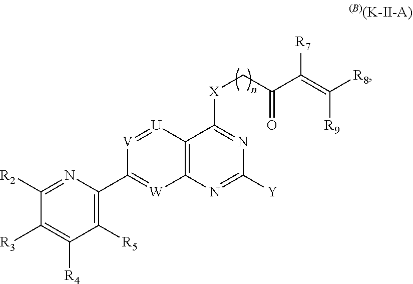

wherein,
R₂ is selected from the group consisting of H, OH, NH₂, halo, C₁₋₆ alkyl, C₁₋₆ haloalkyl, cyclopropyl, and —NHR, wherein R is selected from the group consisting of C₁₋₆ alkyl, C₁₋₆ alkoxy, C₁₋₆ alkanoyl, C₁₋₆ hydroxyalkanoyl, C₁₋₆ cyanoalkyl, C₁₋₆ alkylamino, —(C₁₋₆
alkylenyl)NH(CH₃)—(C₁₋₆ alkylenyl)N(CH₃)₂, and —(C₁₋₃ alkylenyl)(3-7 membered-heterocyclyl);
R₃ and R₄ are each independently selected from the group consisting of H, NH₂, halo, C₁₋₆ alkyl, C₁₋₆ haloalkyl, C₁₋₆ alkoxy, C₁₋₆ haloalkoxy, C₁₋₆ alkylthio, C₁₋₆ haloalkylthio, C₁₋₆ alkylamino, and cyclopropyl;
R₅ is selected from the group consisting of H, NH₂, halo, C₁₋₆ alkyl, C₁₋₆ haloalkyl, C₁₋₆ alkoxy, C₁₋₆ haloalkoxy, C₁₋₆ alkylthio, C₁₋₆ haloalkylthio, C₁₋₆ alkylamino, and C₃₋₇ cycloalkyl,
wherein at least one of R₂, R₃, R₄, and R₅ is other than H; or
R₂ and R₃, R₃ and R₄, or R₄ and R₅, together with the atoms to which they are each bonded, form a C₃₋₇ cycloalkyl, 3 to 7 membered heterocycloalkyl, C₆₋₁₄ aryl, or 5- to 10-membered heteroaryl; each of which is optionally substituted with 1 to 4 substituents, wherein each substituent is independently selected from the group consisting of OH, NH₂, halo, C₁₋₃ alkyl, C₁₋₃ haloalkyl, C₁₋₃ alkoxy, and C₁₋₃ haloalkoxy;
R₇ is selected from the group consisting of H, cyano, and halo; and R₈ and R₉ are each independently selected from the group consisting of H, C₁₋₆ alkyl, C₁₋₆ haloalkyl, C₁₋₆ hydroxyalkyl, cyano, and halo; wherein C₁₋₆ alkyl is optionally substituted with one substituent selected from the group consisting of: methanesulfonyl (mesyl), p-toluenesulfonyl (tosyl), an alkyl or aryl sulfonate leaving group, C₁₋₆ alkanoylamino, C₁₋₆ alkoxy, C₁₋₆ alkylamino, C₁₋₆ alkylsulfonylamino, C₆₋₁₂ dialkylamino, and C₁₋₆ haloalkoxy;
or
R₇ and R₈ together form a triple bond between the carbons to which they are attached, or R₇ and R₈ together with the carbons to which they are each bonded form a C₃₋₇ cycloalkenyl optionally substituted with one or two halo substituents; and R₉ is selected from the group consisting of H, C₁₋₆ alkyl, C₁₋₆ haloalkyl, cyano, and halo; wherein C₁₋₆ alkyl is optionally substituted with one substituent selected from the group consisting of: C₁₋₆ alkanoylamino, C₁₋₆ alkoxy, C₁₋₆ alkylamino, C₁₋₆ alkylsulfonylamino, C₆₋₁₂ dialkylamino, and C₁₋₆ haloalkoxy;
X is selected from the group consisting of NH₂, C₁₋₆ alkoxy, C₁₋₆ alkyl, C₁₋₆ alkylamino, C₁₋₆ alkylsulfanyl, C₁₋₆ alkylsulfonyl, C₁₋₆ alkylthio, C₃₋₇ cycloalkyl, 4- to 7-membered heterocyclyl, and 4- to 7-membered heterocyclylamino; each of which is optionally substituted with 1 to 4 substituents, wherein each substituent is independently selected from the group consisting of OH, NH₂, halo, cyano, carboxy, carbamoyl, C₁₋₆ alkyl, C₁₋₆ aminoalkyl, C₁₋₆ carbamoylalkyl, C₁₋₆ carboxyalkyl, C₁₋₆ cyanoalkyl, C₁₋₆ haloalkyl, C₁₋₆ hydroxyalkyl, and 4- to 7-membered heterocyclyl; wherein two geminal substituents may be taken together to form C₃₋₇ spirocycloalkyl or 4- to 7-membered spiroheterocyclyl;
Y is selected from the group consisting of -L-Y₁ or Y₁;
Y₁ is selected from the group consisting of H, NH₂, halo, cyano, carbamoyl, C₂₋₆ alkenyl, C₁₋₆ alkoxy, C₁₋₆ alkyl, C₁₋₆ alkyl substituted with a 4- to 10-membered heterocyclyl that is optionally substituted with 1-4 Y₁ₐ substituents, C₁₋₆ alkyl substituted with a C₁₋₆ dialkylamino substituent, C₁₋₆ alkyl substituted with a C₁₋₆ dialkylamino cyclopropyl, C₁₋₆ alkylsulfanyl, C₁₋₆ alkylsulfonyl, C₁₋₆ alkylthio, C₂₋₆ alkynyl, C₁₋₆ alkylamino, C₆₋₁₄ aryl, C₆₋₁₄ aryl substituted with a C₁₋₆ alkyl, C₁₋₆ aminoalkyl, C₁₋₆ carbamoylalkyl, C₁₋₆ carboxyalkyl, C₁₋₆ cyanoalkyl, C₃₋₇ cycloalkyl, C₃₋₇ cycloalkyl substituted with a C₁₋₆ dialkylamino, C₁₋₆ haloalkoxy, C₁₋₆ haloalkyl, 5- to 10-membered heteroaryl, 4- to 10-membered heterocyclyl, 4- to 10-membered heterocyclyl substituted with methyl, hydroxy, and oxo;
each Y₁ₐ is independently selected from the group consisting of halo, C₁₋₆ alkyl, C₁₋₆ alkoxy, 3- to 7-membered heterocyclyl, C₁₋₆alkoxyC₁₋₆alkyl, C₁₋₆ haloalkyl, oxo, hydroxy, NH₂, cyano, C₁₋₆ carboxyalkyl, C₁₋₆ cyanoalkyl, C₁₋₆ hydroxyalkyl, and C₁₋₆ haloalkoxy;
L is selected from the group consisting of a bond, O, S, and N(L^{a});
L^{a} is selected from the group consisting of hydrogen and C₁₋₃ alkyl;
U is C(R₆ₐ);
V is C(R_{6b});
W is C(R_{6c}) or N;
each of R₆ₐ, R_{6b}, and R_{6c} are independently selected from the group consisting of H, OH, NH₂, halo, cyano, carbamoyl, C₂₋₆ alkenyl, C₁₋₆ alkoxy, C₁₋₆ alkyl, C₁₋₆ alkyl substituted with a 4- to 10-membered heterocyclyl substituent, C₁₋₆ alkylsulfanyl, C₁₋₆ alkylsulfonyl, C₁₋₆ alkylthio, C₁₋₆ haloalkylthio, C₂₋₆ alkynyl, C₁₋₆ alkylamino, C₆₋₁₄ aryl, C₁₋₆ aminoalkyl, C₁₋₆ carbamoylalkyl, C₁₋₆ carboxyalkyl, C₁₋₆ cyanoalkyl, C₃₋₇ cycloalkyl, C₁₋₆ haloalkoxy, C₁₋₆ haloalkyl, 5- to 10-membered heteroaryl, and 4- to 10-membered heterocyclyl; and
n is selected from the group consisting of 0, 1, and 2;
or a stereoisomer or tautomer thereof, or a pharmaceutically acceptable salt of any of the foregoing.

Formula (K-II-A) is described as Formula (II) in, e.g., paragraph [0137] of US2021/0230142A9, which paragraphs and description of Formula (II) and methods of making compounds of Formula (II) are hereby incorporated herein by reference. Moieties of formula (K-II-A), such as U, V, W, X, Y, R¹, R², R³, R⁴, R⁵, R⁷, R⁸, and R⁹ are as defined in US2021/0230142A9, including any variations or embodiments thereof.

In some embodiments, in conjunction with embodiments above or below, the one or more KRAS inhibitors comprise a compound of formula (K-II-B) or (K-II-C):

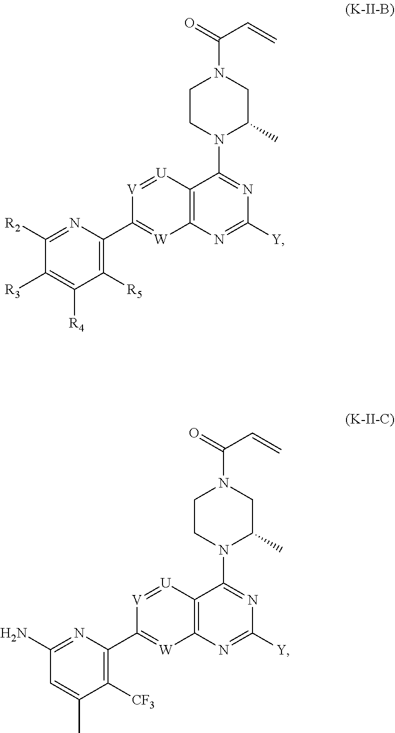

or a stereoisomer or tautomer thereof, or a pharmaceutically acceptable salt of any of the foregoing, wherein U, V, W, Y, R₂, R₃, R₄, and R₅ are as defined in Formula (K-II). It is understood that U, V, W, Y, R₂, R₃, R₄, and R₅ of such embodiments of compounds of Formulae (K-II-B) and (K-II-C) may include U, V, W, Y, R₂, R₃, R₄, and R₅ as described for Formula (K-II). Formulae (K-II-B) and (K-II-C) are described as Formulae (Ib) and (IVb), respectively, in, e.g., paragraphs [0277] and [0285] of US2021/0230142A9, which paragraphs and description of Formula (Ib) or (IVb) and methods of making compounds of Formula (Ib) or (IVb) are hereby incorporated herein by reference. Moieties of formulae (K-II-B) and (K-II-C), such as U, V, W, Y, R², R³, R⁴, and R⁵ are as defined in US2021/0230142A9, including any variations or embodiments thereof.

In some embodiments, in conjunction with embodiments above or below, a compound of formula (K-II), (K-II-A), (K-II-B), or (K-II-C) is Compound K2, or a stereoisomer or tautomer thereof, or a pharmaceutically acceptable salt of any of the foregoing. Compound K2 is chemically described as 1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one, having the structure below:

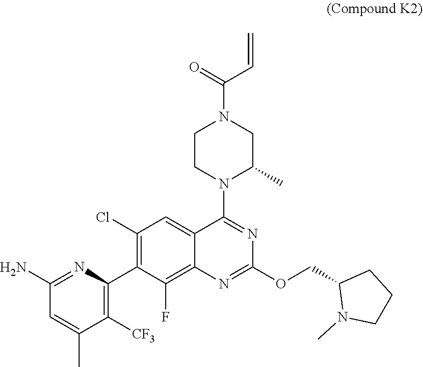

Description of Compound K2 and methods of making Compound K2 can be found in, e.g., Example 17a & 17b on pages 130 to 135 of US2021/0230142A9.

In some embodiments, in conjunction with embodiments above or below, the one or more KRAS inhibitors comprise a compound of formula (K-III):

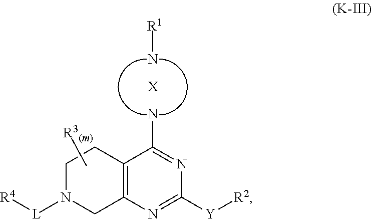

wherein:
X is a 4-12 membered saturated or partially saturated monocyclic, bridged or spirocyclic ring, wherein the saturated or partially saturated monocyclic ring is optionally substituted with one or more R⁸,⋅
Y is a bond, O, S or NR⁵;
R¹ is —C(O)C(R^{A}) [symbol] C(R^{B})ₚ or —SO₂C(R^{A}) [symbol] C(R^{B})ₚ;
R² is hydrogen, alkyl, hydroxyalkyl, dihydroxyalkyl, alkylaminylalkyl, dialkylaminylalkyl, —Z—NR⁵R¹⁰, heterocyclyl, heterocyclylalkyl, aryl, heteroaryl, or heteroarylalkyl, wherein each of the Z, heterocyclyl, heterocyclylalkyl, aryl, heteroaryl, and heteroarylalkyl may be optionally substituted with one or more R⁹;
each Z is C₁-C₄ alkylene;
each R³ is independently C₁-C₃ alkyl, oxo, haloalkyl, hydroxyl or halogen;
L is a bond, —C(O)—, or C₁-C₃ alkylene;
R⁴ is hydrogen, cycloalkyl, heterocyclyl, aryl, aralkyl or heteroaryl, wherein each of the cycloalkyl, heterocyclyl, aryl, aralkyl and heteroaryl may be optionally substituted with one or more R⁶, R⁷ or R⁸;
each R⁵ is independently hydrogen or C1-C3 alkyl;
R⁶ is cycloalkyl, heterocyclyl, heterocyclylalkyl, aryl, or heteroaryl, wherein each of the cycloalkyl, heterocyclyl, aryl, or heteroaryl may be optionally substituted with one or more R⁷;
each R⁷ is independently halogen, hydroxyl, C1-C6 alkyl, cycloalkyl, alkoxy, haloalkyl, amino, cyano, heteroalkyl, hydroxyalkyl or Q-haloalkyl, wherein Q is O or S;
R⁸ is oxo, C1-C3 alkyl, C2-C4 alkynyl, heteroalkyl, cyano, —C(O)OR⁵, —C(O)N(R⁵)₂, —N(R⁵)₂, wherein the C1-C3 alkyl may be optionally substituted with cyano, halogen, —OR⁵, —N(R₅)₂, or heteroaryl;
each R⁹ is independently hydrogen, oxo, acyl, hydroxyl, hydroxyalkyl, cyano, halogen, C1-C6 alkyl, aralkyl, haloalkyl, heteroalkyl, cycloalkyl, heterocyclyl, heterocyclylalkyl, alkoxy, dialkylaminyl, dialkylamidoalkyl, or dialkylaminylalkyl, wherein the C1-C6 alkyl may be optionally substituted with cycloalkyl;
each R¹⁰ is independently hydrogen, acyl, C1-C3 alkyl, heteroalkyl or hydroxyalkyl;
R¹¹ is haloalkyl;
R^{A} is absent, hydrogen, deuterium, cyano, halogen, C1-C3 alkyl, haloalkyl, heteroalkyl, —C(O)N(R⁵)₂, or hydroxyalkyl;
each R^{B} is independently hydrogen, deuterium, cyano, C1-C3 alkyl, hydroxyalkyl, heteroalkyl, C1-C3 alkoxy, halogen, haloalkyl, —ZNR⁵R¹¹, —C(O)N(R⁵)₂, —NHC(O)C1-C3 alkyl, —CH₂NHC(O)C1-C3 alkyl, heteroaryl, heteroarylalkyl, dialkylaminylalkyl, or heterocyclylalkyl wherein the heterocyclyl portion is substituted with one or more substituents independently selected from halogen, hydroxyl, alkoxy and C1-C3 alkyl, wherein the heteroaryl or the heteroaryl portion of the heteroarylalkyl is optionally substituted with one or more R⁷;
or when [symbol] is a double bond and p is two, one R^{B} is hydrogen and R^{A} and one R^{B} and the carbon atoms to which they are attached form a 4-8 membered partially saturated cycloalkyl substituted with oxo;
m is zero or an integer between 1 and 2;
p is one or two; and wherein,
when [symbol] is a triple bond then R^{A} is absent, p equals one and R^{B} is hydroxyalkyl,
or when [symbol] is a double bond then R^{A} is present, R^{B} is present and p equals two, wherein when R^{A} is hydrogen or C₁-C₃ alkyl, at least one R_{B} is deuterium, cyano, halogen, haloalkyl, hydroxyalkyl, heteroalkyl, heteroaryl, heteroarylalkyl, —ZNR⁵R¹¹, —C(O)N(R⁵)₂, —NHC(O)C1-C3 alkyl, —CH₂NHC(O)C1-C3 alkyl or heterocyclylalkyl, wherein the heterocyclyl portion is substituted with one or more substituents independently selected from halogen, hydroxyl, alkoxy and C1-C3 alkyl; or when each R^{B} is hydrogen, then R^{A} is deuterium, cyano, halogen, haloalkyl, —C(O)N(R⁵)₂, hydroxyalkyl or heteroalkyl;
or a stereoisomer or tautomer thereof, or a pharmaceutically acceptable salt of any of the foregoing.

Description of formula (K-III) can be found in US2019/0144444A1, the entirety of which is incorporated herein by reference. Formula (K-III) is described as Formula (II) in US2019/0144444A1 (see, e.g., paragraphs [0169]-[0193]), which paragraphs and description of Formula (II) and methods of making compounds of Formula (II) are hereby incorporated herein by reference. Moieties of formula (K-III), such as X, Y, L, m, R¹, R², R³, and R⁴ are as defined in US2019/0144444A1, including any variations or embodiments thereof.

In some embodiments, in conjunction with embodiments above or below, the one or more KRAS inhibitors comprise a compound of formula (K-III-A):

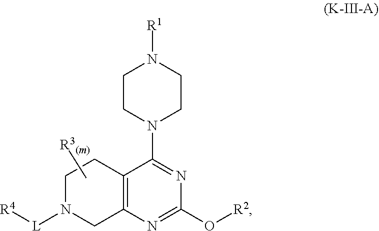

where the piperazinyl ring is optionally substituted with R⁸; or a stereoisomer or tautomer thereof, or a pharmaceutically acceptable salt of any of the foregoing, wherein R¹, R³, R⁴, R¹, L, and m are as defined in Formula (K-III). It is understood that R¹, R³, R⁴, R⁸, L, and m of such embodiments of compounds of Formula (K-III-A) may include R¹, R³, R⁴, R⁸, L, and m as described for Formula (K-III). Formula (K-III-A) is described as Formula (II-B) in, e.g., paragraphs [0231]-[0241] of US2019/0144444A1, which paragraphs and description of Formula (II-B) and methods of making compounds of Formula (II-B) are hereby incorporated herein by reference. Moieties of formula (K-III-A), such as L, m, R¹, R², R³, and R⁴ are as defined in US2019/0144444A1, including any variations or embodiments thereof.

In some embodiments, in conjunction with embodiments above or below, a compound of formula (K-III) or (K-III-A) is adagrasib (Compound K3), or a stereoisomer or tautomer thereof, or a pharmaceutically acceptable salt of any of the foregoing. Adagrasib is chemically described as 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoroacryloyl)piperazin-2-yl)acetonitrile, having the structure below:

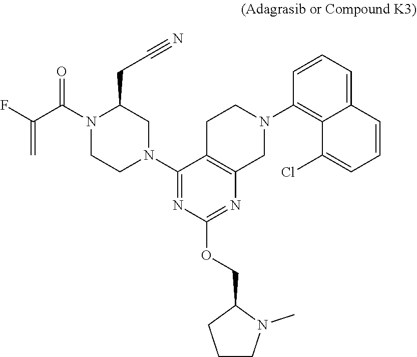 [SMILES: C=C(F)C(=O)N1CCN(c2nc(OC[C@@H]3CCCN3C)nc3c2CCN(c2cccc4cccc(Cl)c24)C3)C[C@@H]1CC#N]

Description of adagrasib (Compound K3) and methods of making adagrasib can be found in, e.g., Example 478 on pages 668-669 of US2019/0144444A1.

In some embodiments, in conjunction with embodiments above or below, the one or more KRAS inhibitors comprise a compound of formula (K-IV):

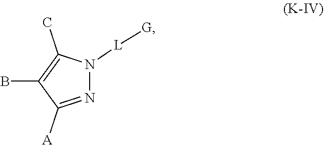

wherein,
A is selected from the group consisting of:
(a) C₅-C₇ cycloalkylene which is unsubstituted or substituted with one or more, preferably 1, 2 or 3, substituents independently selected from fluoro and C₁-C₄ alkyl;
(b) a 5-7 membered unsaturated heterocyclyl containing one carbon-carbon double bond and one oxygen atom as ring member, wherein said heterocyclyl is unsubstituted or substituted with one or more, preferably 1, 2 or 3, substituents, independently selected from fluoro and C₁-C₄ alkyl, preferably 1, 2 or 3, C₁-C₄ alkyl;
(c) C₆-C₁₀ aryl which is unsubstituted or substituted with 1, 2 or 3 R^{A2};
(d) a 5-6 membered heteroaryl ring containing 1, 2 or 3 heteroatoms independently selected from N, O and S as ring members, wherein said heteroaryl ring is unsubstituted or substituted on one or more (e.g., 1, 2 or 3) carbon atoms with R^{A3}, and wherein a nitrogen atom, when present in the heteroaryl ring, is unsubstituted or substituted with a substituent selected from the group consisting of: C₁-C₄ alkyl, —(CH₂)₁₋₂—C₃₋₄-cycloalkyl, C₃-C₆ cycloalkyl, hydroxy-C₁-C₄ alkyl, fluoro-C₁-C₄ alkyl, C₁-C₄ alkoxy-C₁-C₄ alkyl, N(R⁹)(R¹⁰)—C₁-C₄ alkyl, —SO₂—C₁-C₄ alkyl, —SO₂—C₃-C₄ cycloalkyl, —(CH₂)ₚ-Het^{py}, and —(CH₂)ₚ—N(R⁹)(R¹⁰);
(e) an 8-10 membered heteroaryl ring containing 1 to 3 heteroatoms independently selected from nitrogen, oxygen and sulfur, or an 8-10 membered partially saturated hetero-bicyclic ring containing 1 to 3 heteroatoms or heteroatom groups independently selected from 0-3 nitrogen atoms, 0-2 oxygen atoms, 0-1 sulfur atom and 0-1 S(═O)₂ group in the hetero-bicyclic ring, wherein said heteroaryl ring or hetero-bicyclic ring is unsubstituted or substituted on a carbon atom with 1, 2, 3, 4, or 5 R^{A4}, and wherein the hetero-bicyclic ring is further optionally substituted on a carbon atom by oxo and wherein a nitrogen atom, when present, is unsubstituted or substituted with a substituent which is —(CO)—C₁-C₄ alkyl or C₁-C₄ alkyl, and wherein said C₁-C₄ alkyl is optionally substituted with 1 or 2 substituents independently selected from cyano, hydroxy, oxo, fluoro, C₁-C₄ alkoxy, C₁-C₄ alkoxy-C₁-C₄ alkyl-oxy, Het^{b} and NR⁹R¹⁰; and
wherein Het^{b} is a 4- or 5- or 6-membered heterocyclic ring comprising 1 or 2 heteroatoms or groups independently selected from N, O, S, SO and SO₂, wherein said heterocyclic ring Het^{b} is unsubstituted or substituted on a carbon atom with one or two substituents independently selected from C₁-C₄ alkyl, hydroxy, cyano, fluoro, C₁-C₄ alkoxy-hydroxy-C₁-C₄ alkyl, hydroxy-C₁-C₄ alkyl, C₁-C₄ alkoxy, fluoro-C₁-C₄ alkoxy and fluoro C₁-C₄ alkyl, and wherein said heterocyclic ring Het^{b} is further optionally substituted on a carbon atom by oxo, and wherein the nitrogen atom when present in Het^{b} is optionally further substituted with C₁-C₄ alkyl which is optionally substituted with 1 to 3 substituents independently selected from fluoro, hydroxy and C₁-C₄ alkoxy;
wherein A is attached to the rest of the compound of Formula (I) by a carbon atom on A which is sp² hybridized;
wherein
B is selected from the group consisting of B¹ and B²;
wherein B¹ is C₆₋₁₀ aryl which is unsubstituted or substituted with 1, 2, 3 or 4 R^{Ba};
B² is a 6-13 membered heteroaryl which comprises 1, 2 or 3 nitrogen atoms, wherein B² is unsubstituted or substituted with 1, 2, 3 or 4 R^{Bb};
C is selected from the group consisting of hydrogen, C₁-C₃ alkyl, C₃-C₅ cycloalkyl, fluoro-C₁-C₃ alkyl, cyano, —CH₂—CN, —CH(CN)—CH₃, —CH₂—OH, —CH(OH)—CH₃ and halo;
L is selected from the group consisting of:

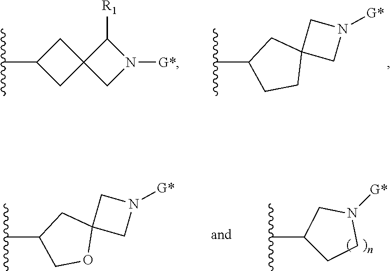

wherein n is 1, 2 or 3,
R_{L} is selected from hydrogen, methyl, ethyl, —CH₂—CN and —CH₂—OH, where
G* represents the point of attachment to G;
G is selected from the group consisting of

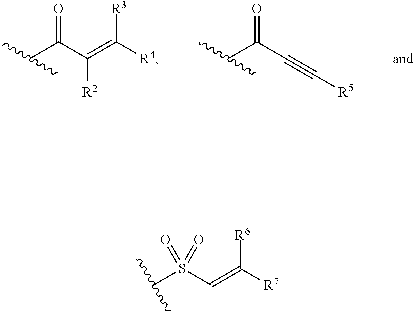

wherein
R² is selected from hydrogen, C₁-C₃ alkyl, —C(O)—C₁-C₃ alkyl, and fluoro;
R³ is hydrogen;
R⁴ is selected from hydrogen, methyl, —CH₂F, —CH₂—OCH₃ and —CH₂—N(CH₃)₂;
R⁵ is selected from hydrogen and methyl;
R⁶ is hydrogen;
R⁷ is selected from hydrogen and methyl;
wherein R^{A2} is independently selected from the group consisting of: NR⁹R¹⁰, cyano, —(CH₂)ₚ—CN, halo, OH, hydroxy-C₁-C₄ alkyl, —(COOH), —(CH₂)ₚ—COOH, C₁-C₄ alkyl, fluoro-C₁-C₄ alkyl, C₁-C₄ alkoxy, C₁-C₄ alkoxy-C₁-C₄ alkyl, N(R⁹)(R¹⁰)—C₁-C₄alkyl, N(R⁹)(R¹⁰)—C₁-C₄ alkyl-oxy, N(R⁹)(R¹⁰)—C₁-C₄ alkoxy, C₁-C₄ alkyl-carbonyl-oxy-C₁-C₄ alkyl-oxy, hydroxy-C₁-C₄ alkyl-oxy, C₁-C₄ alkoxy-C₁-C₄ alkyl-oxy, C₁-C₄ alkoxy-C₁-C₄ alkyl-oxy-C₁-C₄ alkyl, —SO₂—C₁-C₄ alkyl, —SO₂—C₃-C₄ cycloalkyl, —(CH₂)₁₋₂—C₃-C₄ cycloalkyl, Het^{py}, —(CH₂)ₚ-Het^{py}, —C(═O)—NR⁹R¹⁰, —(CH₂)ₚ—C(═O)NR⁹R¹⁰;
wherein R^{A3} is independently selected from the group consisting of oxo, NR⁹R¹⁰, cyano, —(CH₂)ₚ—CN, halo, OH, hydroxy-C₁-C₄ alkyl, —(COOH), —(CH₂)ₚ—COOH, C₁-C₄alkyl, fluoro-C₁-C₄ alkyl, C₁-C₄ alkoxy, C₁-C₄ alkoxy-C₁-C₄ alkyl, N(R⁹)(R¹⁰)—C₁-C₄ alkyl, N(R⁹)(R¹⁰)—C₁-C₄ alkyl-oxy, N(R⁹)(R¹⁰)—C₁-C₄ alkoxy, C₁-C₄ alkyl-carbonyl-oxy-C₁-C₄ alkyl-oxy, hydroxy-C₁-C₄ alkyl-oxy, C₁-C₄ alkoxy-C₁-C₄ alkyl-oxy, C₁-C₄ alkoxy-C₁-C₄ alkyl-oxy-C₁-C₄ alkyl, —SO₂—C₁-C₄ alkyl, —SO₂—C₃-C₄ cycloalkyl, —(CH₂)₁₋₂—C₃-C₄ cycloalkyl, Het^{py}, —(CH₂)ₚ-Het^{P}Y, —C(═O)—NR⁹R¹⁰, —(CH₂)ₚ—C(═O)NR⁹R¹⁰, (CH₂)ₚ—NR⁹R¹⁰;
wherein R^{A4} is independently selected from the group consisting of cyano, CO₂H, halo, C₁-C₄ alkyl, fluoro-C₁-C₄ alkyl, hydroxy, hydroxy-C₁-C₄ alkyl, hydroxy-C₁-C₄ alkyl-oxy, C₁-C₄ alkoxy, C₁-C₄ alkoxy-C₁-C₄ alkyl, C₁-C₄ alkoxy-C₁-C₄ alkyl-oxy, NR⁹R¹⁰, N(R⁹)(R¹⁰)—C₁-C₄ alkyl, N(R⁹)(R¹⁰)—C₁-C₄ alkyl-oxy, —(CO)—C₁-C₄ alkyl, and R⁹R¹⁰N—C₁-C₄ alkyl-oxy-(CO)—C₁-C₄ alkyl;
wherein
p is 1 or 2 or 3;
R⁹ is selected from hydrogen and C₁-C₄ alkyl;
R¹⁰ is selected from the group consisting of hydrogen, C₁-C₄ alkyl, hydroxyl-C₁-C₄ alkyl, C₁-C₄ alkoxy-C₁-C₄ alkyl and di-C₁-C₄ alkyl-amino-C₁-C₄ alkyl;
Het^{py} is a 4-, 5-, 6- or 7-membered saturated heterocyclic ring comprising one or two heteroatoms independently selected from O, N and S, or comprising an S-oxide (SO) or S-dioxide (SO₂) group, and wherein said heterocyclic ring is optionally substituted with oxo on one carbon atom, and wherein said heterocyclic ring is optionally further substituted on one or more carbon atoms with 1, 2 or 3 substituents independently selected from C₁-C₄ alkoxy, halo, C₁-C₄ alkyl, hydroxy-C₁-C₄ alkyl, and fluoro-C₁-C₄ alkyl, and wherein the nitrogen atom, if present in said heterocycle, is optionally further substituted with R¹⁰;
or Het^{py} is a 5- or 6-membered heteroaryl ring, comprising 1, 2 or 3 nitrogen atoms and wherein said heteroaryl ring is optionally substituted with one or more (e.g., 1, 2 3) substituents independently selected from NR⁹R¹⁰, —C(═O)—NR⁹R¹⁰, halo, C₁-C₄ alkyl, hydroxy-C₁-C₄ alkyl, fluoro-C₁-C₄ alkyl, cyano, OH, and C₁-C₄ alkoxy;
each R^{Ba} is independently selected from the group consisting of hydroxy, NH₂, C₁-C₄ alkyl and halo;
each R^{Bb} is independently selected from the group consisting of C₁-C₄ alkyl, cyclopropyl, fluoro-C₁-C₃ alkyl, cyano, halo, NH₂, and C₁-C₃ alkoxy,
or a stereoisomer or tautomer thereof, or a pharmaceutically acceptable salt of any of the foregoing.

Description of formula (K-IV) can be found in WO2021/124222A1, the entirety of which is incorporated herein by reference. Formula (K-IV) is described as Formula (I) in WO2021/124222A1 (see, e.g., pages 5-13 and Embodiment 1 pages 29-32), which paragraphs and description of Formula (I) and methods of making compounds of Formula (I) are hereby incorporated herein by reference. Moieties of formula (K-IV), such as A, B, C, L, and G are as defined in WO2021/124222A1, including any variations or embodiments thereof.

In some embodiments, in conjunction with embodiments above or below, the one or more KRAS inhibitors comprise a compound of formula (K-IV-A):

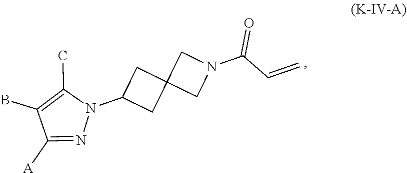

or a stereoisomer or tautomer thereof, or a pharmaceutically acceptable salt of any of the foregoing, wherein A, B, and C are as defined in formula (K-IV). It is understood that A, B, and C of such embodiments of compounds of Formula (K-IV-A) may include A, B, and C as described for Formula (K-IV). Formula (K-IV-A) is described as formula (Ia) in, e.g., Embodiment 21, of WO2021/124222A1, which paragraphs and description of formula (Ia) and methods of making compounds of formula (Ia) are hereby incorporated herein by reference. Moieties of formula (K-IV-A), such as A, B, and C are as defined in WO2021/124222A1, including any variations or embodiments thereof.

In some embodiments, in conjunction with embodiments above or below, the one or more KRAS inhibitors comprise a compound of formula (K-IV-B) or (K-IV-C):

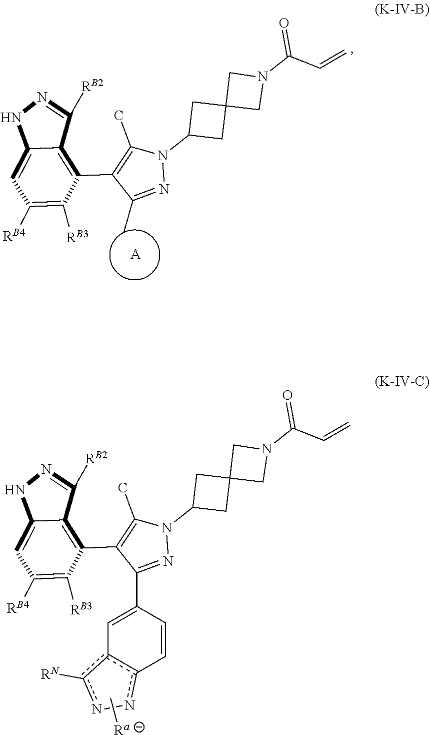

wherein,
R^{B2} is independently selected from hydrogen, halo, C₁-C₄-alkyl, cyclopropyl and NH₂;
R^{B3} is independently selected from hydrogen, halo, cyclopropyl and C₁-C₄-alkyl;
R^{B4} is independently selected from hydrogen, halo and C₁-C₄-alkyl, or R^{B3} and R^{B4} together with the atoms to which they are attached, form a 4-6 membered ring fused to the aromatic ring to which R^{B3} and R^{B4} are attached;
R^{N} is hydrogen, halo, C₁₋₄alkyl, or halo or fluoro-C₁₋₄alkyl;
R^{ae} is selected from the group consisting of hydrogen and C₁₋₄alkyl, wherein said alkyl is optionally substituted with 1 or 2 substituents selected from cyano, hydroxyl, fluoro, C₁₋₄alkoxy, C₁₋₄alkoxy-C₁₋₄alkyl-oxy, Het^{b} and NR⁹R¹⁰;
R⁹ is selected from hydrogen and C₁₋₄alkyl;
R¹⁰ is selected from hydrogen, C₁₋₄alkyl, hydroxy-C₁₋₄alkyl, C₁₋₄alkoxy-C₁₋₄alkyl and di-C₁₋₄ alkyl-amino-C₁₋₄alkyl;
wherein Het^{b} is a 4- or 5- or 6-membered heterocyclic ring comprising 1 or 2 heteroatoms or groups independently selected from N, O, S, SO and SO₂, wherein said heterocyclic ring Het^{b} is unsubstituted or substituted on a carbon atom with one or two substituents independently selected from C₁₋₄alkyl, hydroxy, cyano, fluoro, hydroxy-C₁₋₄alkyl, C₁₋₄alkoxy and fluoro-C₁₋₄alkyl, and wherein said heterocyclic ring Het^{b} is further optionally substituted on a carbon atom by oxo, and wherein the nitrogen atom when present in Het^{b} is optionally further substituted with C₁₋₄alkyl which is optionally substituted with 1 to 3 substituents independently selected from fluoro, hydroxy and C₁₋₄alkoxy;
or a stereoisomer or tautomer thereof, or a pharmaceutically acceptable salt of any of the foregoing, wherein A and C are as defined in Formula (K-IV). It is understood that A and C of such embodiments of compounds of Formulae (K-IV-B) and (K-IV-C) may include A and C as described for Formula (K-IV).

Formulae (K-IV-B) and (K-IV-C) are described as formula (Ib*) and (Id*), respectively in, e.g., Embodiment 39 and 41, of WO2021/124222A1, which paragraphs and description of formula (Ib*) or (Id*) and methods of making compounds of formula (Ib*) or (Id*) are hereby incorporated herein by reference. Moieties of formula (K-IV-B) or (K-IV-C), such as A, C, R^{B2}, R^{B3}, R^{B4}, R^{N}, and R^{ae} are as defined in WO2021/124222A1, including any variations or embodiments thereof.

In some embodiments, in conjunction with embodiments above or below, a compound of formula (K-IV), (K-IV-A), (K-IV-B), or (K-IV-C) is Compound K4, or a stereoisomer or tautomer thereof, or a pharmaceutically acceptable salt of any of the foregoing. Compound K4 is chemically described as 1-[6-[4-(5-chloro-6-methyl-1H-indazol-4-yl)-5-methyl-3-(1-methylindazol-5-yl)pyrazol-1-yl]-2-azaspiro[3.3]heptan-2-yl]prop-2-en-1-one, having the structure below:

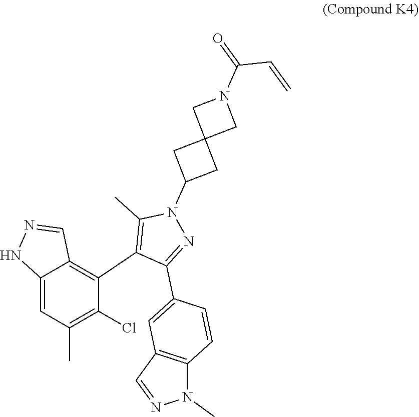 [SMILES: C=CC(=O)N1CC2(CC(n3nc(-c4ccc5c(cnn5C)c4)c(-c4c(Cl)c(C)cc5[nH]ncc45)c3C)C2)C1]

Description of Compound K4 and methods of making Compound K4 can be found in, e.g., Method 1-Synthetic Scheme on pages 111 to 114 of WO2021/124222A1.

In some embodiments, the one or more KRAS inhibitors comprise

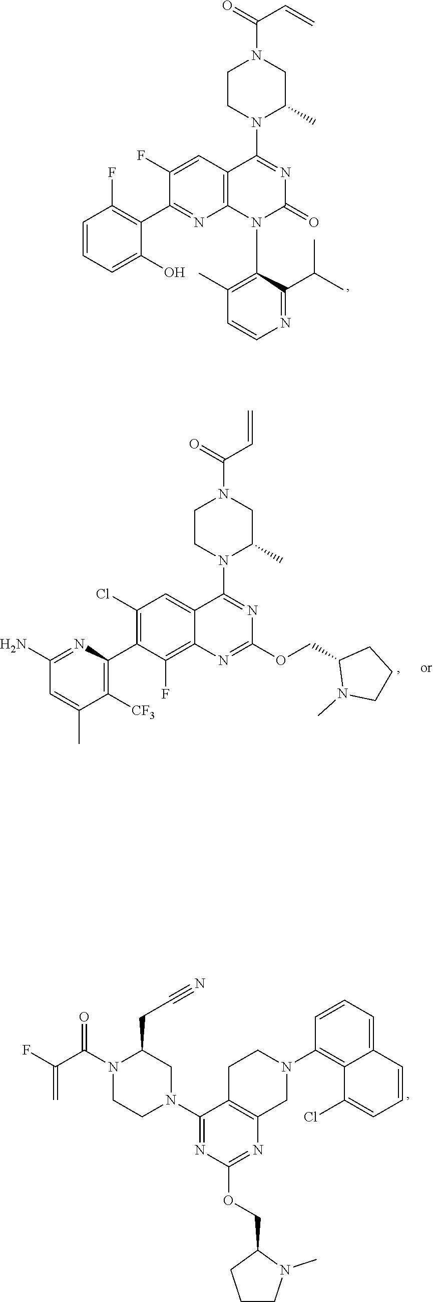

or any combination thereof, or a stereoisomer or tautomer thereof, or a pharmaceutically acceptable salt of any of the foregoing.

In some embodiments, in conjunction with embodiments above or below, the one or more KRAS inhibitors comprise

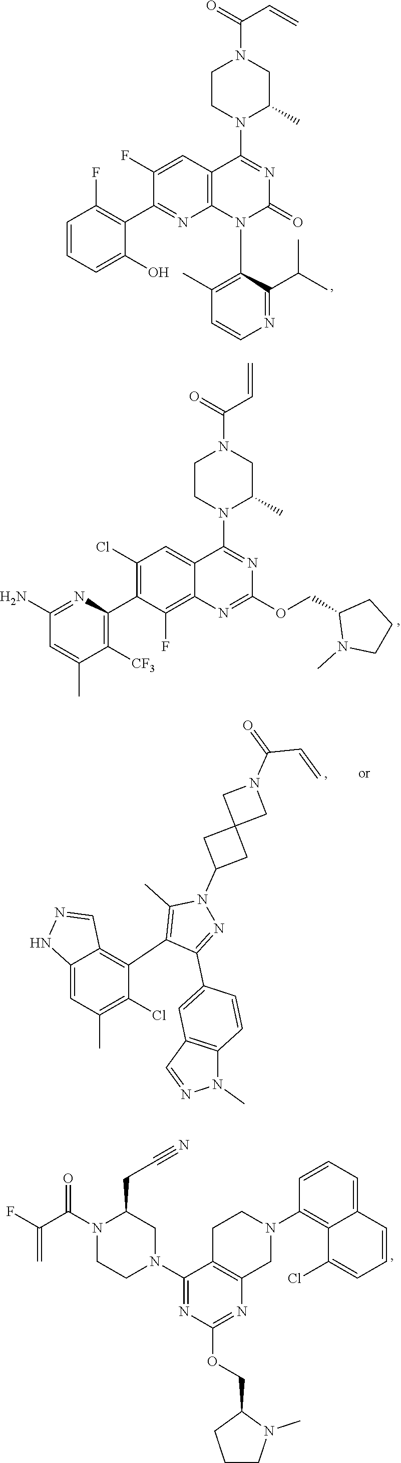

or any combination thereof, or a stereoisomer or tautomer thereof, or a pharmaceutically acceptable salt of any of the foregoing.

In some embodiments, the one or more KRAS inhibitors comprise

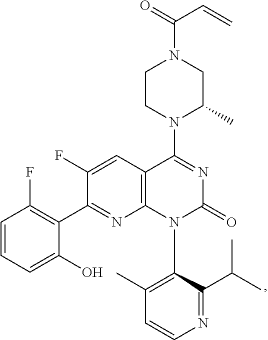

or a stereoisomer or tautomer thereof, or a pharmaceutically acceptable salt of any of the foregoing. In some embodiments, the one or more KRAS inhibitors comprise sotorasib, or a stereoisomer or tautomer thereof, or a pharmaceutically acceptable salt of any of the foregoing.

In some embodiments, the one or more KRAS inhibitors comprise

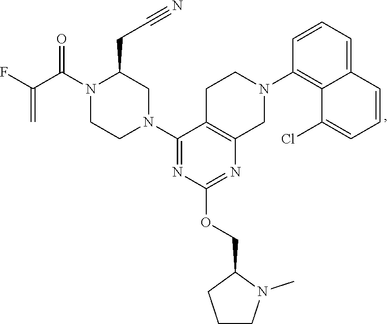 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing. In some embodiments, the one or more KRAS inhibitors comprise adagrasib, or a stereoisomer or tautomer thereof, or a pharmaceutically acceptable salt of any of the foregoing.

In some embodiments the one or more KRAS inhibitors comprise

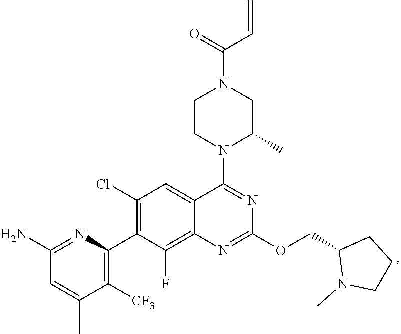

or a stereoisomer or tautomer thereof, or a pharmaceutically acceptable salt of any of the foregoing.

In some embodiments the one or more KRAS inhibitors comprise

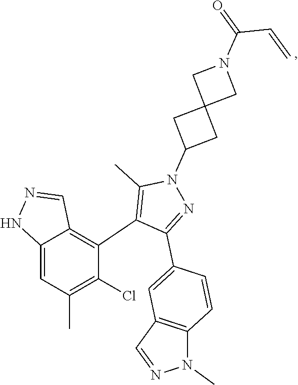 [SMILES: C=CC(=O)N1CC2(CC(n3nc(-c4ccc5c(cnn5C)c4)c(-c4c(Cl)c(C)cc5[nH]ncc45)c3C)C2)C1]

or a stereoisomer or tautomer thereof, or a pharmaceutically acceptable salt of any of the foregoing.

In some embodiments, in conjunction with embodiments above or below, the one or more KRAS inhibitors comprise a G12C KRAS inhibitor (e.g., any one of Compound K1, Compound K2, Compound K3, and Compound K4). G12C KRAS inhibitors are described in, for example, Hallin et al. (Cancer Discov, 2020, 10(1): 54-71), Skoulidis et al. (N. Engl. J. Med., 2021, 384(25): 2371-2381), and Hong et al. (N. Engl. J. Med., 2020, 383(13): 1207-1217), each of which is incorporated herein by reference in its entirety and specifically with respect to G12C KRAS inhibitors described therein.

### Combination:

Provided herein are compositions, methods and kits comprising one or more TEAD inhibitors (e.g., a compound of formula (I), (II), (III), (IV), (V), (VI), (VII), or (VIII), or any variation or embodiment thereof) and one or more KRAS inhibitors (e.g., a compound of formula (K-I), (K-II), (K-III), or (K-IV), or any variation or embodiment thereof). Each and every combination of TEAD inhibitor and KRAS inhibitor is intended the same as if each and every combination is specifically and individually listed. Thus, for example, it is intended that any combination of: (1) a compound of formula (I), (II), (III), (IV), (V), (VI), (VII), or (VIII), or any variation or embodiment thereof; and (2) a compound of formula (K-I), (K-II), (K-III), or (K-IV), or any variation or embodiment thereof, is provided herein.

Also provided herein are compositions, methods and kits comprising one or more TEAD inhibitors (e.g., a TEAD palmitate pocket binding inhibitor, a covalent TEAD inhibitor, or a compound of formula (I)) and one or more KRAS inhibitors (e.g., a G12C KRAS inhibitor, or a compound of formula (K-II)). Each and every combination of TEAD inhibitor and KRAS inhibitor is intended the same as if each and every combination is specifically and individually listed. Thus, for example, it is intended that any combination of: (1) a TEAD palmitate pocket binding inhibitor (e.g., Compound T1, Compound T2, Compound T3, Compound T4, Compound T5, Compound T6, Compound T7, Compound T8, Compound T9, or Compound T10) or a covalent TEAD inhibitor (e.g., Compound T2, Compound T3, or Compound T4); and (2) a G12C KRAS inhibitor (e.g., Compound K1, Compound K2, Compound K3, or Compound K4), is provided herein.

In some embodiments, the one or more TEAD inhibitors comprise a TEAD palmitate pocket binding inhibitor and the one or more KRAS inhibitors comprise a G12C KRAS inhibitor. In some embodiments, the one or more TEAD inhibitors comprise a covalent TEAD inhibitor and the one or more KRAS inhibitors comprise a G12C KRAS inhibitor.

In some embodiments, the one or more TEAD inhibitors comprise a compound of formula (I), (I-A), (I-B), (I-B1), or (I-C) (e.g., Compound T1 or T9) and the one or more KRAS inhibitors comprise a compound of formula (K-I), (K-II), (K-III), or (K-IV). In some embodiments, the one or more TEAD inhibitors comprise a compound of formula (II), (II-A), (II-A1), or (II-B) (e.g. Compound T2 or T3) and the one or more KRAS inhibitors comprise a compound of formula (K-I), (K-II), (K-III), or (K-IV). In some embodiments, the one or more TEAD inhibitors comprise a compound of formula (III), (III-A), or (III-A1) (e.g. Compound T4) and the one or more KRAS inhibitors comprise a compound of formula (K-I), (K-II), (K-III), or (K-IV). In some embodiments, the one or more TEAD inhibitors comprise a compound of formula (IV), (IV-A), (IV-B), (IV-C), (IV-D), (IV-E), or (IV-F) (e.g. Compound T5) and the one or more KRAS inhibitors comprise a compound of formula (K-I), (K-II), (K-III), or (K-IV). In some embodiments, the one or more TEAD inhibitors comprise a compound of formula (V) (e.g. Compound T6) and the one or more KRAS inhibitors comprise a compound of formula (K-I), (K-II), (K-III), or (K-IV). In some embodiments, the one or more TEAD inhibitors comprise a compound of formula (VI) or (VI-A) (e.g. Compound T7) and the one or more KRAS inhibitors comprise a compound of formula (K-I), (K-II), (K-III), or (K-IV). In some embodiments, the one or more TEAD inhibitors comprise a compound of formula (VII), (VII-A), or (VII-B) (e.g. Compound T8) and the one or more KRAS inhibitors comprise a compound of formula (K-I), (K-II), (K-III), or (K-IV). In some embodiments, the one or more TEAD inhibitors comprise a compound of formula (VIII) or (VIII-A) (e.g. Compound T10) and the one or more KRAS inhibitors comprise a compound of formula (K-I), (K-II), (K-III), or (K-IV).

In some embodiments, the one or more TEAD inhibitors comprise a compound of formula (I), (II), (III), (IV), (V), (VI), (VII), or (VIII), and the one or more KRAS inhibitors comprise a compound of formula (K-I) or (K-I-A) (e.g., Compound K1). In some embodiments, the one or more TEAD inhibitors comprise a compound of formula (I), (II), (III), (IV), (V), (VI), (VII), or (VIII), and the one or more KRAS inhibitors comprise a compound of formula (K-II), (K-II-A), (K-II-B), or (K-II-C) (e.g., Compound K2). In some embodiments, the one or more TEAD inhibitors comprise a compound of formula (I), (II), (III), (IV), (V), (VI), (VII), or (VIII), and the one or more KRAS inhibitors comprise a compound of formula (K-III) or (K-III-A) (e.g., Compound K3). In some embodiments, the one or more TEAD inhibitors comprise a compound of formula (I), (II), (III), (IV), (V), (VI), (VII), or (VIII), and the one or more KRAS inhibitors comprise a compound of formula (K-IV), (K-IV-A), (K-IV-B), or (K-IV-C) (e.g., Compound K4).

In some embodiments, the compositions, methods, or kits described herein comprise Compound T1 and Compound K1. In some embodiments, the compositions, methods, or kits described herein comprise Compound T2 and Compound K1. In some embodiments, the compositions, methods, or kits described herein comprise Compound T3 and Compound K1. In some embodiments, the compositions, methods, or kits described herein comprise Compound T4 and Compound K1. In some embodiments, the compositions, methods, or kits described herein comprise Compound T5 and Compound K1. In some embodiments, the compositions, methods, or kits described herein comprise Compound T6 and Compound K1. In some embodiments, the compositions, methods, or kits described herein comprise Compound T7 and Compound K1. In some embodiments, the compositions, methods, or kits described herein comprise Compound T8 and Compound K1. In some embodiments, the compositions, methods, or kits described herein comprise Compound T9 and Compound K1. In some embodiments, the compositions, methods, or kits described herein comprise Compound T10 and Compound K1.

In some embodiments, the compositions, methods, or kits described herein comprise Compound T1 and Compound K2. In some embodiments, the compositions, methods, or kits described herein comprise Compound T2 and Compound K2. In some embodiments, the compositions, methods, or kits described herein comprise Compound T3 and Compound K2. In some embodiments, the compositions, methods, or kits described herein comprise Compound T4 and Compound K2. In some embodiments, the compositions, methods, or kits described herein comprise Compound T5 and Compound K2. In some embodiments, the compositions, methods, or kits described herein comprise Compound T6 and Compound K2. In some embodiments, the compositions, methods, or kits described herein comprise Compound T7 and Compound K2. In some embodiments, the compositions, methods, or kits described herein comprise Compound T8 and Compound K2. In some embodiments, the compositions, methods, or kits described herein comprise Compound T9 and Compound K2. In some embodiments, the compositions, methods, or kits described herein comprise Compound T10 and Compound K2.

In some embodiments, the compositions, methods, or kits described herein comprise Compound T1 and Compound K3. In some embodiments, the compositions, methods, or kits described herein comprise Compound T2 and Compound K3. In some embodiments, the compositions, methods, or kits described herein comprise Compound T3 and Compound K3. In some embodiments, the compositions, methods, or kits described herein comprise Compound T4 and Compound K3. In some embodiments, the compositions, methods, or kits described herein comprise Compound T5 and Compound K3. In some embodiments, the compositions, methods, or kits described herein comprise Compound T6 and Compound K3. In some embodiments, the compositions, methods, or kits described herein comprise Compound T7 and Compound K3. In some embodiments, the compositions, methods, or kits described herein comprise Compound T8 and Compound K3. In some embodiments, the compositions, methods, or kits described herein comprise Compound T9 and Compound K3. In some embodiments, the compositions, methods, or kits described herein comprise Compound T10 and Compound K3.

In some embodiments, the compositions, methods, or kits described herein comprise Compound T1 and Compound K4. In some embodiments, the compositions, methods, or kits described herein comprise Compound T2 and Compound K4. In some embodiments, the compositions, methods, or kits described herein comprise Compound T3 and Compound K4. In some embodiments, the compositions, methods, or kits described herein comprise Compound T4 and Compound K4. In some embodiments, the compositions, methods, or kits described herein comprise Compound T5 and Compound K4. In some embodiments, the compositions, methods, or kits described herein comprise Compound T6 and Compound K4. In some embodiments, the compositions, methods, or kits described herein comprise Compound T7 and Compound K4. In some embodiments, the compositions, methods, or kits described herein comprise Compound T8 and Compound K4. In some embodiments, the compositions, methods, or kits described herein comprise Compound T9 and Compound K4. In some embodiments, the compositions, methods, or kits described herein comprise Compound T10 and Compound K4.

In some embodiments, provided herein is a composition, comprising (i) one or more YAP/TAZ-TEAD inhibitors, wherein the one or more YAP/TAZ-TEAD inhibitors comprise a compound of formula (I), or a stereoisomer or tautomer thereof, or a pharmaceutically acceptable salt of any of the foregoing; and (ii) one or more KRAS inhibitors. In some embodiments, provided herein is a composition comprising (i) one or more YAP/TAZ-TEAD inhibitors, wherein the one or more YAP/TAZ-TEAD inhibitors comprise a compound of formula (II), or a stereoisomer or tautomer thereof, or a pharmaceutically acceptable salt of any of the foregoing; and (ii) one or more KRAS inhibitors. In some embodiments, provided herein is a composition comprising (i) one or more YAP/TAZ-TEAD inhibitors, wherein the one or more YAP/TAZ-TEAD inhibitors comprise a compound of formula (III), or a stereoisomer or tautomer thereof, or a pharmaceutically acceptable salt of any of the foregoing; and (ii) one or more KRAS inhibitors. In some embodiments, provided herein is a composition comprising (i) one or more YAP/TAZ-TEAD inhibitors, wherein the one or more YAP/TAZ-TEAD inhibitors comprise a compound of formula (I), formula (II), or formula (III), or any variation or embodiment thereof, or any combination of the foregoing; and (ii) one or more KRAS inhibitors.

In some embodiments, provided herein is a composition, comprising (i) one or more YAP/TAZ-TEAD inhibitors selected from the group consisting of

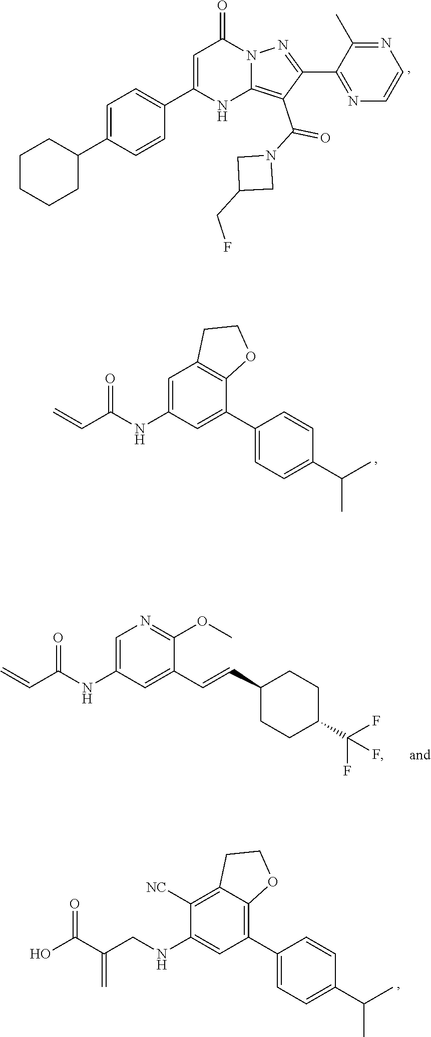

stereoisomer or tautomer thereof, or a pharmaceutically acceptable salt of any of the foregoing; and (ii) one or more KRAS inhibitors selected from the group consisting of

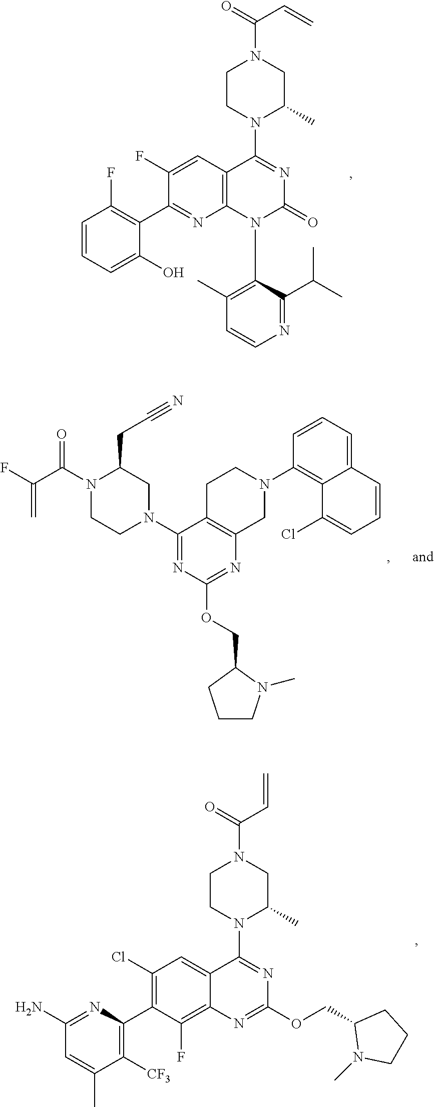

or a stereoisomer or tautomer thereof, or a pharmaceutically acceptable salt of any of the foregoing.

In some embodiments, provided herein is a composition, comprising (i) one or more YAP/TAZ-TEAD inhibitors selected from the group consisting of:

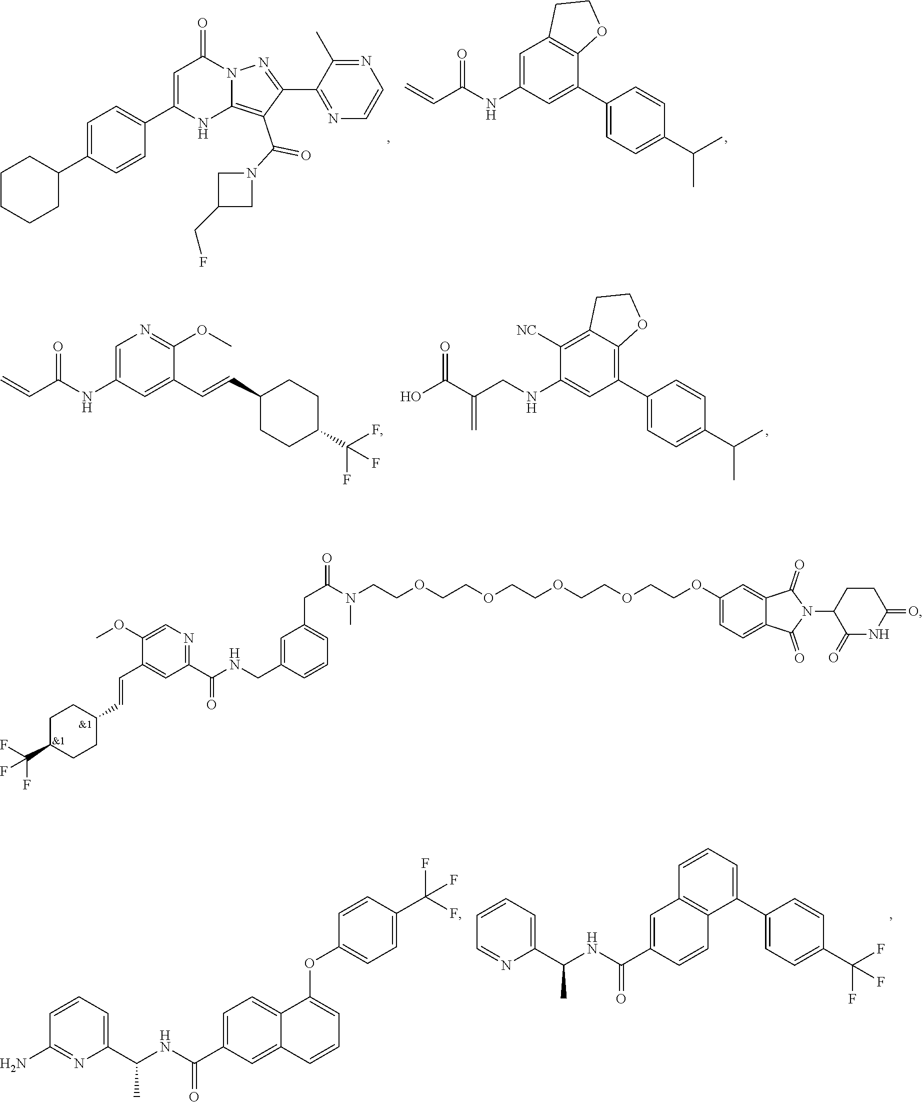

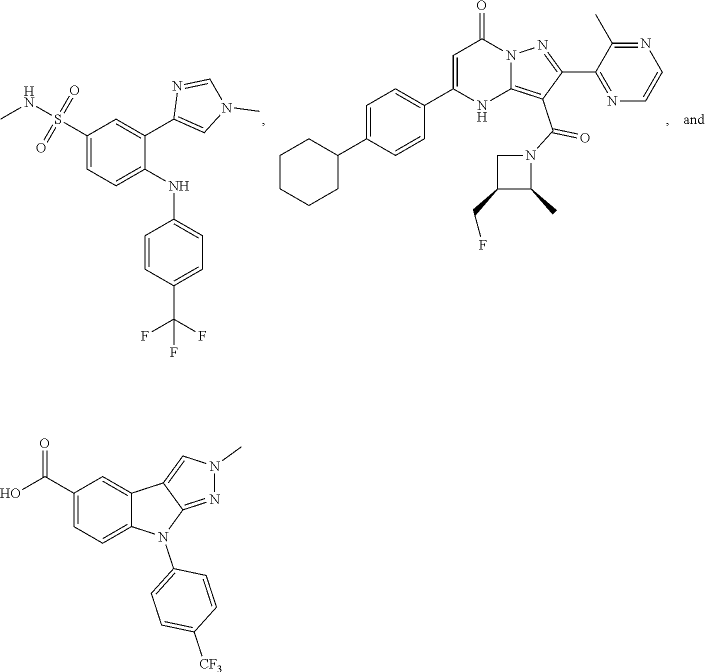

or a stereoisomer or tautomer thereof, or a pharmaceutically acceptable salt of any of the foregoing; and (ii) one or more KRAS inhibitors selected from the group consisting of:

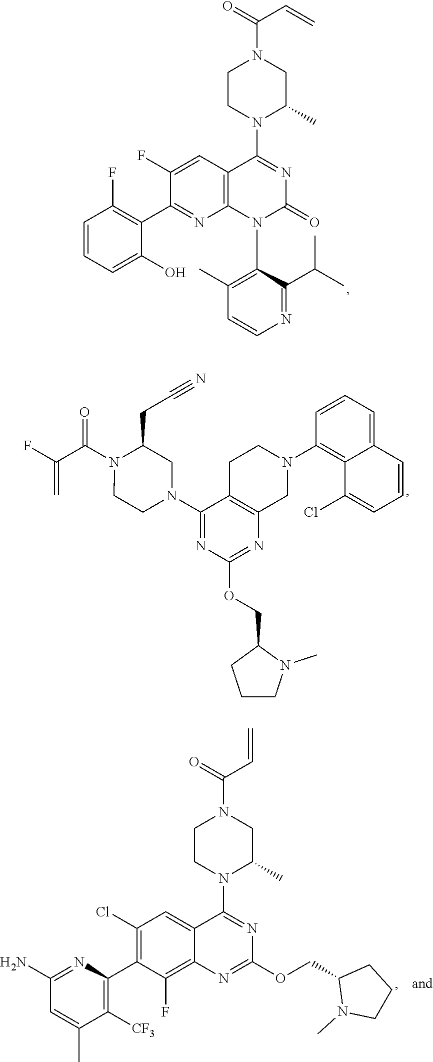

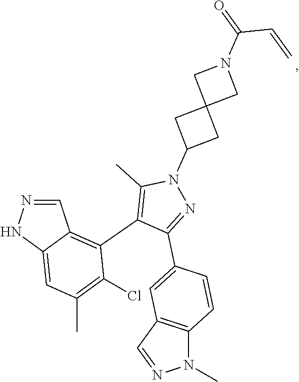 [SMILES: C=CC(=O)N1CC2(CC(n3nc(-c4ccc5c(cnn5C)c4)c(-c4c(Cl)c(C)cc5[nH]ncc45)c3C)C2)C1]

or a stereoisomer or tautomer thereof, or a pharmaceutically acceptable salt of any of the foregoing.

In some embodiments, provided herein is a composition, comprising

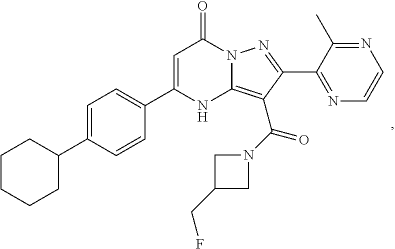 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing, and

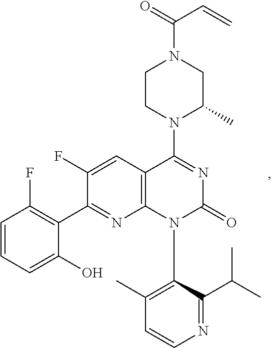

or a stereoisomer or tautomer thereof, or a pharmaceutically acceptable salt of any of the foregoing.

In some embodiments, provided herein is a composition, comprising

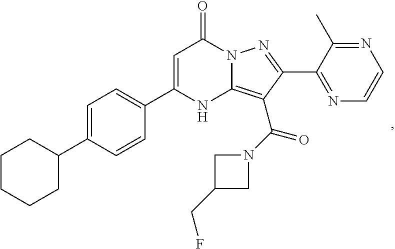 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing, and

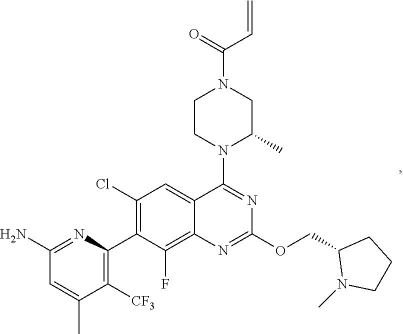

or a stereoisomer or tautomer thereof, or a pharmaceutically acceptable salt of any of the foregoing.

In some embodiments, provided herein is a composition, comprising

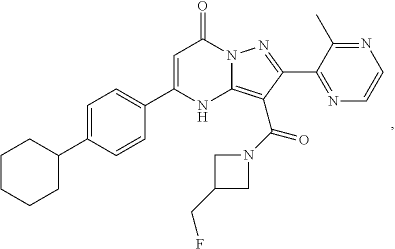 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing, and

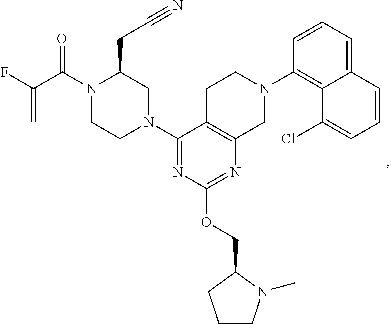 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.

In some embodiments, provided herein is a composition, comprising

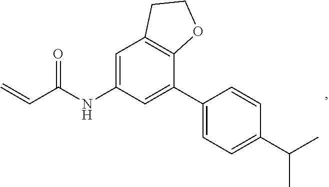 [SMILES: C=CC(=O)Nc1cc2c(c(-c3ccc(C(C)C)cc3)c1)OCC2]

or a stereoisomer or tautomer thereof, or a pharmaceutically acceptable salt of any of the foregoing, and

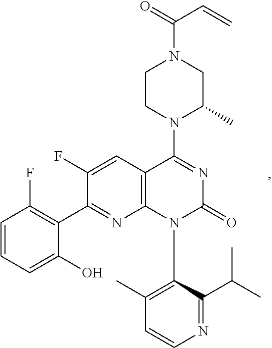

or a stereoisomer or tautomer thereof, or a pharmaceutically acceptable salt of any of the foregoing.

In some embodiments, provided herein is a composition, comprising

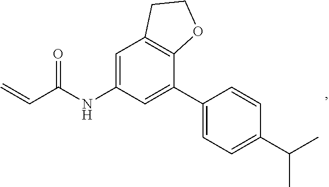 [SMILES: C=CC(=O)Nc1cc2c(c(-c3ccc(C(C)C)cc3)c1)OCC2]

or a stereoisomer or tautomer thereof, or a pharmaceutically acceptable salt of any of the foregoing, and,

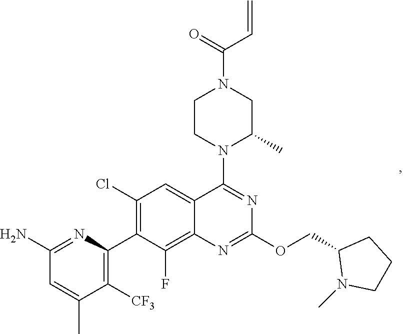

or a stereoisomer or tautomer thereof, or a pharmaceutically acceptable salt of any of the foregoing.

In some embodiments, provided herein is a composition, comprising

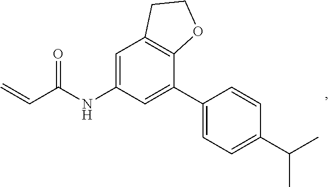 [SMILES: C=CC(=O)Nc1cc2c(c(-c3ccc(C(C)C)cc3)c1)OCC2]

or a stereoisomer or tautomer thereof, or a pharmaceutically acceptable salt of any of the foregoing, and,

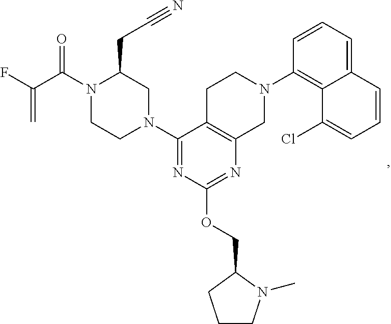 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.

In some embodiments, provided herein is a composition, comprising

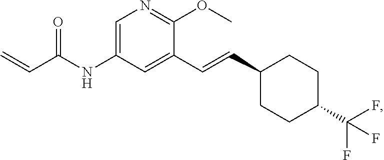 [SMILES: C=CC(=O)Nc1cnc(OC)c(/C=C/[C@H]2CC[C@H](C(F)(F)F)CC2)c1]

or a stereoisomer or tautomer thereof, or a pharmaceutically acceptable salt of any of the foregoing, and

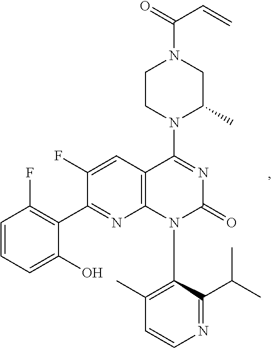

or a stereoisomer or tautomer thereof, or a pharmaceutically acceptable salt of any of the foregoing.

In some embodiments, provided herein is a composition, comprising

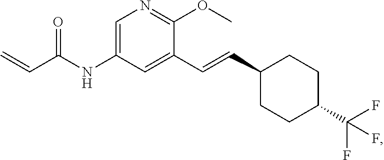 [SMILES: C=CC(=O)Nc1cnc(OC)c(/C=C/[C@H]2CC[C@H](C(F)(F)F)CC2)c1]

or a stereoisomer or tautomer thereof, or a pharmaceutically acceptable salt of any of the foregoing, and

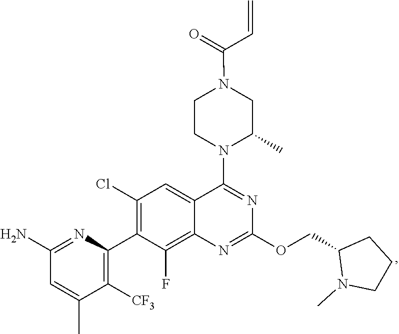

or a stereoisomer or tautomer thereof, or a pharmaceutically acceptable salt of any of the foregoing.

In some embodiments, provided herein is a composition, comprising

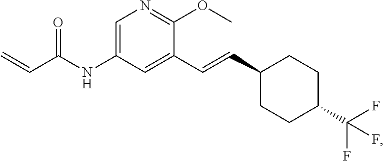 [SMILES: C=CC(=O)Nc1cnc(OC)c(/C=C/[C@H]2CC[C@H](C(F)(F)F)CC2)c1]

or a stereoisomer or tautomer thereof, or a pharmaceutically acceptable salt of any of the foregoing, and

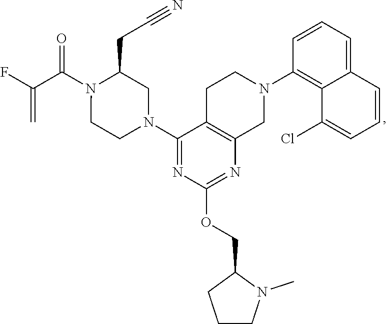 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.

In some embodiments, provided herein is a composition, comprising

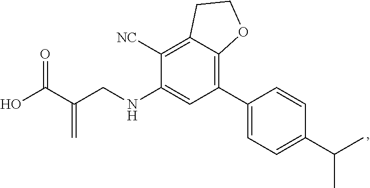 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing, and

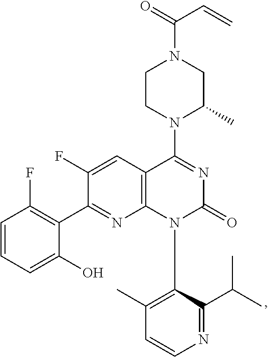

or a stereoisomer or tautomer thereof, or a pharmaceutically acceptable salt of any of the foregoing.

In some embodiments, provided herein is a composition, comprising

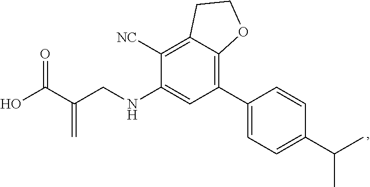 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing, and

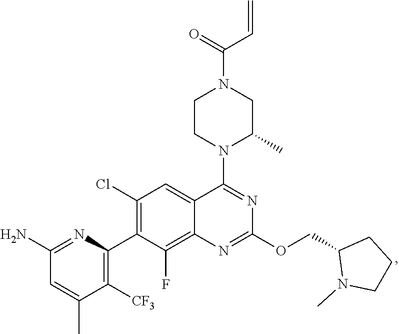

or a stereoisomer or tautomer thereof, or a pharmaceutically acceptable salt of any of the foregoing.

In some embodiments, provided herein is a composition, comprising

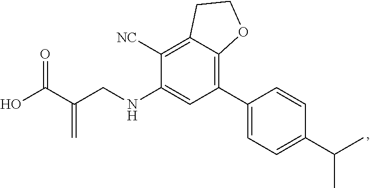 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing, and

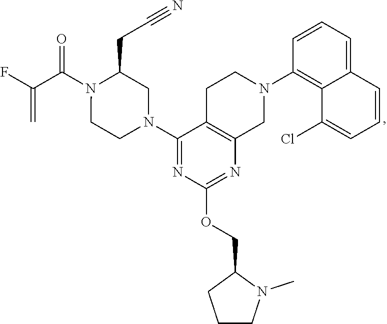 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.

In some embodiments, the YAP/TAZ-TEAD inhibitors are selected from the group consisting of compounds T1, T2, T3, and T4, as listed in Table 1, or a stereoisomer or tautomer thereof, or a pharmaceutically acceptable salt of any of the foregoing. In some embodiments, the one or more KRAS inhibitors are selected from the group consisting of compounds K1, K2, and K3, as listed in Table 1, or a stereoisomer or tautomer thereof, or a pharmaceutically acceptable salt of any of the foregoing.

In some embodiments, the YAP/TAZ-TEAD inhibitors are selected from the group consisting of compounds T1, T2, T3, T4, T5, T6, T7, T8, T9, and T10 as listed in Table 1, or a stereoisomer or tautomer thereof, or a pharmaceutically acceptable salt of any of the foregoing. In some embodiments, the one or more KRAS inhibitors are selected from the group consisting of compounds K1, K2, K3, and K4 as listed in Table 1, or a stereoisomer or tautomer thereof, or a pharmaceutically acceptable salt of any of the foregoing.

TABLE 1

| Compound | | |
|---|---|---|
| Number | Structure | Chemical name |
| T1 | 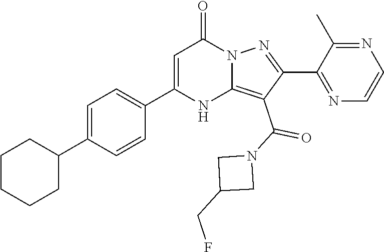 [SMILES: Cc1nccnc1-c1nn2c(=O)cc(-c3ccc(C4CCCCC4)cc3)[nH]c2c1C(=O)N1CC(CF)C1] | 5-(4-cyclohexylphenyl)-3-(3- (fluoromethyl)azetidine-1-carbonyl)- 2-(3-methylpyrazin-2- yl)pyrazolo[1,5-a]pyrimidin-7(4H)- one |
| T2 | 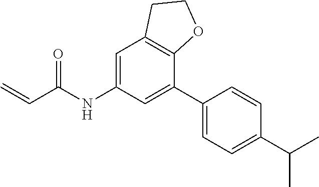 [SMILES: C=CC(=O)Nc1cc2c(c(-c3ccc(C(C)C)cc3)c1)OCC2] | N-(7-(4-isopropylphenyl)-2,3- dihydrobenzofuran-5-yl)acrylamide |
| T3 | 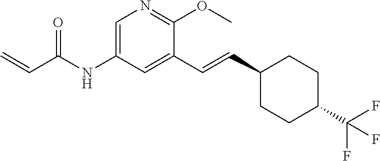 [SMILES: C=CC(=O)Nc1cnc(OC)c(/C=C/[C@H]2CC[C@H](C(F)(F)F)CC2)c1] | N-(6-methoxy-5-((E)-2-((1r,4r)-4- (trifluoromethyl)cyclohexyl)vinyl) pyridin-3-yl)acrylamide |
| T4 | 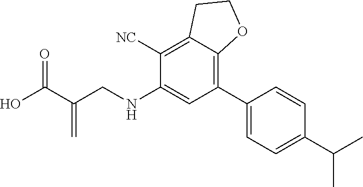 [SMILES: C=C(CNc1cc(-c2ccc(C(C)C)cc2)c2c(c1C#N)CCO2)C(=O)O] | 2-(((4-cyano-7-(4-isopropylphenyl)- 2,3-dihydrobenzofuran-5- yl)amino)methyl)acrylic acid |
| T5 | 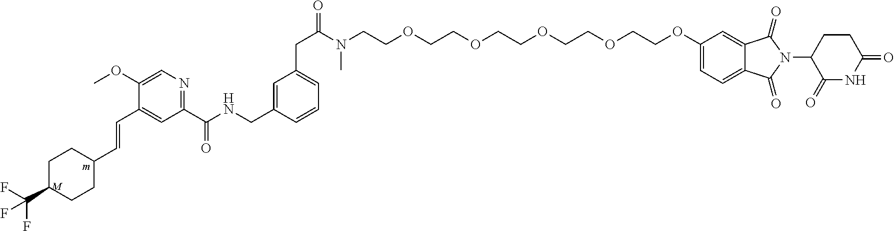 | N-[[3-[2-[2-[2-[2-[2-[2-[2-(2,6- dioxo-3-piperidyl)-1,3-dioxo- isoindolin-5- yl]oxyethoxy]ethoxy]ethoxy]ethoxy] ethyl-methyl-amino]-2-oxo- ethyl]phenyl]methyl]-5-methoxy-4- [rac-(E)-2-[4- (trifluoromethyl)cyclohexyl]vinyl] pyridine-2-carboxamide |
| T6 | 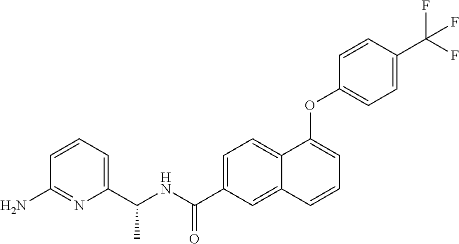 [SMILES: C[C@@H](NC(=O)c1ccc2c(Oc3ccc(C(F)(F)F)cc3)cccc2c1)c1cccc(N)n1] | N-[(1R)-1-(6-amino-2-pyridyl)ethyl]- 5-[4- (trifluoromethyl)phenoxy]naphthalene- 2-carboxamide |
| T7 | 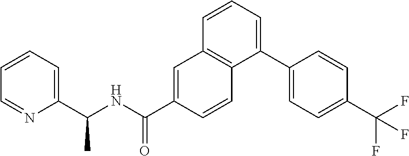 [SMILES: C[C@H](NC(=O)c1ccc2c(-c3ccc(C(F)(F)F)cc3)cccc2c1)c1ccccn1] | N-[(1S)-1-(2-pyridyl)ethyl]-5-[4- (trifluoromethyl)phenyl]naphthalene- 2-carboxamide |
| T8 | 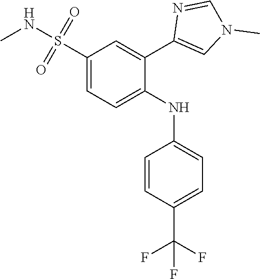 | N-methyl-3-(1-methylimidazol-4-yl)- 4-[4- (trifluoromethyl)anilino] benzenesulfonamide |
| T9 | 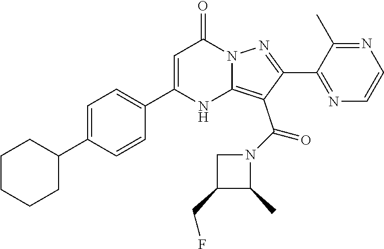 [SMILES: Cc1nccnc1-c1nn2c(=O)cc(-c3ccc(C4CCCCC4)cc3)[nH]c2c1C(=O)N1C[C@H](CF)[C@@H]1C] | 5-(4-cyclohexylphenyl)-2-(3- methylpyrazin-2-yl)-3-[rac-(2S,3S)- 3-(fluoromethyl)-2-methyl-azetidine- 1-carbonyl]-4H-pyrazolo[1,5- a]pyrimidin-7-one |
| T10 | 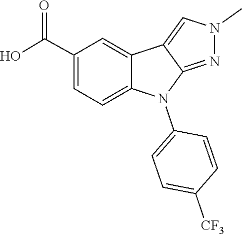 [SMILES: Cn1cc2c3cc(C(=O)O)ccc3n(-c3ccc(C(F)(F)F)cc3)c2n1] | 2-methyl-8-[4- (trifluoromethyl)phenyl]-2H,8H- pyrazolo[3,4-blindole-5-carboxylic acid |
| K1 | 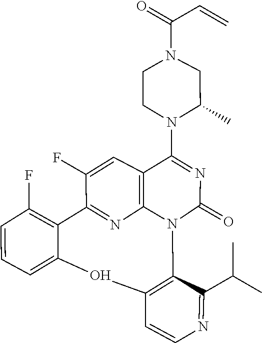 | 4-((S)-4-acryloyl-2-methylpiperazin- 1-yl)-6-fluoro-7-(2-fluoro-6- hydroxyphenyl)-1-(2-isopropyl-4- methylpyridin-3-yl)pyrido[2,3- d]pyrimidin-2(1H)-one |
| K2 | 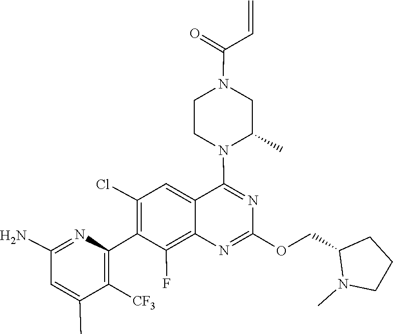 | 1-((S)-4-((R)-7-(6-amino-4-methyl-3- (trifluoromethyl)pyridin-2-yl)-6- chloro-8-fluoro-2-(((S)-1- methylpyrrolidin-2- yl)methoxy)quinazolin-4-yl)-3- methylpiperazin-1-yl)prop-2-en-1- one |
| K3 | 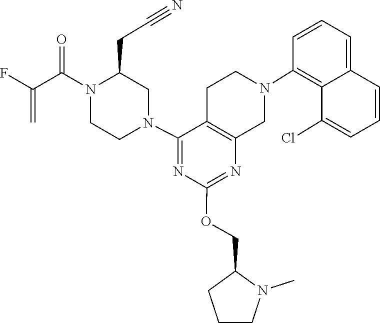 [SMILES: C=C(F)C(=O)N1CCN(c2nc(OC[C@@H]3CCCN3C)nc3c2CCN(c2cccc4cccc(Cl)c24)C3)C[C@@H]1CC#N] | 2-((S)-4-(7-(8-chloronaphthalen-1- yl)-2-(((S)-1-methylpyrrolidin-2- yl)methoxy)-5,6,7,8- tetrahydropyrido[3,4-d]pyrimidin-4- yl)-1-(2-fluoroacryloyl)piperazin-2- yl)acetonitrile |
| K4 | 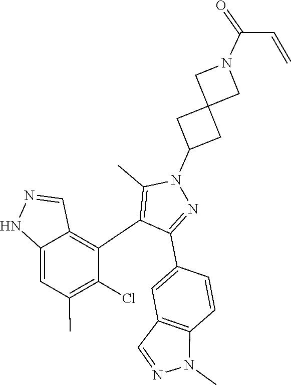 [SMILES: C=CC(=O)N1CC2(CC(n3nc(-c4ccc5c(cnn5C)c4)c(-c4c(Cl)c(C)cc5[nH]ncc45)c3C)C2)C1] | 1-[6-[4-(5-chloro-6-methyl-1H-indazol-4- yl)-5-methyl-3-(1-methylindazol-5- yl)pyrazol-1-yl]-2-azaspiro[3.3]heptan-2- yl]prop-2-en-1-one |

In some embodiments, the YAP/TAZ-TEAD inhibitors are selected from the group consisting of:
5-(4-cyclohexylphenyl)-3-(3-(fluoromethyl)azetidine-1-carbonyl)-2-(3-methylpyrazin-2-yl)pyrazolo[1,5-a]pyrimidin-7(4H)-one;
N-(7-(4-isopropylphenyl)-2,3-dihydrobenzofuran-5-yl)acrylamide;
N-(6-methoxy-5-(2-(4-(trifluoromethyl)cyclohexyl)vinyl)pyridin-3-yl)acrylamide;
2-(((4-cyano-7-(4-isopropylphenyl)-2,3-dihydrobenzofuran-5-yl)amino)methyl)acrylic acid;
N-(3-(17-((2-(2,6-dioxopiperidin-3-yl)-1,3-dioxoisoindolin-5-yl)oxy)-3-methyl-2-oxo-6,9,12,15-tetraoxa-3-azaheptadecyl)benzyl)-5-methoxy-4-(2-(4-(trifluoromethyl)cyclohexyl)vinyl)picolinamide;
N-(1-(6-aminopyridin-2-yl)ethyl)-5-(4-(trifluoromethyl)phenoxy)-2-naphthamide;
N-(1-(pyridin-2-yl)ethyl)-5-(4-(trifluoromethyl)phenyl)-2-naphthamide;
N-methyl-3-(1-methylimidazol-4-yl)-4-[4-(trifluoromethyl)anilino]benzenesulfonamide;
5-(4-cyclohexylphenyl)-3-(3-(fluoromethyl)-2-methylazetidine-1-carbonyl)-2-(3-methylpyrazin-2-yl)pyrazolo[1,5-a]pyrimidin-7(4H)-one; and
2-methyl-8-[4-(trifluoromethyl)phenyl]-2H,8H-pyrazolo[3,4-blindole-5-carboxylic acid,
or a stereoisomer or tautomer thereof, or a pharmaceutically acceptable salt of any of the foregoing.

In some embodiments, the one or more KRAS inhibitors are selected from the group consisting of:
4-(4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one;
2-(4-(7-(8-chloronaphthalen-1-yl)-2-((1-methylpyrrolidin-2-yl)methoxy)-5,6,7,8-tetrahydropyrido[3,4-d]pyrimidin-4-yl)-1-(2-fluoroacryloyl)piperazin-2-yl)acetonitrile;
1-(4-(7-(6-amino-4-methyl-3-(trifluoromethyl)pyridin-2-yl)-6-chloro-8-fluoro-2-((1-methylpyrrolidin-2-yl)methoxy)quinazolin-4-yl)-3-methylpiperazin-1-yl)prop-2-en-1-one; and
1-[6-[4-(5-chloro-6-methyl-1H-indazol-4-yl)-5-methyl-3-(1-methylindazol-5-yl)pyrazol-1-yl]-2-azaspiro[3.3]heptan-2-yl]prop-2-en-1-one,
   or a stereoisomer or tautomer thereof, or a pharmaceutically acceptable salt of any of the foregoing.

Also provided herein are, where applicable, any and all stereoisomers of the TEAD inhibitors and KRAS inhibitors depicted herein, including geometric isomers (e.g., cis/trans isomers or E/Z isomers), enantiomers, diastereomers, or mixtures thereof in any ratio, including racemic mixtures.

In some embodiments, the TEAD inhibitors provided herein, such as compounds of formula (I), (II), (III), (IV), (V), (VI), (VII), or (VIII) or any variation or embodiment thereof, are isotopically-labeled by having one or more atoms therein replaced by an atom having a different atomic mass or mass number. Such isotopically-labeled (e.g., radiolabeled) compounds are considered to be within the scope of this disclosure. Examples of isotopes that can be incorporated into the TEAD inhibitors provided herein include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O , ¹⁷O, ¹⁸O, ³¹P, ³²P, ³⁵S, ¹⁸F, ³⁶Cl, ¹²³I, and ¹²⁵I, respectively. Certain isotopically-labeled TEAD inhibitor compounds, for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a TEAD inhibitor provided herein can be enriched with 1, 2, 5, 10, 25, 50, 75, 90, 95, or 99 percent of a given isotope.

For each of the TEAD inhibitors described herein, such as a compound of formula (I), (II), (III), (IV), (V), (VI), (VII), or (VIII) or any variation or embodiment thereof, substitution with heavier isotopes such as deuterium, i.e. ²H, may afford certain therapeutic advantages resulting from greater metabolic stability, for example, increased in vivo half-life or reduced dosage requirements. It is to be understood that any hydrogen (¹H) atom present in any of the compounds disclosed herein may be replaced by a deuterium (²H) atom. In any given compound provided herein, any number of hydrogen atoms may be replaced by the same number of deuterium atoms.

For each of the TEAD inhibitors described herein, such as a compound of formula (I), (II), (III), (IV), (V), (VI), (VII), or (VIII) or any variation or embodiment thereof, substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can generally be prepared by conventional techniques known to those skilled in the art or by processes analogous to those described in the Examples as set out below using an appropriate isotopically-labeled reagent in place of the non-labeled reagent previously employed.

In addition to salt forms, the present disclosure provides TEAD inhibitors, such as a compound of formula (I), (II), (III), (IV), (V), (VI), (VII), (VIII) or any variation or embodiment thereof, which are in a prodrug form. As used herein the term “prodrug” refers to those compounds that readily undergo chemical changes under physiological conditions to provide the TEAD inhibitors of the present disclosure. Additionally, prodrugs can be converted to the TEAD inhibitors of the present disclosure by chemical or biochemical methods in an ex vivo environment. For example, prodrugs can be slowly converted to the TEAD inhibitors of the present disclosure when placed in a transdermal patch reservoir with a suitable enzyme or chemical reagent.

Prodrugs of the TEAD inhibitors provided herein may include phosphates, phosphate esters, alkyl phosphates, alkyl phosphate esters, acyl ethers, or other prodrug moieties as discussed below. In some embodiments, the prodrug moiety is:

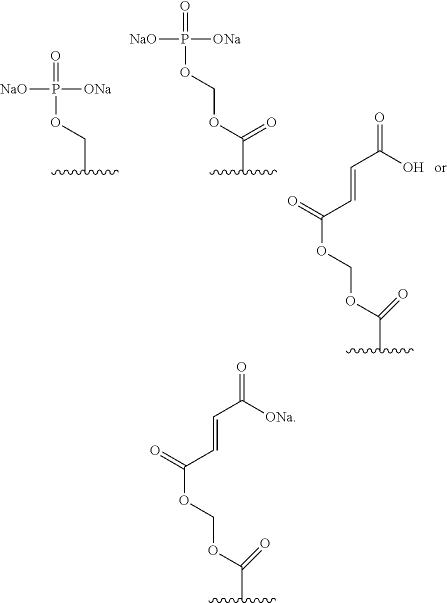

Additional types of prodrugs of the TEAD inhibitors provided herein are also encompassed. For example, where an amino acid residue, or a polypeptide chain of two or more (e.g., two, three or four) amino acid residues, is covalently joined through an amide or ester bond to a free amino, hydroxy or carboxylic acid group of a compound of the present disclosure. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methylalanine, para-benzoylphenylalanine, phenylglycine, propargylglycine, sarcosine, methionine sulfone and tert-butylglycine.

Additional types of prodrugs of the TEAD inhibitors provided herein are also encompassed. For instance, a free carboxyl group of a compound of the disclosure can be derivatized as an amide or alkyl ester. As another example, TEAD inhibitors of this disclosure comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxyl groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N—(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives that may be suitable for the TEAD inhibitors provided herein, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Additionally, the present disclosure provides for metabolites of the TEAD inhibitors of the disclosure, such as a compound of formula (I), (II), (III), (IV), (V), (VI), (VII), or (VIII) or any variation or embodiment thereof. As used herein, a “metabolite” refers to a product produced through metabolism in the body of a specified compound or salt thereof. Such products can result for example from the oxidation, reduction, hydrolysis, amidation, deamidation, esterification, deesterification, enzymatic cleavage, and the like, of the administered compound. Metabolite products typically are identified by preparing a radiolabeled (e.g., ¹⁴C or ³H) isotope of a TEAD inhibitor of the disclosure, administering it parenterally in a detectable dose (e.g., greater than about 0.5 mg/kg) to an animal such as rat, mouse, guinea pig, monkey, or to human, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolite products, so long as they are not otherwise found in vivo, are useful in diagnostic assays for therapeutic dosing of the TEAD inhibitors of the disclosure.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure can exist in multiple crystalline or amorphous forms. In general, all physical forms are equivalent for the uses contemplated by the present disclosure and are intended to be within the scope of the present disclosure.

### III. Pharmaceutical Compositions and Administration

Also disclosed is a pharmaceutical composition, comprising: (i) one or more YAP/TAZ-TEAD inhibitors; (ii) one or more KRAS inhibitors; and (iii) one or more therapeutically inert carriers. The pharmaceutical composition may comprise any of the YAP/TAZ-TEAD inhibitors and any of the KRAS inhibitors described elsewhere herein. Another aspect includes (i) one or more YAP/TAZ-TEAD inhibitors; (ii) one or more KRAS inhibitors; and (iii) one or more pharmaceutically acceptable carriers. In one embodiment, disclosed is a pharmaceutical composition, comprising: (i) one or more YAP/TAZ-TEAD inhibitors; (ii) one or more KRAS inhibitors; and (iii) one or more pharmaceutically acceptable carriers, adjuvants, or vehicles. In another embodiment, the composition comprises an amount of one or more YAP/TAZ-TEAD inhibitors effective to measurably disrupt the YAP:TEAD protein:protein interaction. In another embodiment, the composition comprises an amount of one or more KRAS inhibitors effective to measurably disrupt the activity of oncogenic KRAS genes. In certain embodiments, the composition is formulated for administration to a patient in need thereof. In another embodiment, the disclosure provides for a pharmaceutical composition, comprising a therapeutically effective amount of a composition, comprising: (i) one or more YAP/TAZ-TEAD inhibitors; (ii) one or more KRAS inhibitors; and (iii) one or more pharmaceutically acceptable carriers, diluents and/or excipients. In some embodiments of the foregoing, the one or more YAP/TAZ-TEAD inhibitors comprise a compound of formula (I), formula (II), or formula (III), or a stereoisomer or tautomer thereof, or a pharmaceutically acceptable salt of any of the foreoing, or any combination thereof. In some embodiments, the one or more YAP/TAZ-TEAD inhibitors comprise one or more of compounds T1, T2, T3, and T4, or a pharmaceutically acceptable salt of any of the foregoing, or any combination thereof. In some embodiments, the one or more KRAS inhibitors comprise one or more of compounds K1, K2, and K3, or a pharmaceutically acceptable salt of any of the foregoing, or any combination thereof. In some embodiments, the one or more KRAS inhibitors comprise sotorasib, or a stereoisomer or tautomer thereof, or a pharmaceutically acceptable salt of any of the foregoing. In some embodiments, the one or more KRAS inhibitors comprise adagrasib, or a stereoisomer or tautomer thereof, or a pharmaceutically acceptable salt of any of the foregoing.

In some embodiments of the foregoing, the one or more YAP/TAZ-TEAD inhibitors comprise a compound of formula (I), formula (II), formula (III), formula (IV), formula (V), formula (VI), formula (VII), or formula (VIII), or a stereoisomer or tautomer thereof, or a pharmaceutically acceptable salt of any of the foreoing, or any combination thereof. In some embodiments, the one or more YAP/TAZ-TEAD inhibitors comprise one or more of compounds T1, T2, T3, T4, T5, T6, T7, T8, T9, and T10, or a pharmaceutically acceptable salt of any of the foregoing, or any combination thereof. In some embodiments, the one or more KRAS inhibitors comprise one or more of compounds K1, K2, K3, and K4, or a pharmaceutically acceptable salt of any of the foregoing, or any combination thereof. In some embodiments, the one or more KRAS inhibitors comprise sotorasib, or a stereoisomer or tautomer thereof, or a pharmaceutically acceptable salt of any of the foregoing. In some embodiments, the one or more KRAS inhibitors comprise adagrasib, or a stereoisomer or tautomer thereof, or a pharmaceutically acceptable salt of any of the foregoing.

Pharmaceutically acceptable carriers, adjuvants, or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions disclosed herein may be administered orally, parenterally, by inhalation spray, topically, transdermally, rectally, nasally, buccally, sublingually, vaginally, intraperitoneal, intrapulmonary, intradermal, epidural or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

In one embodiment, the compositions disclosed herein are formulated as a solid dosage form for oral administration. Solid dosage forms for oral administration include capsules, tablets, pills, powders, and granules. In certain embodiments, the solid oral dosage form comprising a composition as described herein further comprises one or more of (i) an inert, pharmaceutically acceptable excipient or carrier, such as sodium citrate or dicalcium phosphate, and (ii) filler or extender such as starches, lactose, sucrose, glucose, mannitol, or silicic acid, (iii) binders such as carboxymethylcellulose, alginates, gelatin, polyvinylpyrrolidinone, sucrose or acacia, (iv) humectants such as glycerol, (v) disintegrating agent such as agar, calcium carbonate, potato or tapioca starch, alginic acid, certain silicates or sodium carbonate, (vi) solution retarding agents such as paraffin, (vii) absorption accelerators such as quaternary ammonium salts, (viii) a wetting agent such as cetyl alcohol or glycerol monostearate, (ix) absorbent such as kaolin or bentonite clay, and (x) lubricant such as talc, calcium stearate, magnesium stearate, polyethylene glycols or sodium lauryl sulfate. In certain embodiments, the solid oral dosage form is formulated as capsules, tablets or pills. In certain embodiments, the solid oral dosage form further comprises buffering agents. In certain embodiments, such compositions for solid oral dosage forms may be formulated as fillers in soft and hard-filled gelatin capsules comprising one or more excipients such as lactose or milk sugar, polyethylene glycols and the like.

In certain embodiments, tablets, dragees, capsules, pills and granules of the compositions described herein optionally comprise coatings or shells such as enteric coatings. They may optionally comprise opacifying agents and can also be of a composition that they release the active ingredient(s) only, or preferentially, in a certain part of the intestinal tract, optionally, in a delayed manner. Examples of embedding compositions include polymeric substances and waxes, which may also be employed as fillers in soft and hard-filled gelatin capsules using such excipients as lactose or milk sugar as well as high molecular weight polyethylene glycols and the like.

In another embodiment, compositions comprise liquid dosage formulations comprising a composition described herein for oral administration, and optionally further comprise one or more of pharmaceutically acceptable emulsions, microemulsions, solutions, suspensions, syrups and elixirs. In certain embodiments, the liquid dosage form optionally, further comprise one or more of an inert diluent such as water or other solvent, a solubilizing agent, and an emulsifier such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols or fatty acid esters of sorbitan, and mixtures thereof. In certain embodiments, liquid oral compositions optionally further comprise one or more adjuvant, such as a wetting agent, a suspending agent, a sweetening agent, a flavoring agent and a perfuming agent.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a composition as described herein, it is often desirable to slow the absorption of the components from the composition from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

In certain embodiments, the composition for rectal or vaginal administration are formulated as suppositories which can be prepared by mixing a composition as described herein with suitable non-irritating excipients or carriers such as cocoa butter, polyethylene glycol or a suppository wax, for example, those which are solid at ambient temperature but liquid at body temperature and therefore melt in the rectum or vaginal cavity and release the composition described herein.

Example dosage forms for topical or transdermal administration of a composition as described herein include ointments, pastes, creams, lotions, gels, powders, solutions, sprays, inhalants or patches. The composition is admixed under sterile conditions with a pharmaceutically acceptable carrier, and optionally preservatives or buffers. Additional formulation examples include an ophthalmic formulation, ear drops, eye drops, and transdermal patches. Transdermal dosage forms can be made by dissolving or dispensing the composition in medium, for example, ethanol or dimethylsulfoxide. Absorption enhancers can also be used to increase the flux of the compound across the skin. The rate can be controlled by either providing a rate controlling membrane or by dispersing the compound in a polymer matrix or gel.

Nasal aerosol or inhalation formulations of a composition as described herein may be prepared as solutions in saline, employing benzyl alcohol or other suitable preservatives, absorption promotors to enhance bioavailability, fluorocarbons, and/or other conventional solubilizing or dispersing agents.

Specific dosage and treatment regimen for any particular patient will depend upon a variety of factors, including age, body weight, general health, sex, diet, time of administration, rate of excretion, drug combination, the judgment of the treating physician, and the severity of the particular disease being treated. The amount of the composition described herein that is provided will also depend upon the particular compound in the composition.

In one embodiment, the therapeutically effective amount of the combination or composition of the disclosure administered parenterally per dose will be in the range of about 0.01-100 mg/kg, alternatively about 0.1 to 20 mg/kg of patient body weight per day, with the typical initial range of compound used being 0.3 to 15 mg/kg/day. In another embodiment, oral unit dosage forms, such as tablets and capsules, contain from about 5 to about 500 mg of the compound of the disclosure.

In one embodiment, the therapeutically effective amount of the combination or composition of the disclosure is administered at an amount of about 5 mg-600 mg, 5 mg-500 mg, 5 mg-400 mg, 5 mg-300 mg, 5 mg-250 mg, 5 mg-200 mg, 5 mg-150 mg, 5 mg-100 mg, 5 mg-50 mg, 5 mg-25 mg, 25 mg-600 mg, 25 mg-500 mg, 25 mg-400 mg, 25 mg-300 mg, 25 mg-250 mg, 25 mg-200 mg, 25 mg-150 mg, 25 mg-100 mg, 25 mg-50 mg, 50 mg-600 mg, 50 mg-500 mg, 50 mg-400 mg, 50 mg-300 mg, 50 mg-250 mg, 50 mg-200 mg, 50 mg-150 mg, or 50 mg-100 mg QD. In another embodiment, the therapeutically effective amount of the combination or composition of the disclosure is administered at an amount of about 5 mg, 25 mg, 50 mg, 100 mg, 150 mg, 200 mg, 250 mg, 300 mg, 400 mg or 500 mg.

In some embodiments of the methods of treatment provided herein, the one or more YAP/TAZ-TEAD inhibitors and the one or more KRAS inhibitors are administered simultaneously. In some embodiments, wherein the one or more YAP/TAZ-TEAD inhibitors and the one or more KRAS inhibitors are administered simultaneously, the one or more YAP/TAZ-TEAD inhibitors and the one or more KRAS inhibitors are administered in a single composition. By way of illustration, and not limitation, in one embodiment, the one or more YAP/TAZ-TEAD inhibitors and the one or more KRAS inhibitors are administered together in a single tablet. In some embodiments, wherein the one or more YAP/TAZ-TEAD inhibitors and the one or more KRAS inhibitors are administered simultaneously, the one or more YAP/TAZ-TEAD inhibitors and the one or more KRAS inhibitors are administered in a separate compositions. By way of illustration, and not limitation, in one embodiment, the one or more YAP/TAZ-TEAD inhibitors and the one or more KRAS inhibitors are administered simultaneously in separate tablets.

In some embodiments of the methods of treatment provided herein, the one or more YAP/TAZ-TEAD inhibitors and the one or more KRAS inhibitors are administered sequentially. In some embodiments, the one or more YAP/TAZ-TEAD inhibitors are administered before the one or more KRAS inhibitors. In some embodiments, the one or more KRAS inhibitors are administered before the one or more YAP/TAZ-TEAD inhibitors. In some embodiments, the sequential administrations are separated in time by a matter of minutes, hours, days, or weeks.

### IV. Methods of Treatment and Indications

In some embodiments, certain compounds present in the combinations and compositions described herein are inhibitors of the YAP:TEAD protein-protein interaction that bind to TEAD and disrupt the YAP:TEAD protein-protein interaction (“YAP:TEAD inhibitors”). In embodiments, the certain disclosed compounds are useful for the treatment of cancers, including cancers characterized by solid tumors, through their ability to inhibit YAP:TEAD protein-protein interaction. Compounds present in the combinations and compositions of the present disclosure are small molecule YAP:TEAD inhibitors. Small molecule YAP:TEAD inhibitors are useful, e.g., for the diagnosis or treatment of cancer, including with no limitations, lung cancer, breast cancer, head and neck cancer, colon cancer, ovarian cancer, liver cancer, brain cancer and prostate cancer, mesotheliomas, sarcomas and/or leukemia. In other embodiments, small molecule YAP:TEAD inhibitors are useful for the diagnosis or treatment of cancers characterized by solid tumors, including with no limitations lung, liver, ovarian, breast and/or squamous cancers. In some embodiments, the solid tumors have YAP/TAZ amplification or Nf2 deletion/mutation.

In some embodiments, certain compounds disclosed herein are inhibitors of oncogenic Ras genes, e.g., inhibitors of KRAS.

In some embodiments, the disclosed compounds, and any combinations thereof, are for use as therapeutically active substance.

In some embodiments, the disclosed compounds, and any combinations thereof, are for the therapeutic and/or prophylactic treatment of cancer.

In some embodiments, the disclosed compounds, and any combinations thereof, are for the preparation of a medicament for the therapeutic treatment of cancer.

In some embodiments, the disclosed compounds, and any combinations thereof, are for use in the therapeutic treatment of cancer.

The present disclosure is directed to a method for the therapeutic treatment of cancer in a subject. The method comprises administering to the subject an effective amount of any of the compositions described elsewhere herein.

In some aspects, provided is a method of modulating YAP/TAZ-TEAD activity, or KRAS activity, or both, in a cell, comprising administering to the cell an effective amount of a combination, comprising: (i) one or more YAP/TAZ-TEAD inhibitors; and (ii) one or more KRAS inhibitors.

In some aspects, provided is a method of inhibiting YAP/TAZ-TEAD activity, or KRAS activity, or both, in a cell, comprising administering to the cell an effective amount of a combination, comprising: (i) one or more YAP/TAZ-TEAD inhibitors; and (ii) one or more KRAS inhibitors.

In some aspects, provided herein is a method of sensitizing (or re-sensitizing) a cell that is resistant to KRAS inhibitors, comprising administering to the cell an effective amount of a combination, comprising: (i) one or more YAP/TAZ-TEAD inhibitors; and (ii) one or more KRAS inhibitors. In some embodiments, the cell has always been resistant to KRAS inhibitors. In some embodiments, the cell has developed resistance to KRAS inhibitors over time.

In some aspects, provided herein is a method of treating cancer in a subject in need thereof, comprising administering to the subject an effective amount of a combination, comprising: (i) one or more YAP/TAZ-TEAD inhibitors; and (ii) one or more KRAS inhibitors.

In some embodiments of the foregoing, the method described makes use of any of the combinations or compositions described elsewhere herein. In some embodiments of the foregoing methods, the one or more YAP/TAZ-TEAD inhibitors comprise a compound of formula (I), formula (II), formula (III), formula (IV), formula (V), formula (VI), formula (VII), or formula (VIII) or any variation or embodiment thereof (including, for example, a stereoisomer, tautomer, or pharmaceutically acceptable salt thereof), or any combination of the foregoing.

In some embodiments, the one or more YAP/TAZ-TEAD inhibitors comprise a compound selected from the group consisting of

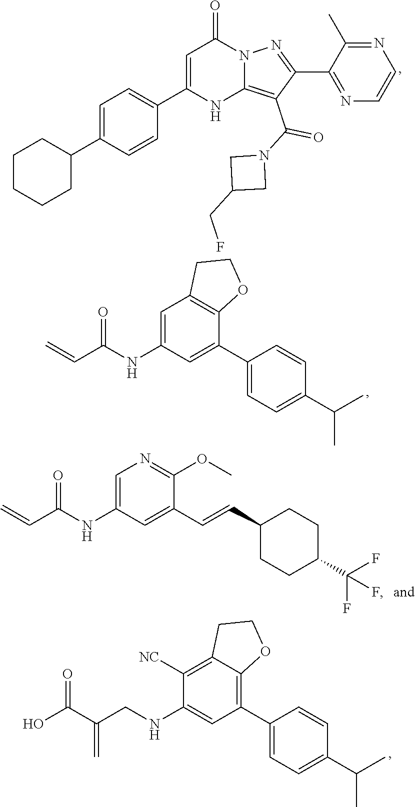

or a stereoisomer or tautomer thereof, or a pharmaceutically acceptable salt of any of the foregoing.

In some embodiments, the one or more KRAS inhibitors comprise a compound selected from the group consisting of

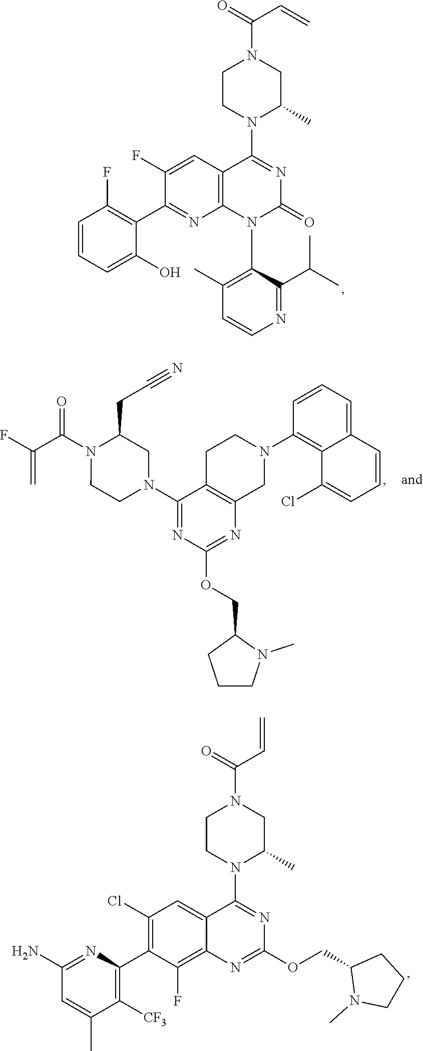

or a stereoisomer or tautomer thereof, or a pharmaceutically acceptable salt of any of the foregoing. In some embodiments, the one or more KRAS inhibitors comprise sotorasib, or a stereoisomer or tautomer thereof, or a pharmaceutically acceptable salt of any of the foregoing. In some embodiments, the one or more KRAS inhibitors comprise adagrasib, or a stereoisomer or tautomer thereof, or a pharmaceutically acceptable salt of any of the foregoing.

In some embodiments, the cancer is a solid tumor.

In some embodiments, the cancer is associated with YAP, TAZ, TEAD, and/or the YAP:TEAD protein-protein interaction.

Embodiments of the present disclosure provide a method of inhibiting Ras-mediated cell signaling, comprising contacting a cell with a therapeutically effective amount of one or more combinations or compositions disclosed herein. Inhibition of Ras-mediated signal transduction can be assessed and demonstrated by a wide variety of ways known in the art. Non-limiting examples include a showing of (a) a decrease in GTPase activity of Ras; (b) a decrease in GTP binding affinity or an increase in GDP binding affinity; (c) an increase in K off of GTP or a decrease in K off of GDP; (d) a decrease in the levels of signaling transduction molecules downstream in the Ras pathway, such as a decrease in pMEK level; and/or (e) a decrease in binding of Ras complex to downstream signaling molecules including but not limited to Raf. Kits and commercially available assays can be utilized for determining one or more of the above.

Embodiments also provide methods of using the combinations or compositions of the present disclosure to treat disease conditions, including, but not limited to, conditions implicated by G12C K-Ras mutation, G12C H-Ras mutation and/or G12C N-Ras mutation (e.g., cancer). In some embodiments, the cancer is associated with a Ras mutation. In some embodiments, the cancer is associated with a KRAS mutation. In some embodiments, the cancer is associated with a KRAS G12C mutant.

In some embodiments the disclosure provides a method of treating a disorder in a subject in need thereof, wherein the said method comprises determining if the subject has a K-Ras, H-Ras or N-Ras G12C mutation and if the subject is determined to have a K-Ras, H-Ras or N-Ras G12C mutation, then administering to the subject a therapeutically effective amount of at least one compound of the present disclosure, or a pharmaceutically acceptable salt thereof.

K-Ras, H-Ras or N-Ras G12C mutations have also been identified in hematological malignancies (e.g., cancers that affect blood, bone marrow, and/or lymph nodes). Accordingly, certain embodiments are directed to administration of a disclosed combination or composition of the present disclosure (e.g., in the form of a pharmaceutical composition) to a patient in need of treatment of a hematological malignancy. Such malignancies include, but are not limited to leukemias and lymphomas. For example, the presently disclosed combinations and compositions can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the combinations and compositions of the present disclosure are useful for treatment of lymphomas such as all subtypes of Hodgkin's lymphoma or non-Hodgkin's lymphoma.

Determining whether a tumor or cancer comprises a G12C K-Ras, H-Ras or N-Ras mutation can be undertaken by assessing the nucleotide sequence encoding the K-Ras, H-Ras or N-Ras protein, by assessing the amino acid sequence of the K-Ras, H-Ras or N-Ras protein, or by assessing the characteristics of a putative K-Ras, H-Ras or N-Ras mutant protein. The sequences of wild-type human K-Ras (e.g. Accession No. NP203524), H-Ras (e.g. Accession No. NP001123914) and N-Ras (e.g. Accession No. NP002515) are known in the art.

Methods for detecting a mutation in a K-Ras, H-Ras or N-Ras nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C K-Ras, H-Ras or N-Ras mutations by real-time PCR. In real-time PCR, fluorescent probes specific for the K-Ras, H-Ras or N-Ras G12C mutation are used. When a mutation is present, the probe binds and fluorescence is detected. In some embodiments, the K-Ras, H-Ras or N-Ras G12C mutation is identified using a direct sequencing method of specific regions (e.g., exon 2 and/or exon 3) in the K-Ras, H-Ras or N-Ras gene. This technique will identify all possible mutations in the region sequenced.

Methods for detecting a mutation in a K-Ras, H-Ras or N-Ras protein are known by those of skill in the art. These methods include, but are not limited to, detection of a K-Ras, H-Ras or N-Ras mutant using a binding agent (e.g., an antibody) specific for the mutant protein, protein electrophoresis and Western blotting, and direct peptide sequencing. Methods for determining whether a tumor or cancer comprises a G12C K-Ras, H-Ras or N-Ras mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

Embodiments also relate to a method of treating a hyperproliferative disorder in a mammal that comprises administering to said mammal a therapeutically effective amount of combinations and compositions of the present disclosure. In some embodiments, said method relates to the treatment of cancer such as acute myeloid leukemia, cancer in adolescents, childhood adrenocortical carcinoma, AIDS-related cancers (e.g. lymphoma and Kaposi's sarcoma), anal cancer, appendix cancer, astrocytomas, atypical teratoid rhabdoid tumor, basal cell carcinoma, bile duct cancer, bladder cancer, bone cancer, brain stem glioma, brain tumor, breast cancer, bronchial tumors, Burkitt lymphoma, carcinoid tumor, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small-cell lung cancer (NSCLC), oral cancer, oropharyngeal cancer, ovarian cancer, pancreatic cancer, papillomatosis, paraganglioma, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer. In some embodiments, said method relates to the treatment of a non-cancerous hyperproliferative disorder such as benign hyperplasia of the skin (e.g., psoriasis), restenosis, or benign prostatic hyperplasia (BPH).

In certain particular embodiments, the disclosure relates to methods for treatment of lung cancers, the methods comprise administering a therapeutically effective amount of a combination or composition of the present disclosure to a subject in need thereof. In certain embodiments the lung cancer is a non-small-cell lung carcinoma (NSCLC), for example adenocarcinoma, squamous-cell lung carcinoma or large-cell lung carcinoma. In other embodiments, the lung cancer is a small cell lung carcinoma. Other lung cancers treatable with the disclosed compounds include, but are not limited to, glandular tumors, carcinoid tumors and undifferentiated carcinomas.

In some embodiments, the disclosure provides methods of inhibiting K-Ras, H-Ras, or N-Ras G12C activity in a cell by contacting said cell with an amount of a combination or composition of the present disclosure sufficient to inhibit the activity of K-Ras, H-Ras or N-Ras G12C in said cell. In some embodiments, the disclosure provides methods of inhibiting K-Ras, H-Ras or N-Ras G12C activity in a tissue by contacting said tissue with an amount of a combination or composition of the present disclosure sufficient to inhibit the activity of K-Ras, H-Ras or N-Ras G12C in said tissue. In some embodiments, the disclosure provides methods of inhibiting K-Ras, H-Ras or N-Ras G12C activity in an organism by contacting said organism with an amount of a combination or composition of the present disclosure sufficient to inhibit the activity of K-Ras, H-Ras or N-Ras G12C in said organism. In some embodiments, the disclosure provides methods of inhibiting K-Ras, H-Ras or N-Ras G12C activity in an animal by contacting said animal with an amount of a combination or composition of the present disclosure sufficient to inhibit the activity of K-Ras, H-Ras or N-Ras G12C in said animal. In some embodiments, the disclosure provides methods of inhibiting K-Ras, H-Ras or N-Ras G12C activity in a mammal by contacting said mammal with an amount of a combination or composition of the present disclosure sufficient to inhibit the activity of K-Ras, H-Ras or N-Ras G12C in said mammal. In some embodiments, the disclosure provides methods of inhibiting K-Ras, H-Ras or N-Ras G12C activity in a human by contacting said human with an amount of a combination or composition of the present disclosure sufficient to inhibit the activity of K-Ras, H-Ras or N-Ras G12C in said human. In other embodiments, the present disclosure provides methods of treating a disease mediated by K-Ras, H-Ras or N-Ras G12C activity in a subject in need of such treatment.

In some embodiments, the disclosure provides methods of treating cancer comprising administering to an individual in need thereof a therapeutically effective amount of the combination or composition of the present disclosure. In some embodiments, the individual is a human. In some embodiments, the administering is via the oral route. In some embodiments, the administering is via injection. In some embodiments, the cancer is mediated by a K-Ras G12C, H-Ras G12C or N-Ras G12C mutation. In some embodiments, the cancer is mediated by a K-Ras G12C mutation. In some embodiments, the cancer is a hematological cancer, pancreatic cancer, MYH-associated polyposis, colorectal cancer or lung cancer. In one embodiment, the cancer is lung cancer, colorectal cancer, appendicial cancer, or pancreatic cancer. In one embodiment, the cancer is colorectal cancer. In another embodiment, the cancer is pancreatic cancer. In some embodiments, the cancer is lung adenocarcinoma.

In some embodiments, the disclosure provides methods for regulating activity of a mutant protein selected from the group consisting of K-Ras G12C, H-Ras G12C and N-Ras G12C, the method comprising reacting the mutant protein with the combination or composition of the present disclosure.

In some embodiments, the disclosure provides methods for inhibiting proliferation of a cell population, the method comprising contacting the cell population with the combination or composition of the present disclosure. In some embodiments, the inhibition of proliferation is measured as a decrease in cell viability of the cell population.

In some embodiments, the disclosure provides methods for treating a disorder mediated by a mutation selected from the group consisting of K-Ras G12C, H-Ras G12C and N-Ras G12C in an individual in need thereof, the method comprising: determining if the individual has the mutation; and if the individual is determined to have the mutation, then administering to the individual a therapeutically effective amount of the combination or composition of the present disclosure. In some embodiments, the disorder is mediated by a K-Ras G12C mutation. In some embodiments, the disorder is a cancer. In some embodiments, the cancer is a hematological cancer, pancreatic cancer, MYH-associated polyposis, colorectal cancer or lung cancer. In one embodiment, the cancer is lung cancer, colorectal cancer, appendicial cancer, or pancreatic cancer. In one embodiment, the cancer is colorectal cancer. In another embodiment, the cancer is pancreatic cancer. In some embodiments, the cancer is lung adenocarcinoma.

In some embodiments, the disclosure provides methods for preparing a labeled K-Ras G12C, H-Ras G12C or N-Ras G12C mutant protein, the method comprising reacting a K-Ras G12C, H-Ras G12C or N-Ras G12C mutant protein with a combination or composition of the present disclosure to result in the labeled K-Ras G12C, H-Ras G12C or N-Ras G12C mutant protein.

In some embodiments, the disclosure provides methods for inhibiting tumor metastasis comprising administering to an individual in need thereof a therapeutically effective amount of the combination or composition of the present disclosure.

In some embodiments, the disclosure provides uses of a combination or composition of the present disclosure in the manufacture of a medicament for treating cancer. In some embodiments, the medicament is formulated for oral administration. In some embodiments, the medicament is formulated for injection. In some embodiments, the cancer is mediated by a K-Ras G12C, H-Ras G12C or N-Ras G12C mutation. In some embodiments, the cancer is mediated by a K-Ras G12C mutation. In some embodiments, the cancer is mediated by a H-Ras G12C mutation. In some embodiments, the cancer is mediated by a N-Ras G12C mutation. In some embodiments, the cancer is a hematological cancer, pancreatic cancer, MYH-associated polyposis, colorectal cancer or lung cancer. In one embodiment, the cancer is lung cancer, colorectal cancer, appendicial cancer, or pancreatic cancer. In one embodiment, the cancer is colorectal cancer. In another embodiment, the cancer is pancreatic cancer. In some embodiments, the cancer is lung adenocarcinoma. In some embodiments, the disclosure provides uses of a compound of the present disclosure, or a pharmaceutically acceptable salt thereof, in the manufacture of a medicament for inhibiting tumor metastasis.

In some embodiments, the disclosure provides a combination or composition of the present disclosure for use in a method of treatment of the human or animal body by therapy. In some embodiments, the disclosure provides a combination or composition of the present disclosure for use in a method of treating cancer. In some embodiments, the cancer is mediated by a K-Ras G12C, H-Ras G12C or N-Ras G12C mutation. In some embodiments, the cancer is mediated by a K-Ras G12C mutation. In some embodiments, the cancer is mediated by a H-Ras G12C mutation. In some embodiments, the cancer is mediated by a N-Ras G12C mutation. In some embodiments, the cancer is a hematological cancer, pancreatic cancer, MYH-associated polyposis, colorectal cancer or lung cancer. In one embodiment, the cancer is lung cancer, colorectal cancer, appendicial cancer, or pancreatic cancer. In one embodiment, the cancer is colorectal cancer. In another embodiment, the cancer is pancreatic cancer. In some embodiments, the cancer is lung adenocarcinoma. In some embodiments, the disclosure provides a compound of the present disclosure, or a pharmaceutically acceptable salt thereof, or a pharmaceutical composition of the present disclosure, for use in a method of inhibiting tumor metastasis.

Further provided herein are methods of treating lung cancer in a patient having lung cancer, comprising administering a therapeutically effective amount of a combination or composition described herein to the patient. In one embodiment, the lung cancer is non-small-cell lung carcinoma (NSCLC). The NSCLC can be, for example, adenocarcinoma, squamous-cell lung carcinoma or large-cell lung carcinoma. In another embodiment, the lung cancer is small cell lung carcinoma. In still another embodiment, the lung cancer is glandular tumors, carcinoid tumors or undifferentiated carcinomas. The lung cancer can be stage I or II lung cancer. In one embodiment, the lung cancer is stage III or IV lung cancer. The methods provided herein include administration of the compound as a 1 L therapy. In one embodiment, the lung cancer comprises a G12C KRas mutation.

Still further provided herein are methods of treating pancreatic cancer in a patient having pancreatic cancer, the method comprising administering a therapeutically effective amount of a combination or composition described herein to the patient. In one embodiment, the patient has been previously treated with radiation and one or more chemotherapy agents. In one embodiment, the pancreatic cancer is stage 0, I, or II. In another embodiment, the pancreatic cancer is stage III or stage IV. In one embodiment, the pancreatic cancer comprises a KRas mutation.

Still further provided herein are methods of treating colon cancer in a patient having colon cancer, the method comprising administering a therapeutically effective amount of a combination or composition described herein to the patient. In one embodiment, the colon cancer is stage I or II. In another embodiment, the colon cancer is stage III or stage IV. In one embodiment, the colon cancer comprises a G12C KRas mutation.

Further provided herein are methods of treating tumor agnostic G12C mutant KRas mediated cancer. In one embodiment of such methods, the method comprises:
a. determining the absence or presence of a KRas G12C mutation in a sample taken from a patient with a suspected diagnosed cancer; and
b. administering to the patient a therapeutically effective amount of a combination or composition described herein.

In one embodiment of such methods, the patient is diagnosed with a cancer described herein. In another embodiment of such methods, the sample is a tumor sample taken from the subject. In one such embodiment, the sample is taken before administration of any therapy. In another such embodiment, the sample is taken before administration of a compound of pharmaceutically acceptable salt thereof described herein and after administration of another chemotherapeutic agent. In another embodiment of such methods, the compound or pharmaceutically acceptable salt thereof described herein is administered as provided herein (e.g. orally).

In some embodiments, the cancer is lung cancer. In some embodiments, the cancer is non-small-cell lung cancer (NSCLC). In another such embodiment, the cancer is NSCLC, adenocarcinoma, squamous-cell lung carcinoma, large-cell lung carcinoma, or SCLC mediated by a KRAS G12C mutation.

In some embodiments, the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germmoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles (dysplastic nevi), lipoma, angioma, dermatofibroma, keloids, psoriasis; and Adrenal glands: neuroblastoma.

### Breast Cancer

Combinations and compositions of the disclosure can be administered alone or they can be used in a combination therapy for the treatment of breast cancer. For instance, the combination therapy includes administering a combination or composition of the disclosure and administering at least one additional therapeutic agent (e.g. one, two, three, four, five, or six additional therapeutic agents) for the treatment of breast cancer.

Standard of care for breast cancer is determined by both disease (tumor, stage, pace of disease, etc.) and patient characteristics (age, by biomarker expression and intrinsic phenotype). General guidance on treatment options are described in the NCCN Guidelines (e.g., NCCN Clinical Practice Guidelines in Oncology, Breast Cancer, version 2.2016, National Comprehensive Cancer Network, 2016, pp. 1-202), and in the ESMO Guidelines (e.g., Senkus, E., et al. Primary Breast Cancer: ESMO Clinical Practice Guidelines for diagnosis, treatment and follow-up. Annals of Oncology 2015; 26(Suppl. 5): v8-v30; and Cardoso F., et al. Locally recurrent or metastatic breast cancer: ESMO Clinical Practice Guidelines for diagnosis, treatment and follow-up. Annals of Oncology 2012; 23 (Suppl. 7):vii11-vii19.).

In some aspects, the combinations and compositions of the present disclosure are for use in a combination therapy for the treatment of breast cancer in combination with one or more other therapeutic agents. In a further aspect, the combinations and compositions of the present disclosure are for use in a combination therapy for the treatment of early breast cancer or locally advanced breast cancer. In a further aspect, the combinations and compositions of the present disclosure are for use in a combination therapy for the treatment of advanced breast cancer or metastatic breast cancer.

In particular, the combinations and compositions of the present disclosure of the disclosure can be used either alone or in combination with standard of care treatment options for breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

Thus, in one embodiment, the combination therapy comprises administering a combinations and compositions of the present disclosure of the present disclosure and administering at least one additional therapeutic agent such as doxorubicin, epirubicin, cyclophosphamide, docetaxel, paclitaxel, methotrexate, and/or 5-fluorouracil.

In one embodiment, the combination therapy comprises administering a combinations and compositions of the present disclosure and administering doxorubicin and cyclophosphamide (AC chemotherapy). In one embodiment, the combination therapy comprises administering a combination or composition of the present disclosure and administering docetaxel, doxorubicin and cyclophosphamide (TAC chemotherapy). In one embodiment, the combination therapy comprises administering a combination or composition of the present disclosure and administering cyclophosphamide, methotrexate and 5-fluorouracil (CMF chemotherapy). In one embodiment, the combination therapy comprises administering a combination or composition of the present disclosure and administering epirubicin and cyclophosphamide (EC chemotherapy). In one embodiment, the combination therapy comprises administering a combination or composition of the present disclosure and administering 5-fluorouracil, epirubicin and cyclophosphamide (FEC chemotherapy). In one embodiment, the combination therapy comprises administering a combination or composition of the present disclosure and administering 5-fluorouracil, doxorubicin and cyclophosphamide (FAC chemotherapy). In one embodiment, the combination therapy comprises administering a combination or composition of the present disclosure and administering taxane, in particular docetaxel or paclitaxel.

In one embodiment, when the combinations or compositions of the disclosure are for use in the treatment of metastatic breast cancer, the combination therapy comprises administering a combination or composition of the present disclosure and administering at least one additional therapeutic agent such as doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, Ixabepilone, methotrexate, and/or 5-fluorouracil (5-FU). In one embodiment, the combination therapy comprises administering a combination or composition of the present disclosure and administering docetaxel and capecitabine for use in the treatment of metastatic breast cancer. In one embodiment, the combination therapy comprises administering a combination or composition of the present disclosure and administering gemcitabine and paclitaxel for use in the treatment of metastatic breast cancer.

Breast Cancer—Hormone Receptor Positive (ER+ and/or PR+)

In a further aspect, the disclosure provides a method for treating hormone receptor positive (HR+) breast cancer (also called estrogen receptor positive (ER+) breast cancer or estrogen receptor positive and/or progesterone receptor positive (PR+) breast cancer), by administering an effective amount of a combination or composition of the present disclosure. In a further aspect of the embodiment, the breast cancer is early or locally advanced hormone receptor positive (HR+) breast cancer, also named early or locally advanced ER+ breast cancer. In a further aspect, the breast cancer is advanced hormone receptor positive (HR+) breast cancer or metastatic hormone receptor positive (HR+) breast cancer, also named advanced ER+ breast cancer or metastatic ER+ breast cancer.

In some aspects, the combinations or compositions are for use in a combination therapy for the treatment of hormone receptor positive (HR+) breast cancer or estrogen receptor positive (ER+) breast cancer. In a further aspect, the combinations or compositions are for use in a combination therapy for the treatment of early or locally advanced hormone receptor positive (HR+) breast cancer, also named early or locally advanced ER+ breast cancer. In a further aspect of the embodiment, the combinations or compositions are for use in a combination therapy for the treatment of advanced hormone receptor positive (HR+) breast cancer or metastatic hormone receptor positive (HR+) breast cancer, also named advanced ER+ breast cancer or metastatic ER+ breast cancer. In one embodiment, the method comprises administering to an individual having hormone receptor positive (HR+) breast cancer or estrogen receptor positive (ER+) breast cancer an effective amount of a combination or composition of the present disclosure in combination with one or more other therapeutic agents.

In particular, a combination or composition of the disclosure can be used either alone or in combination with standard of care treatment options for hormone receptor positive (HR+) breast cancer or estrogen receptor positive (ER+) breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

In one embodiment, combinations or compositions of the disclosure are for use in the treatment of hormone receptor positive (HR+) breast cancer or estrogen receptor positive (ER+) breast cancer in combination with endocrine therapy. In one embodiment, the combination therapy comprises administering a combination or composition of the present disclosure and administering tamoxifen. In one embodiment, the combination therapy comprises administering an a combination or composition of the present disclosure and administering an aromatase inhibitor, such as anastrozole, letrozole or exemestane for use in the treatment of hormone receptor positive (HR+) breast cancer or estrogen receptor positive (ER+) breast cancer. In one embodiment, the combination therapy comprises administering a combination or composition of the present disclosure and administering at least one additional therapeutic agent such as anastrozole, letrozole, exemestane and everolimus, palbociclib and letrozole, palbociclib and letrozole, fulvestrant, tamoxifen, toremifene, megestrol acetate, fluoxemesterone, and/or ethinyl estradiol for use in the treatment of hormone receptor positive (HR+) breast cancer or estrogen receptor positive (ER+) breast cancer.

In one embodiment, combinations or compositions of the disclosure are for use in the treatment of hormone receptor positive (HR+) breast cancer or estrogen receptor positive (ER+) breast cancer in combination with one or more chemotherapeutic agents. In one embodiment, the combination therapy comprises administering a combination or composition of the present disclosure and administering at least one additional therapeutic agent such as doxorubicin, epirubicin, cyclophosphamide, docetaxel, paclitaxel, methotrexate, and/or 5-fluorouracil for use in the treatment of hormone receptor positive (HR+) breast cancer or estrogen receptor positive (ER+) breast cancer.

In one aspect, combinations or compositions of the disclosure are for use in combination with doxorubicin and cyclophosphamide (AC chemotherapy). In one embodiment, the combination therapy comprises administering a combination or composition of the present disclosure and administering docetaxel, doxorubicin and cyclophosphamide (TAC chemotherapy). In one embodiment, the combination therapy comprises administering a combination or composition of the present disclosure and administering cyclophosphamide, methotrexate and 5-fluorouracil (CMF chemotherapy). In one embodiment, the combination therapy comprises administering a combination or composition of the present disclosure and administering epirubicin and cyclophosphamide (EC chemotherapy). In one embodiment, the combination therapy comprises administering a combination or composition of the present disclosure and administering 5-fluorouracil, epirubicin and cyclophosphamide (FEC chemotherapy). In one embodiment, the combination therapy comprises administering a combination or composition of the present disclosure and administering 5-fluorouracil, doxorubicin and cyclophosphamide (FAC chemotherapy). In one embodiment, the combination therapy comprises administering a combination or composition of the present disclosure and administering a taxane, such as docetaxel or paclitaxel.

In one embodiment, compounds of the disclosure are for use in the treatment of metastatic breast cancer. In one embodiment, the combination therapy comprises administering an a combination or composition of the present disclosure and administering doxorubicin, pegylated liposomal doxorubicin, epirubicin, cyclophosphamide, carboplatin, cisplatin, docetaxel, paclitaxel, albumin-bound paclitaxel, capecitabine, gemcitabine, vinorelbine, eribulin, ixabepilone, methotrexate and 5-fluorouracil (5-FU) for use in the treatment of metastatic breast cancer. In one embodiment, the combination therapy comprises administering a combination or composition of the present disclosure and administering docetaxel and capecitabine for use in the treatment of metastatic breast cancer. In one embodiment, the combination therapy comprises administering a combination or composition of the present disclosure and administering gemcitabine and paclitaxel for use in the treatment of metastatic breast cancer.

### Breast Cancer—HER2+

In a further aspect, the disclosure provides a method for treating Her2+ positive breast cancer, by administering an effective amount of a combination or composition of the present disclosure. In a further aspect of the embodiment, the breast cancer is early or locally advanced Her2+ positive breast cancer, also named early or locally advanced Her2+ positive breast cancer. In a further aspect, the breast cancer is advanced breast cancer, also named advanced Her2+ positive breast cancer or metastatic ER+ breast cancer.

In some aspects, the combinations or compositions are for use in a combination therapy for treatment of Her2+ positive breast cancer. In a further aspect, the combinations or composition are for use in a combination therapy for treatment of early or locally advanced Her2+ positive breast cancer, also named early or locally advanced Her2+ positive breast cancer. In a further aspect of the embodiment, the combinations or compositions are for use in a combination therapy for treatment of advanced Her2+ positive breast cancer, also named advanced Her2+ positive breast cancer or metastatic ER+ breast cancer. In one embodiment, the method comprises administering to an individual having Her2+ positive breast cancer an effective amount of a combination or composition of the present disclosure in combination with one or more other therapeutic agents.

In particular, compounds of the disclosure can be used either alone or in combination with standard of care treatment options for Her2+ positive breast cancer, which in general include surgery, systemic chemotherapy (either pre- or post-operatively) and/or radiation therapy. Depending on tumor and patient characteristics, systemic chemotherapy may be administered as adjuvant (post-operative) therapy or as neoadjuvant (pre-operative) therapy.

In one embodiment, the combination therapy comprises administering a combination or composition of the present disclosure and administering a Her2 antibody to treat Her2+ positive breast cancer. In one aspect, the combination therapy comprises administering a combination or composition of the present disclosure and administering trastuzumab or pertuzumab to treat Her2+ positive breast cancer. In another aspect, the combination therapy comprises administering a combination or composition of the present disclosure and administering a chemotherapy to treat Her2+ positive breast cancer. In one embodiment, the combination therapy comprises administering a combination or composition of the present disclosure and administering doxorubicin and cyclophosphamide followed by trastuzumab to treat Her2+ positive breast cancer. In a further embodiment, combinations or compositions of the disclosure are for use in the treatment of Her2+ positive breast cancer in combination with chemotherapy followed by a taxane and trastuzumab to treat Her2+ positive breast cancer. In another aspect, combinations or composition of the disclosure are for use in the treatment of Her2+ positive breast cancer in combination with trastuzumab (Herceptin) and pertuzumab (Perjeta) to treat Her2+ positive breast cancer.

In another aspect, combinations or compositions of the disclosure are used in combination with docetaxel, carboplatin and trastuzumab (TCH chemotherapy). In a further aspect, compounds of the disclosure are administered in combination with docetaxel, carboplatin, trastuzumab and pertuzumab. In a further aspect, combinations or compositions of the disclosure are administered in combination with 5-fluorouracil, epirubicin and cyclophosphamide (FEC chemotherapy and pertuzumab, trastuzumab and docetaxel or paclitaxel. In another aspect, combinations or compositions of the disclosure are used in combination with paclitaxel and trastuzumab. In a further aspect, combinations or compositions of the disclosure are administered in combination with Pertuzumab and trastuzumab and paclitaxel or docetaxel.

If the combinations or compositions of the disclosure are for use in the treatment of metastatic Her2+ positive breast cancer, they can also be used in combination with one or more chemotherapeutic agents selected from the group consisting of doxorubicin (A) (Adriamycin), pegylated liposomal doxorubicin (Doxil), epirubicin (E) (Ellence), cyclophosphamide (C) (Cytoxan), carboplatin (Platinol), cisplatin (Paraplatin), docetaxel (T) (Taxotere), paclitaxel (Taxol), albumin-bound paclitaxel (Abraxane), capecitabine (Xeloda), gemcitabine (Cynzar), vinorelbine (Navelbine), eribulin (Halaven), and Ixabepilone (Ixempra), In one aspect, the combinations or compositions of the disclosure are for use in the treatment of metastatic Her2+ positive breast cancer in combination with ado-trastuzumab emtansine (T-DM1).

In a particular aspect, combinations or compositions of the disclosure are for use in the treatment of metastatic Her2+ positive breast cancer in combination with trastuzumab and pertuzumab and a taxane. In one aspect, the taxane is docetaxel. In another aspect, the taxane is paclitaxel.

### Breast Cancer—Triple Negative

Combinations or compositions of the disclosure can be used either alone or in a combination therapy with standard of care treatment options for triple negative breast cancer (TNBC), which in general include surgery, systemic chemotherapy (either pre- or post-operatively) and/or radiation therapy.

Standard of care for TNBC is determined by both disease (stage, pace of disease, etc.) and patient (age, co-morbidities, symptoms, etc.) characteristics. General guidance on treatment options are described in the NCCN Guidelines (e.g., NCCN Clinical Practice Guidelines in Oncology, Breast Cancer, version 2.2016, National Comprehensive Cancer Network, 2016, pp. 1-202), and in the ESMO Guidelines (e.g., Senkus, E., et al. Primary Breast Cancer: ESMO Clinical Practice Guidelines for diagnosis, treatment and follow-up. Annals of Oncology 2015; 26(Suppl. 5): v8-v30; and Cardoso F., et al. Locally recurrent or metastatic breast cancer: ESMO Clinical Practice Guidelines for diagnosis, treatment and follow-up. Annals of Oncology 2012; 23 (Suppl. 7):vii11-vii19.). See also, Rodler, E, et al. Breast Disease. 2010/2011; 32:99-122.

### Metastatic TNBC

Systemic chemotherapy is the standard treatment for patients with metastatic TNBC, although no standard regimen or sequence exists. Single-agent cytotoxic chemotherapeutic agents as shown in Table 2 are generally regarded as the primary option for patients with metastatic TNBC, although combination chemotherapy regimens such as those shown in Table 3 may be used, for instance when there is aggressive disease and visceral involvement. Additional details on chemotherapy combinations that can be utilized are provided below in the section on early and locally advanced treatment options. Treatment may also involve sequential rounds of different single agent treatments. Palliative surgery and radiation may be utilized as appropriate to manage local complications.

The methods provided herein include administering a combination or composition of the present disclosure to a patient with metastatic TNBC in combination with one of the single-agent chemotherapy agents listed in Table 2 or in combination with sequential rounds of different chemotherapy agents listed in Table 2. Such methods may optionally be combined with surgery and/or radiation treatment.

TABLE 2
Single agent chemotherapy regimens

| Class | Typical agents |
|---|---|
| Anthracyclines | Doxorubicin |
| | Pegylated liposomal doxorubicin |
| | Epirubicin |
| Taxanes | Paclitaxel |
| | Docetaxel |
| | Albumin-bound paclitaxel (nab-paclitaxel) |
| Anti-metabolites | Capecitabine |
| | Gemcitabine |
| Non-taxane | Vinorelbine |
| microtubule inhibitors | Eribulin |
| | Ixabepilone |
| Platinum | Carboplatin |
| | Cisplatin |
| Alkylating agent | Cyclophosphamide |

In one embodiment, the combination therapy comprises administering a combination or composition of the present disclosure and administering an anthracycline such as doxorubicin, pegylated liposomal doxorubicin, or epirubicin.

In one embodiment, the combination therapy comprises administering a combination or composition of the present disclosure and administering a taxane such as paclitaxel, docetaxel or albumin-bound paclitaxel (e.g., nab-paclitaxel).

In one embodiment, the combination therapy comprises administering a combination or composition of the present disclosure and administering an anti-metabolite, including, for example, capecitabine or gemcitabine.

In one embodiment, the combination therapy comprises administering a combination or composition of the present disclosure and administering a non-taxane microtubule inhibitor, such as vinorelbine, eribulin or ixabepilone.

In one embodiment, the combination therapy comprises administering a combination or composition of the present disclosure and administering a platinum compound, such as carboplatin or cisplatin.

In one embodiment, the combination therapy comprises administering a combination or composition of the present disclosure and administering an alkylating agent such as cyclophosphamide.

In some embodiments, a combination or composition of the present disclosure is administered with a combination of chemotherapy agents as summarized in Table 3 below.

Additional guidance for treating metastatic TNBC is provided in Jones S E, et al. J Clin Concol. 2006; 24:5381-5387; Heemskerk-Gerritsen B A M, et al. Ann Surg. Oncol. 2007; 14:3335-3344; and Kell M R, et al. MBJ. 2007; 334:437-438.

### Early and Locally Advanced TNBC

Patients with early and potentially resectable locally advanced TNBC (i.e. without distant metastatic disease) are managed with locoregional therapy (surgical resection with or without radiation therapy) with or without systemic chemotherapy.

Surgical treatment can be breast-conserving (e.g., a lumpectomy, which focuses on removing the primary tumor with a margin), or can be more extensive (e.g., mastectomy, which aims for complete removal of all of the breast tissue). Radiation therapy is typically administered post-surgery to the breast/chest wall and/or regional lymph nodes, with the goal of killing microscopic cancer cells left post-surgery. In the case of a breast conserving surgery, radiation is administered to the remaining breast tissue and sometimes to the regional lymph nodes (including axillary lymph nodes). In the case of a mastectomy, radiation may still be administered if factors that predict higher risk of local recurrence are present.

In one embodiment, a combination or composition of the present disclosure is administered in combination with surgical treatment, either as a neoadjuvant or adjuvant therapy. In another embodiment, a combination or composition of the present disclosure is administered before or after radiation treatment. In still another embodiment, a combination or composition of the present disclosure is administered in combination with surgical and radiation treatment.

Depending on tumor and patient characteristics, chemotherapy may be administered in the adjuvant (post-operative) or neoadjuvant (pre-operative) setting. Examples of adjuvant/neoadjuvant chemotherapy regimens used to treat TNBC recommended by current guidelines are shown in Table 3. A combination or composition of the present disclosure can be combined with any of the regimens shown in Table 3.

TABLE 3
Combination chemotherapy regimens

| Class | Typical agents | Shorthand |
|---|---|---|
| Anthracycline and | Doxorubicin + cyclophosphamide | AC→ T |
| alkylating agent | followed by a taxane (e.g., | |
| followed by | docetaxel or paclitaxel) | |
| taxane | | |
| Anthracycline and | Doxorubicin + cyclophosphamide (or | AC |
| alkylating agent | liposomal doxorubicin + | |
| | cyclophosphamide) | |
| | Epirubicin + cyclophosphamide | EC |
| Taxane, | Docetaxel + doxorubicin + | TAC |
| anthracycline, and | cyclophosphamide | |
| alkylating agent | | |
| Taxane and | Docetaxel + cyclophosphamide | TC |
| alkylating agent | | |
| Alkylating agent, | Cyclophosphamide + methotrexate + | CMF |
| methotrexate and | fluorouracil | |
| anti-metabolite | | |
| Anti-metabolite, | Fluorouracil + doxorubicin + | FAC |
| anthracycline, and | cyclophosphamide | |
| alkylating agent | Fluorouracil + epirubicin + | FEC |
| | cyclophosphamide | |
| Anti-metabolite, | Fluorouracil + epirubicin + | FEC/ |
| anthracycline, and | cyclophosphamide followed by | CEF→T |
| alkylating agent | docetaxel or paclitaxel | |
| followed by | Fluorouracil + doxorubicin + | FAC→T |
| taxane | cyclophosphamide followed by paclitaxel | |
| Taxane and anti- | Docetaxel + capecitabine, or | GT |
| metabolite | Paclitaxel + gemcitabine | |
| Anti-metabolite | Gemcitabine + carboplatin | |
| and platinum | | |
| Anti-metabolite | Capecitibine + vinorelbine | |
| and non-taxane | Gemcitabine + vinorelbine | |
| microtubule | | |
| inhibitor | | |
| Taxane and | Paclitaxel + bevacizumab | |
| VEGF inhibitor | | |
| (e.g., anti-VEGF | | |
| antibody) | | |

In one embodiment, the combination therapy comprises administering a combination or composition of the present disclosure and administering an anthracycline and an alkylating agent, optionally followed by a taxane. In one such embodiment, the combination or composition of the present disclosure is administered with doxorubicin and cyclophosphamide followed by a taxane (e.g., docetaxel or paclitaxel), which is a chemotherapy regimen designated as AC→T.

In one embodiment, the combination therapy comprises administering a combination or composition of the present disclosure and administering an anthracycline and an alkylating agent. For example, in one embodiment, the combination therapy comprises administering a combination or composition of the present disclosure and administering doxorubicin or liposomal doxorubicin and cyclophosphamide, which is designated as AC. In another embodiment, the combination therapy comprises administering a combination or composition of the present disclosure and administering epirubicin and cyclophosphamide, which is a chemotherapy regimen referred to as EC.

In one embodiment, the combination therapy comprises administering a combination or composition of the present disclosure and administering a taxane, an anthracycline, and an alkylating agent. For instance, in one embodiment, the combination therapy comprises administering a compound of the present disclosure and administering docetaxel, doxorubicin and cyclophosphamide, a chemotherapy regimen which is denoted as TAC.

In another embodiment, the combination therapy comprises administering a combination or composition of the present disclosure and administering taxane and an alkylating agent. In one such embodiment, the combination therapy comprises administering a combination or composition of the present disclosure and administering docetaxel and cyclophosphamide, which is a chemotherapy regimen referred to as TC.

In still another embodiment, the combination therapy comprises administering a combination or composition of the present disclosure and administering taxane and an alkylating agent. For instance, in one embodiment, the combination therapy comprises administering a combination or composition of the present disclosure and administering docetaxel and cyclophosphamide, a chemotherapy regimen designated as TC.

In one embodiment, the combination therapy comprises administering a combination or composition of the present disclosure and administering an alkylating agent, methotrexate, and an anti-metabolite. As an example, in one embodiment, the combination therapy comprises administering a combination or composition of the present disclosure and administering an alkylating agent, methotrexate and an anti-metabolite. In one such embodiment, the combination therapy comprises administering a combination or composition of the present disclosure and administering cyclophosphamide, methotrexate and fluorouracil, a chemotherapy regimen which is referred to as CMF.

In another embodiment, the combination therapy comprises administering a combination or composition of the present disclosure and administering an anti-metabolite, an anthracycline, and an alkylating agent. In one such embodiment, the combination therapy comprises administering a combination or composition of the present disclosure and administering fluorouracil, doxorubicin and cyclophosphamide, which is a chemotherapy regimen denoted as FAC. In another such embodiment, the combination therapy comprises administering a combination or composition of the present disclosure and administering fluorouracil, epirubicin and cyclophosphamide, a chemotherapy regimen designated as FEC.

In still another embodiment, the combination therapy comprises administering a combination or composition of the present disclosure and administering an anti-metabolite, an anthracycline, and an alkylating agent followed by taxane. As an example, in one embodiment, the combination therapy comprises administering a combination or composition of the present disclosure and administering fluorouracil, epirubicin and cyclophosphamide followed by docetaxel or paclitaxel, a chemotherapy regimen referred to as FEC (or CEF)→T. In another embodiment, the combination therapy comprises administering a combination or composition of the present disclosure and administering fluorouracil, doxorubicin and cyclophosphamide followed by paclitaxel, which is a chemotherapy regimen designated as FAC→T.

In yet another embodiment, the combination therapy comprises administering a combination or composition of the present disclosure and administering taxane and an anti-metabolite. As an example, in one embodiment, the combination therapy comprises administering a combination or composition of the present disclosure and administering docetaxel and capecitabine. In another example the combination therapy comprises administering a combination or composition of the present disclosure and administering paclitaxel and gemcitabine, a chemotherapy regimen referred to as GT.

In one embodiment, the combination therapy comprises administering a combination or composition of the present disclosure and administering an anti-metabolite and a platinum compound. For instance, in one embodiment, the combination therapy comprises administering a combination or composition of the present disclosure and administering gemcitabine and carboplatin.

In another embodiment, the combination therapy comprises administering a combination or composition of the present disclosure and administering an anti-metabolite and a non-taxane microtubule inhibitor. In one such embodiment, the combination therapy comprises administering a combination or composition of the present disclosure and administering capecitibine and vinorelbine. In another such embodiment, the combination therapy comprises administering a combination or composition of the present disclosure and administering gemcitabine and vinorelbine.

In still another embodiment, the combination therapy comprises administering a combination or composition of the present disclosure and administering a taxane and a VEGF inhibitor (e.g., anti-VEGF antibody). For instance, in one embodiment, the combination therapy comprises administering a combination or composition of the present disclosure and administering paclitaxel and bevacizumab.

Additional guidance for treating early and locally advanced TNBC is provided in Solin L J., Clin Br Cancer. 2009; 9:96-100; Freedman G M, et al. Cancer. 2009; 115:946-951; Heemskerk-Gerritsen B A M, et al. Ann Surg Oncol. 2007; 14:3335-3344; and Kell M R, et al. MBJ. 2007; 334:437-438.

### Non-Small-Cell Lung Cancer (NSCLC)

Combinations or compositions of the disclosure can be administered alone or they can be used in a combination therapy. For instance, the combination therapy includes administering a combination or composition of the disclosure and administering at least one additional therapeutic agent (e.g. one, two, three, four, five, or six additional therapeutic agents).

In some aspects, the combinations or compositions are for use in a combination therapy for the treatment of non-small-cell lung cancer NSCLC, such as a squamous cell carcinoma, adenocarcinoma, large cell carcinoma, adenosquamous carcinoma, undifferentiated carcinoma, or a combination thereof.

In one embodiment, the NSCLC is associated with a KRAS mutation. In one embodiment, the NSCLC is associated with a KRAS G12C mutation.

In one embodiment, the NSCLC is in occult stage, stage 0, I, II, III, or IV.

In one embodiment, the NSLCL is in occult stage, stage 0, IA, IB, IIA, IIB, IIIA, IIIB, or IV.

The present disclosure is directed to use of disclosed combinations or compositions for an adjuvant or neo-adjuvant treatment.

The present disclosure is directed to use of disclosed combinations or compositions for a first line, second line, or third line treatment.

The present disclosure is directed to use of disclosed combinations or compositions for a single agent treatment.

The present disclosure is directed to use of disclosed combinations or compositions for a treatment of a stage IV or a recurrent disease.

The present disclosure is directed to use of disclosed combinations or compositions for a treatment which is combined with surgery, radiation therapy, or a combination thereof.

In one embodiment, the combination therapy comprises administering a combination or composition of the present disclosure and administering at least one additional therapeutic agent such as cisplatin, carboplatin, paclitaxel, paclitaxel protein bound, docetaxel, gemcitabine, vinorelbine, etoposide, nintedanib, vinblastine, and/or pemetrexed.

In one embodiment, the combination therapy comprises administering a combination or composition of the present disclosure and administering at least one additional therapeutic agent such as afatinib, bevacizumab, cabozantinib, ceritinib, crizotinib, erlotinib hydrochloride, osimertinib, ramucirumab, gefitinib, alectinib, trastuzumab, cetuximab, ipilimumab, trametinib, dabrafenib, vemurafenib, dacomitinib, tivantinib, and/or onartuzumab.

In one embodiment, the combination therapy comprises administering a combination or composition of the present disclosure and administering at least one additional therapeutic agent such as afatinib, crizotinib, erlotinib hydrochloride, and/or gefitinib.

In one embodiment, the combination therapy comprises administering a combination or composition of the present disclosure and administering checkpoint inhibitor agents, such as pembrolizumab, atezolizumab, and/or nivolumab.

In one embodiment, the combination therapy comprises administering a combination or composition of the present disclosure and administering at least one additional therapeutic agent such as cisplatin, carboplatin, paclitaxel, paclitaxel protein bound, docetaxel, gemcitabine, vinorelbine, etoposide, nintedanib, vinblastine, pemetrexed, afatinib, bevacizumab, cabozantinib, ceritinib, crizotinib, erlotinib hydrochloride, osimertinib, ramucirumab, gefitinib, necitumumab, alectinib, trastuzumab, cetuximab, ipilimumab, trametinib, dabrafenib, vemurafenib, dacomitinib, tivantinib, onartuzumab, pembrolizumab, atezolizumab, and/or nivolumab.

### Small Cell Lung Cancer (SCLC)

Combinations or compositions of the disclosure can be administered alone or they can be used in a combination therapy. For instance, the combination therapy includes administering a combination or composition of the disclosure and administering at least one additional therapeutic agent (e.g. one, two, three, four, five, or six additional therapeutic agents).

In some aspects, the combinations or compositions are for use in a combination therapy for the treatment of Small Cell Lung Cancer (SCLC).

In one embodiment, the SCLC is a small cell carcinoma (oat cell cancer), mixed small cell/large cell carcinoma or combined small cell carcinoma.

In one embodiment, the SCLC is in occult stage, stage 0, I, II, III, or IV.

In one embodiment, the SLCL is in occult stage, stage 0, IA, IB, IIA, IIB, IIIA, IIIB, or IV.

In one embodiment, the SLCL is in stage I-III (limited stage).

The present disclosure is directed to use of disclosed combinations or compositions for a first line treatment of stage IV (extensive stage).

The present disclosure is directed to use of disclosed combinations or compositions for a second line treatment of stage IV (relapsed or refractory disease).

The present disclosure is directed to use of disclosed combinations or compositions for a third line treatment of stage IV (relapsed or refractory disease).

In one embodiment, a combination or composition of the present disclosure is administered with one or more additional therapeutic agents selected from Etoposide, a platinum compound, Irinotecan, Topotecan, vinca alkaloids, alkylating agents, Doxorubicin, taxanes, and Gemcitabine. In another embodiment, the platinum compound is Cisplatin or Carboplatin. In another embodiment, the vinca alkaloid is Vinblastine, Vincristine, or Vinorelbine. In another embodiment, the alkylating agent is Cyclophosphamide or Ifosfamide. In another embodiment, the taxane is Docetaxel or Paclitaxel.

### Ovarian Cancer

In a further aspect, the disclosure provides a method for treating an ovarian cancer (such as epithelial ovarian cancer (EOC), ovarian germ cell tumors, or ovarian stromal tumors) by administering an effective amount of a combination or composition of the present disclosure. In a further aspect of the embodiment, the ovarian cancer is an epithelial ovarian cancer (EOC). In a further aspect of the embodiment, the ovarian cancer is an ovarian germ cell tumor. In a further aspect of the embodiment, the ovarian cancer is an ovarian stromal cell tumor. In one embodiment, the method comprises administering to an individual having ovarian cancer an effective amount of a combination or composition of the present disclosure.

Combinations or compositions of the disclosure can be administered alone or they can be used in a combination therapy to treat ovarian cancer. For instance, the combination therapy includes administering a combination or composition of the disclosure and administering at least one additional therapeutic agent (e.g. one, two, three, four, five, or six additional therapeutic agents).

In some aspects, the combination or compositions are for use in a combination therapy for the treatment of an ovarian cancer (such as epithelial ovarian cancer (EOC), ovarian germ cell tumors, or ovarian stromal tumors). In one embodiment, the combination therapy comprises administering a combination or composition of the present disclosure and administering at least one additional therapeutic agent such as a platinum compound (such as carboplatin, cisplatin, less often oxaliplatin or iproplatin), and/or a taxane (such as paclitaxel or docetaxel, or albumin bound paclitaxel (nab-paclitaxel)). In one embodiment, the combination therapy comprises administering a combination or composition of the present disclosure and administering carboplatin and a taxane (such as paclitaxel or docetaxel or Albumin bound paclitaxel (nab-paclitaxel)).

In one embodiment, the combination therapy comprises administering a combination or composition of the present disclosure and administering at least one additional therapeutic agent such as albumin bound paclitaxel (nab-paclitaxel), altretamine, capecitabine, cyclophosphamide, etoposide, gemcitabine, ifosfamide, irinotecan, liposomal doxorubicin, melphalan, pemetrexed, topotecan, vinorelbine, bevacizumab, a platinum compound (such as carboplatin, cisplatin, oxaliplatin, or iproplatin), and/or a taxane (such as paclitaxel or docetaxel, or albumin bound paclitaxel (nab-paclitaxel)).

In one embodiment, the combination therapy comprises administering a combination or composition of the present disclosure and administering bevacizumab and a taxane (such as paclitaxel or docetaxel, or albumin bound paclitaxel (nab-paclitaxel)).

In one embodiment, the combination therapy comprises administering a combination or composition of the present disclosure and administering at least one additional therapeutic agent such as cisplatin, etoposide, and/or bleomycin.

In one embodiment, the combination therapy comprises administering a combination or composition of the present disclosure and administering cisplatin (Platinol), etoposide, and bleomycin (PEB (or BEP)).

In one embodiment, the combination therapy comprises administering a combination or composition of the present disclosure and administering paclitaxel (Taxol), ifosfamide, and cisplatin (TIP).

In one embodiment, the combination therapy comprises administering a combination or composition of the present disclosure and administering vinblastine, ifosfamide, and cisplatin (VeIP).

In one embodiment, the combination therapy comprises administering a combination or composition of the present disclosure and administering etoposide (VP-16), ifosfamide, and cisplatin (VIP).

Any of the methods detailed herein, such as a method of treating cancer, in some embodiments, comprise use of a combination of a TEAD inhibitor and a KRAS inhibitor provided herein, such as any of the combination of a TEAD inhibitor and a KRAS inhibitor provided under the header of “Combination”. For example, in some embodiments, provided here is a method of treating cancer in a subject in need thereof, comprising administering to the subject an effective amount of a TEAD inhibitor (e.g., a TEAD palmitate pocket binding inhibitor, a covalent TEAD inhibitor, a compound of formula (I), etc.) and an effective amount of a KRAS inhibitor (e.g., a G12C KRAS inhibitor, a compound of formula (K-II), etc.). In some embodiments, the method comprises administering to the subject an effective amount of a TEAD palmitate pocket binding inhibitor and an effective amount of a KRAS inhibitor. In some embodiments, the method comprises administering to the subject an effective amount of a covalent TEAD inhibitor and an effective amount of a KRAS inhibitor. In some embodiments, the method comprises administering to the subject an effective amount of a compound of formula (I) and an effective amount of a KRAS inhibitor. In some embodiments, the method comprises administering to the subject an effective amount of a TEAD palmitate pocket binding inhibitor and an effective amount of a G12C KRAS inhibitor. In some embodiments, the method comprises administering to the subject an effective amount of a covalent TEAD inhibitor and an effective amount of a G12C KRAS inhibitor. In some embodiments, the method comprises administering to the subject an effective amount of a compound of formula (I) and an effective amount of a G12C KRAS inhibitor. In some embodiments, the method comprises administering to the subject an effective amount of a TEAD palmitate pocket binding inhibitor and an effective amount of a compound of formular (K-II). In some embodiments, the method comprises administering to the subject an effective amount of a covalent TEAD inhibitor and an effective amount of a compound of formular (K-II). In some embodiments, the method comprises administering to the subject an effective amount of a compound of formula (I) and an effective amount of a compound of formular (K-II).

### V. Kits

In some embodiments, provided herein is a kit, comprising (i) an effective amount of a combination comprising one or more YAP/TAZ-TEAD inhibitors and one or more KRAS inhibitors; and (ii) instructions for administering the combination to treat cancer in a subject in need thereof. The kits may comprise an effective amount of any composition or combination described elsewhere herein. In some embodiments, the one or more YAP/TAZ-TEAD inhibitors comprise a compound of formula (I), (II), or (III), or any variation or embodiment thereof. In some embodiments, the one or more YAP/TAZ-TEAD inhibitors comprise one or more of compounds T1, T2, T3, and T4 in Table 1, or a stereoisomer or tautomer thereof, or a pharmaceutically acceptable salt of any of the foregoing. In some embodiments, the one or more KRAS inhibitors comprise one or more of compounds K1, K2, and K3 in Table 1, or a stereoisomer or tautomer thereof, or a pharmaceutically acceptable salt of any of the foregoing.

In some embodiments, provided herein is a kit, comprising (i) an effective amount of a combination comprising one or more YAP/TAZ-TEAD inhibitors and one or more KRAS inhibitors; and (ii) instructions for administering the combination to treat cancer in a subject in need thereof. The kits may comprise an effective amount of any composition or combination described elsewhere herein. In some embodiments, the one or more YAP/TAZ-TEAD inhibitors comprise a compound of formula (I), (II), (III), (IV), (V), (VI), (VII), or (VIII) or any variation or embodiment thereof. In some embodiments, the one or more YAP/TAZ-TEAD inhibitors comprise one or more of compounds T1, T2, T3, T4, T5, T6, T7, T8, T9, and T10 in Table 1, or a stereoisomer or tautomer thereof, or a pharmaceutically acceptable salt of any of the foregoing. In some embodiments, the one or more KRAS inhibitors comprise one or more of compounds K1, K2, K3, and K4 in Table 1, or a stereoisomer or tautomer thereof, or a pharmaceutically acceptable salt of any of the foregoing.

### VI. Examples

In another embodiment, processes for making the subject combinations and compositions, including the compounds contained therein, are provided. The following synthetic reaction schemes detailed in the Examples are merely illustrative of some of the methods by which the compounds of the present disclosure (or an embodiment or aspect thereof) can be synthesized. Various modifications to these synthetic reaction schemes can be made and will be suggested to one skilled in the art having referred to the disclosure contained in this Application.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Intermediates and final compounds were purified by either flash chromatography, and/or by reverse-phase preparative HPLC (high performance liquid chromatography), and/or by supercritical fluid chromatography (SFC).

An exemplary synthesis of Compound K2 is described in US2021/0230142A9. For example, method of making Compound K2 can be found in Example 17a & 17b on pages 130 to 135 of US2021/0230142A9.

An exemplary synthesis of Compound K1 is described in US2018/0334454A1 (see, e.g., Example 41 on pages 210-212 of US2018/0334454A1).

An exemplary synthesis of Compound K3 is described in US2019/0144444A1 (see, e.g., Example 478 on pages 668-669 of US2019/0144444A1).

An exemplary synthesis of Compound K4 is described in WO2021/124222A1 (see, e.g., Method 1 Synthetic Scheme as described on pages 111 to 114 of WO2021/124222A1).

An exemplary synthesis of Compound T1 is described in WO2021/108483A1 (see, e.g., Example 27 on pages 140-142 of WO2021/108483A1).

An exemplary synthesis of Compound T2 is described in WO2021/097110A1 (see, e.g., Example 33 on pages 245-246 of WO2021/097110A1).

An exemplary synthesis of Compound T3 is described in WO2021/097110A1 (see, e.g., Example 2 on page 192 of WO2021/097110A1).

An exemplary synthesis of Compound T5 is described in WO2021/178339A1 (see, e.g., Example 4 on pages 123-126 of WO2021/178339A1).

An exemplary synthesis of Compound T6 is described in US2020/0347009A1 (see, e.g., Example 55 on pages 112-115 of US2020/0347009A1).

An exemplary synthesis of Compound T7 is described in WO2020/097389A1 (see, e.g., Example 84 on pages 195-196 of WO2020/097389A1).

An exemplary synthesis of Compound T8 is described in US2020/0354325A1 (see, e.g., Example 113 on pages 157-158 of US2020/0354325A1).

An exemplary synthesis of Compound T9 is described in WO2021/108483A1 (see, e.g., Example 41 on pages 156-158 of WO2021/108483A1).

An exemplary synthesis of Compound T10 is described in WO2021/224291A1 (see, e.g., Example 2-4 on page 152 of WO2021/224291A1).

Any references detailed in this section are incorporated herein by reference in their entirety, and specifically with respect to methods of making compounds detailed therein.

### Example 1

### Preparation of 5-(4-Cyclohexylphenyl)-3-(3-(fluoromethyl) azetidine-1-carbonyl)-2-(5-methylpyrazin-2-yl) pyrazolo [1,5-a]pyrimidin-7(4H)-one (Compound T1)

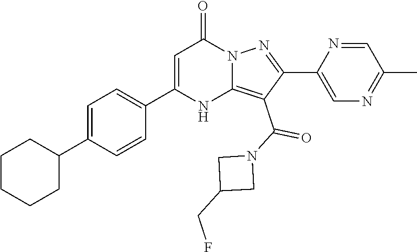 [SMILES: Cc1cnc(-c2nn3c(=O)cc(-c4ccc(C5CCCCC5)cc4)[nH]c3c2C(=O)N2CC(CF)C2)cn1]

### Step 1: 2-Methyl-5-(tributylstannyl)pyrazine

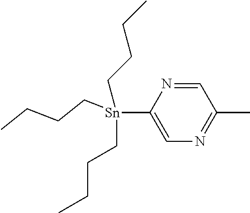 [SMILES: CCC[CH2][Sn]([CH2]CCC)([CH2]CCC)[c]1cnc(C)cn1]

To a solution of 2-bromo-5-methyl-pyrazine (1 g, 5.78 mmol) and tributylchlorostannane (3.16 g, 9.71 mmol) in THF (15 mL) was added n-BuLi (2.8 mL, 7.0 mmol) dropwise at −78° C. and stirred for a further 2 hours at this temperature. The reaction was quenched with water (50 mL) and extracted with hexanes (50 mL×2). The combined organics were dried over Na₂SO₄, filtered and concentrated. The crude was purified by column chromatography (0-10% ethyl acetate in petroleum ether) to afford the title compound (600 mg, 27%) as a colorless oil. ¹H NMR (400 MHz, CDCl₃): δ 8.62 (s, 1H), 8.42 (s, 1H), 2.51 (s, 3H), 1.59-1.49 (m, 6H), 1.36-1.30 (m, 6H), 1.15 (t, J=8.0 Hz, 6H), 0.88 (t, J=7.2 Hz, 9H).

### Step 2: 5-(4-Cyclohexylphenyl)-3-(3-(fluoromethyl) azetidine-1-carbonyl)-2-(5-methyl pyrazin-2-yl)pyrazolo[1,5-a]pyrimidin-7(4H)-one

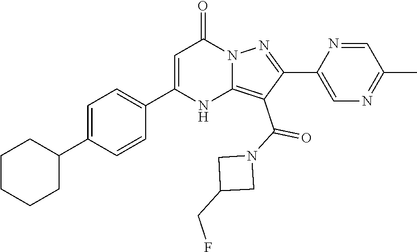 [SMILES: Cc1cnc(-c2nn3c(=O)cc(-c4ccc(C5CCCCC5)cc4)[nH]c3c2C(=O)N2CC(CF)C2)cn1]

The title compound (34 mg, 17%) was prepared from 2-bromo-5-(4-cyclohexylphenyl)-3-(3-(fluoromethyl)azetidine-1-carbonyl)pyrazolo[1,5-a]pyrimidin-7(4H)-one (200 mg, 0.41 mmol) and 2-methyl-5-(tributylstannyl)pyrazine (314 mg, 0.82 mmol) following the procedure outlined for Example 7, Step 2. ¹H NMR (400 MHz, CDCl₃): δ 10.57 (s, 1H), 9.27 (s, 1H), 8.61 (s, 1H), 7.66 (d, J=8.0 Hz, 2H), 7.40 (d, J=8.0 Hz, 2H), 6.31 (s, 1H), 4.42 (dd, J=46.8, 4.8 Hz, 2H), 4.25-3.68 (m, 4H), 2.82-2.79 (m, 1H), 2.70 (s, 3H), 2.63-2.58 (m, 1H), 1.93-1.89 (m, 4H), 1.83-1.79 (m, 1H), 1.47-1.44 (m, 4H), 1.36-1.27 (m, 1H). LCMS (ESI) m/z 501.3 (M+H)⁺.

Compound T1 was prepared from 2-bromo-5-(4-cyclohexylphenyl)-3-(3-(fluoromethyl)azetidine-1-carbonyl)pyrazolo[1,5-a]pyrimidin-7(4H)-one and stannane reagents following the procedure outlined for Example 1, Step 2. The corresponding stannane reagents are prepared from aryl bromide and n-BuLi following the procedure outlined for Example 1, Step 1.

| Product Structure | | LCMS | |
|---|---|---|---|
| (Compound T1) | Name | [M + H]⁺ | ¹H NMR |
| 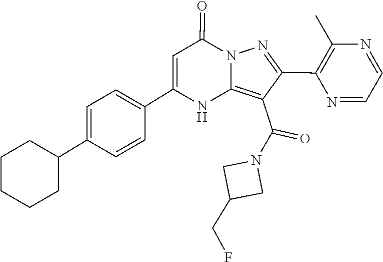 [SMILES: Cc1nccnc1-c1nn2c(=O)cc(-c3ccc(C4CCCCC4)cc3)[nH]c2c1C(=O)N1CC(CF)C1] | 5-(4- Cyclohexylphenyl)- 3-(3- (fluoromethyl)azeti- dine-1-carbonyl)- 2-(3- methylpyrazin-2- yl)pyrazolo[1,5- a]pyrimidin- 7(4H)-one | 501.2 | ¹H NMR (400 MHz, CDCl₃): δ 10.84 (s, 1H), 8.67-8.54 (m, 2H), 7.67 (d, J = 8.4 Hz, 2H), 7.41 (J = 8.4 Hz, 2H), 6.33 (s, 1H), 4.46- 4.18 (m, 6H), 2.82-2.71 (m, 4H), 2.64-2.60 (m, 1H), 1.93- 1.87 (m, 4H), 1.83-1.79 (m, 1H), 1.53-1.39 (m, 4H), 1.32- 1.29 (m, 1H) |

### Example 2

### Preparation of N-(7-(4-Isopropylphenyl)-2,3-dihydrobenzofuran-5-yl)acrylamide (Compound T2)

The overall reaction scheme was as follows:

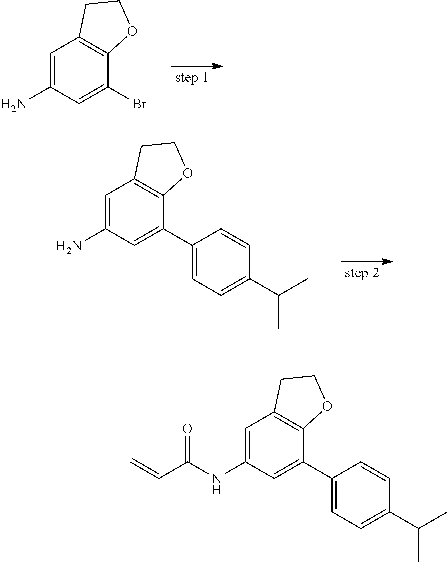

### Step 1: 7-(4-Isopropylphenyl)-2,3-dihydrobenzofuran-5-amine

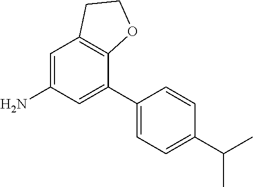 [SMILES: CC(C)c1ccc(-c2cc(N)cc3c2OCC3)cc1]

A mixture of 7-bromo-2,3-dihydrobenzofuran-5-amine (200 mg, 0.93 mmol), (4-isopropylphenyl)boronic acid (184 mg, 1.12 mmol), Pd(dppf)Cl₂ (68 mg, 0.09 mmol), K₂CO₃ (387 mg, 2.8 mmol) in 1,4-dioxane (5 mL) and water (1 mL) was stirred at 100° C. for 3 hours under N₂. The reaction mixture was concentrated under vacuum. The residue was purified by flash column chromatography on silica gel (0-30% ethyl acetate in petroleum ether) to afford the title compound (180 mg, 76%) as a yellow solid. ¹H NMR (400 MHz, DMSO-d₆): δ 7.53 (d, J=8.4 Hz, 2H), 7.25 (d, J=8.4 Hz, 2H), 6.50 (s, 1H), 6.49 (s, 1H), 4.60 (s, 2H), 4.40 (t, J=8.8 Hz, 2H), 3.08 (t, J=8.8 Hz, 2H), 2.95-2.85 (m, 1H), 1.22 (d, J=6.8 Hz, 6H).

### Step 2: N-(7-(4-Isopropylphenyl)-2,3-dihydrobenzofuran-5-yl)acrylamide (Compound T2)

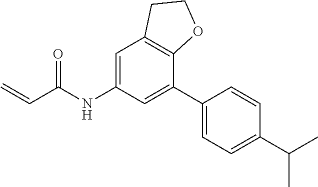 [SMILES: C=CC(=O)Nc1cc2c(c(-c3ccc(C(C)C)cc3)c1)OCC2]

To a mixture of 7-(4-isopropylphenyl)-2,3-dihydrobenzofuran-5-amine (180 mg, 0.71 mmol) and DIPEA (0.25 mL, 1.42 mmol) in DCM (3 mL) was added acryloyl chloride (0.05 mL, 0.64 mmol) at 0° C. The reaction was stirred at 0° C. for 15 minutes. The reaction was quenched with water (20 mL). The mixture was extracted with DCM (30 mL×2) and washed with water (20 mL×3). The organic phase was dried over Na₂SO₄ and concentrated. The residue was purified by pre-HPLC (water (0.2% FA)-ACN, 60%˜90%) to afford the title compound (93.51 mg, 42%) as a white solid. ¹H NMR (400 MHz, DMSO-d₆): δ 10.01 (s, 1H), 7.54-7.51 (m, 4H), 7.27 (d, J=8.0 Hz, 2H), 6.42 (dd, J=16.8, 10.0 Hz, 1H), 6.23 (dd, J=16.8, 2.0 Hz, 1H), 5.71 (d, J=10.0 Hz, 1H), 4.51 (t, J=8.8 Hz, 2H), 3.19 (t, J=8.8 Hz, 2H), 2.92-2.82 (m, 1H), 1.19 (d, J=6.8 Hz, 6H); LCMS (ESI): m/z 308.1 (M+H)⁺.

### Example 3

### Preparation of N-(6-methoxy-5-((E)-2-((1r,4r)-4-(trifluoromethyl)cyclohexyl)vinyl)pyridin-3-yl)acrylamide (Compound T3)

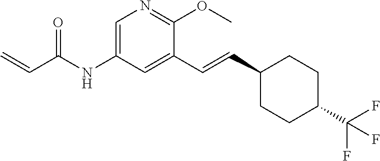 [SMILES: C=CC(=O)Nc1cnc(OC)c(/C=C/[C@H]2CC[C@H](C(F)(F)F)CC2)c1]

### Preparation of 6-Methoxy-5-((E)-2-(trans-4-(trifluoromethyl)cyclohexyl)vinyl)pyridin-3-amine (Intermediate 3-A)

The general reaction scheme was as follows:

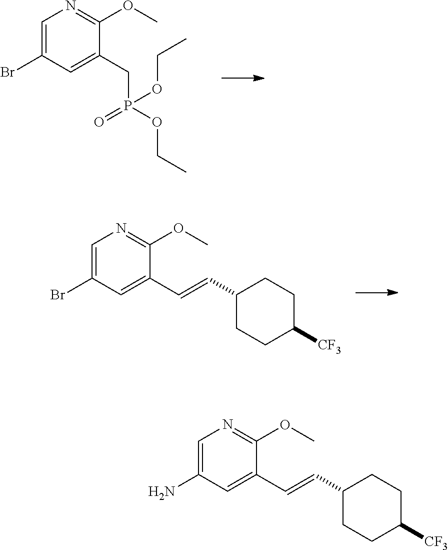 [SMILES: CCOP(=O)(Cc1cc(Br)cnc1OC)OCC ; COc1ncc(Br)cc1/C=C/[C@H]1CC[C@H](C(F)(F)F)CC1 ; COc1ncc(N)cc1/C=C/[C@H]1CC[C@H](C(F)(F)F)CC1]

### Step 1: 5-Bromo-2-methoxy-3-((E)-2-(trans-4 (trifluoromethyl)cyclohexyl)vinyl)pyridine

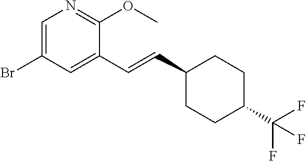 [SMILES: COc1ncc(Br)cc1/C=C/[C@H]1CC[C@H](C(F)(F)F)CC1]

To a solution of diethyl ((5-bromo-2-methoxypyridin-3-yl)methyl)phosphonate (1.15 g, 3.41 mmol) in toluene (15.0 mL) was added sodium tert-pentoxide (0.490 g, 4.43 mmol) at 0° C. After being stirred at 0° C. for 20 minutes, a solution of trans-4-(trifluoromethyl) cyclohexanecarbaldehyde (Intermediate 1, 1.23 g, 6.81 mmol) in THF (15.0 ml) was added dropwise and the reaction mixture was stirred for 1.5 hours at 0° C. The reaction mixture was poured into saturated aqueous NH₄Cl solution (50 mL) and extracted with EtOAc (100 mL×2). The combined organic layers were washed with brine (50 ml), dried over Na₂SO₄ and concentrated. The residue was purified by column chromatography on silica gel (0-10% EtOAc in petroleum ether) to afford the title compound (1.04 g, 83%) as a white solid. ¹H NMR (400 MHz, CDCl₃): δ 8.05 (d, J=2.0 Hz, 1H), 7.72 (d, J=2.0 Hz, 1H), 6.48 (d, J=16.0 Hz, 1H), 6.20 (dd, J=16.0, 6.8 Hz, 1H), 3.95 (s, 3H), 2.16-2.10 (m, 1H), 2.08-1.92 (m, 5H), 1.48-1.33 (m, 2H), 1.31-1.16 (m, 2H).

### Step 2: 6-Methoxy-5-((E)-2-(trans-4-(trifluoromethyl)cyclohexyl)vinyl)pyridin-3-amine

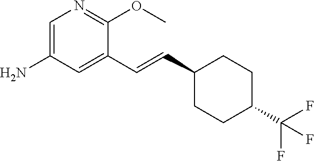 [SMILES: COc1ncc(N)cc1/C=C/[C@H]1CC[C@H](C(F)(F)F)CC1]

To a solution of 5-bromo-2-methoxy-3-((E)-2-(trans-4 (trifluoromethyl)cyclohexyl)vinyl)pyridine (930 mg, 2.55 mmol) in DMSO (16 mL) was added CuI (48.0 mg, 0.26 mmol), K₃PO₄ (2.04 g, 7.66 mmol), NH₃—H₂O (0.570 ml, 7.66 mmol, 25% wt) and N¹,N²-bis(5-methyl-[1,1′-biphenyl]-2-yl)oxalamide (107 mg, 0.26 mmol). The reaction mixture was stirred at 110° C. for 16 hours. The reaction was diluted with water (50 mL), extracted with EtOAc (50 mL×3) and the combined organic layers were dried with Na₂SO₄ and concentrated. The residual was purified by column chromatography on silica gel (0-2% EtOAc in petroleum ether) to afford the title compound (620 mg, 80%) as a white solid. ¹H NMR (400 MHz, CDCl₃): δ 7.52 (d, J=2.4 Hz, 1H), 7.08 (d, J=2.4 Hz, 1H), 6.51 (d, J=16.0 Hz, 1H), 6.14 (d, J=16.0, 6.8 Hz, 1H), 3.89 (s, 3H), 3.32 (s, 2H), 2.10-2.05 (m, 1H), 2.03-1.91 (m, 5H), 1.44-1.09 (m, 4H); LCMS (ESI): m/z 301.2 (M+H)⁺.

### Step 3: N-(6-methoxy-5-((E)-2-((1r,4r)-4-(trifluoromethyl)cyclohexyl)vinyl)pyridin-3-yl)acrylamide (Compound T3)

To a mixture of Intermediate 3-A (200 mg, 0.670 mmol) and DIPEA (0.500 ml, 3.00 mmol) in dichloromethane (2.0 ml) was added acryloyl chloride (0.120 ml, 1.47 mmol) at 0° C. And the reaction was stirred at 0° C. for 2 hours. The reaction mixture was diluted with water (40 mL), and extracted with DCM (40 mL×2). The combined organic layers were dried over Na₂SO₄ and concentrated. The residue was purified by prep-TLC (25% EtOAc in petroleum ether) to afford the title compound (58.37 mg, 23%) as a white solid. ¹H NMR (400 MHz, DMSO-d₆): δ 10.18 (s, 1H), 8.27 (dd, J=9.6, 2.4 Hz, 1H), 8.10 (d, J=2.4 Hz, 1H), 6.53-6.35 (m, 2H), 6.31-6.17 (m, 2H), 5.76 (dd, J=12.0, 2.0 Hz, 1H), 3.86 (s, 3H), 2.21-2.14 (m, 2H), 1.90-1.83 (m, 4H), 1.32-1.20 (m, 4H). LCMS (ESI): m/z 355.2 (M+H)⁺.

### Example 4

### Preparation of 2-(((4-cyano-7-(4-isopropylphenyl)-2,3-dihydrobenzofuran-5-yl)amino)methyl)acrylic acid (Compound T4)

### Intermediate 4-A

### Preparation of 7-(4-isopropylphenyl)-2,3-dihydrobenzofuran-5-amine

The general reaction scheme was as follows:

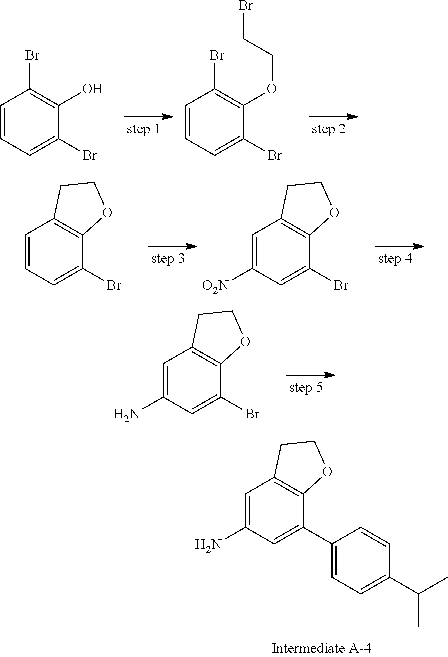

### Step 1: Preparation of 1,3-dibromo-2-(2-bromoethoxy)benzene

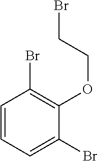 [SMILES: BrCCOc1c(Br)cccc1Br]

A mixture of 2,6-dibromophenol (525 g, 2.08 mol), NaOH (91.7 g, 2.29 mol) and 1,2-dibromoethane (180.43 mL, 2.08 mol) in water (1.5 L) was stirred at 100° C. for 16 hours. After cooling to room temperature, the oil product was separated via a separation funnel, washed with NaOH (1M) (200 mL×2) to remove the starting materials. The product was dissolved in petroleum ether (800 mL), dried over Na₂SO₄, filtered and concentrated to afford the title compound (520 g, 69%) as a yellow liquid. ¹H NMR (400 MHz, DMSO-d₆): δ 7.68 (dd, J=8.0, 2.4 Hz, 2H), 7.07 (t, J=8.0 Hz, 1H), 4.28 (t, J=5.6 Hz, 2H), 3.85 (t, J=5.6 Hz, 2H).

### Step 2: Preparation of 7-bromo-2,3-dihydrobenzofuran

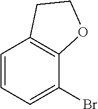

To a mixture of 1,3-dibromo-2-(2-bromoethoxy)benzene (200 g, 557.34 mmol) in THF (1.5 L) was added n-BuLi (227.39 mL, 568.48 mmol, 2.5 mol/L in hexane) at −78° C. dropwise. The mixture was stirred at −78° C. for 1 hour. The reaction was quenched by water (500 mL). The mixture was diluted with water (1 L), extracted with ethyl acetate (1 L×2) and the organic layers were combined. The organic layer was dried over anhydrous sodium sulfate and concentrated under vacuum to afford the title compound (100 g, 90%) as a colorless oil. ¹H NMR (400 MHz, CDCl₃): δ 7.30-7.23 (m, 1H), 7.10 (dd, J=7.2, 1.2 Hz, 1H), 6.71 (t, J=7.6 Hz, 1H), 4.65 (t, J=8.8 Hz, 2H), 3.30 (t, J=8.8 Hz, 2H).

### Step 3: Preparation of 7-Bromo-5-nitro-2,3-dihydrobenzofuran

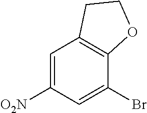 [SMILES: O=[N+]([O-])c1cc(Br)c2c(c1)CCO2]

To a mixture of 7-bromo-2,3-dihydrobenzofuran (100 g, 502.41 mmol) in DCM (1 L) at 0° C. was added a mixture solution of con. aq. H₂SO₄ (70 mL) and con. aq. HNO₃ (68.6 mL). The mixture was stirred at 0° C. for 30 min. The mixture was quenched with water (500 mL), carefully adjusted pH to 9 with 25% NaOH solution and extracted with EtOAc (1 L×3). The organic layer was washed with water (1 L×3), dried over Na₂SO₄, filtered and concentrated to afford the tile compound (98 g, 80%) as a yellow solid. ¹H NMR (400 MHz, CDCl₃): δ 8.30 (d, J=2.4 Hz, 1H), 8.04 (d, J=2.4 Hz, 1H), 4.85 (t, J=8.8 Hz, 2H), 3.43 (t, J=8.8 Hz, 1H).

### Step 4: Preparation of 7-Bromo-2,3-dihydrobenzofuran-5-amine

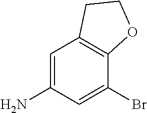 [SMILES: Nc1cc(Br)c2c(c1)CCO2]

A solution of 7-bromo-5-nitro-2,3-dihydrobenzofuran (100 g, 409.77 mmol), NH₄Cl (110 g, 2.05 mol) and iron powder (115 g, 2.05 mol) in water:ethanol (1:1) (2.5 L) was stirred at 80° C. for 3 hours. After cooling to room temperature, the reaction mixture was filtered and concentrated. Then the mixture was extracted with EtOAc (500 mL×3 and the organic layer was washed with water (500 mL×5). The organics were dried over Na₂SO₄, filtered and concentrated. The crude was dissolved in DCM (200 mL) and then petroleum ether (400 mL) was added. The solids where collected to afford the title compound (70.2 g, 80%) as a yellow solid. ¹H NMR (400 MHz, CDCl₃): δ 6.64 (s, 1H), 6.53 (s, 1H), 4.59 (t, J=8.8 Hz, 2H), 3.42 (br s, 2H), 3.23 (t, J=8.8 Hz, 2H).

### Step 5: Preparation of 7-(4-Isopropylphenyl)-2,3-dihydrobenzofuran-5-amine

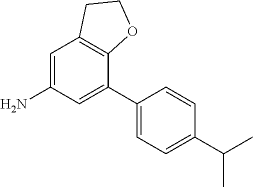 [SMILES: CC(C)c1ccc(-c2cc(N)cc3c2OCC3)cc1]

A mixture of 7-bromo-2,3-dihydrobenzofuran-5-amine (100 g, 467.16 mmol), (4-isopropylphenyl)boronic acid (78.15 g, 476.5 mmol), Pd(dppf)Cl₂ (17.09 g, 23.36 mmol), Na₂CO₃ (149 g, 1.41 mol) in 1,4-Dioxane (1 L) and water (100 mL) was stirred at 100° C. for 2 hours under a N₂ atmosphere. After being cooled to room temperature, the reaction mixture was filtered and the filtrate was concentrated under vacuum. The residue was purified by flash chromatography on silica gel (0-30% ethyl acetate in petroleum ether) to afford the title compound (116 g, 98%) as a yellow solid. ¹H NMR (400 MHz, CDCl₃): δ 7.61 (d, J=8.0 Hz, 2H), 7.29 (d, J=8.0 Hz, 2H), 6.66 (d, J=2.4 Hz, 1H), 6.59 (d, J=2.4 Hz, 1H), 4.56 (t, J=8.8 Hz, 2H), 3.18 (t, J=8.8 Hz, 2H), 3.00-2.92 (m, 1H), 1.30 (d, J=6.8 Hz, 6H); LCMS (ESI): m/z 254.1 (M+H)⁺.

### Intermediate 4-B

### Preparation of 4-bromo-7-(4-isopropylphenyl)-2,3-dihydrobenzofuran-5-amine

The general reaction scheme was as follows:

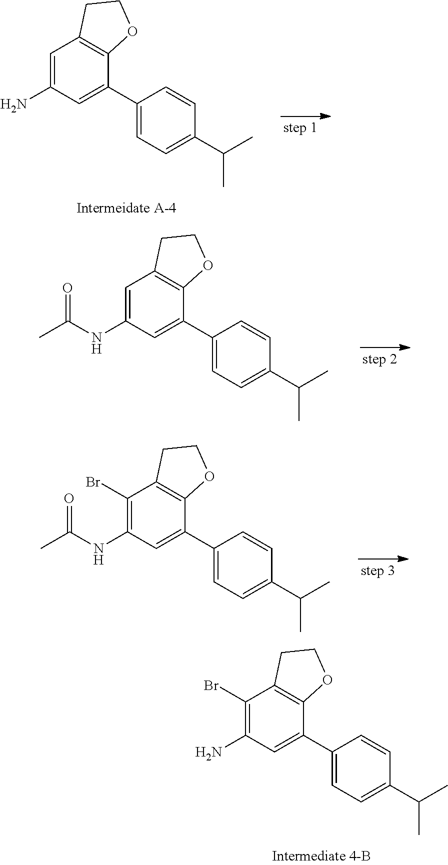

### Step 1: Preparation of N-(7-(4-isopropylphenyl)-2,3-dihydrobenzofuran-5-yl)acetamide

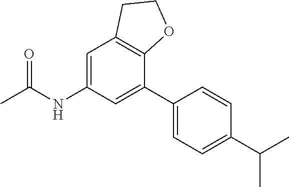 [SMILES: CC(=O)Nc1cc2c(c(-c3ccc(C(C)C)cc3)c1)OCC2]

To a solution of 7-(4-isopropylphenyl)-2,3-dihydrobenzofuran-5-amine (150 g, 592.09 mmol) and TEA (99.03 mL, 710.51 mmol) in DCM (1.5 L) was added acetyl chloride (46.31 mL, 651.3 mmol) at −78° C. dropwise. The reaction was stirred at −78° C. for 2 hours. The reaction was quenched with water (200 mL) and extracted with dichloromethane (1 L×2). The combined organic layers were dried over Na₂SO₄ and concentrated. The residue was triturated with DCM and hexanes (1:10) and filtered to afford the title compound (222 g, 83%) as a white solid. ¹H NMR (400 MHz, CDCl₃): δ 7.58 (d, J=8.0 Hz, 2H), 7.48 (s, 1H), 7.25 (d, J=8.0 Hz, 2H), 7.21 (s, 1H), 7.19 (s, 1H), 4.60 (t, J=8.8 Hz, 2H), 3.24 (t, J=8.8 Hz, 2H), 2.96-2.90 (m, 1H), 2.16 (s, 3H), 1.27 (d, J=6.8 Hz, 6H); LCMS (ESI): m/z 296.1 (M+H)⁺.

### Step 2: Preparation of N-(4-bromo-7-(4-isopropylphenyl)-2,3-dihydrobenzofuran-5-yl)acetamide

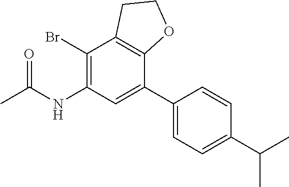 [SMILES: CC(=O)Nc1cc(-c2ccc(C(C)C)cc2)c2c(c1Br)CCO2]

A mixture of N-(7-(4-isopropylphenyl)-2,3-dihydrobenzofuran-5-yl)acetamide (100 g, 338.55 mmol) and bromine (19.08 mL, 372.4 mmol) in Acetic acid (500 mL) was stirred at 50° C. for 10 min. The reaction mixture was diluted with water (1 L) and the pH was adjusted to 7 with a 2M NaOH aqueous solution. The mixture was extracted with EtOAc (1 L×3), the combined organic layers were dried over Na₂SO₄ and concentrated. The residue was dissolved in DCM (200 mL) and MTBE was added until a precipitate appears. The heterogenous mixture was cooled to 0° C. for 20 minutes. Then the precipitate was filtered to afford the title compound (38 g, 30%) as a white solid. ¹H NMR (400 MHz, CDCl₃): δ 8.09 (s, 1H), 7.62 (d, J=8.0 Hz, 2H), 7.27 (d, J=8.0 Hz, 2H), 4.65 (t, J=8.8 Hz, 2H), 3.28 (t, J=8.8 Hz, 2H), 2.93-2.88 (m, 1H), 2.22 (s, 3H), 1.28 (d, J=6.8 Hz, 6H); LCMS (ESI): m/z 374.1 (M+H)⁺.

### Step 3: Preparation of 4-bromo-7-(4-isopropylphenyl)-2,3-dihydrobenzofuran-5-amine

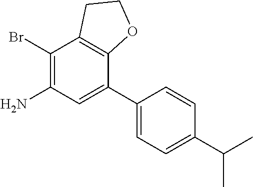 [SMILES: CC(C)c1ccc(-c2cc(N)c(Br)c3c2OCC3)cc1]

A mixture of 12 M aqueous hydrochloric acid (334 mL, 4.01 mol) and N-(4-bromo-7-(4-isopropylphenyl)-2,3-dihydrobenzofuran-5-yl)acetamide (150 g, 400.78 mmol) in ethanol (1.5 L) was stirred at 80° C. for 5 hours. After cooling to room temperature, the solvent was removed under reduced pressure. The residue was diluted with water and the pH was adjusted to 9 with a 2 M NaOH aqueous solution. The mixture was extracted with EtOAc (1 L×3), then the combined organic layers were dried over Na₂SO₄ and evaporated to afford the title compound (124 g, 93%) as a brown solid. ¹H NMR (400 MHz, CDCl₃): δ 7.54 (d, J=8.0 Hz, 2H), 7.25 (d, J=8.0 Hz, 2H), 6.72 (s, 1H), 4.58 (t, J=8.8 Hz, 2H), 3.78 (s, 2H), 3.23 (t, J=8.8 Hz, 2H), 2.93-2.89 (m, 1H), 1.25 (d, J=6.8 Hz, 6H); LCMS (ESI): m/z 332.1 (M+H)⁺.

### Intermediate 4-C

### Preparation of 5-amino-7-(4-isopropylphenyl)-2,3-dihydrobenzofuran-4-carbonitrile

The general reaction scheme was as follows:

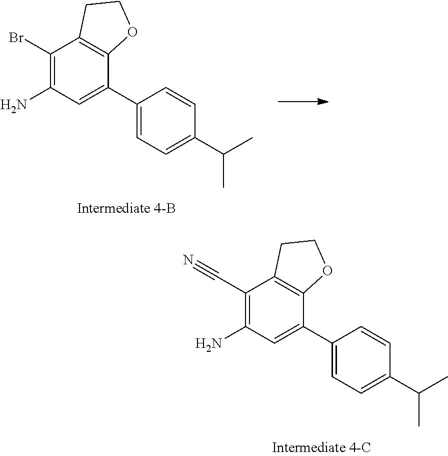 [SMILES: CC(C)c1ccc(-c2cc(N)c(Br)c3c2OCC3)cc1 ; CC(C)c1ccc(-c2cc(N)c(C#N)c3c2OCC3)cc1]

A mixture of t-BuXPhos Pd G3 (19.0 g, 23.92 mmol), Zn(CN)₂ (176.7 g, 1.51 mol) and 4-bromo-7-(4-isopropylphenyl)-2,3-dihydrobenzofuran-5-amine (100 g, 301 mmol) in N,N-dimethylacetamide (1 L) was stirred at 140° C. for 16 hours. After cooling to room temperature, the reaction solution was added into with water (2 L). The mixture solution was filtered and the filter cake was washed with water (2 L). The filter cake was dissolved in EtOAc (2 L), dried over MgSO₄, filtered and concentrated. The residue was purified by flash chromatography silica gel (0-50% ethyl acetate in petroleum ether) to afford 80 g crude product. The crude product was triturated with DCM:hexanes (1:10) and filtered to afford the title compound (59 g, 70%) as a yellow solid. ¹H NMR (400 MHz, CDCl₃): δ 7.59 (dd, J=8.0, 1.6 Hz, 2H), 7.30 (d, J=8.0 Hz, 2H), 6.68 (s, 1H), 4.64 (t, J=8.8 Hz, 2H), 4.08 (br s, 2H), 3.36 (t, J=8.8 Hz, 2H), 2.97-2.95 (m, 1H), 1.28 (d, J=6.8 Hz, 6H); LCMS (ESI): m/z 279.1 (M+H)⁺.

### Preparation of 2-(((4-cyano-7-(4-isopropylphenyl)-2,3-dihydrobenzofuran-5-yl)amino)methyl)acrylic acid (Compound T4)

The general reaction scheme was as follows:

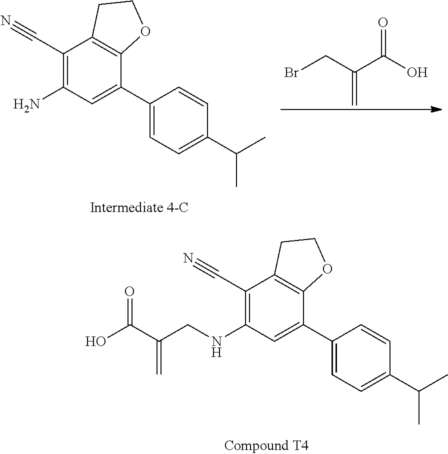

To a mixture of 5-amino-7-(4-isopropylphenyl)-2,3-dihydrobenzofuran-4-carbonitrile (15.0 g, 53.89 mmol) in N,N-Dimethylformamide (150 mL) was added 2-(bromomethyl)acrylic acid (8.89 g, 53.89 mmol). The mixture was stirred at 80° C. for 2 hours at which point the reaction mixture was purified by prep-HPLC (SANPONT C18, 250*80 mm*10 um, 100A, water (0.225% FA)-ACN, 40%-80%) to afford the title compound (8.2 g, 42%) as a yellow solid. ¹H NMR (400 MHz, DMSO-d₆): δ 12.75 (s, 1H), 7.55 (d, J=8.0 Hz, 2H), 7.31 (d, J=8.0 Hz, 2H), 6.46 (s, 1H), 6.11 (s, 1H), 6.01 (t, J=6.0 Hz, 1H), 5.67 (s, 1H), 4.55 (t, J=8.8 Hz, 2H), 4.05 (d, J=5.2 Hz, 2H), 3.30 (t, J=8.8 Hz, 2H), 2.93-2.90 (m, 1H), 1.22 (d, J=6.8 Hz, 6H); LCMS (ESI): m/z 363.2 (M+H)⁺.

### Example 5

### Preparation of N-[[3-[2-[2-[2-[2-[2-[2-[2-(2,6-dioxo-3-piperidyl)-1,3-dioxo-isoindolin-5-yl]oxyethoxy]ethoxy]ethoxy]ethoxy]ethyl-methyl-amino]-2-oxo-ethyl]phenyl]methyl]-5-methoxy-4-[rac-(E)-2-[4-(trifluoromethyl)cyclohexyl]vinyl]pyridine-2-carboxamide (Compound T5/Example 4 of WO2021/178339A1)

The general reaction scheme was as follows:

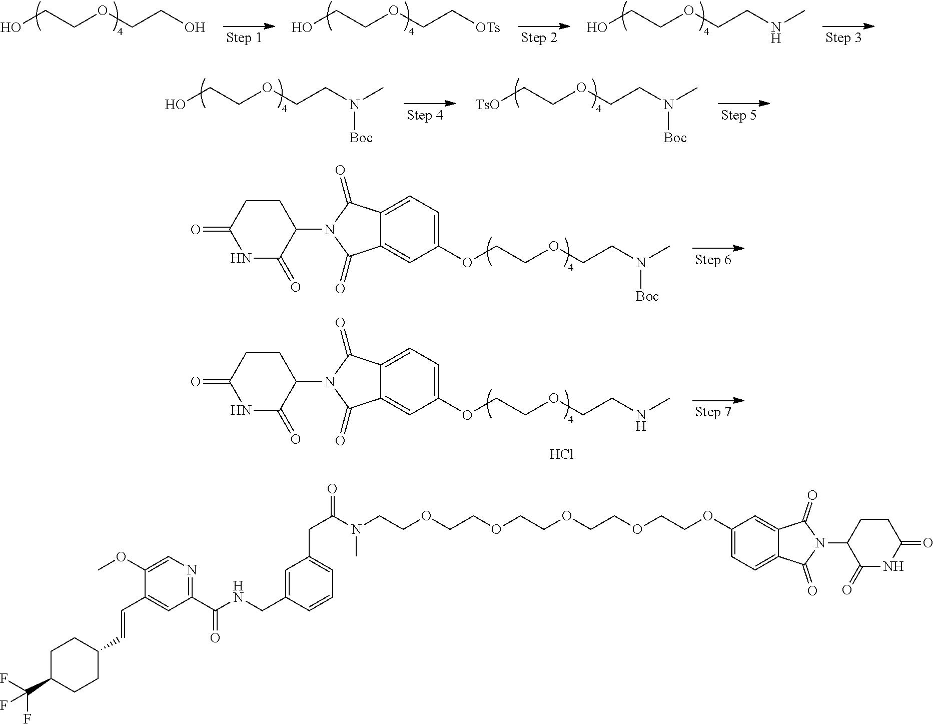

### Step 1: 14-Hydroxy-3,6,9,12-tetraoxatetradecyl 4-methylbenzenesulfonate

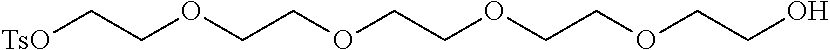 [SMILES: Cc1ccc(S(=O)(=O)OCCOCCOCCOCCOCCO)cc1]

The title compound (6.1 g, 46%) was furnished as a colorless oil. It was prepared from 3,6,9,12-tetraoxatetradecane-1,14-diol (8.0 g, 33.57 mmol) following the procedure outlined for Example 2 of WO2021/178339A1, Step 1. ¹H NMR (400 MHz, CDCl₃): δ 7.80 (d, J=8.0 Hz, 2H), 7.35 (d, J=8.0 Hz, 2H), 4.16 (t, J=4.8 Hz, 2H), 3.72-3.58 (m, 18H), 2.45 (s, 3H).

### Step 2: 5,8,11,14-Tetraoxa-2-azahexadecan-16-ol

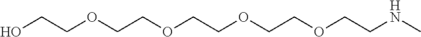 [SMILES: CNCCOCCOCCOCCOCCO]

The title compound (3.9 g, quant.) was furnished as a colorless oil. It was prepared from 14-hydroxy-3,6,9,12-tetraoxatetradecyl 4-methylbenzenesulfonate (6.1 g, 15.54 mmol) following the procedure outlined for Example 1 of WO2021/178339A1, Step 2. ¹H NMR (400 MHz, CDCl₃): δ 3.78-3.73 (m, 4H), 3.65-3.60 (m, 12H), 3.58-3.55 (m, 4H), 3.27-3.25 (m, 2H), 2.76 (s, 3H).

### Step 3: tert-Butyl (14-Hydroxy-3,6,9,12-tetraoxatetradecyl)(methyl)carbamate

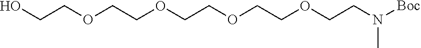 [SMILES: CN(CCOCCOCCOCCOCCO)C(=O)OC(C)(C)C]

The title compound (2.1 g, 39%) was furnished as a colorless oil. It was prepared from 5,8,11,14-tetraoxa-2-azahexadecan-16-ol (3.9 g, 15.52 mmol) following the procedure outlined for Example 1 of WO2021/178339A1, Step 3. ¹H NMR (400 MHz, CDCl₃): δ 3.69-3.64 (m, 2H), 3.62-3.46 (m, 16H), 3.33 (br, 2H), 2.84 (s, 3H), 1.39 (s, 9H).

### Step 4: 2,2,5-Trimethyl-4-oxo-3,8,11,14,17-pentaoxa-5-azanonadecan-19-yl 4-methylbenzenesulfonate

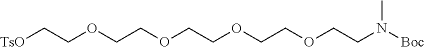 [SMILES: Cc1ccc(S(=O)(=O)OCCOCCOCCOCCOCCN(C)C(=O)OC(C)(C)C)cc1]

The title compound (2.0 g, 70%) was furnished as a colorless oil. It was prepared from tert-butyl (14-hydroxy-3,6,9,12-tetraoxatetradecyl)(methyl)carbamate (2.0 g, 5.69 mmol) following the procedure outlined for Example 1 of WO2021/178339A1, Step 4. LCMS (ESI): m/z 528.1 (M+Na)⁺.

### Step 5: tert-Butyl (14-((2-(2,6-dioxopiperidin-3-yl)-1,3-dioxoisoindolin-5-yl)oxy)-3,6,9,12-tetraoxatetradecyl)(methyl)carbamate

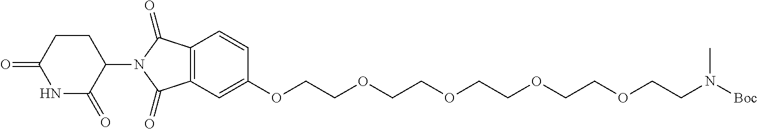 [SMILES: CN(CCOCCOCCOCCOCCOc1ccc2c(c1)C(=O)N(C1CCC(=O)NC1=O)C2=O)C(=O)OC(C)(C)C]

The title compound (120 mg, 46%) was furnished as a colorless oil. It was prepared from 2,2,5-trimethyl-4-oxo-3,8,11,14,17-pentaoxa-5-azanonadecan-19-yl 4-methylbenzenesulfonate (200 mg, 0.43 mmol) following the procedure outlined for Example 1 of WO2021/178339A1, Step 5. LCMS (ESI): m/z 630.3 (M+Na)⁺.

### Step 6: 5-(5,8,11,14-Tetraoxa-2-azahexadecan-16-yloxy)-2-(2,6-dioxopiperidin-3-yl)isoindoline-1,3-dione hydrochloride

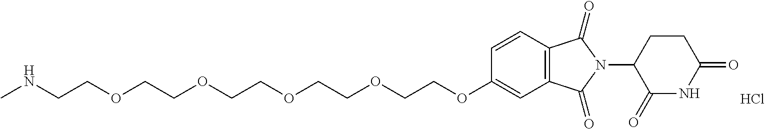 [SMILES: CNCCOCCOCCOCCOCCOc1ccc2c(c1)C(=O)N(C1CCC(=O)NC1=O)C2=O ; Cl]

The title compound (89 mg, 99%) was furnished as a white solid. It was prepared from tert-butyl(14-((2-(2,6-dioxopiperidin-3-yl)-1,3-dioxoisoindolin-5-yl)oxy)-3,6,9,12-tetraoxatetradecyl)(methyl)carbamate (100 mg, 0.16 mmol) following the procedure outlined for Example 1 of WO2021/178339A1, Step 6. LCMS (ESI): m/z 508.2 (M+H)⁺.

### Step 7: N-(3-(17-((2-(2,6-Dioxopiperidin-3-yl)-1,3-dioxoisoindolin-5-yl)oxy)-3-methyl-2-oxo-6,9,12,15-tetraoxa-3-azaheptadecyl)benzyl)-5-methoxy-4-((E)-2-(trans-4-(trifluoromethyl)cyclohexyl)vinyl)picolinamide

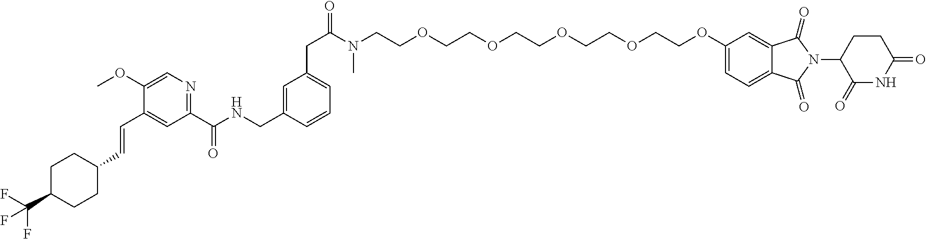 [SMILES: COc1cnc(C(=O)NCc2cccc(CC(=O)N(C)CCOCCOCCOCCOCCOc3ccc4c(c3)C(=O)N(C3CCC(=O)NC3=O)C4=O)c2)cc1/C=C/[C@H]1CC[C@H](C(F)(F)F)CC1]

The title compound (28 mg, 20%) was furnished as a white solid. It was prepared from 5-(5,8,11,14-tetraoxa-2-azahexadecan-16-yloxy)-2-(2,6-dioxopiperidin-3-yl)isoindoline-1,3-dione hydrochloride (88 mg, 0.16 mmol) following the procedure outlined for Example 1 of WO2021/178339A1, Step 7. It was purified by prep-HPLC (Xtimate C18 150*40 mm*10 um, water (0.225% FA)-ACN, 60-90%). ¹H NMR (400 MHz, CD₃OD): δ 8.26 (s, 1H), 8.11 (s, 1H), 7.75 (dd, J=2.8, 8.4 Hz, 1H), 7.37-7.35 (m, 1H), 7.29-7.24 (m, 4H), 7.14 (m, 1H), 6.68 (d, J=16.4 Hz, 1H), 6.53 (dd, J=16.4, 6.8 Hz, 1H), 5.11-5.06 (m, 1H), 4.58 (s, 3H), 4.26-4.24 (m, 2H), 4.00 (s, 3H), 3.87-3.79 (m, 3H), 3.74 (s, 1H), 3.69-3.47 (m, 15H), 3.06-2.92 (s, 3H total), 2.87-2.79 (m, 1H), 2.78-2.65 (m, 2H), 2.23-2.08 (m, 3H), 2.04-1.91 (m, 4H), 1.48-1.21 (m, 5H). LCMS (ESI): m/z 966.5 (M+H)⁺.

### Example 6

### Preparation of (S)—N-(1-(6-aminopyridin-2-yl)ethyl)-5-(4-(trifluoromethyl)phenoxy)-2-naphthamide and N-[(1R)-1-(6-amino-2-pyridyl)ethyl]-5-[4-(trifluoromethyl)phenoxy]naphthalene-2-carboxamide (Compound T6/Example 55 of US2020/0347009A1)

### 1-(4-bromopyridin-2-yl)-N-(2,4-dimethoxybenzyl)ethanamine

To a solution of 1-(6-bromo-2-pyridyl)ethanone (3.2 g, 16.00 mmol, 1 eq) and (2,4-dimethoxyphenyl)methanamine (2.67 g, 16.00 mmol, 2.41 mL, 1 eq) in DCE (30 mL) was added HOAc (4.80 g, 79.99 mmol, 4.57 mL, 5 eq) and stirred at 25° C. for 1 hr, and then NaBH(OAc)₃ (5.09 g, 24.00 mmol, 1.5 eq) was added. The resulting mixture was stirred at 25° C. for 15 hr. Then iced water (30 mL) was added and the mixture was neutralized to pH=9˜10 with aq. NaOH (2 M). The aqueous phase was extracted with EA (30 mL*3). The combined organic phase was washed with brine (60 mL), dried with anhydrous Na₂SO₄, filtered and concentrated in vacuo. The residue was purified by flash silica gel chromatography. Compound 1-(6-bromo-2-pyridyl)-N-[(2,4-dimethoxyphenyl)methyl]ethanamine (1.6 g, 4.42 mmol, 27.6% yield) was obtained.

### N-(1-(6-bromopyridin-2-yl)ethyl)-N-(2,4-dimethoxybenzyl)-5-(4-(trifluoromethyl)phenoxy)-2-naphthamide

A mixture of 5-[4-(trifluoromethyl)phenoxy]naphthalene-2-carboxylic acid (473.0 mg, 1.42 mmol, 1 eq), HATU (1.08 g, 2.85 mmol, 2 eq) in DCM (10 mL) was added DIPEA (551.9 mg, 4.27 mmol, 0.74 mL, 3 eq) at 25° C. After addition, the mixture was stirred at 25° C. for 1 hr, and then 1-(6-bromo-2-pyridyl)-N-[(2,4-dimethoxyphenyl)methyl]ethanamine (500 mg, 1.42 mmol, 1 eq) (in DCM (3 mL)) was added. The resulting mixture was stirred at 25° C. for 15 hr. The residue was poured into H₂O (50 mL) and stirred for 5 min. The aqueous phase was extracted with EA (30 mL*3). The combined organic phase was washed with brine (50 mL), dried with anhydrous Na₂SO₄, filtered and concentrated in vacuum. The residue was purified by flash silica gel chromatography. Compound N-[1-(6-bromo-2-pyridyl)ethyl]-N-[(2,4-dimethoxyphenyl)methyl]-5-[4-(trifluoromethyl)phenoxy]naphthalene-2-carboxamide (650 mg, 0.97 mmol, 68.6% yield) was obtained.

### N-(1-(6-bromopyridin-2-yl)ethyl)-5-(4-(trifluoromethyl)phenoxy)-2-naphthamide

To a solution of N-[1-(6-bromo-2-pyridyl)ethyl]-N-[(2,4-dimethoxyphenyl)methyl]-5-[4-(trifluoromethyl)phenoxy]naphthalene-2-carboxamide (590 mg, 0.88 mmol, 1 eq) in DCM (0.5 mL) was added TFA (9.09 g, 79.69 mmol, 5.90 mL, 89.88 eq). The mixture was stirred at 25° C. for 3 hr. Then iced water (30 mL) was added and the mixture was neutralized to pH=9˜10 with aq. NaOH (2 M). The aqueous phase was extracted with EA (30 mL*3). The combined organic phase was washed with brine (60 mL), dried with anhydrous Na₂SO₄, filtered and concentrated in vacuo to give crude product. The residue was purified by flash silica gel chromatography. Compound N-[1-(6-bromo-2-pyridyl)ethyl]-5-[4-(trifluoromethyl)phenoxy]naphthalene-2-carboxamide (425 mg, 0.82 mmol, 93.0% yield) was obtained.

### N-(1-(6-aminopyridin-2-yl)ethyl)-5-(4-(trifluoromethyl)phenoxy)-2-naphthamide

A mixture of N-[1-(6-bromo-2-pyridyl)ethyl]-5-[4-(trifluoromethyl)phenoxy]naphthalene-2-carboxamide (150 mg, 0.29 mmol, 1 eq), Cu₂O (41.6 mg, 0.29 mmol, 29.7 uL, 1 eq), NH₃H₂O (134.2 mg, 1.46 mmol, 0.14 mL, 38%, 5 eq) in dioxane (1 mL) were loaded in a sealed reaction tube. The reaction temperature was increased to 80° C. and the reaction mixture was stirred at 80° C. for 16 hr. The mixture was poured into H₂O (30 mL) and stirred for 5 min. The aqueous phase was extracted with EA(15 mL*3). The combined organic phase was washed with brine (20 mL), dried with anhydrous Na₂SO₄, filtered and concentrated in vacuo. The residue was purified by flash silica gel chromatography. Compound N-[1-(6-amino-2-pyridyl)ethyl]-5-[4-(trifluoromethyl)phenoxy]naphthalene-2-carboxamide (98 mg, 0.21 mmol, 74.5% yield) was obtained.

### (S)—N-(1-(6-aminopyridin-2-yl)ethyl)-5-(4-(trifluoromethyl)phenoxy)-2-naphthamide (Compound 65 of US2020/0347009A1) and (R)—N-(1-(6-aminopyridin-2-yl)ethyl)-5-(4-(trifluoromethyl)plienoxy)-2-naphthamide (Compound T6/Example 55 of US2020/0347009A1)

The racemic compound N-[1-(6-amino-2-pyridyl)ethyl]-5-[4-(trifluoromethyl)phenoxy]naphthalene-2-carboxamide (85 mg, 0.18 mmol, 1 eq) was purified by SFC to give (S)—N-(1-(6-aminopyridin-2-yl)ethyl)-5-(4-(trifluoromethyl)phenoxy)-2-naphthamide (20.2 mg, 42.0 umol, 22.3% yield) LCMS (ESI): RT=0.776 min, mass calcd for C₂₅H₂₀F₃N₃O₂ 451.44 m/z found 452.1 [M+H]⁺; ¹H NMR (400 MHz, CD₃OD) δ 8.51 (s, 1H), 8.10 (d, J=8.9 Hz, 1H), 7.98-7.87 (m, 2H), 7.66 (d, J=8.5 Hz, 2H), 7.58 (t, J=8.0 Hz, 1H), 7.43 (t, J=7.7 Hz, 1H), 7.23 (d, J=7.6 Hz, 1H), 7.13 (d, J=8.6 Hz, 2H), 6.66 (d, J=7.3 Hz, 1H), 6.46 (d, J=8.1 Hz, 1H), 5.18-5.07 (m, 1H), 1.56 (d, J=7.0 Hz, 3H); and Compound T6/Example 55 of US2020/0347009A1 (17.6 mg, 37.4 umol, 19.8% yield) LCMS (ESI): RT=0.773 min, mass calcd for C₂₅H₂₀F₃N₃O₂ 451.44 m/z found 452.1 [M+H]⁺; ¹H NMR (400 MHz, CD₃OD) δ 8.51 (s, 1H), 8.10 (d, J=8.8 Hz, 1H), 7.98-7.88 (m, 2H), 7.66 (d, J=8.5 Hz, 2H), 7.59 (t, J=7.9 Hz, 1H), 7.42 (t, J=7.8 Hz, 1H), 7.23 (d, J=7.5 Hz, 1H), 7.13 (d, J=8.4 Hz, 2H), 6.66 (d, J=7.5 Hz, 1H), 6.46 (d, J=8.3 Hz, 1H), 5.12 (q, J=6.9 Hz, 1H), 1.56 (d, J=6.9 Hz, 3H).

### Example 7

### Preparation of N-[(1S)-1-(2-pyridyl)ethyl]-5-[4-(trifluoromethyl)phenyl]naphthalene-2-carboxamide (Compound T7/Example 84 of WO2020/097389A1) and (R)—N-(1-(pyridin-2-yl)ethyl)-5-(4-(trifluoromethyl)phenyl)-2-naphthamide

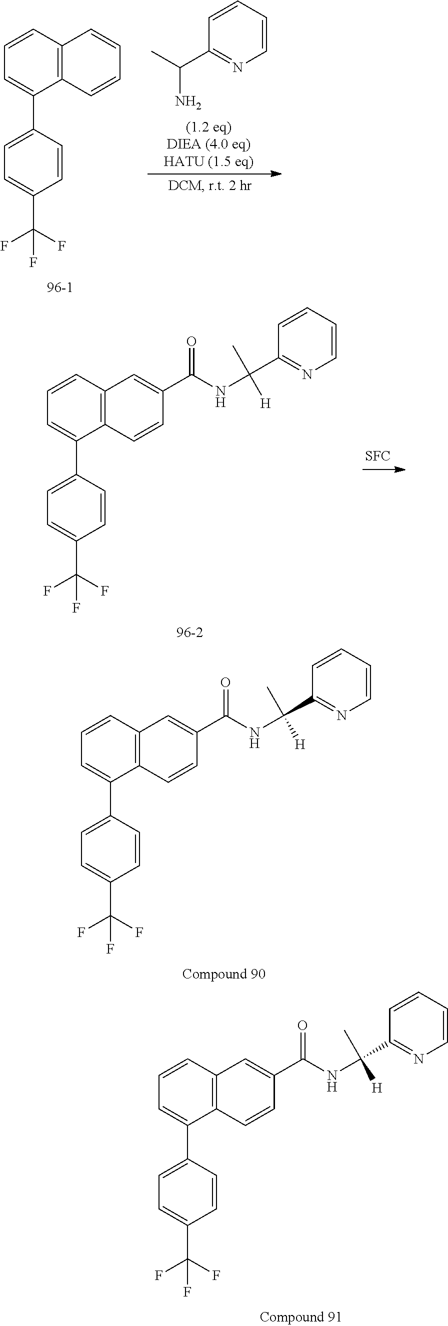

### Step 1: N-(1-(pyridin-2-yl)ethyl)-5-(4-(trifluoromethyl)phenyl)-2-naphthamide

The mixture of compound 90-1 of WO2020/097389A1 (200 mg, 0.63 mmol, 1 eq), HATU (360.6 mg, 0.94 mmol, 1.5 eq) and DIEA (326.9 mg, 2.53 mmol, 0.44 mL, 4 eq) in DCM (3 mL) was stirred at 25° C. for 1 hr. Then 1-(2-pyridyl)ethanamine (92.7 mg, 0.75 mmol, 1.2 eq) was added to the mixture and the mixture was stirred at 25° C. for another 1 hr. LC-MS showed the desired compound was detected. The reaction mixture was diluted with H₂O (10 mL) and the mixture was extracted with EA (10 mL*3). The combined organic phase was washed with brine (10 mL*2), dried with anhydrous Na₂SO₄, filtered and concentrated in vacuum. The residue was purified by column chromatography (SiO₂, Petroleum ether/Ethyl acetate=1/0 to 1:1). Compound 90-2 of WO2020/097389A1 (200 mg, 0.46 mmol, 72.9% yield) was obtained as yellow oil.

### Step 2: (S)—N-(1-(pyridin-2-yl)ethyl)-5-(4-(trifluoromethyl)phenyl)-2-naphthamide and (R)—N-(1-(pyridin-2-yl)ethyl)-5-(4-(trifluoromethyl)phenyl)-2-naphthamide

Compound 90-2 of WO2020/097389A1 (90 mg, 0.21 mmol, 1 eq) was purified by SFC. Compound T7/Example 84 of WO2020/097389A1 (20 mg, 47.5 umol, 22.2% yield) was obtained as white solid. LCMS (ESI): RT=0.881 min, mass calcd for C₂₅H₁₉F₃N₂O 420.43, m/z found 421.1 [M+H]⁺; ¹H NMR (400 MHz, CD₃OD) δ 8.56-8.51 (m, 2H), 8.08 (d, J=8.3 Hz, 1H), 7.93-7.78 (m, 5H), 7.72-7.63 (m, 3H), 7.56 (d, J=6.1 Hz, 1H), 7.51 (d, J=7.9 Hz, 1H), 7.31 (dd, J=4.8, 6.6 Hz, 1H), 5.32 (q, J=7.0 Hz, 1H), 1.63 (d, J=7.0 Hz, 3H). (R)—N-(1-(pyridin-2-yl)ethyl)-5-(4-(trifluoromethyl)phenyl)-2-naphthamide (30 mg, 71.3 umol, 33.3% yield) was obtained as white solid. LCMS (ESI): RT=0.896 min, mass scaled for C₂₅H₁₉F₃N₂O 420.43, m/z found 421.1 [M+H]⁺; ¹H NMR (400 MHz, CD₃OD) δ 8.57-8.54 (m, 2H), 8.08 (d, J=7.9 Hz, 1H), 7.95-7.81 (m, 4H), 7.95-7.79 (m, 1H), 7.70-7.64 (m, 2H), 7.71-7.64 (m, 1H), 7.61-7.55 (m, 1H), 7.58 (t, J=7.9 Hz, 1H), 7.40 (dd, J=5.5, 6.8 Hz, 1H), 5.37-5.28 (m, 1H), 1.65 (d, J=7.5 Hz, 3H).

### Example 8

### Preparation of N-methyl-3-(1-methylimidazol-4-yl)-4-[4-(trifluoromethyl)anilino]benzenesulfonamide (Compound T8/Example 113 of US2020/0354325A1)

### Step 1: 3-Bromo-4-fluoro-N-methylbenzenesulfonamide

To a solution of compound 121-1 of US2020/0354325A1 (1.0 g, 3.7 mmol, 1.0 eq) in DCM (10 mL) was added MeNH₂ (2 M, 3.7 mL, 2.0 eq). The reaction mixture was stirred at 25° C. for 2 hours. The mixture was diluted with water (15 mL) and the resultant mixture was extracted with DCM (30 mL*2). The combined organic layers were dried over Na₂SO₄, filtered and concentrated to dryness under reduced pressure to obtain the title compound 121-2 of US2020/0354325A1 (950 mg, 97% yield).

### Step 2: 4-(Benzylamino)-3-bromo-N-methylbenzenesulfonamide

A solution of compound 121-2 of US2020/0354325A1 (850 mg, 3.17 mmol, 1.0 eq) and compound 121-2a of US2020/0354325A1 (679 mg, 6.34 mmol, 2.0 eq) in DMSO (4 mL) was stirred at 140° C. for 1 hour. The mixture was diluted with water (30 mL) and the resultant mixture was extracted with EA (50 mL*3). The combined organic layers were dried over Na₂SO₄, filtered and concentrated to dryness under reduced pressure. The residue was purified by column chromatography over silica gel to afford the title compound 121-3 of US2020/0354325A1 (1.0 g, 89% yield). LCMS (ESI): RT=0.798 min, mass calcd. for C₁₄H₁₅BrN₂O₂S 354.00, m/z found 356.7 [M+H]⁺.

### Step 3: 4-(Benzylamino)-N-methyl-3-(4,4,5,5-tetramethyl-1,3,2-dioxaborolan-2-yl)benzenesulfonamide

A solution of compound 121-3 of US2020/0354325A1 (850 mg, 2.39 mmol, 1.0 eq), compound 121-3a of US2020/0354325A1 (911 mg, 3.59 mmol, 1.5 eq), Pd(dppf)Cl₂ (88 mg, 0.12 mmol, 0.05 eq) and AcOK (470 mg, 4.79 mmol, 2.0 eq) in Dioxane (10 mL) was heated to 90° C. and stirred at 90° C. for 16 hours under N₂. The reaction mixture was concentrated under reduced pressure. The mixture was diluted with water (30 mL) and the resultant mixture was extracted with EA (50 mL*3). The combined organic layers were dried over Na₂SO₄, filtered and concentrated under reduced pressure. The residue was purified by column chromatography over silica gel to afford the title compound 121-4 of US2020/0354325A1 (800 mg, 68% yield). LCMS (ESI): RT=0.827 min, mass calcd. for C₂₀H₂₇BN₂O₄S 402.18, m/z found 402.9 [M+H]⁺.

### Step 4: 4-(Benzylamino)-N-methyl-3-(1-methyl-1H-imidazol-4-yl)benzenesulfonamide

To a solution of compound 121-4 of US2020/0354325A1 (700 mg, 1.74 mmol, 1.0 eq), compound 121-4a of US2020/0354325A1 (308 mg, 1.91 mmol, 1.1 eq), Cs₂CO₃ (1.13 g, 3.48 mmol, 2.0 eq) in Dioxane (8 mL) and H₂O (2 mL) was added Pd(PPh₃)₄(101 mg, 87.0 umol, 0.05 eq) under N₂. The reaction mixture was stirred at 90° C. for 16 hours. The reaction mixture was concentrated under reduced pressure. The mixture was diluted with water (20 mL) and the resultant mixture was extracted with EA (50 mL*3). The combined organic layers were dried over Na₂SO₄, filtered and concentrated to dryness under reduced pressure. The residue was purified by preparative high performance liquid chromatography. The pure fractions were collected and the volatiles were removed under vacuum. The resulting mixture was lyophilized to dryness to remove the solvent residue completely. The title compound 121-5 of US2020/0354325A1 (180 mg, 28% yield) was obtained. LCMS (ESI): RT=0.592 min, mass calcd. for C₁₈H₂₀N₄O₂S 356.13, m/z found 356.9 [M+H]⁺, ¹H NMR (400 MHz, CDCl₃) δ 9.09 (s, 1H), 7.86 (d, J=2.3 Hz, 1H), 7.49 (dd, J=2.3, 8.8 Hz, 1H), 7.46 (s, 1H), 7.41-7.30 (m, 4H), 7.29-7.26 (m, 2H), 6.62 (d, J=8.8 Hz, 1H), 4.53 (s, 2H), 4.21 (q, J=5.4 Hz, 1H), 3.75 (s, 3H), 2.61 (d, J=5.5 Hz, 3H).

### Step 5: 4-Amino-N-methyl-3-(1-methyl-1H-imidazol-4-yl)benzenesulfonamide

To a solution of compound 121-5 of US2020/0354325A1 (170 mg, 0.477 mmol, 1.0 eq) in MeOH (5 mL) were added Pd/C (50 mg, 10% purity) and HCl (242 mg, 2.38 mmol, 236.79 uL, 36% purity, 5.0 eq) under N₂. The suspension was degassed under vacuum and purged with H₂ several times. The mixture was stirred under H₂ (45 psi) at 25° C. for 16 hours. The reaction mixture was filtered and the filtration was concentrated to obtain the title compound 121-6 of US2020/0354325A1 (140 mg, crude).

### Step 6: N-Methyl-3-(1-methyl-1H-imidazol-4-yl)-4-((4-(trifluoromethyl)phenyl)amino) benzenesulfonamide

To a solution of compound 121-6 of US2020/0354325A1 (140 mg, 0.526 mmol, 1.0 eq), compound 121-6a of US2020/0354325A1 (150 mg, 0.789 mmol, 1.5 eq) and Cu(OAc)₂ (115 mg, 0.631 mmol, 1.2 eq) in DCM (5 mL) was added DIPEA (272 mg, 2.10 mmol, 4.0 eq). The reaction mixture was stirred at 25° C. for 16 hours. The reaction mixture was concentrated under reduced pressure. The mixture was diluted with water (30 mL) and the resultant mixture was extracted with EA (50 mL*3). The combined organic layers were dried over Na₂SO₄, filtered and concentrated to dryness under reduced pressure. The residue was purified by preparative high performance liquid chromatography. The pure fractions were collected and the volatiles were removed under vacuum. The residue was re-suspended in water (10 mL) and the resulting mixture was lyophilized to dryness to remove the solvent residue completely. Compound T8 (Compound 121 of US2020/0354325A1) (16.20 mg, 39.47 umol, 7.5% yield) was obtained. LCMS (ESI): RT=0.653 min, mass calcd. for C₁₈H₁₇F₃N₄O₂S 410.10, m/z found 410.9 [M+H]⁺, ¹H NMR (400 MHz, CDCl₃) δ 10.88 (s, 1H), 7.97 (s, 1H), 7.61-7.51 (m, 4H), 7.48 (d, J=8.8 Hz, 1H), 7.33 (d, J=8.3 Hz, 3H), 4.38-4.30 (m, 1H), 3.79 (s, 3H), 2.67 (d, J=5.5 Hz, 3H).

### Example 9

### Preparation of 5-(4-cyclohexylphenyl)-2-(3-methylpyrazin-2-yl)-3-[rac-(2S,3S)-3-(fluoromethyl)-2-methyl-azetidine-1-carbonyl]-4H-pyrazolo[1,5-a]pyrimidin-7-one (Compound T9/Example 41 of WO2021/108483A1) and 5-(4-cyclohexylphenyl)-3-((2R,3R)-3-(fluoromethyl)-2-methylazetidine-1-carbonyl)-2-(3-methylpyrazin-2-yl)pyrazolo[1,5-a]pyrimidin-7(4H)-one

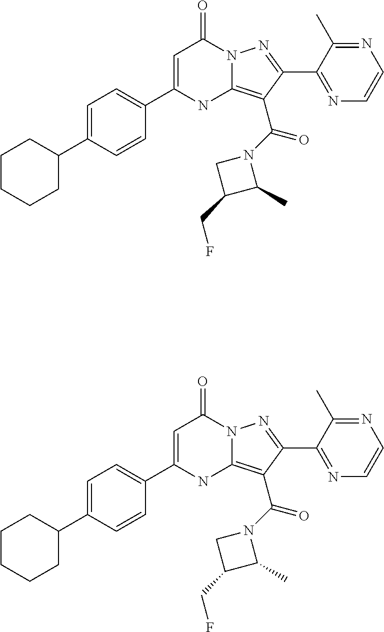 [SMILES: Cc1nccnc1-c1nn2c(=O)cc(-c3ccc(C4CCCCC4)cc3)[nH]c2c1C(=O)N1C[C@H](CF)[C@@H]1C ; Cc1nccnc1-c1nn2c(=O)cc(-c3ccc(C4CCCCC4)cc3)[nH]c2c1C(=O)N1C[C@@H](CF)[C@H]1C]

### Step 1: Cis-5-(4-cyclohexylphenyl)-3-(3-(fluoromethyl)-2-methylazetidine-1-carbonyl)-2-(3-methylpyrazin-2-yl)pyrazolo[1,5-a]pyrimidin-7(4H)-one

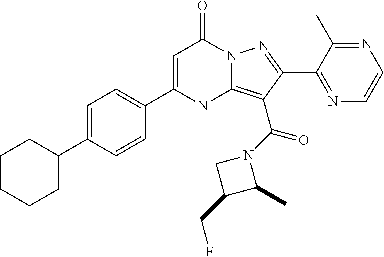 [SMILES: Cc1nccnc1-c1nn2c(=O)cc(-c3ccc(C4CCCCC4)cc3)[nH]c2c1C(=O)N1C[C@H](CF)[C@@H]1C]

The title compound (140 mg, 54%) was prepared from cis-5-(4-cyclohexylphenyl)-3-(3-(fluoromethyl)-2-methylazetidine-1-carbonyl)-2-(pyrazin-2-yl)pyrazolo[1,5-a]pyrimidin-7(4H)-one (250 mg, 0.50 mmol) and 2-methyl-3-(tributylstannyl) pyrazine (286 mg, 0.75 mmol) following the procedure outlined for Example 7 of WO2021/108483A1, Step 2. LCMS (ESI): m/z 515.2 (M+H)⁺.

### Step 2: 5-(4-Cyclohexylphenyl)-3-((2S,3S)-3-(fluoromethyl)-2-methylazetidine-1-carbonyl)-2-(3-methylpyrazin-2-yl)pyrazolo[1,5-a]pyrimidin-7(4H)-one and 5-(4-cyclo hexylphenyl)-3-((2R,3R)-3-(fluoromethyl)-2-methylazetidine-1-carbonyl)-2-(3-methyl pyrazin-2-yl)pyrazolo[1,5-a]pyrimidin-7(4H)-one

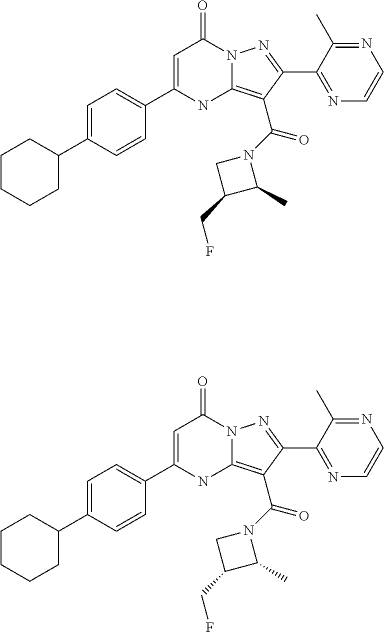 [SMILES: Cc1nccnc1-c1nn2c(=O)cc(-c3ccc(C4CCCCC4)cc3)[nH]c2c1C(=O)N1C[C@H](CF)[C@@H]1C ; Cc1nccnc1-c1nn2c(=O)cc(-c3ccc(C4CCCCC4)cc3)[nH]c2c1C(=O)N1C[C@@H](CF)[C@H]1C]

Cis-5-(4-cyclohexylphenyl)-3-(3-(fluoromethyl)-2-methylazetidine-1-carbonyl)-2-(3-methylpyrazin-2-yl)pyrazolo[1,5-a]pyrimidin-7(4H)-one was separated by SFC (column: DAICEL CHIRALPAK IG (250 mm*30 mm, 10 um); Condition: 0.1% NH₃H₂O ETOH to afford 5-(4-cyclohexylphenyl)-3-((2R,3R)-3-(fluoromethyl)-2-methylazetidine-1-carbonyl)-2-(3-methylpyrazin-2-yl)pyrazolo[1,5-a]pyrimidin-7(4H)-one (fourth peak on SFC, 23.7 mg, 24%) as a white solid and crude second peak which was separated by further SFC (DAICEL CHIRALPAK AD(250 mm*30 mm, 10 um), 0.1% NH₃H₂O ETOH, 40%) to afford 5-(4-cyclohexylphenyl)-3-((2S,3S)-3-(fluoromethyl)-2-methylazetidine-1-carbonyl)-2-(3-methyl pyrazin-2-yl)pyrazolo[1,5-a]pyrimidin-7(4H)-one (Compound T9/Example 41 of WO2021/108483A1; 19.0 mg, 19%) as a white solid.

5-(4-Cyclohexylphenyl)-3-((2S,3S)-3-(fluoromethyl)-2-methylazetidine-1-carbonyl)-2-(3-methylpyrazin-2-yl)pyrazolo[1,5-a]pyrimidin-7(4H)-one (Compound T9/Example 41 of WO2021/108483A1), second peak on SFC; ¹H NMR (400 MHz, DMSO-d₆): δ 12.40 (br s, 1H), 8.54-8.50 (m 2H), 7.79-7.77 (m, 2H), 7.38-7.36 (m, 2H), 6.10 (s, 1H), 4.75-4.27 (m, 3H), 3.86-3.84 (m, 2H), 2.87-2.86 (m, 2H), 2.58-2.56 (m, 3H), 1.80-1.76 (m, 4H), 1.70-1.68 (m, 1H), 1.48-1.21 (m, 8H); LCMS (ESI): m/z 515.3 (M+H)⁺.

5-(4-Cyclohexylphenyl)-3-((2R,3R)-3-(fluoromethyl)-2-methylazetidine-1-carbonyl)-2-(3-methylpyrazin-2-yl)pyrazolo[1,5-a]pyrimidin-7(4H)-one, fourth peak on SFC; ¹H NMR (400 MHz, DMSO-d₆): δ 8.48-8.45 (m, 2H), 7.96-7.64 (m, 2H), 7.33-7.28 (m, 2H), 6.11 (s, 1H), 4.89-4.39 (m, 3H), 3.90-3.86 (m, 2H), 2.97-2.58 (m, 2H), 2.53 (s, 3H), 1.80-1.76 (m, 4H), 1.71-1.68 (m, 1H), 1.49-1.11 (m, 8H). LCMS (ESI): m/z 515.3 (M+H)⁺.

### Example 10

### Preparation of 2-methyl-8-[4-(trifluoromethyl)phenyl1-2H,8H-pyrazolo[3,4-b]indole-5-carboxylic acid (Compound T10/Example 2-4 of WO2021/224291A1)

### Example 10-1: Synthesis of ethyl 3-(3-amino-1-methyl-1H-pyrazol-4-yl)-4-chlorobenzoate

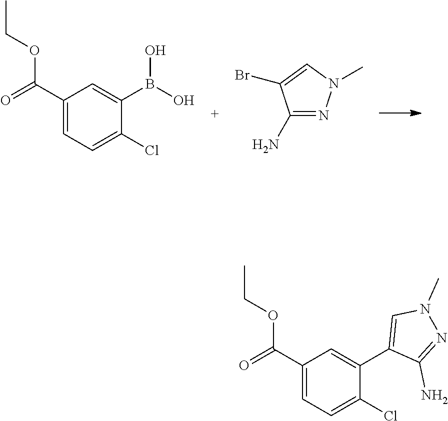

To a suspension of 2-chloro-5-(ethoxycarbonyl)phenyl]boronic acid (500 mg; 2.19 mmol) in dioxane (4 ml) and water (0.4 ml) was added 4-bromo-1-methyl-1H-pyrazol-3-amine (385 mg; 2.19 mmol), K₂CO₃ (605 mg; 4.38 mmol) and Pd(dppf)Cl₂ (160 mg). The mixture was stirred at 60° C. under N₂ atmosphere for 6 h. The mixture was poured into water (5 ml), and then extracted with EA (6 ml*3). The combined organic phase was collected and evaporated under vacuum. The residue was purified by C18 column chromatography (ACN/H₂O=5%-95%) and the purified product could be obtained (500 mg; 74%; white powder).

¹H NMR (400 MHz, DMSO) δ 8.07 (d. J=2.1 Hz, 1H), 7.77 (dd, J=8.4, 2.2 Hz, 1H), 7.67 (s, 1H), 7.63 (d, J=8.4 Hz, 1H), 4.58 (s, 2H), 4.32 (q, J=7.1 Hz, 2H), 3.66 (s, 3H), 1.31 (t, J=7.1 Hz, 3H).

### Example 10-2: Synthesis of ethyl 2-methyl-2H,8H-pyrazolo[3,4-b]indole-5-carboxylate

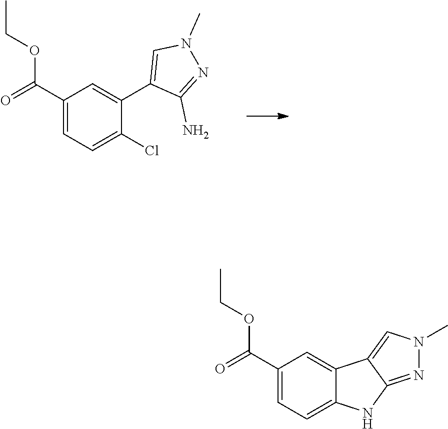 [SMILES: CCOC(=O)c1ccc(Cl)c(-c2cn(C)nc2N)c1 ; CCOC(=O)c1ccc2[nH]c3nn(C)cc3c2c1]

To a suspension of ethyl 3-(3-amino-1-methyl-1H-pyrazol-4-yl)-4-chloro-benzoate (300 mg; 1.1 mmol) in dioxane (15 ml) was added di-tert-butyl[2′,4′,6′-tris(propan-2-yl)-[1,1′-biphenyl]-2-yl]phosphane {2′-amino-[1,1′-biphenyl]-2-yl}palladiumylium methanesulfonate (85 mg; 0.11 mmol) and Cs₂CO₃ (699 mg; 2.14 mmol). The mixture was stirred at 120° C. under N₂ atmosphere for 6 h. The mixture was poured into water (5 ml), and then extracted with EA (6 ml*3). The combined organic phase was collected and evaporated under vacuum. The residue was purified by C18 column chromatography (ACN/H₂O=5%-95%) and the purified product could be obtained. (110 mg; 42%; white solid).

¹H NMR (400 MHz, DMSO) δ 8.31 (d, J=1.7 Hz, 1H), 8.03 (s, 1H), 7.83 (dd, J=8.5, 1.8 Hz, 1H), 7.32 (d, J=8.5 Hz, 1H), 4.31 (q, J=7.1 Hz, 2H), 3.97 (s, 3H), 1.34 (t, J=7.1 Hz, 3H).

### Example 10-3: Synthesis of ethyl 2-methyl-8-[4-(trifluoromethyl)phenyl]-2H,8H-pyrazolo[3,4-b]indole-5-carboxylate

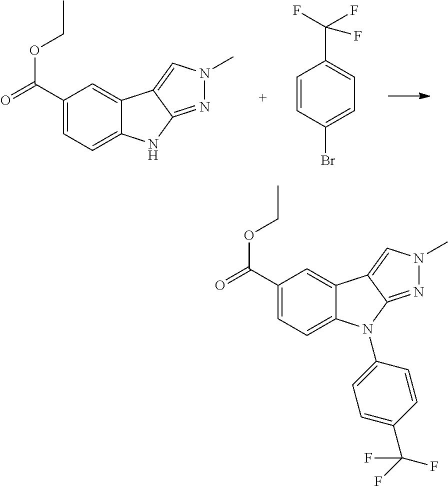 [SMILES: CCOC(=O)c1ccc2[nH]c3nn(C)cc3c2c1 ; FC(F)(F)c1ccc(Br)cc1 ; CCOC(=O)c1ccc2c(c1)c1cn(C)nc1n2-c1ccc(C(F)(F)F)cc1]

A sealed tube was charged with ethyl 2-methyl-2H,8H-pyrazolo[3,4-b]indole-5-carboxylate (85 mg; 0.35 mmol), 1-bromo-4-(trifluoromethyl)benzene (102 mg 0.45 mmol), XPhosPd G2 (17 mg; 0.02 mmol) and Cs₂CO₃ (342 mg; 1.05 mmol) in dioxane (5 ml). The mixture was stirred was stirred under N₂ 100° C. for 2 h. The mixture was fitted and concentrated to get crude product as a black oil. The crude product was purified by C18 (ACN/H₂O=5%-95%) to get the product as a white solid. (92 mg; 62%; white solid).

¹H NMR (400 MHz, DMSO) δ 8.45 (d, J=1.5 Hz, 1H), 8.23 (s, 1H), 8.08 (d, J=8.5 Hz, H), 7.98 (d, J=8.6 Hz, 2H), 7.93 (d, J=1.8 Hz, 1H), 7.80 (d, J=8.7 Hz, 1H), 4.35 (d, J=7.1 Hz, 2H), 4.04 (s, 3H), 1.36 (t, J=7.1 Hz, 2H).

### Example 10-4: Synthesis of 2-methyl-8-[4-(trifluoromethyl)phenyl]-2H,8H-pyrazolo[3,4-b]indole-5-carboxylic acid

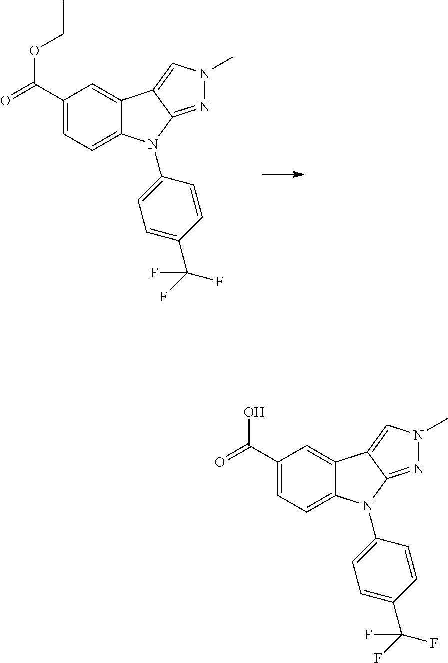 [SMILES: CCOC(=O)c1ccc2c(c1)c1cn(C)nc1n2-c1ccc(C(F)(F)F)cc1 ; Cn1cc2c3cc(C(=O)O)ccc3n(-c3ccc(C(F)(F)F)cc3)c2n1]

To a solution of ethyl 2-methyl-8-[4-(trifluoromethyl)phenyl]-2H,8H-pyrazolo[3,4-b]indole-5-carboxylate (90 mg; 0.21 mmol) in MeOH (40 ml) was added an aqueous solution of 1M sodium hydroxide (1 ml). The mixture was stirred under N₂ at 60° C. for 6 h. The mixture was concentrated and adjusted by 1N hydrochloric acid to pH=1-2. The mixture was purified by C18 (0.1% TFA/H₂O=20%-95%) to get the product (Compound T10). (59 mg; 73%; white solid).

1H NMR (400 MHz, DMSO) δ 12.74 (s, 1H), 8.43 (d, J=1.7 Hz, 1H), 8.22 (s, 1H), 8.08 (d, J=8.5 Hz, 2H), 7.98 (d, J=8.6 Hz, 2H), 7.93 (dd, J=8.7, 1.8 Hz, 1H), 7.78 (d, J=8.7 Hz, 1H), 4.03 (s, 3H).

### Example 11

### Preparation of 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one (Compound K1/Example 41 of US2018/0334454A1)

### Step 1: 2,6-Dichloro-5-fluoronicotinamide (Intermediate S)

To a mixture of 2,6-dichloro-5-fluoronicotinic acid (4.0 g, 19.1 mmol, AstaTech Inc., Bristol, Pa.) in dichloromethane (48 mL) was added oxalyl chloride (2M solution in DCM, 11.9 mL, 23.8 mmol), followed by a catalytic amount of DMF (0.05 mL). The reaction was stirred at room temperature overnight and then was concentrated. The residue was dissolved in 1,4-dioxane (48 mL) and cooled to 0° C. Ammonium hydroxide solution (28.0-30% NH3 basis, 3.6 mL, 28.6 mmol) was added slowly via syringe. The resulting mixture was stirred at 0° C. for 30 min and then was concentrated. The residue was diluted with a 1:1 mixture of EtOAc/Heptane and agitated for 5 min, then was filtered. The filtered solids were discarded, and the remaining mother liquor was partially concentrated to half volume and filtered. The filtered solids were washed with heptane and dried in a reduced-pressure oven (45° C.) overnight to provide 2,6-dichloro-5-fluoronicotinamide. ¹H NMR (400 MHz, DMSO-d₆) δ ppm 8.23 (d, J=7.9 Hz, 1H) 8.09 (br s, 1H) 7.93 (br s, 1H). m/z (ESI, +ve ion): 210.9 (M+H)⁺.

### Step 2: 2,6-Dichloro-5-fluoro-N-((2-isopropyl-4-methylpyridin-3-yl)carbamoyl)nicotinamide

To an ice-cooled slurry of 2,6-dichloro-5-fluoronicotinamide (Intermediate S, 5.0 g, 23.9 mmol) in THF (20 mL) was added oxalyl chloride (2 M solution in DCM, 14.4 mL, 28.8 mmol) slowly via syringe. The resulting mixture was heated at 75° C. for 1 h, then heating was stopped, and the reaction was concentrated to half volume. After cooling to 0° C., THF (20 mL) was added, followed by a solution of 2-isopropyl-4-methylpyridin-3-amine (Intermediate R, 3.59 g, 23.92 mmol) in THF (10 mL), dropwise via cannula. The resulting mixture was stirred at 0° C. for 1 h and then was quenched with a 1:1 mixture of brine and saturated aqueous ammonium chloride. The mixture was extracted with EtOAc (3×) and the combined organic layers were dried over anhydrous sodium sulfate and concentrated to provide 2,6-dichloro-5-fluoro-N-((2-isopropyl-4-methylpyridin-3-yl)carbamoyl)nicotinamide. This material was used without further purification in the following step. m/z (ESI, +ve ion): 385.1 (M+H)⁺.

### Step 3: 7-Chloro-6-fluoro-1-(2-isopropyl-4-methyl-pyridin-3-yl)pyrido[2,3-d]pyrimidine-2,4(1H,3H)-dione

To an ice-cooled solution of 2,6-dichloro-5-fluoro-N-((2-isopropyl-4-methylpyridin-3-yl)carbamoyl)nicotinamide (9.2 g, 24.0 mmol) in THF (40 mL) was added KHMDS (1 M solution in THF, 50.2 mL, 50.2 mmol) slowly via syringe. The ice bath was removed and the resulting mixture was stirred for 40 min at room temperature. The reaction was quenched with saturated aqueous ammonium chloride and extracted with EtOAc (3×). The combined organic layers were dried over anhydrous sodium sulfate and concentrated. The residue was purified by silica gel chromatography (eluent: 0-50% 3:1 EtOAc-EtOH/heptane) to provide 7-chloro-6-fluoro-1-(2-isopropyl-4-methylpyridin-3-yl)pyrido[2,3-d]pyrimidine-2,4(1H,3H)-dione. ¹H NMR (400 MHz, DMSO-d₆) δ ppm 12.27 (br s, 1H), 8.48-8.55 (m, 2H), 7.29 (d, J=4.8 Hz, 1H), 2.87 (quin, J=6.6 Hz, 1H), 1.99-2.06 (m, 3H), 1.09 (d, J=6.6 Hz, 3H), 1.01 (d, J=6.6 Hz, 3H). ¹⁹F NMR (376 MHz, DMSO-d₆) δ: −126.90 (s, 1F). m/z (ESI, +ve ion): 349.1 (M+H)⁺.

### Step 4: 4,7-Dichloro-6-fluoro-1-(2-isopropyl-4-methylpyridin-3-yl)pyrido[2,3-d]pyrimidin-2(1H)-one

To a solution of 7-chloro-6-fluoro-1-(2-isopropyl-4-methylpyridin-3-yl)pyrido[2,3-d]pyrimidine-2,4(1H,3H)-dione (4.7 g, 13.5 mmol) and DIPEA (3.5 mL, 20.2 mmol) in acetonitrile (20 mL) was added phosphorus oxychloride (1.63 mL, 17.5 mmol), dropwise via syringe. The resulting mixture was heated at 80° C. for 1 h, and then was cooled to room temperature and concentrated to provide 4,7-dichloro-6-fluoro-1-(2-isopropyl-4-methylpyridin-3-yl)pyrido[2,3-d]pyrimidin-2(1H)-one. This material was used without further purification in the following step. m/z (ESI, +ve ion): 367.1 (M+H)⁺.

### Step 5: (S)-tert-Butyl 4-(7-chloro-6-fluoro-1-(2-isopropyl-4-methylpyridin-3-yl)-2-oxo-1,2-dihydro-pyrido[2,3-d]pyrimidin-4-yl)-3-methylpiperazine-1-carboxylate

To an ice-cooled solution of 4,7-dichloro-6-fluoro-1-(2-isopropyl-4-methylpyridin-3-yl)pyrido[2,3-d]pyrimidin-2(1H)-one (13.5 mmol) in acetonitrile (20 mL) was added DIPEA (7.1 mL, 40.3 mmol), followed by (S)-4-N-Boc-2-methyl piperazine (3.23 g, 16.1 mmol, Combi-Blocks, Inc., San Diego, Calif., USA). The resulting mixture was warmed to room temperature and stirred for 1 h, then was diluted with cold saturated aqueous sodium bicarbonate solution (200 mL) and EtOAc (300 mL). The mixture was stirred for an additional 5 min, the layers were separated, and the aqueous layer was extracted with more EtOAc (1×). The combined organic layers were dried over anhydrous sodium sulfate and concentrated. The residue was purified by silica gel chromatography (eluent: 0-50% EtOAc/heptane) to provide (S)-tert-butyl 4-(7-chloro-6-fluoro-1-(2-isopropyl-4-methylpyridin-3-yl)-2-oxo-1,2-dihydropyrido[2,3-d]pyrimidin-4-yl)-3-methylpiperazine-1-carboxylate. m/z (ESI, +ve ion): 531.2 (M+H)⁺.

### Step 6: (3S)-tert-Butyl 4-(6-fluoro-7-(2-fluoro-6-hydroxyphenyl)-1-(2-isopropyl-4-methylpyridin-3-yl)-2-oxo-1,2-dihydropyrido [2,3-d]pyrimidin-4-yl)-3-methylpiperazine-1-carboxylate

A mixture of (S)-tert-butyl 4-(7-chloro-6-fluoro-1-(2-isopropyl-4-methylpyridin-3-yl)-2-oxo-1,2-dihydropyrido[2,3-d]pyrimidin-4-yl)-3-methylpiperazine-1-carboxylate (4.3 g, 8.1 mmol), potassium trifluoro(2-fluoro-6-hydroxyphenyl)borate (Intermediate Q, 2.9 g, 10.5 mmol), potassium acetate (3.2 g, 32.4 mmol) and [1,1′-bis(diphenylphosphino)ferrocene]dichloropalladium(II), complex with dichloromethane (661 mg, 0.81 mmol) in 1,4-dioxane (80 mL) was degassed with nitrogen for 1 min. De-oxygenated water (14 mL) was added, and the resulting mixture was heated at 90° C. for 1 h. The reaction was allowed to cool to room temperature, quenched with half-saturated aqueous sodium bicarbonate, and extracted with EtOAc (2×) and DCM (1×). The combined organic layers were dried over anhydrous sodium sulfate and concentrated. The residue was purified by silica gel chromatography (eluent: 0-60% 3:1 EtOAc-EtOH/heptane) to provide (3S)-tert-butyl 4-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-3-methylpiperazine-1-carboxylate. ¹H NMR (400 MHz, DMSO-d₆) δ ppm 10.19 (br s, 1H), 8.38 (d, J=5.0 Hz, 1H), 8.26 (dd, J=12.5, 9.2 Hz, 1H), 7.23-7.28 (m, 1H), 7.18 (d, J=5.0 Hz, 1H), 6.72 (d, J=8.0 Hz, 1H), 6.68 (t, J=8.9 Hz, 1H), 4.77-4.98 (m, 1H), 4.24 (brt, J=14.2 Hz, 1H), 3.93-4.08 (m, 1H), 3.84 (br d, J=12.9 Hz, 1H), 3.52-3.75 (m, 1H), 3.07-3.28 (m, 1H), 2.62-2.74 (m, 1H), 1.86-1.93 (m, 3H), 1.43-1.48 (m, 9H), 1.35 (dd, J=10.8, 6.8 Hz, 3H), 1.26-1.32 (m, 1H), 1.07 (dd, J=6.6, 1.7 Hz, 3H), 0.93 (dd, J=6.6, 2.1 Hz, 3H). ¹⁹F NMR (376 MHz, DMSO-d₆) δ: −115.65 (s, 1F), −128.62 (s, 1F). m/z (ESI, +ve ion): 607.3 (M+H)⁺.

### Step 7: 6-Fluoro-7-(2-fluoro-6-hydroxyphenyl)-1-(4-methyl-2-(2-propanyl)-3-pyridinyl)-4-((2S)-2-methyl-4-(2-propenoyl)-1-piperazinyl)pyrido[2,3-d]pyrimidin-2(1H)-one

Trifluoroacetic acid (25 mL, 324 mmol) was added to a solution of (3S)-tert-butyl 4-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-3-methylpiperazine-1-carboxylate (6.3 g, 10.4 mmol) in DCM (30 mL). The resulting mixture was stirred at room temperature for 1 h and then was concentrated. The residue was dissolved in DCM (30 mL), cooled to 0° C., and sequentially treated with DIPEA (7.3 mL, 41.7 mmol) and a solution of acryloyl chloride (0.849 mL, 10.4 mmol) in DCM (3 mL; added dropwise via syringe). The reaction was stirred at 0° C. for 10 min, then was quenched with half-saturated aqueous sodium bicarbonate and extracted with DCM (2×). The combined organic layers were dried over anhydrous sodium sulfate and concentrated. The residue was purified by silica gel chromatography (eluent: 0-100% 3:1 EtOAc-EtOH/heptane) to provide 4-((S)-4-acryloyl-2-methylpiperazin-1-yl)-6-fluoro-7-(2-fluoro-6-hydroxyphenyl)-1-(2-isopropyl-4-methylpyridin-3-yl)pyrido[2,3-d]pyrimidin-2(1H)-one (Compound K1). ¹H NMR (400 MHz, DMSO-d₆) δ ppm 10.20 (s, 1H), 8.39 (d, J=4.8 Hz, 1H), 8.24-8.34 (m, 1H), 7.23-7.32 (m, 1H), 7.19 (d, J=5.0 Hz, 1H), 6.87 (td, J=16.3, 11.0 Hz, 1H), 6.74 (d, J=8.6 Hz, 1H), 6.69 (t, J=8.6 Hz, 1H), 6.21 (br d, J=16.2 Hz, 1H), 5.74-5.80 (m, 1H), 4.91 (br s, 1H), 4.23-4.45 (m, 2H), 3.97-4.21 (m, 1H), 3.44-3.79 (m, 2H), 3.11-3.31 (m, 1H), 2.67-2.77 (m, 1H), 1.91 (s, 3H), 1.35 (d, J=6.8 Hz, 3H), 1.08 (d, J=6.6 Hz, 3H), 0.94 (d, J=6.8 Hz, 3H). ¹⁹F NMR (376 MHz, DMSO-d₆) δ ppm −115.64 (s, 1F), −128.63 (s, 1F). m/z (ESI, +ve ion): 561.2 (M+H)⁺.

### Example 12

### Preparation of 1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one (K2/Example 17a&17b of US2021/0230142A9) and 1-((S)-4-((S)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one (K2-S)

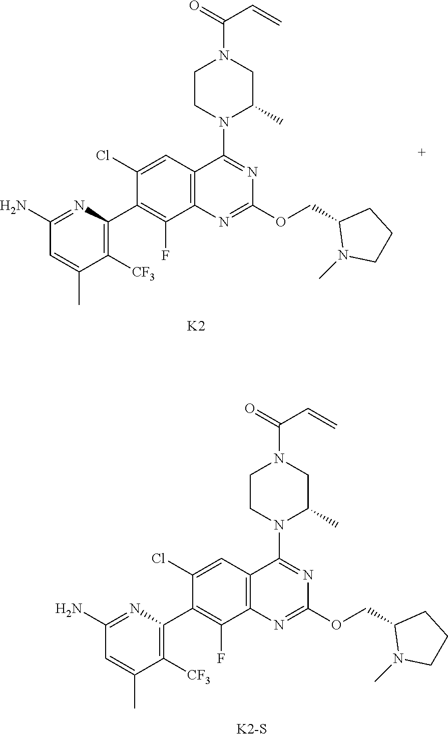

### Synthetic Route

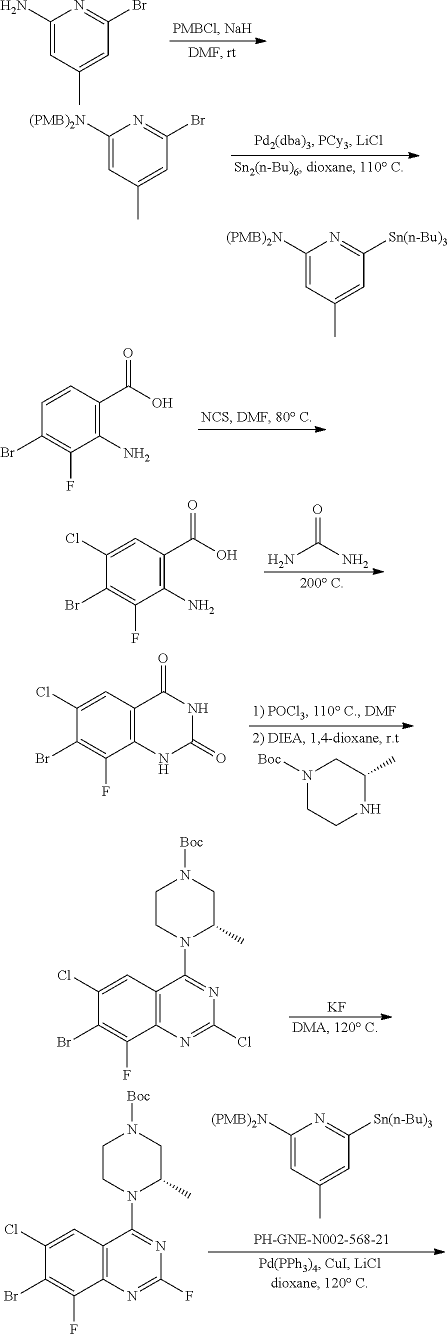

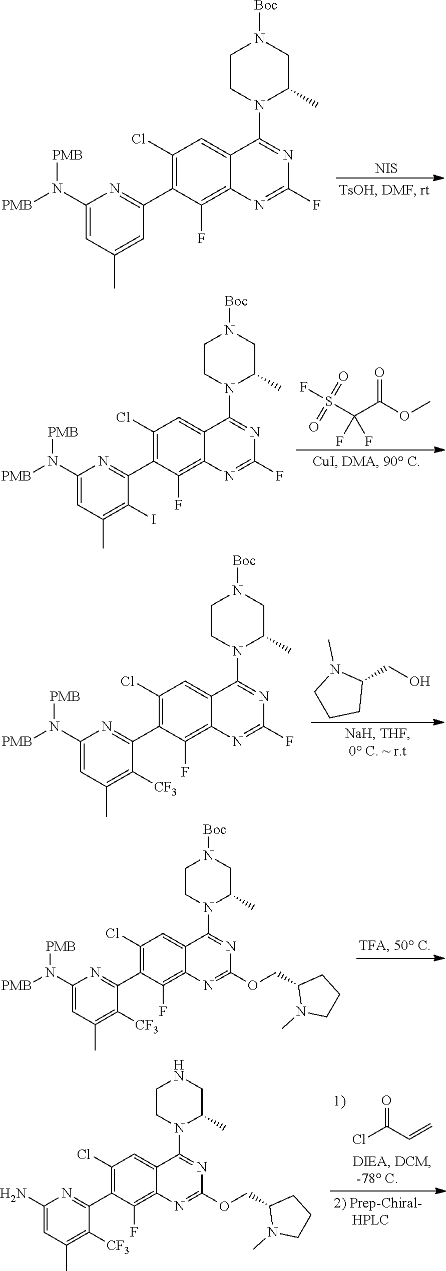

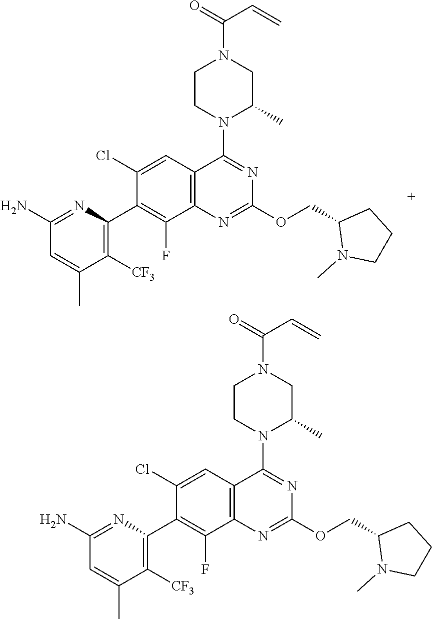

### Step 1: 6-bromo-N,N-bis(4-methoxybenzyl)-4-methylpyridin-2-amine

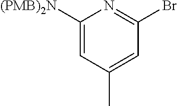

To a solution of 6-bromo-4-methylpyridin-2-amine (30.0 g, 160 mmol) in N,N-dimethylformamide (500 mL) was added slowly sodium hydride (19.0 g, 792 mmol) at 0° C. and stirred at 25° C. for 1 hour. Then 4-methoxybenzylchloride (56.0 g, 359 mmol) was added into the reaction system and stirred at 25° C. for 2 hours. After completion, the reaction system was quenched with saturated ammonium chloride solution (500 mL) and diluted with ethyl acetate (2.5 L). The mixture was washed with brine (5×500 mL) and the organic layers were combined, dried with Na₂SO₄, evaporated under vacuum. The residue was applied onto a silica gel column eluting with petroleum ether/ethyl acetate (15%) to afford 6-bromo-N,N-bis(4-methoxybenzyl)-4-methylpyridin-2-amine (60 g, 140 mmol, 87.5% yield) as an off-white solid. LC-MS: (ESI, m/z): 427.1 [M+H]⁺.

### Step 2: N,N-bis[(4-methoxyphenyl)methyl]-4-methyl-6-tributylstannyl-pyridin-2-amine

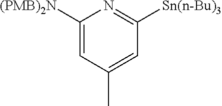

Under nitrogen, a solution of 6-bromo-N,N-bis[(4-methoxyphenyl)methyl]-4-methyl-pyridin-2-amine (35.0 g, 82 mmol), hexabutylditin (143.0 g, 247 mmol), tris(dibenzylideneacetone)dipalladium (7.53 g, 8.2 mmol), tricyclohexyl phosphine (4.6 g, 16.4 mmol) and Lithium chloride (17.3 g, 412 mmol) in 1,4-dioxane (220 mL) was stirred at 110° C. for 5 hours. After completion, the reaction system was concentrated under vacuum. The residue was purified by flash chromatography on silica gel eluting with petroleum ether/ethyl acetate (10/1) to afford N,N-bis[(4-methoxyphenyl)methyl]-4-methyl-6-tributylstannyl-pyridin-2-amine (45 g, 71 mmol, 86.2% yield) as a red oil. LC-MS: (ESI, m/z): 639.3 [M+H]⁺.

### Step 3: 2-amino-4-bromo-5-chloro-3-fluoro-benzoic acid

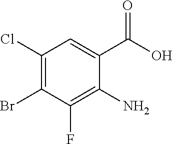 [SMILES: Nc1c(C(=O)O)cc(Cl)c(Br)c1F]

A solution of 2-amino-4-bromo-3-fluoro-benzoic acid (100.0 g, 427 mmol) and N-chlorosuccinimide (66.0 g, 494 mmol) in N,N-dimethylformamide (1 L) was stirred at 80° C. for 2 hours. After completion, the system was poured into water (2.0 L), a large amount of solids were precipitated. Then the solids were collected after filtration. The solids were washed with hot water (1 L). Then the solids were dried under infrared lamp to afford 2-amino-4-bromo-5-chloro-3-fluoro-benzoic acid (100 g, 373 mmol, 87.2% yield) as off-white solid. LC-MS: (ESI, m/z): 265.9 [M−H]⁺.

### Step 4: 7-bromo-6-chloro-8-fluoroquinazoline-2,4(1H,3H)-dione

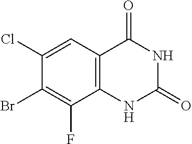 [SMILES: O=c1[nH]c(=O)c2cc(Cl)c(Br)c(F)c2[nH]1]

A solution of 2-amino-4-bromo-5-chloro-3-fluoro-benzoic acid (120.0 g, 447 mmol) in urea (806.0 g, 13.4 mol) was stirred at 200° C. for 1.5 hours. After completion, the reaction system was cooled to 80° C., and water (1.5 L) was added into the system with stirring for 20 mins. After filtration, the solids were collected and washed with hot water (1 L). Then the solids were dried under infrared lamp to afford 7-bromo-6-chloro-8-fluoroquinazoline-2,4(1H,3H)-dione (120 g, 409 mmol, 91.5% yield) as a light brown solid. LC-MS: (ESI, m/z): 290.9 [M−H]⁺.

### Step 5: tert-butyl (3S)-4-(7-bromo-2,6-dichloro-8-fluoroquinazolin-4-yl)-3-methylpiperazine-1-carboxylate

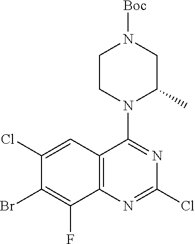 [SMILES: C[C@H]1CN(C(=O)OC(C)(C)C)CCN1c1nc(Cl)nc2c(F)c(Br)c(Cl)cc12]

A solution of 7-bromo-6-chloro-8-fluoro-quinazoline-2,4-diol (65.0 g, 222 mmol) and DMF (500.0 mg, 6.85 mmol) in POCl₃ (1.0 L) was stirred at 110° C. for 60 hours. After the starting material was completely, the resulting mixture was concentrated under vacuum. Then 1,4-dioxane (1.0 L), N,N-diisopropylethylamine (286.0 g, 2217 mmol) and tert-butyl (3S)-3-methyl-1-piperazinecarboxylate (90.0 g, 449 mmol) was added into the reaction system and stirred at 25° C. for 1 hours. After completion, the solvent was concentrated under vacuum. The residue was purified by flash chromatography on silica gel eluting with petroleum ether/ethyl acetate (20%) to afford tert-butyl (3S)-4-(7-bromo-2,6-dichloro-8-fluoroquinazolin-4-yl)-3-methylpiperazine-1-carboxylate (65 g, 132 mmol, 59.4% yield) as a yellow solid. LC-MS: (ESI, m/z): 493.0 [M+H]⁺.

### Step 6: tert-butyl (3S)-4-(7-bromo-6-chloro-2,8-difluoroquinazolin-4-yl)-3-methylpiperazine-1-carboxylate

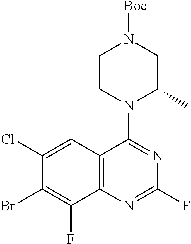 [SMILES: C[C@H]1CN(C(=O)OC(C)(C)C)CCN1c1nc(F)nc2c(F)c(Br)c(Cl)cc12]

A mixture of tert-butyl (3S)-4-(7-bromo-2,6-dichloro-8-fluoroquinazolin-4-yl)-3-methylpiperazine-1-carboxylate (30.0 g, 61 mmol) and potassium fluoride (71.0 g, 1224 mmol) in N,N-dimethylacetamide (300 mL) was stirred at 120° C. for 18 hours. After completion, the reaction system was cooled to room temperature. Then ethyl acetate (1.5 L) was added into the system and the mixture was washed with water (3×500 mL). The organic layer was dried over anhydrous sodium sulfate and concentrated under vacuum. The residue was purified by flash chromatography on silica gel eluting with petroleum ether/ethyl acetate (20%) to afford tert-butyl (3S)-4-(7-bromo-6-chloro-2,8-difluoroquinazolin-4-yl)-3-methylpiperazine-1-carboxylate (23 g, 48 mmol, 79.3% yield) as a yellow solid. LC-MS: (ESI, m/z): 477.0 [M+H]⁺.

### Step 7: tert-butyl (3S)-4-(7-(6-(bis(4-methoxybenzyl)amino)-4-methylpyridin-2-yl)-6-chloro-2,8-difluoroquinazolin-4-yl)-3-methylpiperazine-1-carboxylate

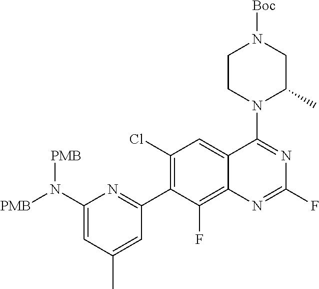

Under nitrogen, a solution of tert-butyl (3S)-4-(7-bromo-6-chloro-2,8-difluoroquinazolin-4-yl)-3-methylpiperazine-1-carboxylate (23.0 g, 48 mmol), N,N-bis[(4-methoxyphenyl)methyl]-4-methyl-6-tributylstannyl-pyridin-2-amine (62.0 g, 97 mmol), tetrakis(triphenylphosphine)palladium (11.2 g, 9.7 mmol), cuprous iodide (2.8 g, 15 mmol) and Lithium chloride (5.0 g, 119 mmol) in 1,4-dioxane (320 mL) was stirred at 120° C. for 16 hours. After completion, the reaction system was diluted with water (100 mL) and extracted with ethyl acetate (100 mL). Then the organic layers were combined, dried over anhydrous sodium sulfate and concentrated under vacuum. The residue was purified by flash chromatography on silica gel eluting with petroleum ether/ethyl acetate (30%) to afford tert-butyl (3S)-4-(7-(6-(bis(4-methoxybenzyl)amino)-4-methylpyridin-2-yl)-6-chloro-2,8-difluoroquinazolin-4-yl)-3-methylpiperazine-1-carboxylate (18.5 g, 25 mmol, 51.6% yield) as a yellow solid. LC-MS: (ESI, m/z): 745.3 [M+H]⁺.

### Step 8: tert-butyl (3S)-4-(7-(6-(bis(4-methoxybenzyl)amino)-3-iodo-4-methylpyridin-2-yl)-6-chloro-2,8-difluoroquinazolin-4-yl)-3-methylpiperazine-1-carboxylate

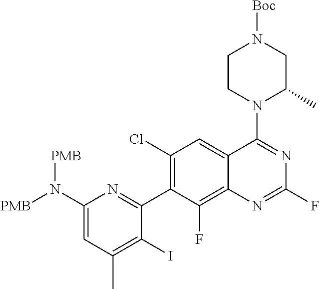

A solution of tert-butyl (3S)-4-(7-(6-(bis(4-methoxybenzyl)amino)-4-methylpyridin-2-yl)-6-chloro-2,8-difluoroquinazolin-4-yl)-3-methylpiperazine-1-carboxylate (18.5 g, 25 mmol), p-toluenesulfonic acid (171.0 mg, 0.99 mmol) and N-iodosuccinimide (28.0 g, 125 mmol) in N,N-dimethylformamide (350 mL) was stirred at 25° C. for 5 hours. After completion, the reaction system was diluted with ethyl acetate (1.5 L) and washed with saturated sodium thiosulfate solution (4×350 mL). The organic layer was dried over anhydrous sodium sulfate and concentrated under vacuum. The residue was purified by flash chromatography on silica gel eluting with petroleum ether/ethyl acetate (25%) to afford tert-butyl (3S)-4-(7-(6-(bis(4-methoxybenzyl)amino)-3-iodo-4-methylpyridin-2-yl)-6-chloro-2,8-difluoroquinazolin-4-yl)-3-methylpiperazine-1-carboxylate (16 g, 18.4 mmol, 74% yield) as a yellow solid. LC-MS: (ESI, m/z): 871.2 [M+H]⁺.

### Step 9: tert-butyl (3S)-4-(7-(6-(bis(4-methoxybenzyl)amino)-4-methyl-3-(trifluoromethyl)pyridin-2-yl)-6-chloro-2,8-difluoroquinazolin-4-yl)-3-methylpiperazine-1-carboxylate

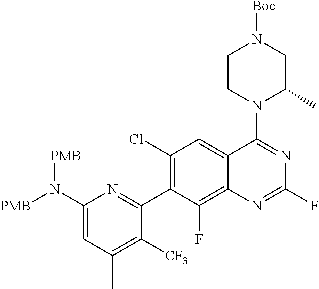

Under nitrogen, a solution of tert-butyl (3S)-4-(7-(6-(bis(4-methoxybenzyl)amino)-3-iodo-4-methylpyridin-2-yl)-6-chloro-2,8-difluoroquinazolin-4-yl)-3-methylpiperazine-1-carboxylate (16.0 g, 18.4 mmol), methyl 2,2-difluoro-2-(fluorosulfonyl)acetate (88.3 g, 460 mmol) and cuprous iodide (42.0 g, 221 mmol) in N,N-dimethylacetamide (400 mL) was stirred at 90° C. for 18 hours. After completion, the reaction system was diluted with ethyl acetate (2.0 L) and washed with brine (4×350 mL). The organic layer was dried over anhydrous sodium sulfate and concentrated under vacuum. The residue was purified by flash chromatography on silica gel eluting with petroleum ether/ethyl acetate (30%) to afford tert-butyl (3S)-4-(7-(6-(bis(4-methoxybenzyl)amino)-4-methyl-3-(trifluoromethyl)pyridin-2-yl)-6-chloro-2,8-difluoroquinazolin-4-yl)-3-methylpiperazine-1-carboxylate (12.2 g, 15 mmol, 81.7% yield) as a yellow solid. LC-MS: (ESI, m/z): 813.3 [M+H]⁺.

### Step 10: tert-butyl (3S)-4-(7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazine-1-carboxylate

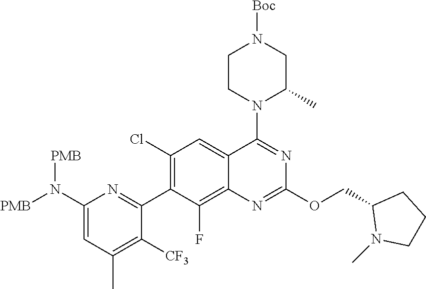

To a solution of (S)-(1-methylpyrrolidin-2-yl)methanol (4.32 g, 37.5 mmol) in tetrahydrofuran (300 mL) was added slowly sodium hydride (2.1 g, 87.5 mmol) at 0° C. and stirred for 1 h at 25° C. Then tert-butyl (3S)-4-(7-(6-(bis(4-methoxybenzyl)amino)-4-methyl-3-(trifluoromethyl)pyridin-2-yl)-6-chloro-2,8-difluoroquinazolin-4-yl)-3-methylpiperazine-1-carboxylate (12.2 g, 15 mmol) was added into the reaction system and stirred at 25° C. for 1 hours. After completion, the reaction system was quenched with methanol (50 mL). Then the mixture was concentrated under vacuum and the residue was purified by flash chromatography on silica gel eluting with dichloromethane/methanol (6/94) to afford tert-butyl (3S)-4-(7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazine-1-carboxylate (8.6 g, 9.5 mmol, 63.1% yield) as a brown solid. LC-MS: (ESI, m/z): 908.4 [M+H]⁺.

### Step 11: 6-(6-chloro-8-fluoro-4-((S)-2-methylpiperazin-1-yl)-2-(((S)-1-methylpyrrolidin-2-yl)methoxy)quinazolin-7-yl)-4-methyl-5-(trifluoromethyl)pyridin-2-amine

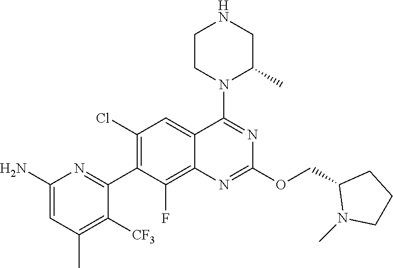 [SMILES: Cc1cc(N)nc(-c2c(Cl)cc3c(N4CCNC[C@@H]4C)nc(OC[C@@H]4CCCN4C)nc3c2F)c1C(F)(F)F]

A solution of tert-butyl (3S)-4-(7-(6-(bis(4-methoxybenzyl)amino)-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazine-1-carboxylate (8.6 g, 9.5 mmol) in trifluoroacetic acid (100 mL) was stirred at 50° C. for 4 hours. After completion, the reaction system was concentrated under vacuum. The residue was dissolved with dichloromethane (50 mL) and the pH was adjusted to pH=9 with N,N-diisopropylethylamine. After concentrated under vacuum, the residue was purified by a reversed-phase chromatography directly with the following conditions: Column, C18 silica gel; mobile phase, A: water, B: ACN, B % (5%˜40% in 30 min); Detector, UV 254 nm to afford 6-(6-chloro-8-fluoro-4-((S)-2-methylpiperazin-1-yl)-2-(((S)-1-methylpyrrolidin-2-yl)methoxy)quinazolin-7-yl)-4-methyl-5-(trifluoromethyl)pyridin-2-amine (3.5 g, 6.17 mmol, 65.1% yield) as a yellow solid. LC-MS: (ESI, m/z): 568.2 [M+H]⁺.

### Step 12: 1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one (K2) and 1-((S)-4-((S)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one (K2-S)

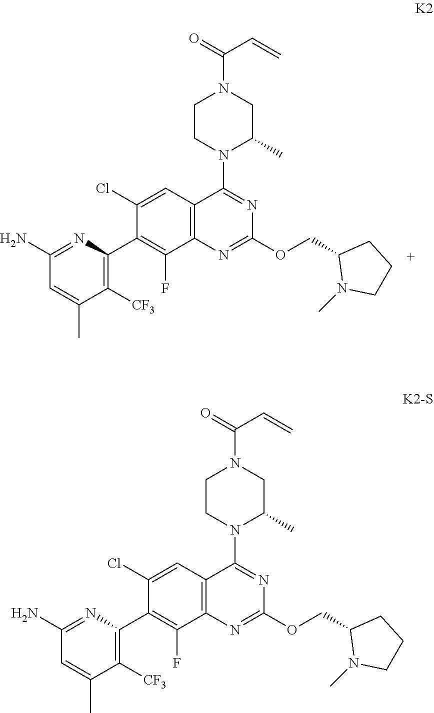

To a solution of 6-(6-chloro-8-fluoro-4-((S)-2-methylpiperazin-1-yl)-2-(((S)-1-methylpyrrolidin-2-yl)methoxy)quinazolin-7-yl)-4-methyl-5-(trifluoromethyl)pyridin-2-amine (2.5 g, 4.4 mmol) and N,N-diisopropylethylamine (2.9 g, 22.5 mmol) in dichloromethane (120 mL) was added acryloyl chloride (359.0 mg, 3.97 mmol) at −78° C. and stirred at −78° C. for 25 mins. The reaction was quenched by water and extracted with dichloromethane. The organic layers were combined. The organic layer was dried over anhydrous sodium sulfate and concentrated under vacuum. The residue was purified by a reversed-phase chromatography directly with the following conditions: Column, C18 silica gel; mobile phase, A: water, B: ACN, B % (5%˜60% in 30 min); Detector, UV 254 nm to afford 1-[(3S)-4-[7-[6-amino-4-methyl-3-(trifluoromethyl)-2-pyridyl]-6-chloro-8-fluoro-2-[[(2S)-1-methylpyrrolidin-2-yl]methoxy]quinazolin-4-yl]-3-methyl-piperazin-1-yl]prop-2-en-1-one (1.3 g, 2.09 mmol, 47.5% yield) as a brown solid. The mixture of diastereoisomer was separated by Prep-Chiral-HPLC with the following condition: Column, CHIRALPAK IC-3 0.46*5 Cm 3 um; mobile phase, (Hex:dichloromethane=3:1) (0.1% DEA):EtOH=50:50; Detector, 254 nm; Flow, 1.0 ml/min; Temperature: 25° C. to afford 657.7 mg of 1-((S)-4-((R)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one (K2) as a white solid and 352.1 mg of 1-((S)-4-((S)-7-(6-amino-4-methyl-3-(trifluoromethyl)pyridin-2-yl)-6-chloro-8-fluoro-2-(((S)-1-methylpyrrolidin-2-yl)methoxy)quinazolin-4-yl)-3-methylpiperazin-1-yl)prop-2-en-1-one (K2-S) as a white solid.

K2: LC-MS: (ESI, m/z): 622.2 [M+H]⁺, ¹H NMR: (400 MHz, CDCl₃, ppm) δ 7.64 (s, 1H), 6.70-6.55 (m, 1H), 6.48 (s, 1H), 6.42-6.35 (m, 1H), 5.82-5.75 (m, 1H), 4.90-4.79 (m, 2H), 4.78-4.40 (m, 3H), 4.35-4.28 (m, 1H), 4.18-4.00 (m, 1H), 3.99-3.76 (m, 1H), 3.72-3.45 (m, 2H), 3.31-2.98 (m, 2H), 2.81-2.70 (m, 1H), 2.55-2.45 (m, 6H), 2.35-2.25 (m, 1H), 2.11-2.01 (m, 1H), 1.95-1.72 (m, 3H), 1.36-1.34 (m, 3H).

K2-S: LC-MS: (ESI, m/z): 622.2 [M+H]⁺, ¹H NMR: (400 MHz, CDCl₃, ppm) δ 7.63 (s, 1H), 6.70-6.55 (m, 1H), 6.50 (s, 1H), 6.42-6.35 (m, 1H), 5.82-5.75 (m, 1H), 4.85-4.70 (m, 2H), 4.78-4.68 (m, 2H), 4.65-4.55 (m, 1H), 4.50-4.40 (m, 1H), 4.30-4.10 (m, 1H), 4.05-3.75 (m, 1H), 3.80-3.76 (m, 2H), 3.25-3.08 (m, 2H), 2.85-2.75 (m, 1H), 2.60-2.45 (m, 6H), 2.40-2.25 (m, 1H), 2.15-2.05 (m, 1H), 1.95-1.72 (m, 3H), 1.45-1.32 (m, 3H).

| Exam- | | |
|---|---|---|
| ple | | MS |
| No. | ¹H NMR | (M + H)⁺ |
| K2 | ¹H NMR: (400 MHz, CDCl₃, ppm) δ 7.64 (s, 1H), | 622.2 |
| | 6.70-6.55 (m, 1H), 6.48 (s, 1H), 6.42-6.35 (m, 1H), | |
| | 5.82-5.75 (m, 1H), 4.90-4.79 (m, 2H), 4.78-4.40 | |
| | (m, 3H), 4.35-4.28 (m, 1H), 4.18-4.00 (m, 1H), | |
| | 3.99-3.76 (m, 1H), 3.72-3.45 (m, 2H), 3.31-2.98 | |
| | (m, 2H), 2.81-2.70 (m, 1H), 2.55-2.45 (m, 6H), | |
| | 2.35-2.25 (m, 1H), 2.11-2.01 (m, 1H), 1.95-1.72 | |
| | (m, 3H), 1.36-1.34 (m, 3H). | |
| K2-S | ¹H NMR: (400 MHz, CDCl₃, ppm) δ 7.63 (s, 1H), | 622.2 |
| | 6.70-6.55 (m, 1H), 6.50 (s, 1H), 6.42-6.35 (m, 1H), | |
| | 5.82-5.75 (m, 1H), 4.85-4.70 (m, 2H), 4.78-4.68 | |
| | (m, 2H), 4.65-4.55 (m, 1H), 4.50-4.40 (m, 1H), | |
| | 4.30-4.10 (m, 1H), 4.05-3.75 (m, 1H), 3.80-3.76 | |
| | (m, 2H), 3.25-3.08 (m, 2H), 2.85-2.75 (m, 1H), | |
| | 2.60-2.45 (m, 6H), 2.40-2.25 (m, 1H), 2.15-2.05 | |
| | (m, 1H), 1.95-1.72 (m, 3H), 1.45-1.32 (m, 3H). | |

### Example 13

### Preparation of 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoroacryloyl)piperazin-2-yl)acetonitrile (Compound K3/Example 478 of US2019/0144444A1)

### 2-fluoroprop-2-enoyl chloride

To a solution of 2-fluoroprop-2-enoic acid (400 mg, 4.44 mmol, 1 eq) in DCM (4 mL) was added (COCl)₂ (846 mg, 6.66 mmol, 583 uL, 1.5 eq) and DMF (32.5 mg, 444 umol, 34.2 uL, 0.1 eq). The mixture was stirred at 25° C. for 2 hrs. The reaction mixture was concentrated under reduced pressure to remove a part of solvent and give a residue in DCM. Compound 2-fluoroprop-2-enoyl chloride (400 mg, crude) was obtained as a yellow liquid and used into the next step without further purification.

### Step A: 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile

To a solution of 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]piperazin-2-yl]acetonitrile (300 mg, 528 umol, 1 eq, HCl) in DCM (5 mL) was added DIEA (1.73 g, 13.4 mmol, 2.33 mL, 25.4 eq) and 2-fluoroprop-2-enoyl chloride (286 mg, 2.64 mmol, 5 eq) in DCM (5 mL). The mixture was stirred at 0° C. for 1 hour. The reaction mixture was concentrated under reduced pressure to give a residue. The residue was purified by column chromatography (Al₂O₃, Dichloromethane/Methanol=10/1 to 10/1). The residue was purified by prep-HPLC (column: Gemini 150*25 5 u; mobile phase: [water (0.05% ammonia hydroxide v/v)—ACN]; B %: 55%-85%, 12 min). The residue was purified by prep-HPLC (column: Phenomenex Synergi C18 150*30 mm *4 um; mobile phase: [water (0.225% FA)—ACN]; B %: 20%-50%, 10.5 min). The residue was concentrated under reduced pressure to remove ACN, and then lyophlization. Title compound 2-((S)-4-(7-(8-chloronaphthalen-1-yl)-2-(((S)-1-methylpyrrolidin-2-yl)methoxy)-5,6,7,8-tetrahydropyrido[3,4-d]pyrimidin-4-yl)-1-(2-fluoroacryloyl)piperazin-2-yl)acetonitrile (Compound K3/EXAMPLE 478 of US2019/0144444A1, 24.1 mg, 36.7 umol, 7% yield, 99.1% purity, FA) was obtained as a brown solid.

SFC condition: “AD-3S_3_5_40_3ML Column: Chiralpak AD-3 100×4.6 mm I.D., 3 um Mobile phase: methanol (0.05% DEA) in CO₂ from 5% to 40% Flow rate: 3 mL/min Wavelength: 220 nm”.

¹H NMR (400 MHz, Acetic) δ=7.82 (d, J=8.0 Hz, 1H), 7.69 (d, J=8.0 Hz, 1H), 7.56 (d, J=7.6 Hz, 1H), 7.49 (t, J=7.6 Hz, 1H), 7.41-7.30 (m, 2H), 5.58-5.25 (m, 2H), 5.17-4.59 (m, 4H), 4.57-4.28 (m, 3H), 4.24-3.78 (m, 4H), 3.67-3.13 (m, 7H), 3.08 (br d, J=2.4 Hz, 3H), 2.98 (br d, J=6.4 Hz, 1H), 2.83-2.61 (m, 1H), 2.45-2.29 (m, 1H), 2.24-2.08 (m, 3H).

### Example 14

### Preparation of a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one and a(S)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one (Compound K4/Example 1 of WO2021/124222A1)

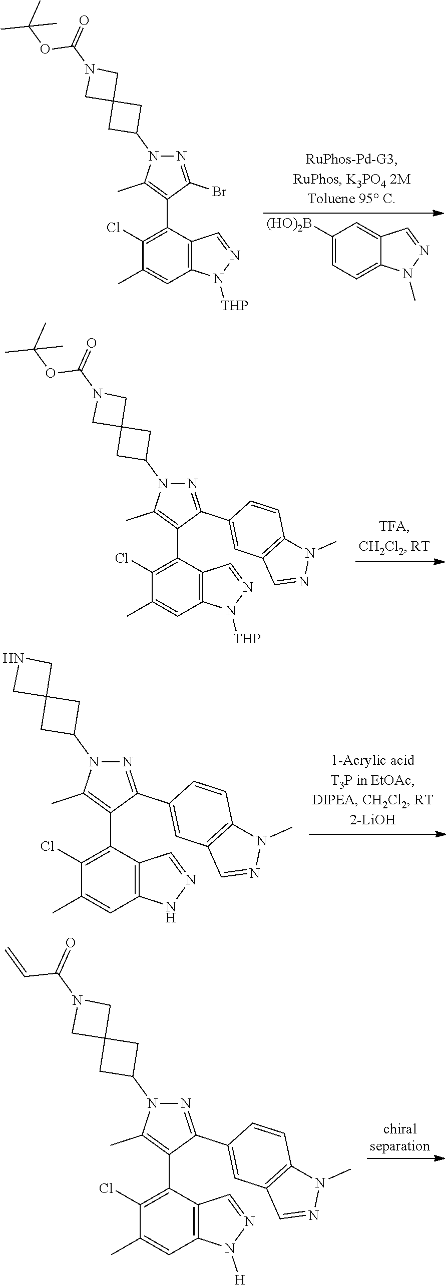

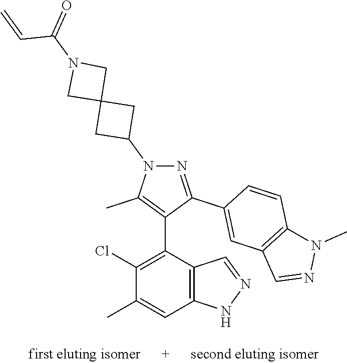 [SMILES: C=CC(=O)N1CC2(CC(n3nc(-c4ccc5c(cnn5C)c4)c(-c4c(Cl)c(C)cc5[nH]ncc45)c3C)C2)C1]

### Step 1: Tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate

In a 500 mL flask, tert-butyl 6-(3-bromo-4-(5-chloro-6-methyl-1-tetrahydro-2H-pyran-2-yl)-1H-indazol-4-yl)-5-methyl-1H-pyrazol-1-yl)-2-azaspiro[3.3]heptane-2-carboxylate (Intermediate C1, 10 g, 16.5 mmol), (1-methyl-1H-indazol-5-yl)boronic acid (6.12 g, 33.1 mmol), RuPhos (1.16 g, 2.48 mmol) and RuPhos-Pd-G3 (1.66 g, 1.98 mmol) were suspended in toluene (165 mL) under argon. K₃PO₄ (2M, 24.8 mL, 49.6 mmol) was added and the reaction mixture was placed in a preheated oil bath (95° C.) and stirred for 45 min. The reaction mixture was poured into a sat. aq. NH4Cl solution and was extracted with EtOAc (x3). The combined organic layers were washed with a sat. aq. NaHCO₃ solution, dried (phase separator) and concentrated under reduced pressure. The crude residue was diluted with THE (50 mL), SiliaMetS® Thiol (15.9 mmol) was added and the mixture swirled for 1 h at 40° C. The mixture was filtered, the filtrate was concentrated and the crude residue was purified by normal phase chromatography (eluent: MeOH in CH₂Cl₂ from 0 to 2%), the purified fractions were again purified by normal phase chromatography (eluent: MeOH in CH₂Cl₂ from 0 to 2%) to give the title compound as a beige foam. UPLC-MS-3: Rt=1.23 min; MS m/z [M+H]⁺; 656.3/658.3.

### Step 2: 5-Chloro-6-methyl-4-(5-methyl-3-(1-methyl-1H-indazol-5-yl)-1-(2-azaspiro[3.3]heptan-6-yl)-1H-pyrazol-4-yl)-1-H-indazole

TFA (19.4 mL, 251 mmol) was added to a solution of tert-butyl 6-(4-(5-chloro-6-methyl-1-(tetrahydro-2H-pyran-2-yl)-1H-indazol-4-yl)-5-methyl-3-(1-methyl-1H-indazol-5-yl)-1H-pyrazol-1-yl)-2-azaspiro[3.3]heptane-2-carboxylate (Step 1, 7.17 g, 10.0 mmol) in CH₂Cl₂ (33 mL). The reaction mixture was stirred at RT under nitrogen for 1.5 h. The RM was concentrated under reduced pressure to give the title compound as a trifluoroacetate salt, which was used without purification in the next step. UPLC-MS-3: Rt=0.74 min; MS m/z [M+H]⁺; 472.3/474.3.

### Step 3: 1-(6-(4-(5-Chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one

A mixture of acrylic acid (0.69 mL, 10.1 mmol), propylphosphonic anhydride (50% in EtOAc, 5.94 mL, 7.53 mmol) and DIPEA (21.6 mL, 126 mmol) in CH₂Cl₂ (80 mL) was stirred for 20 min at RT and then added (dropping funnel) to an ice-cooled solution of 5-chloro-6-methyl-4-(5-methyl-3-(1-methyl-1H-indazol-5-yl)-1-(2-azaspiro[3.3]heptan-6-yl)-1H-pyrazol-4-yl)-1H-indazole trifluoroacetate (Step 2, 6.30 mmol) in CH₂Cl₂ (40 mL). The reaction mixture was stirred at RT under nitrogen for 15 min. The RM was poured into a sat. aq. NaHCO₃ solution and extracted with CH₂Cl₂ (×3). The combined organic layers were dried (phase separator) and concentrated. The crude residue was diluted with THF (60 mL) and LiOH (2N, 15.7 mL, 31.5 mmol) was added. The mixture was stirred at RT for 30 min until disappearance (UPLC) of the side product resulting from the reaction of the acryloyl chloride with the free NH group of the indazole then was poured into a sat. aq. NaHCO₃ solution and extracted with CH₂Cl₂ (3×). The combined organic layers were dried (phase separator) and concentrated. The crude residue was purified by normal phase chromatography (eluent: MeOH in CH₂Cl₂ from 0 to 5%) to give the title compound. The isomers were separated by chiral SFC (C-SFC-1; mobile phase: CO₂/[IPA+0.1% Et₃N]: 69/31) to give Example 1 a: a(R)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one as the second eluting peak (white powder): ¹H NMR (600 MHz, DMSO-d₆.) δ 13.1 (s, 1H), 7.89 (s, 1H), 7.59 (s, 1H), 7.55 (s, 1H), 7.42 (m, 2H), 7.30 (d, 1H), 6.33 (m, 1H), 6.12 (m, 1H), 5.68 (m, 1H), 4.91 (m, 1H), 4.40 (s, 1H), 4.33 (s, 1H), 4.11 (s, 1H), 4.04 (s, 1H), 3.95 (s, 3H), 2.96-2.86 (m, 2H), 2.83-2.78 (m, 2H), 2.49 (s, 3H), 2.04 (s, 3H); UPLC-MS-4: Rt=4.22 min; MS m/z [M+H]⁺ 526.3/528.3: C-SFC-3 (mobile phase: CO₂/[IPA+0.1% Et₃N]: 67/33): Rt=2.23 min. The compound of Example 1a of WO2021/124222A1 is also referred to as “Compound X” of WO2021/124222A1.

The other isomer Example 1b of WO2021/124222A1; a(S)-1-(6-(4-(5-chloro-6-methyl-1H-indazol-4-yl)-5-methyl-3-(1-methyl-1H-indazol-5-yl)-1H-pyrazol-1-yl)-2-azaspiro[3.3]heptan-2-yl)prop-2-en-1-one was obtained as the first eluting peak: C-SFC-3 (mobile phase: CO₂/[IPA+0.1% Et₃N]: 67/33): Rt=1.55 min.

### Biological Examples

### Example B-1: Drug Combination Assay

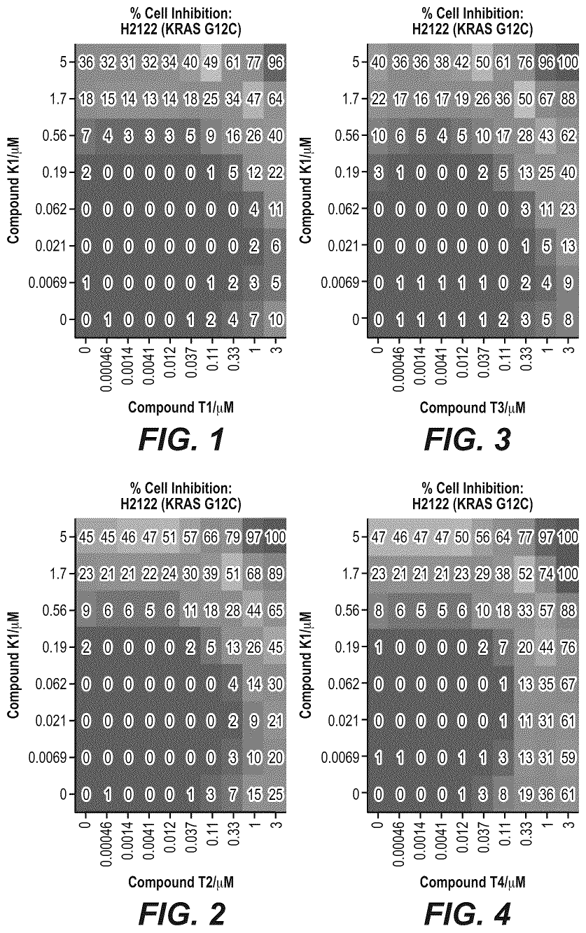

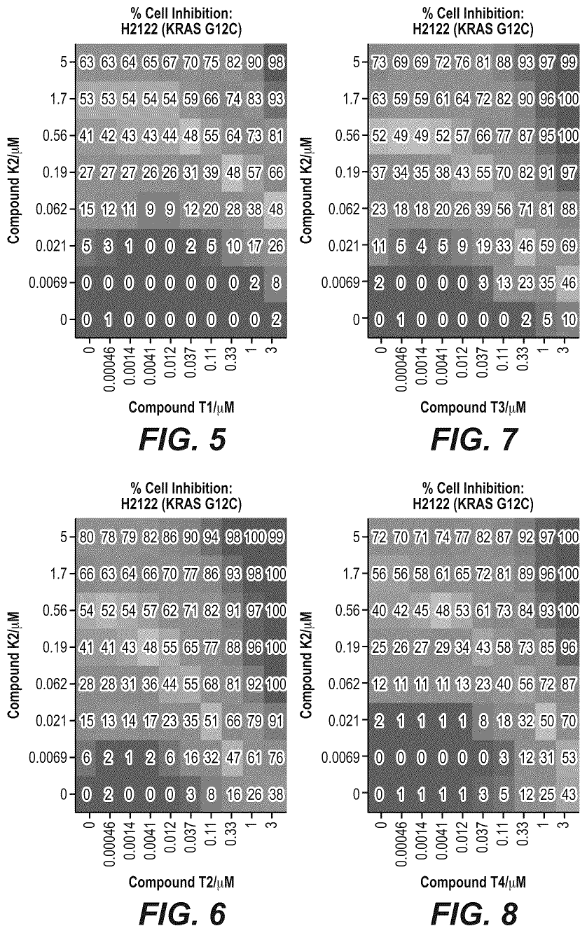

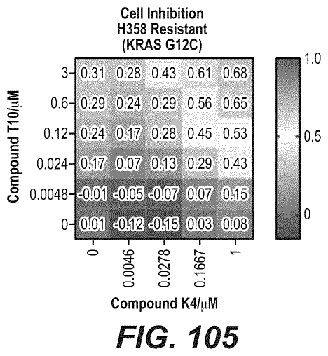

Cells were seeded in either 96-well or 384-well plates 16 h before treatment at a density of 1000 cells per well. Then, cells were treated with varying concentrations of compound(s) as indicated in FIGS. 1-8 (384-wells) and FIGS. 10-105 (96-wells), either a single agent or in combination, for six days. The relative number of viable cells was estimated using CellTiter-Glo Luminescent Cell Viability Assay (Promega, G7573). Total luminescence was detected on a Wallac Multilabel Reader (Perkin-Elmer).

### Example B-2: Soft Agar 3D Culture

### Block/Plate/Medium/Agarose Prep

Two heat blocks (1× without blocks, 1× with 50 ml tube block) were heated to 44° C. Flat-bottom 96-well non-tissue culture treated assay plates (Falcon 353219) and fluid reservoirs were kept at 37° C. 2×RPMI+20% FCS (which can be stored at 4° C.) were made and warmed to 37° C. 100 ml 1.2% agarose and 0.58% agarose solution was made by adding 105 ml sterile water to a sterile 500 ml bottle and then carefully adding 1.2 gram/0.58 gram agarose (using normal out-of-the-hood weight), avoiding bubbles, foam, and sticking of undiluted agarose to walls as much as possible. The solution was then microwaved until fully dissolved and bubbling. The solution was then cooled for 10-15 minutes at room temperature and incubated at 37° C. The solution can be stored at room temperature for future use.

### Base: 1.2% Agarose

For each plate, 2 ml 1.2% agarose was mixed with 2 ml 2×RPMI+20% FCS in a 50 ml tube in a heat block by swirling the tube (minimizing pipetting up and down, as this cools the agarose too much). By double-tap (pipet is pushed in completely, liquid is taken up, then pushed out until first stop only), 40 μl 1.2% agarose/96-well was added, excluding edge 96-wells. The agarose was set at “waterpas” level and stored at 4° C. until further use. 1.2% agarose-containing plate was placed on ice for 10 minutes before plating cells.

### Cell Plating in 0.58% Agarose

Cells were trypsinized and counted. A cell suspension (per plate: 150.000 cells in 2 ml 2×RPMI+20% FCS) was made and warmed to 37° C. 4 ml 0.58% agarose+ cells per plate was made by adding 2 ml 0.58% agarose to 2 ml cell suspension in 50 ml tube in heat block by swirling the tube (minimizing pipetting up and down, as this cools the agarose too much). 40 μl 0.58% agarose+ cells/96-well were added by double-tap on top of ice-cold 1.2% agarose (150,000 cells in (2+2=) 4 ml=1500 cells/96-well), placed on ice for 10 minutes at “waterpas” level, and then placed at 37° C. until further use.

### Treatment

3× stocks of compound in 1×RPMI+8% FCS were prepared at 40 μl/96-well. Using double-tap, 1.2% agarose/0.58% agarose+ cells were overlayed with 40 μl 3× stock compound in 1×RPMI+8% FCS (total volume/96-well=120 μl). Plate edge 96-wells were filled with with 200 μl PBS and cultured at 37° C. After a 7 day incubation period at 37° C., 40 μl compound in 1×RPMI+8% FCS was added (by making stocks for 40 μl/96-well, so that 1× stock was for a volume of 160 μl).

### Readout

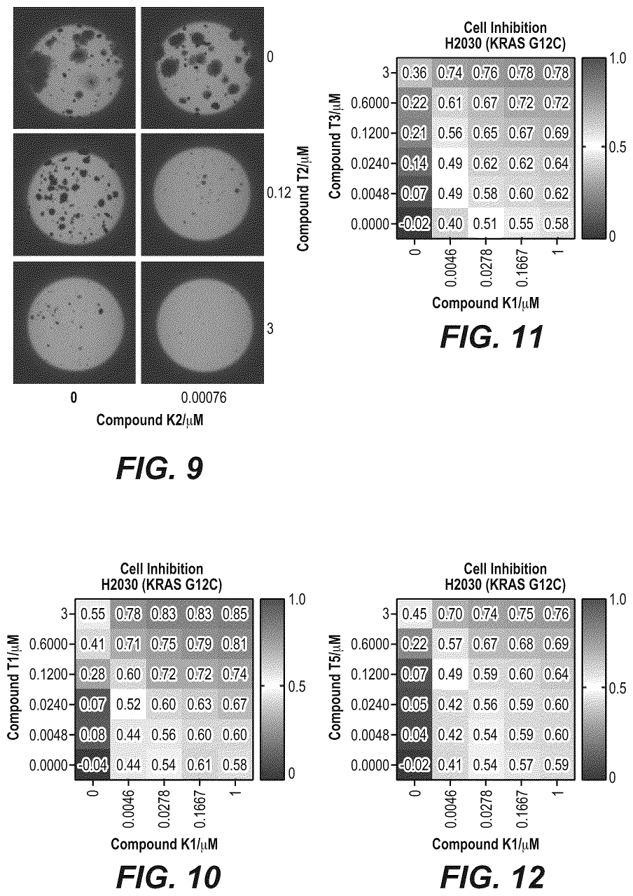

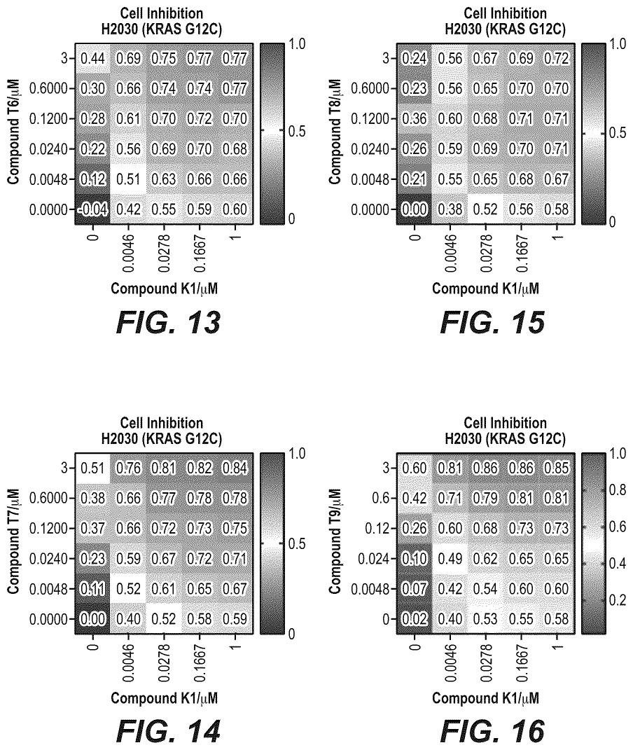

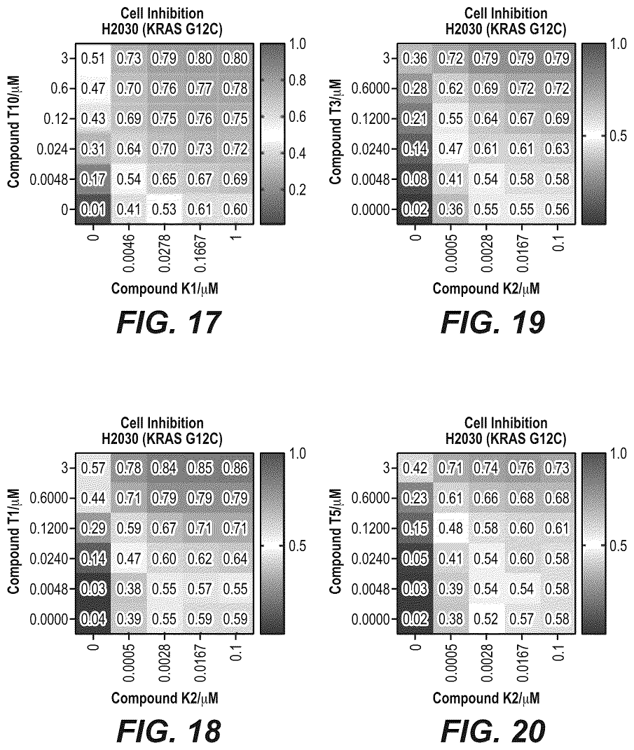

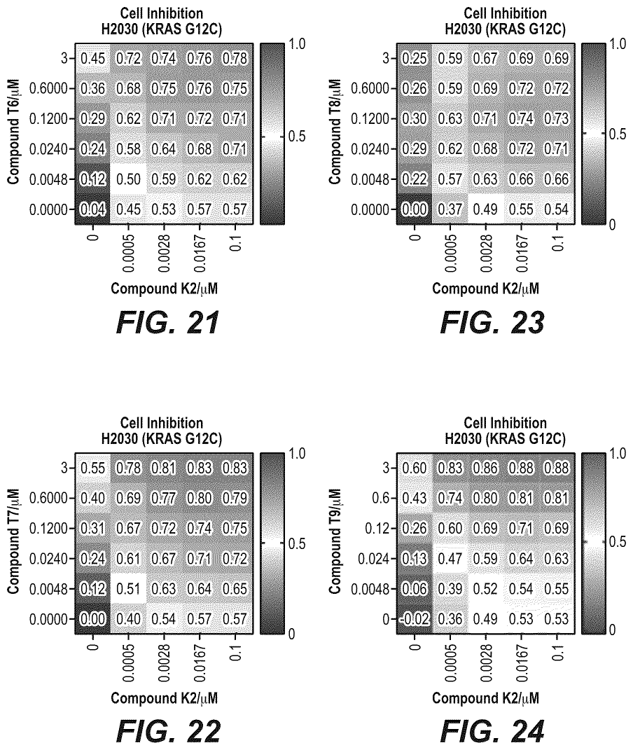

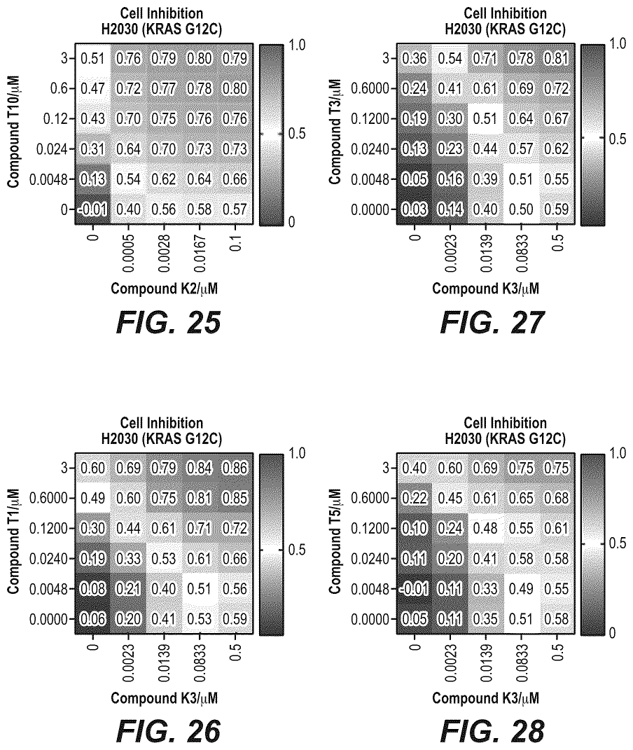

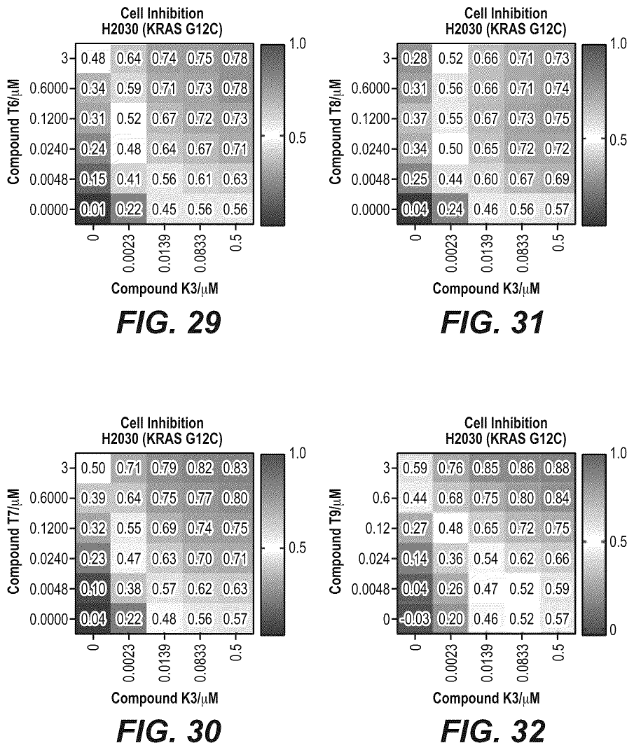

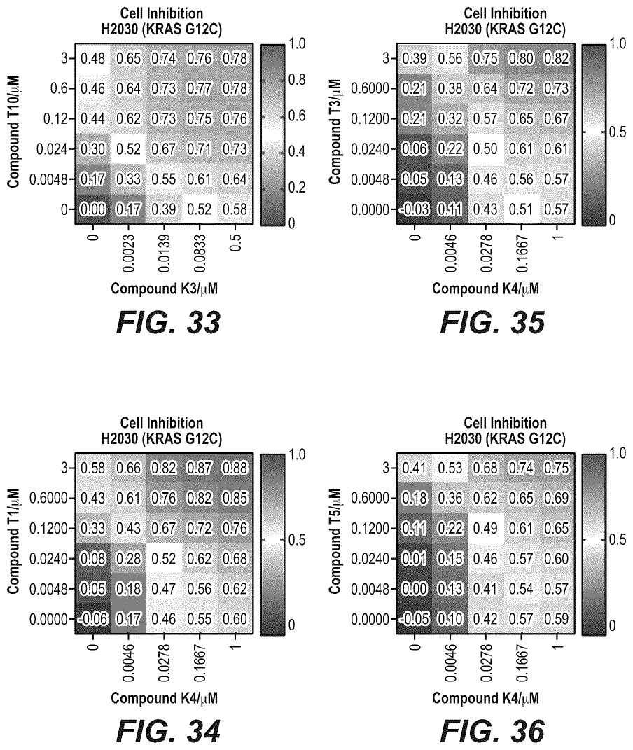

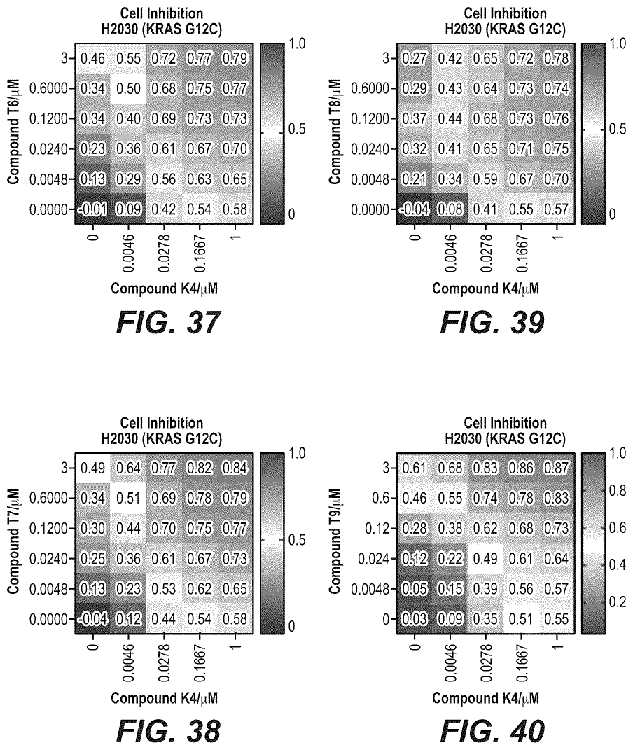

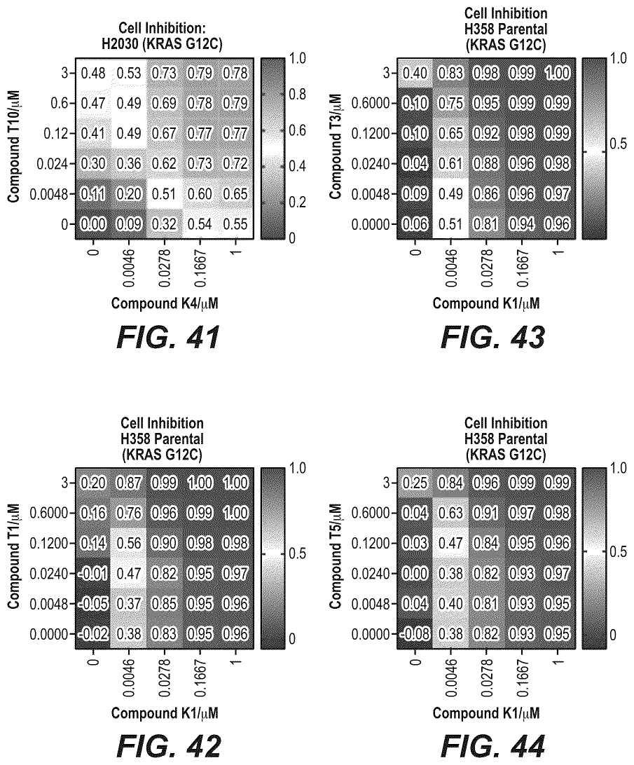

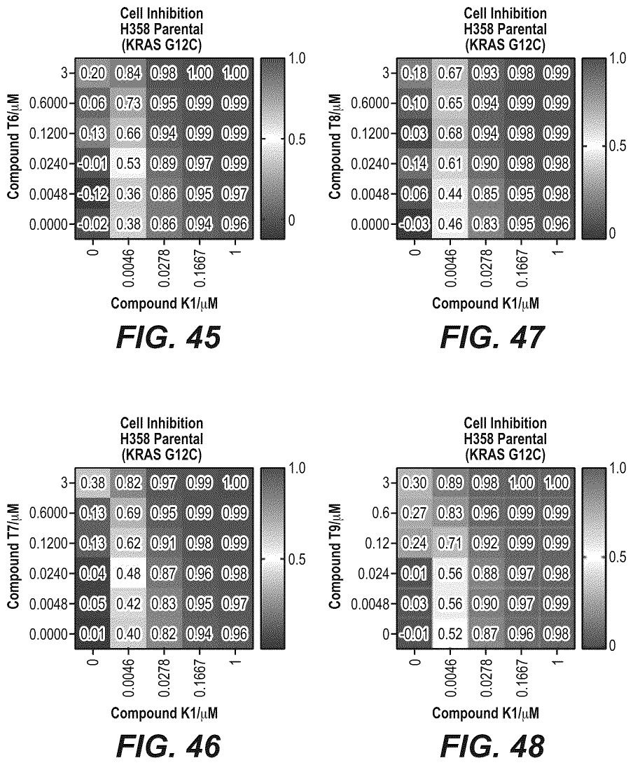

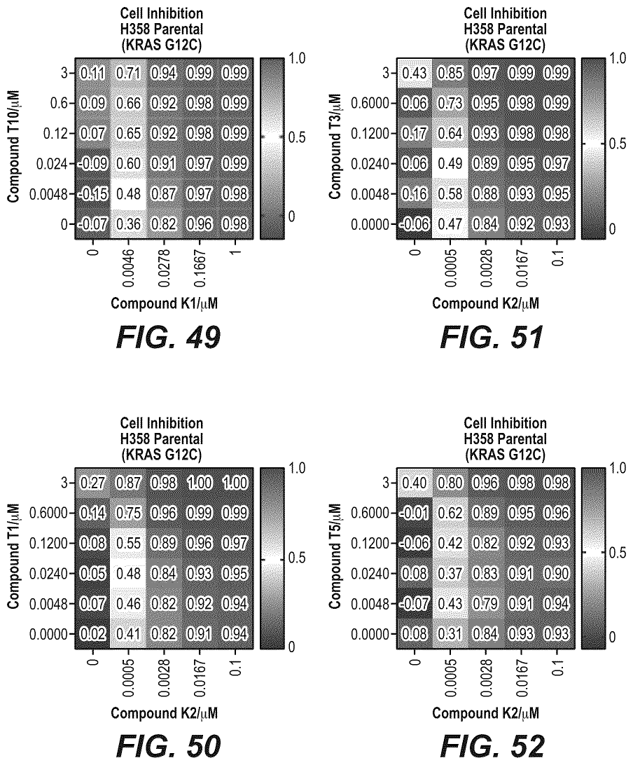

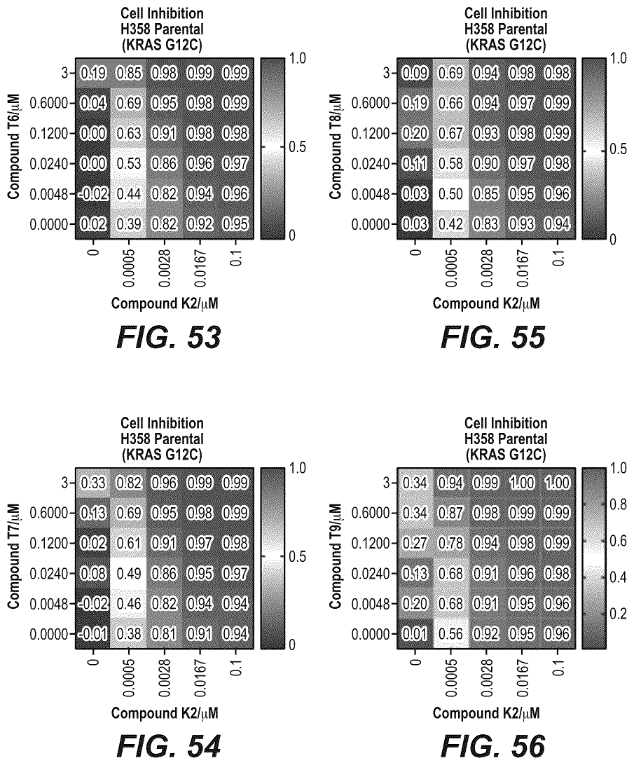

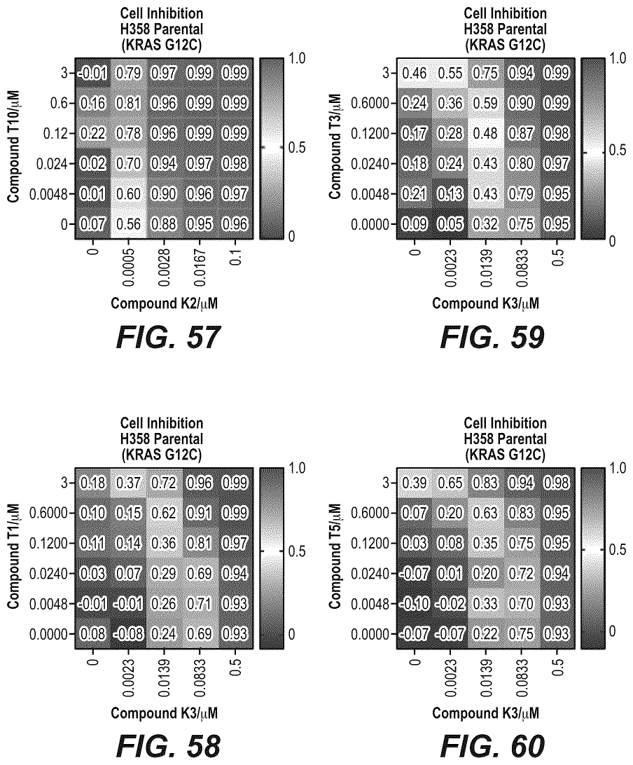

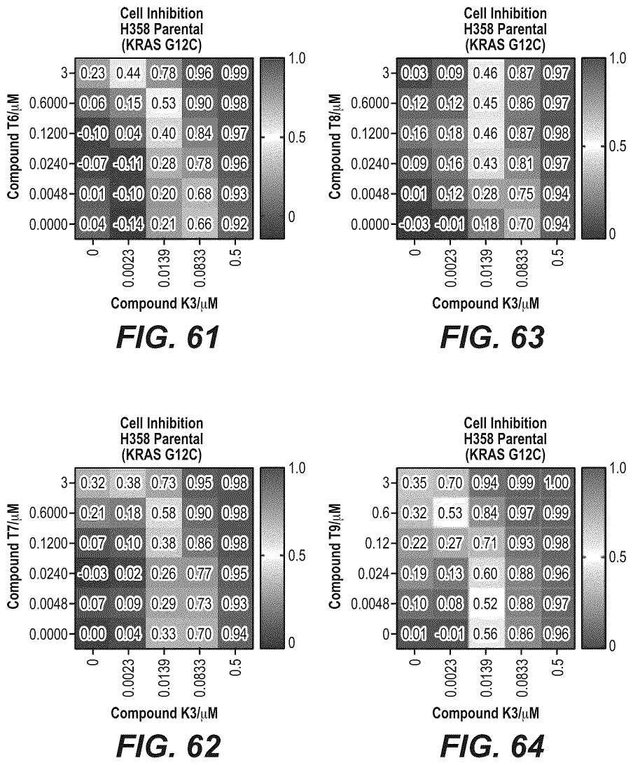

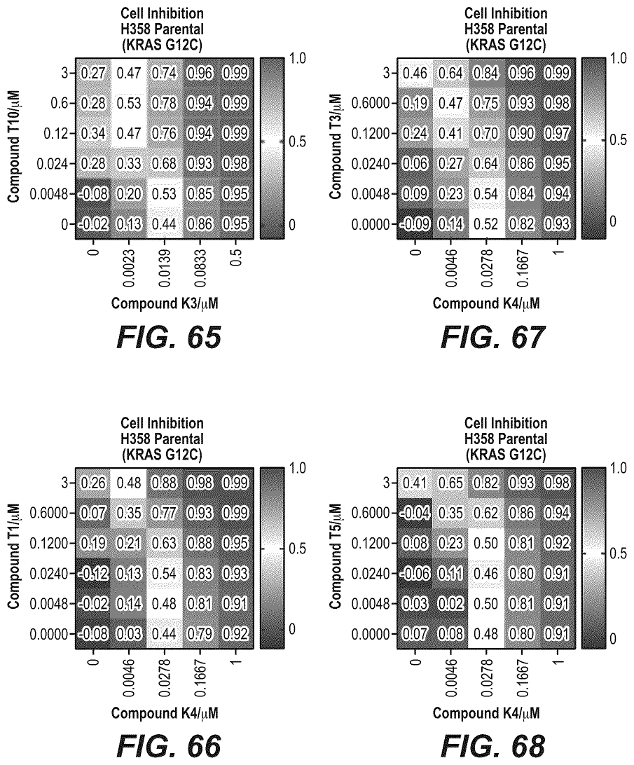

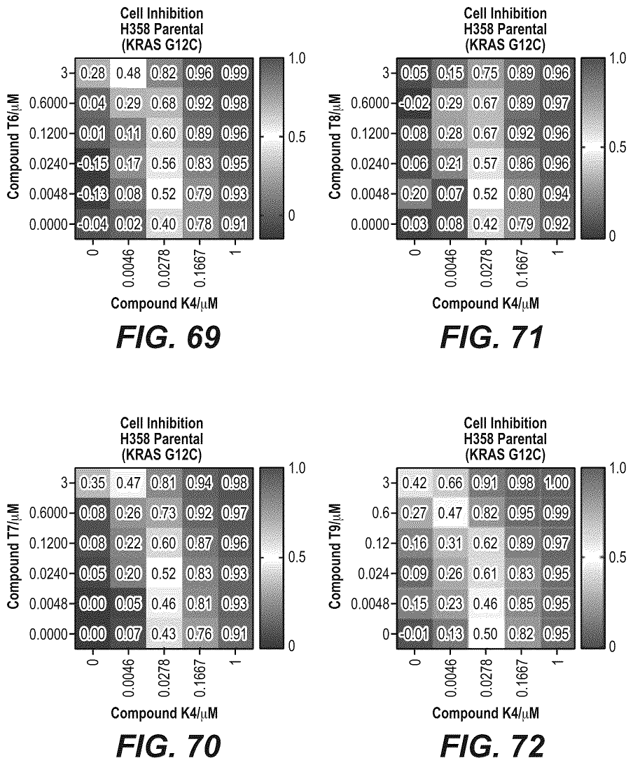

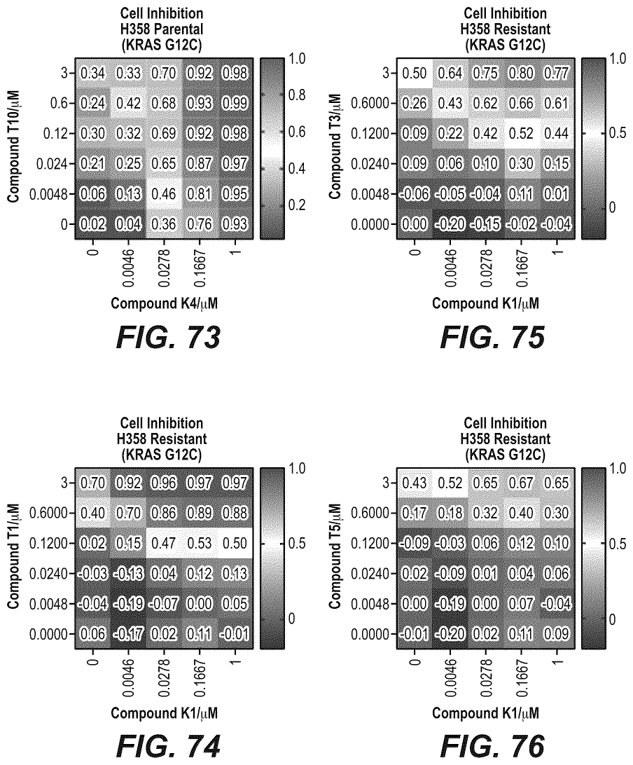

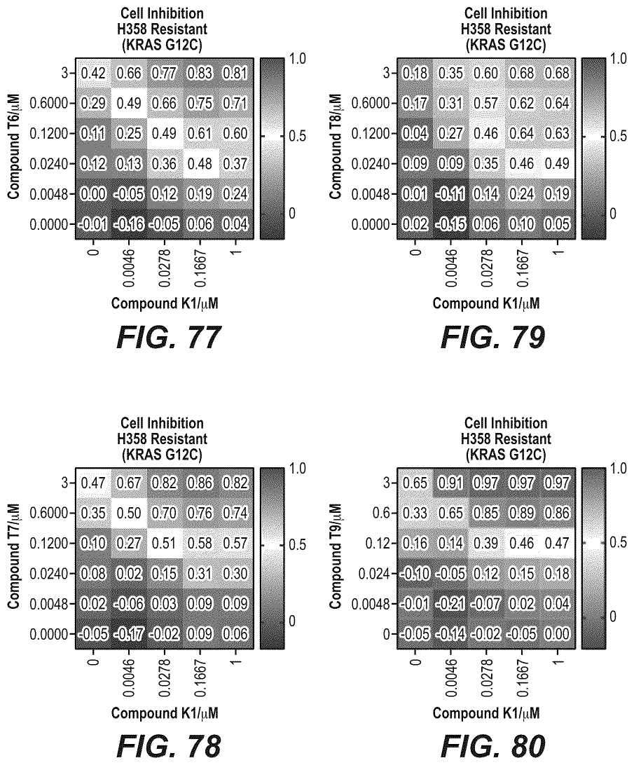

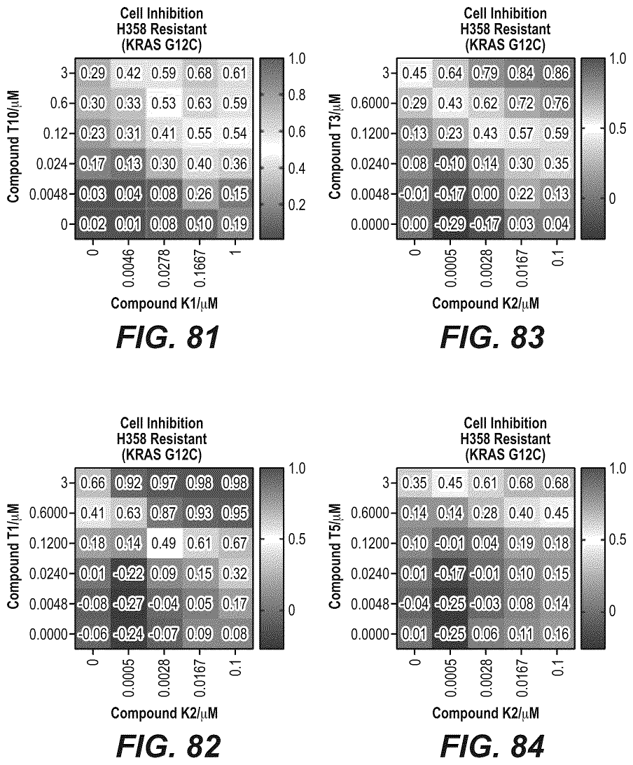

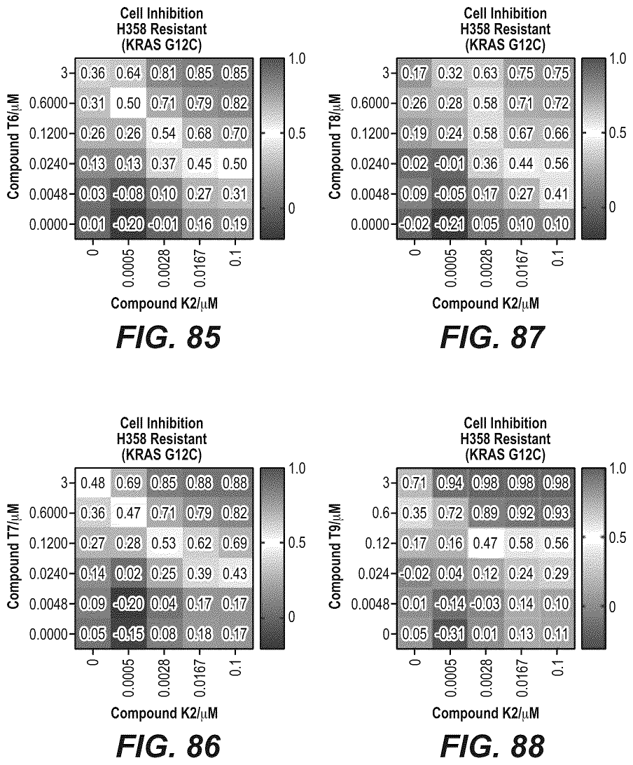

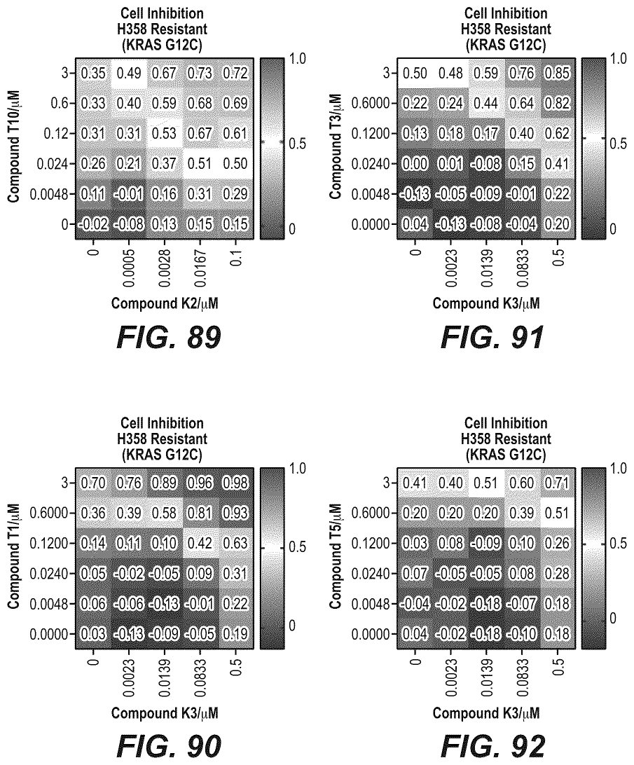

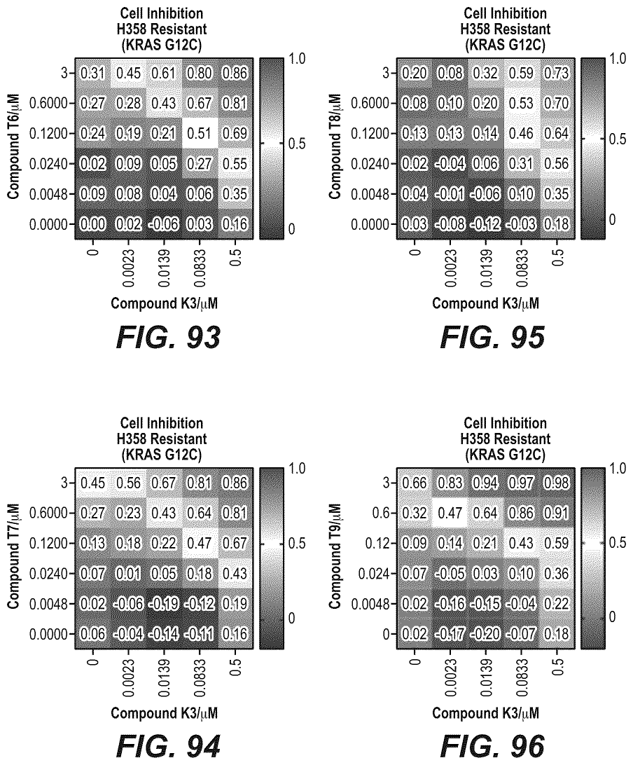

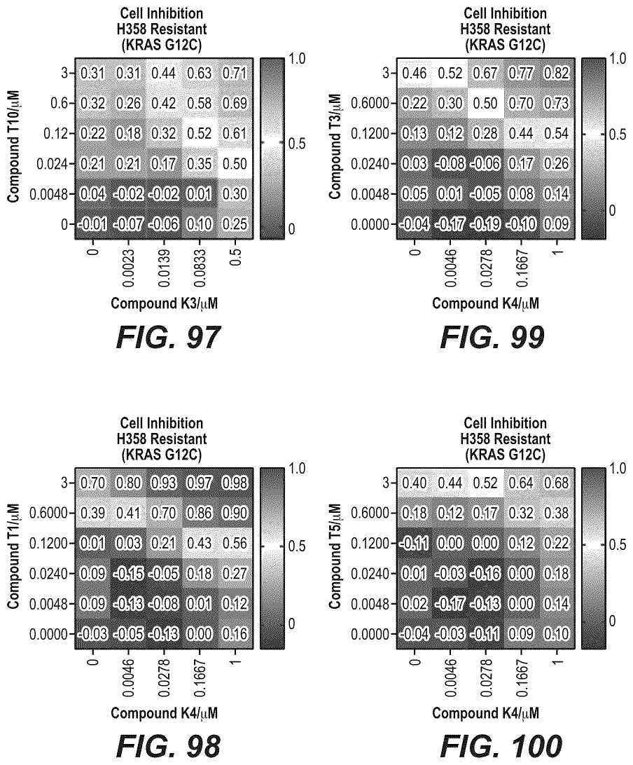

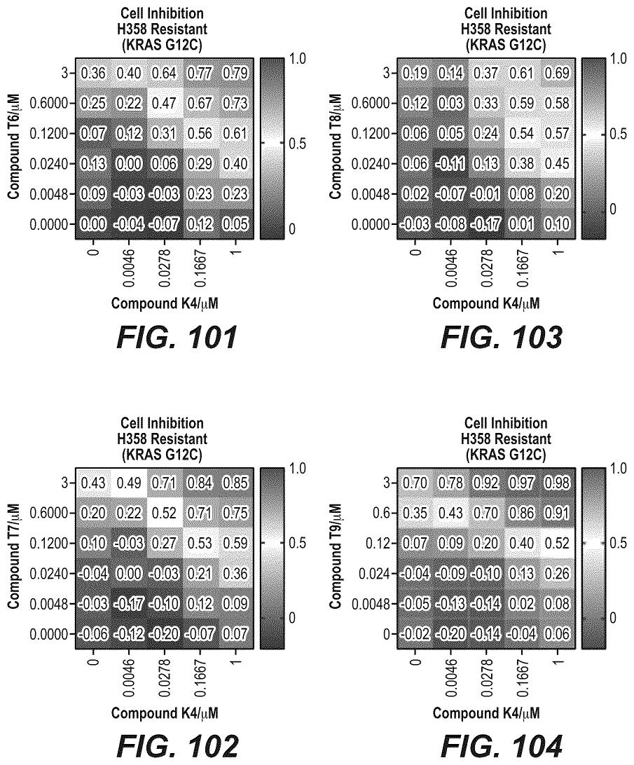

After an 11-14 day incubation period at 37° C., plates were taken out and incubated at room temperature for 30 minutes (making sure lids do not fog up and are free of condensation). The bottom of the plates was cleaned with 70% EtOH. The plates were then placed in an Oxford Optronix Gelcount machine for cell counting. Exemplary data are shown in FIG. 9.

### ABBREVIATIONS

The following abbreviations may be used in the synthetic examples provided herein:
AcOH Acetic acid
Cs₂CO₃ Cesium carbonate
DCE 1,2-Dichloroethane
DCM Dichloromethane
DIAD Diisopropyl azodicarboxylate
DMF N,N-Dimethylformamide
DMSO Dimethyl sulfoxide
EtOAc Ethyl acetate
EtOH Ethanol
HATU 1-[Bis(dimethylamino)methylene]-1H-1,2,3-triazolo[4,5-b]pyridinium 3-oxide hexafluorophosphate
HCl Hydrochloric acid
H₂O Water
KOAc Potassium acetate
MeOH Methanol
NaH Sodium hydride
NaHCO₃ Sodium bicarbonate
Na₂CO₃ Sodium carbonate
Na₂SO₃ Sodium sulfite
Na₂SO₄ Sodium sulfate
NBS N-bromosuccinimide
p-TsOH·H₂O p-Toluenesulfonic acid monohydrate
Pd(dppf)Cl₂ 1,1′-Bis(diphenylphosphino)ferrocene palladium(II) dichloride
PMB p-methoxybenzyl
PPh₃ Triphenylphosphine
THF Tetrahydrofuran

It is to be understood that the disclosure is not limited to the particular embodiments and aspects of the disclosure described above, as variations of the particular embodiments and aspects may be made and still fall within the scope of the appended claims. All documents cited to or relied upon herein are expressly incorporated herein by reference in their entirety.

### Enumerated Embodiments

Embodiment 1. A method of modulating YAP/TAZ-TEAD activity, or KRAS activity, or both, in a cell, comprising administering to the cell an effective amount of a combination, comprising: (i) one or more YAP/TAZ-TEAD inhibitors; and (ii) one or more KRAS inhibitors.
Embodiment 2. A method of inhibiting YAP/TAZ-TEAD activity, or KRAS activity, or both, in a cell, comprising administering to the cell an effective amount of a combination, comprising: (i) one or more YAP/TAZ-TEAD inhibitors; and (ii) one or more KRAS inhibitors.
Embodiment 3. A method of treating cancer in a subject in need thereof, comprising administering to the subject an effective amount of a combination, comprising: (i) one or more YAP/TAZ-TEAD inhibitors; and (ii) one or more KRAS inhibitors.
Embodiment 4. The method of embodiment 3, wherein the cancer is selected from the group consisting of hematological cancer, pancreatic cancer, MYH-associated polyposis, colorectal cancer, and lung cancer.
Embodiment 5. The method of embodiment 3, wherein the cancer is lung cancer.
Embodiment 6. The method of embodiment 5, wherein the lung cancer is non-small-cell lung cancer.
Embodiment 7. The method of any one of embodiments 1-6, wherein the one or more YAP/TAZ-TEAD inhibitors comprise:
   (a) a compound of formula (I):

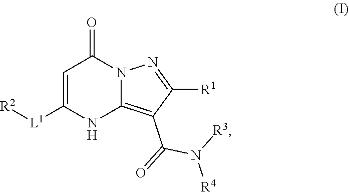

or a stereoisomer or tautomer thereof, or a pharmaceutically acceptable salt of any of the foregoing, wherein:
R¹ is selected from the group consisting of —C(O)—N(R^{a})(R^{b}), C₆₋₂₀aryl, 5-20 membered heteroaryl, 5-20 membered heterocyclyl, and C₁₋₆alkyl, wherein the C₆₋₂₀aryl, 5-20 membered heteroaryl and 5-20 membered heterocyclyl of R¹ are independently optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl, and wherein the C₁₋₆alkyl of R¹ is optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl;
   R^{a} and R^{b} are each independently H or C₁₋₆alkyl, wherein the C₁₋₆alkyl of R^{a} or R^{b} is optionally substituted with one or more substituents selected from the group consisting of halo, —OH, —CN, C₁₋₆alkyl, C₁₋₆haloalkyl, and C₁₋₆alkoxy,
   or R^{a} and R^{b} are taken, together with the atoms to which they attached, to form a 3-10 membered heterocyclyl, wherein the 3-10 membered heterocyclyl is optionally substituted with one or more substituents selected from the group consisting of halo, —OH, —CN, C₁₋₆alkyl, C₁₋₆haloalkyl, and C₁₋₆alkoxy;
   L¹ is absent or is *—O—CH₂—**, *—CH₂—O—**, or —O—, wherein ** denotes the point of attachment to the R² moiety and * denotes the point of attachment to the remainder of the molecule;
   R² is C₂₋₁₂alkyl, C₂₋₁₂alkenyl, or C₆₋₁₀aryl, wherein the C₂₋₁₂alkyl, C₂₋₁₂alkenyl, and C₆₋₁₀aryl of R² are independently optionally substituted with one or more substituents selected from the group consisting of halo, —OH, —CN, C₁₋₆alkyl, C₁₋₆haloalkyl, C₁₋₆alkoxy, C₆₋₁₀aryl, and C₃₋₁₀cycloalkyl, wherein the C₁₋₆alkyl, C₁₋₆alkoxy, and C₃₋₁₀cycloalkyl are independently optionally substituted with one or more halo, C₁₋₆haloalkyl, C₆₋₁₀aryl, or C₃₋₁₀cycloalkyl; and
R³ and R⁴ are each independently H or C₁₋₆alkyl, wherein the C₁₋₆alkyl of R³ or R⁴ is optionally substituted with one or more substituents selected from the group consisting of halo, —OH, —CN, C₁₋₆alkyl, C₁₋₆haloalkyl, and C₁₋₆alkoxy,
or R³ and R⁴, together with the atoms to which they are attached, form a 3-10 membered heterocyclyl, wherein the 3-10 membered heterocyclyl is optionally substituted with one or more substituents selected from the group consisting of halo, —OH, —CN, and C₁₋₆alkyl, wherein the C₁₋₆alkyl is optionally substituted with one or more substituents selected from the group consisting of halo, —OH, —CN, C₁₋₆alkyl, C₁₋₆haloalkyl, and C₁₋₆alkoxy;
(b) a compound of formula (II):

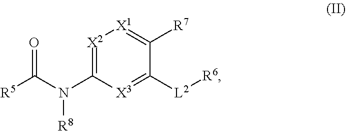

or a stereoisomer or tautomer thereof, or a pharmaceutically acceptable salt of any of the foregoing, wherein:
X¹ is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h}), or
the R⁹ of X¹ is taken together with R⁷, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH;
X² is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h}
X₃ is N or C—H,
   provided that, when X³ is N, and R⁵ is

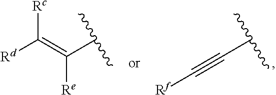

then at least one of X¹ and X² is N;
R⁵ is:
(i) oxiranyl or oxetanyl, wherein the oxiranyl or oxetanyl of R⁵ is optionally substituted with one or more C₁₋₆alkyl, wherein the C₁₋₆alkyl is independently optionally substituted with one or more —C(O)—NH₂, and L² is absent or is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule, or
(ii) —N(R^{g})(CN), and
   L² is absent or is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule, or
(iii)

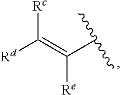

wherein R^{c}, R^{d}, and R^{e} are each independently selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{c}, R^{d}, or R^{e} is optionally substituted with one or more —OH, provided that at least two of R^{c}, R^{d}, and R^{e} are H, and
   L² is absent or is selected from the group consisting of *—CH₂—O—**,
   *—O—CH₂—**, —CH═CH—, and —C≡C—, wherein ** indicates the attachment point to the R⁶ moiety and * indicates the attachment point to the remainder of the molecule, or
(iv)

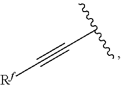

wherein R^{f} is selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{f} is optionally substituted with —OH, and
   L² is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule; R⁶ is C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl, wherein
   the C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), —O(R^{g}), and —SF₅,
   provided that, when R⁶ is C₁₋₁₂alkyl, wherein the C₁₋₁₂alkyl is independently optionally substituted with one or two substituents selected from the group consisting of —CN, halo, C₁₋₆alkyl, C₁₋₆haloalkyl, C₃₋₁₀cycloalkyl, —NO₂, —N(R^{g})(R^{h}), and —O(R^{g}), then L² is —CH═CH— or —C≡C—;
R⁷ is —CN, C₁₋₆alkyl, C₁₋₄alkoxy, or C₂₋₄alkenyl, wherein the C₂₋₄alkenyl of R⁷ is optionally substituted with —N(R^{g})(R^{h}), or
R⁷ is taken together with R⁹ of X¹, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH, or
R⁷ is taken together with the carbon atom of *—CH₂—O—** of L², and the atoms to which they are attached, to form a C₆aryl or a 6-membered heteroaryl,
   provided that:
(i) when R⁷ is —CN, C₁₋₆alkyl, C₁₋₄alkoxy, or C₂₋₄alkenyl, wherein the C₂₋₄alkenyl is optionally substituted with —N(R^{g})(R^{h}), and
   R⁵ is

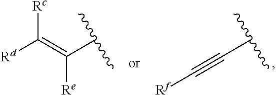

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}), then L² is *—CH₂—O—**, —CH═CH—, or —C≡C—, wherein ** indicates the attachment point to the R⁶ moiety and * indicates the attachment point to the remainder of the molecule, and
(ii) when R⁷ is taken together with R⁹ of X¹, and the atoms to which they are attached, to form a 5-membered heterocyclyl or a 5-membered heteroaryl, provided that X³ is CH, and
   R⁵ is

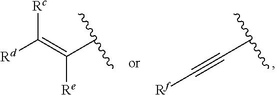

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is absent or is *—CH₂—O—**, —CH═CH—, or —C≡C—, wherein ** indicates the attachment point to the R⁶ moiety and * indicates the attachment point to the remainder of the molecule, and
(iii) when R⁷ is taken together with the carbon atom of *—CH₂—O—** of L², and the atoms to which they are attached, to form a C₆aryl or a 6-membered heteroaryl, and
   R⁵ is

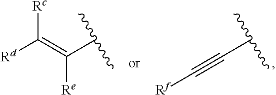

then R⁶ is 3-10 membered saturated heterocyclyl or 5-20 membered heteroaryl, wherein the 3-10 membered saturated heterocyclyl or 5-20 membered heteroaryl is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g});
R⁸ is H or C₁₋₆alkyl, wherein the C₁₋₆alkyl is optionally substituted with one or more —OH; and
R^{g} and R^{h} are, independently of each other and independently at each occurrence, selected from the group consisting of H, —CN, —OH, C₁₋₆alkyl, C₂-6alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl of R^{g} and R^{h} are each independently optionally substituted with one or more substituents independently selected from the group consisting of C₁₋₆alkyl, C₁₋₆haloalkyl, C₁₋₆alkoxy, oxo, —CN, halo, —NO₂, and —OH; or
(c) a compound of formula (III):

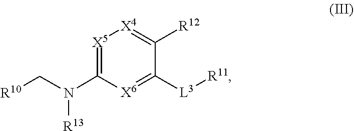

or a stereoisomer or tautomer thereof, or a pharmaceutically acceptable salt of any of the foregoing, wherein:
X⁴ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ), or
the R¹⁴ of X⁴ is taken together with R¹², and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl;
X⁵ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ);
X₆ is N or C—H;
R¹⁰ is:
   (i) a 3-5 membered saturated heterocyclyl comprising at least one annular oxygen atom, wherein the 3-5 membered saturated heterocyclyl is optionally substituted with one or more C₁₋₆alkyl, or
   (ii) —N(R^{m})(Rⁿ), or
   (iii)

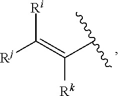

wherein Rⁱ, R^{j}, and R^{k} are each independently selected from the group consisting of H, halo, —CN, —OH, —B(OH)₂, —C(O)—OH,
—C(O)—N(R^{m})(Rⁿ), —C(O)—C₁₋₆alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆alkyl of Rⁱ, R^{j}, or R^{k} is optionally substituted with one or more —OH, or
(iv)

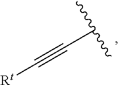

wherein R^{t} is selected from the group consisting of H, halo, —CN, —OH, —B(OH)₂, —C(O)—OH, —C(O)—N(R^{m})(Rⁿ), —C(O)—C₁₋₆alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆ alkyl is optionally substituted with one or more —OH;
L³ is absent or is selected from the group consisting of —O—, *—CH₂—O—**, *—O—CH₂—**, —CH═CH—, and —C≡C—, wherein ** indicates the attachment point to the R¹¹ moiety and * indicates the attachment point to the remainder of the molecule;
R¹¹ is C₁₋₁₂alkyl, C₃₋₁₀cycloalkyl, 3-10 membered heterocyclyl, C₅₋₁₃spirocyclyl, C₆₋₂₀aryl, or 5-20 membered heteroaryl,
   wherein the C₁₋₁₂alkyl, C₃₋₁₀cycloalkyl, 3-10 membered saturated heterocyclyl, C₅₋₁₃spirocyclyl, C₆₋₂₀aryl, or 5-20 membered heteroaryl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}),
   provided that, when R¹¹ is C₁₋₁₂alkyl, wherein the C₁₋₁₂alkyl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}), then L³ is —CH═CH— or —C≡C—;
R¹² is —CN, C₁₋₆alkyl, C₁₋₄alkoxy, or C₂₋₄alkenyl, wherein the C₂₋₄alkenyl is optionally substituted with —N(R^{m})(Rⁿ), or
R¹² is taken together with R¹⁴ of X⁴, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, or
R¹² is taken together with the carbon atom of *—CH₂—O—** of L³, and the atoms to which they are attached, to form a C₆aryl or a 6-membered heteroaryl; R¹³ is H or C₁₋₆alkyl, wherein the C₁₋₆alkyl of R¹³ is optionally substituted with one or more —OH; and
R^{m} and Rⁿ are, independently of each other and independently at each occurrence, selected from the group consisting of H, —CN, —OH, C₁₋₆alkyl, C₂₋₆alkenyl, C₂₋₆alkynyl, C₃₋₁₀cycloalkyl, —C₁₋₆alkyl-C₃₋₁₀cycloalkyl, 3-10 membered heterocyclyl, C₅₋₁₃spirocyclyl, C₆₋₂₀aryl, and 5-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₅₋₁₃spirocyclyl, C₆₋₂₀aryl, and 5-20 membered heteroaryl of R^{m} and Rⁿ are each independently optionally substituted with one or more substituents independently selected from the group consisting of C₁₋₆alkyl, C₁₋₆haloalkyl, C₁₋₆alkoxy, oxo, —CN, halo, —NO₂, and —OH,
or any combination thereof.
Embodiment 8. The method of embodiment 7, wherein the one or more YAP/TAZ-TEAD inhibitors comprise a compound of formula (I), or a stereoisomer or tautomer thereof, or a pharmaceutically acceptable salt of any of the foregoing.
Embodiment 9. The method of embodiment 8, wherein the one or more YAP/TAZ-TEAD inhibitors comprise

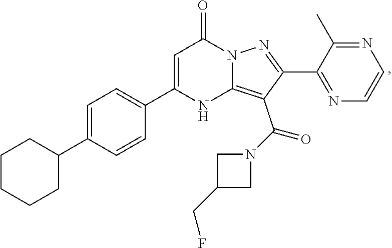 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing.
Embodiment 10. The method of any one of embodiments 7-9, wherein the one or more YAP/TAZ-TEAD inhibitors comprise a compound of formula (II), or a stereoisomer or tautomer thereof, or a pharmaceutically acceptable salt of any of the foregoing.
Embodiment 11. The method of embodiment 10, wherein, for the compound of formula (II), R⁵ is oxiranyl, wherein the oxiranyl of R⁵ is optionally substituted with one or more C₁₋₆ alkyl, wherein the C₁₋₆alkyl is optionally substituted with one or more —C(O)—NH₂.
Embodiment 12. The method of embodiment 10 or embodiment 11, wherein the one or more YAP/TAZ-TEAD inhibitors comprise

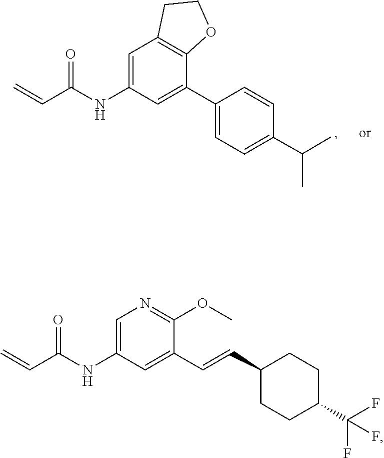

or both, or a stereoisomer or tautomer thereof, or a pharmaceutically acceptable salt of any of the foregoing.
Embodiment 13. The method of any one of embodiments 7-12, wherein the one or more YAP/TAZ-TEAD inhibitors comprise a compound of formula (III), or a stereoisomer or tautomer thereof, or a pharmaceutically acceptable salt of any of the foregoing.
Embodiment 14. The method of embodiment 13, wherein, for the compound of formula (III), R¹⁰ is oxiranyl, wherein the oxiranyl of R¹⁰ is optionally substituted with one or more C₁₋₆alkyl.
Embodiment 15. The method of embodiment 13 or embodiment 14, wherein the one or more YAP/TAZ-TEAD inhibitors comprise

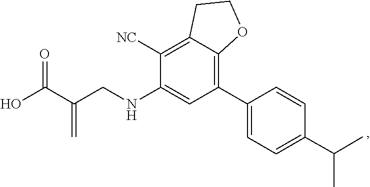 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing.
Embodiment 16. The method of any one of embodiments 1-15, wherein the one or more KRAS inhibitors comprise one or more inhibitors of the KRAS G12C mutant.
Embodiment 17. The method of any one of embodiments 1-16, wherein the one or more KRAS inhibitors comprise

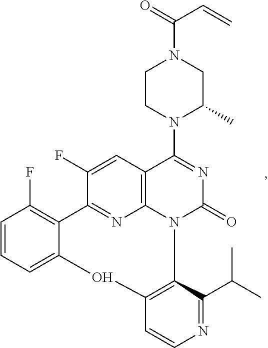

or a stereoisomer or tautomer thereof, or a pharmaceutically acceptable salt of any of the foregoing.
Embodiment 18. The method of any one of embodiments 1-17, wherein the one or more KRAS inhibitors comprise

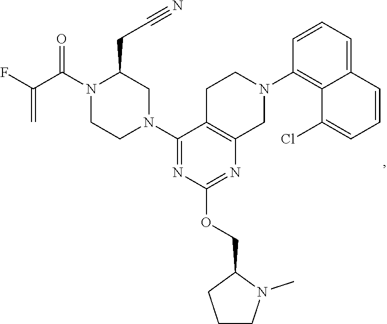 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.
Embodiment 19. The method of any one of embodiments 1-18, wherein the one or more KRAS inhibitors comprise

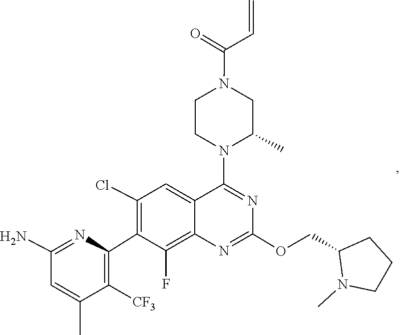

or a stereoisomer or tautomer thereof, or a pharmaceutically acceptable salt of any of the foregoing.
Embodiment 20. The method of any one of embodiments 1-19, wherein the combination further comprises one or more pharmaceutically acceptable excipients.
Embodiment 21. The method of any one of embodiments 1-20, wherein the one or more YAP/TAZ-TEAD inhibitors and the one or more KRAS inhibitors are administered simultaneously.
Embodiment 22. The method of any one of embodiments 1-20, wherein the one or more YAP/TAZ-TEAD inhibitors and the one or more KRAS inhibitors are administered sequentially.
Embodiment 23. A composition, comprising: (i) one or more YAP/TAZ-TEAD inhibitors; and (ii) one or more KRAS inhibitors.
Embodiment 24. The composition of embodiment 23, wherein the one or more YAP/TAZ-TEAD inhibitors comprise:
   (a) a compound of formula (I):

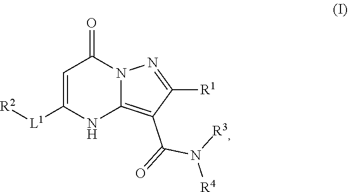

or a stereoisomer or tautomer thereof, or a pharmaceutically acceptable salt of any of the foregoing, wherein:
R¹ is selected from the group consisting of —C(O)—N(R^{a})(R^{b}), C₆₋₂₀aryl, 5-20 membered heteroaryl, 5-20 membered heterocyclyl, and C₁₋₆alkyl, wherein the C₆₋₂₀aryl, 5-20 membered heteroaryl and 5-20 membered heterocyclyl of R¹ are independently optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl, and wherein the C₁₋₆alkyl of R¹ is optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl;
R^{a} and R^{b} are each independently H or C₁₋₆alkyl, wherein the C₁₋₆alkyl of R^{a} or R^{b} is optionally substituted with one or more substituents selected from the group consisting of halo, —OH, —CN, C₁₋₆alkyl, C₁₋₆haloalkyl, and C₁₋₆alkoxy,
or R^{a} and R^{b} are taken, together with the atoms to which they attached, to form a 3-10 membered heterocyclyl, wherein the 3-10 membered heterocyclyl is optionally substituted with one or more substituents selected from the group consisting of halo, —OH, —CN, C₁₋₆alkyl, C₁₋₆haloalkyl, and C₁₋₆alkoxy;
L¹ is absent or is *—O—CH₂—**, *—CH₂—O—**, or —O—, wherein ** denotes the point of attachment to the R² moiety and * denotes the point of attachment to the remainder of the molecule;
R² is C₂₋₁₂alkyl, C₂₋₁₂alkenyl, or C₆₋₁₀aryl, wherein the C₂₋₁₂alkyl, C₂₋₁₂alkenyl, and C₆₋₁₀aryl of R² are independently optionally substituted with one or more substituents selected from the group consisting of halo, —OH, —CN, C₁₋₆alkyl, C₁₋₆haloalkyl, C₁₋₆alkoxy, C₆₋₁₀aryl, and C₃₋₁₀cycloalkyl, wherein the C₁₋₆alkyl, C₁₋₆alkoxy, and C₃₋₁₀cycloalkyl are independently optionally substituted with one or more halo, C₁₋₆haloalkyl, C₆₋₁₀aryl, or C₃₋₁₀cycloalkyl; and
R³ and R⁴ are each independently H or C₁₋₆alkyl, wherein the C₁₋₆alkyl of R³ or R⁴ is optionally substituted with one or more substituents selected from the group consisting of halo, —OH, —CN, C₁₋₆alkyl, C₁₋₆haloalkyl, and C₁₋₆alkoxy,
or R³ and R⁴, together with the atoms to which they are attached, form a 3-10 membered heterocyclyl, wherein the 3-10 membered heterocyclyl is optionally substituted with one or more substituents selected from the group consisting of halo, —OH, —CN, and C₁₋₆alkyl, wherein the C₁₋₆alkyl is optionally substituted with one or more substituents selected from the group consisting of halo, —OH, —CN, C₁₋₆alkyl, C₁₋₆haloalkyl, and C₁₋₆alkoxy;
(b) a compound of formula (II):

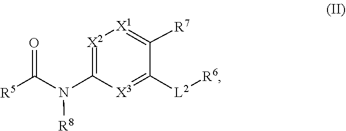

or a stereoisomer or tautomer thereof, or a pharmaceutically acceptable salt of any of the foregoing, wherein:
X¹ is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h}), or
the R⁹ of X¹ is taken together with R⁷, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH;
X² is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h});
X³ is N or C—H,
   provided that, when X³ is N, and R⁵ is

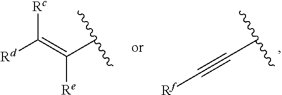

then at least one of X¹ and X² is N;
R⁵ is:
(i) oxiranyl or oxetanyl, wherein the oxiranyl or oxetanyl of R⁵ is optionally substituted with one or more C₁₋₆alkyl, wherein the C₁₋₆alkyl is independently optionally substituted with one or more —C(O)—NH₂, and
   L² is absent or is selected from the group consisting of —O—, *—CH₂—O—**,
   *—O—CH₂—**, —CH═CH—, and —C≡C—, wherein ** indicates the attachment point to the R⁶ moiety and * indicates the attachment point to the remainder of the molecule, or
(ii) —N(R^{g})(CN), and
   L² is absent or is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule, or
(iii)

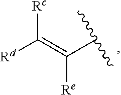

wherein R^{c}, R^{d}, and R^{e} are each independently selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{c}, R^{d}, or R^{e} is optionally substituted with one or more —OH, provided that at least two of R^{c}, R^{d}, and R^{e} are H, and
   L² is absent or is selected from the group consisting of *—CH₂—O—**,
   *—O—CH₂—**, —CH═CH—, and —C≡C—, wherein ** indicates the attachment point to the R⁶ moiety and * indicates the attachment point to the remainder of the molecule, or
(iv)

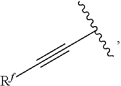

wherein R^{f} is selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{f} is optionally substituted with —OH, and
   L² is selected from the group consisting of —O—, *—CH₂—O—**, *—O—CH₂—**
   —CH═CH—, and —C≡C—, wherein ** indicates the attachment point to the R⁶ moiety and * indicates the attachment point to the remainder of the molecule;
R⁶ is C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl, wherein:
   the C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl of R⁶ is independently optionally substituted with one or two substituents independently selected from the group consisting of —CN, halo, C₁₋₆alkyl, C₁₋₆ haloalkyl, C₃₋₁₀cycloalkyl, —NO₂, —N(R^{g})(R^{h}), —O(R^{g}), and —SF₅,
   provided that, when R⁶ is C₁₋₁₂alkyl, wherein the C₁₋₁₂alkyl is independently optionally substituted with one or two substituents selected from the group consisting of —CN, halo, C₁₋₆alkyl, C₁₋₆haloalkyl, C₃₋₁₀cycloalkyl, —NO₂, —N(R^{g})(R^{h}), and —O(R^{g}), then L² is —CH═CH— or —C≡C—;
R⁷ is —CN, C₁₋₆alkyl, C₁₋₄alkoxy, or C₂₋₄alkenyl, wherein the C₂₋₄alkenyl of R⁷ is optionally substituted with —N(R^{g})(R^{h}), or
R⁷ is taken together with R⁹ of X¹, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH, or
R⁷ is taken together with the carbon atom of *—CH₂—O—** of L², and the atoms to which they are attached, to form a C₆aryl or a 6-membered heteroaryl,
   provided that:
(i) when R⁷ is —CN, C₁₋₆alkyl, C₁₋₄alkoxy, or C₂₋₄alkenyl, wherein the C₂₋₄alkenyl is optionally substituted with —N(R^{g})(R^{h}), and
   R⁵ is

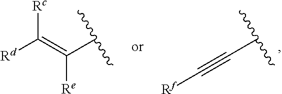

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is *—CH₂—O—**, —CH═CH—, or —C≡C—, wherein ** indicates the attachment point to the R⁶ moiety and * indicates the attachment point to the remainder of the molecule, and
(ii) when R⁷ is taken together with R⁹ of X¹, and the atoms to which they are attached, to form a 5-membered heterocyclyl or a 5-membered heteroaryl, provided that X³ is CH, and
   R⁵ is

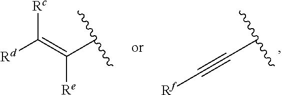

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is absent or is *—CH₂—O—**, —CH═CH—, or —C≡C—, wherein ** indicates the attachment point to the R⁶ moiety and * indicates the attachment point to the remainder of the molecule, and
(iii) when R⁷ is taken together with the carbon atom of *—CH₂—O—** of L², and the atoms to which they are attached, to form a C₆aryl or a 6-membered heteroaryl, and
   R⁵ is

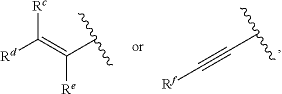

then R⁶ is 3-10 membered saturated heterocyclyl or 5-20 membered heteroaryl, wherein the 3-10 membered saturated heterocyclyl or 5-20 membered heteroaryl is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R⁹);
R⁸ is H or C₁₋₆alkyl, wherein the C₁₋₆alkyl is optionally substituted with one or more —OH; and
R^{g} and R^{h} are, independently of each other and independently at each occurrence, selected from the group consisting of H, —CN, —OH, C₁₋₆alkyl, C₂-6alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl of R^{g} and R^{h} are each independently optionally substituted with one or more substituents independently selected from the group consisting of C₁₋₆alkyl, C₁₋₆haloalkyl, C₁₋₆alkoxy, oxo, —CN, halo, —NO₂, and —OH; or
(c) a compound of formula (III):

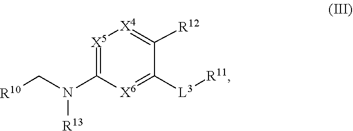

or a stereoisomer or tautomer thereof, or a pharmaceutically acceptable salt of any of the foregoing, wherein:
X⁴ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ), or
the R¹⁴ of X⁴ is taken together with R¹², and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl;
X⁵ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ);
X₆ is N or C—H;
R¹⁰ is:
   (i) a 3-5 membered saturated heterocyclyl comprising at least one annular oxygen atom, wherein the 3-5 membered saturated heterocyclyl is optionally substituted with one or more C₁₋₆alkyl, or
   (ii) —N(R^{m})(Rⁿ), or
   (iii)

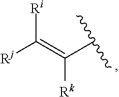

wherein Rⁱ, R^{j}, and R^{k} are each independently selected from the group consisting of H, halo, —CN, —OH, —B(OH)₂, —C(O)—OH,
—C(O)—N(R^{m})(Rⁿ), —C(O)—C₁₋₆alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆alkyl of Rⁱ, R^{j}, or R^{k} is optionally substituted with one or more —OH, or
(iv)

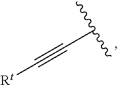

wherein R^{t} is selected from the group consisting of H, halo, —CN, —OH, —B(OH)₂, —C(O)—OH, —C(O)—N(R^{m})(Rⁿ), —C(O)—C₁₋₆ alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆ alkyl is optionally substituted with one or more —OH;
L³ is absent or is selected from the group consisting of —O—, *—CH₂—O—**, *—O—CH₂—**, —CH═CH—, and —C≡C—, wherein ** indicates the attachment point to the R¹¹ moiety and * indicates the attachment point to the remainder of the molecule;
R¹¹ is C₁₋₁₂alkyl, C₃₋₁₀cycloalkyl, 3-10 membered heterocyclyl, C₅₋₁₃spirocyclyl, C₆₋₂₀aryl, or 5-20 membered heteroaryl,
   wherein the C₁₋₁₂alkyl, C₃₋₁₀cycloalkyl, 3-10 membered saturated heterocyclyl, C₅₋₁₃spirocyclyl, C₆₋₂₀aryl, or 5-20 membered heteroaryl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}),
   provided that, when R¹¹ is C₁₋₁₂alkyl, wherein the C₁₋₁₂alkyl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}), then L³ is —CH═CH— or —C≡C—;
R¹² is —CN, C₁₋₆alkyl, C₁₋₄alkoxy, or C₂₋₄alkenyl, wherein the C₂₋₄alkenyl is optionally substituted with —N(R^{m})(Rⁿ), or
R¹² is taken together with R¹⁴ of X⁴, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, or
R¹² is taken together with the carbon atom of *—CH₂—O—** of L³, and the atoms to which they are attached, to form a C₆aryl or a 6-membered heteroaryl;
R¹³ is H or C₁₋₆alkyl, wherein the C₁₋₆alkyl of R¹³ is optionally substituted with one or more —OH; and
R^{m} and Rⁿ are, independently of each other and independently at each occurrence, selected from the group consisting of H, —CN, —OH, C₁₋₆alkyl, C₂₋₆alkenyl, C₂₋₆alkynyl, C₃₋₁₀cycloalkyl, —C₁₋₆alkyl-C₃₋₁₀cycloalkyl, 3-10 membered heterocyclyl, C₅₋₁₃spirocyclyl, C₆₋₂₀aryl, and 5-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₅₋₁₃spirocyclyl, C₆₋₂₀aryl, and 5-20 membered heteroaryl of R^{m} and Rⁿ are each independently optionally substituted with one or more substituents independently selected from the group consisting of C₁₋₆alkyl, C₁₋₆haloalkyl, C₁₋₆alkoxy, oxo, —CN, halo, —NO₂, and —OH,
or any combination thereof.
Embodiment 25. The composition of embodiment 24, wherein the one or more YAP/TAZ-TEAD inhibitors comprise a compound of formula (I), or a stereoisomer or tautomer thereof, or a pharmaceutically acceptable salt of any of the foregoing.
Embodiment 26. The composition of embodiment 24 or embodiment 25, wherein the one or more YAP/TAZ-TEAD inhibitors comprise

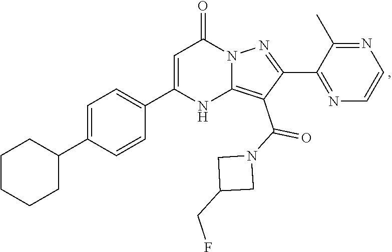 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing.
Embodiment 27. The composition of any one of embodiments 24-26, wherein the one or more YAP/TAZ-TEAD inhibitors comprise a compound of formula (II), or a stereoisomer or tautomer thereof, or a pharmaceutically acceptable salt of any of the foregoing.
Embodiment 28. The composition of embodiment 27, wherein, for the compound of formula (II), R⁵ is oxiranyl, wherein the oxiranyl of R⁵ is optionally substituted with one or more C₁₋₆alkyl, wherein the C₁₋₆alkyl is optionally substituted with one or more —C(O)—NH₂.
Embodiment 29. The composition of embodiment 27 or embodiment 28, wherein the one or more YAP/TAZ-TEAD inhibitors comprise

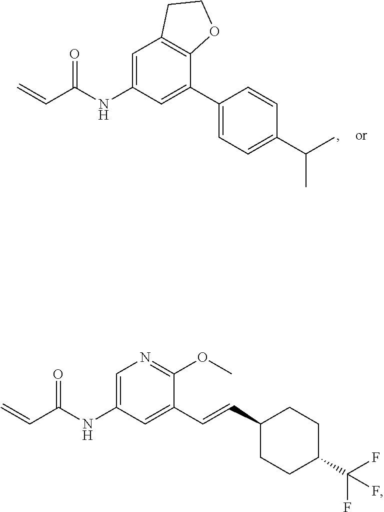

or both, or a stereoisomer or tautomer thereof, or a pharmaceutically acceptable salt of any of the foregoing.
Embodiment 30. The composition of any one of embodiments 24-29, wherein the one or more YAP/TAZ-TEAD inhibitors comprise a compound of formula (III), or a stereoisomer or tautomer thereof, or a pharmaceutically acceptable salt of any of the foregoing.
Embodiment 31. The composition of embodiment 30, wherein, for the compound of formula (III), R¹⁰ is oxiranyl, wherein the oxiranyl of R¹⁰ is optionally substituted with one or more C₁₋₆alkyl.
Embodiment 32. The composition of embodiment 30 or embodiment 31, wherein the one or more YAP/TAZ-TEAD inhibitors comprise

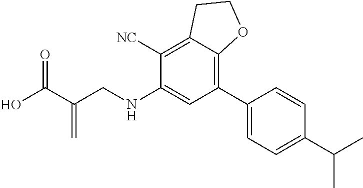 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing.
Embodiment 33. The composition of any one of embodiments 23-32, wherein the one or more KRAS inhibitors comprise one or more inhibitors of the KRAS G12C mutant.
Embodiment 34. The composition of any one of embodiments 23-33, wherein the one or more KRAS inhibitors comprise

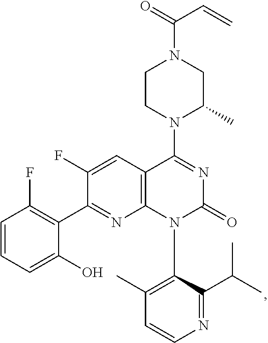

or a stereoisomer or tautomer thereof, or a pharmaceutically acceptable salt of any of the foregoing.
Embodiment 35. The composition of any one of embodiments 23-34, wherein the one or more KRAS inhibitors comprise

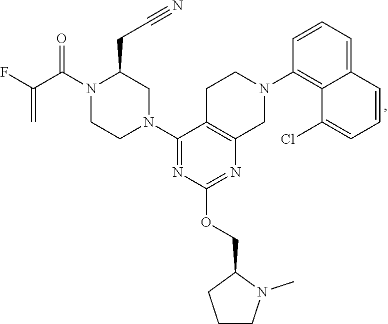 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.
Embodiment 36. The composition of any one of embodiments 23-35, wherein the one or more KRAS inhibitors comprise

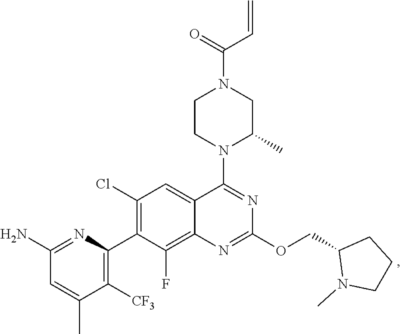

or a stereoisomer or tautomer thereof, or a pharmaceutically acceptable salt of any of the foregoing.
Embodiment 37. The composition of any one of embodiments 23-36, wherein the composition further comprises one or more pharmaceutically acceptable excipients.
Embodiment 38. A kit, comprising (i) an effective amount of a combination comprising one or more YAP/TAZ-TEAD inhibitors and one or more KRAS inhibitors; and (ii) instructions for administering the combination to treat cancer in a subject in need thereof.
Embodiment 39. The kit of embodiment 38, wherein the cancer is selected from the group consisting of hematological cancer, pancreatic cancer, MYH-associated polyposis, colorectal cancer, and lung cancer.
Embodiment 40. The kit of embodiment 38, wherein the cancer is lung cancer.
Embodiment 41. The kit of embodiment 40, wherein the lung cancer is non-small-cell lung cancer.
Embodiment 42. The kit of any one of embodiments 38-41, wherein the one or more YAP/TAZ-TEAD inhibitors comprise:
   (a) a compound of formula (I):

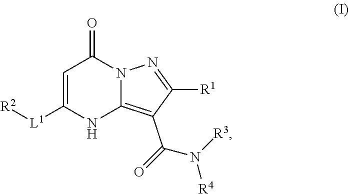

or a stereoisomer or tautomer thereof, or a pharmaceutically acceptable salt of any of the foregoing, wherein:
R¹ is selected from the group consisting of —C(O)—N(R^{a})(R^{b}), C₆₋₂₀aryl, 5-20 membered heteroaryl, 5-20 membered heterocyclyl, and C₁₋₆alkyl, wherein the C₆₋₂₀aryl, 5-20 membered heteroaryl and 5-20 membered heterocyclyl of R¹ are independently optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl, and wherein the C₁₋₆alkyl of R¹ is optionally substituted with one or more substituents selected from the group consisting of halo, —OH, —CN, C₁₋₆alkyl, C₁₋₆haloalkyl, C₁₋₆alkoxy, C₃₋₈cycloalkyl, and C₆₋₁₀aryl;
R^{a} and R^{b} are each independently H or C₁₋₆alkyl, wherein the C₁₋₆alkyl of R^{a} or R^{b} is optionally substituted with one or more substituents selected from the group consisting of halo, —OH, —CN, C₁₋₆alkyl, C₁₋₆haloalkyl, and C₁₋₆alkoxy,
or R^{a} and R^{b} are taken, together with the atoms to which they attached, to form a 3-10 membered heterocyclyl, wherein the 3-10 membered heterocyclyl is optionally substituted with one or more substituents selected from the group consisting of halo, —OH, —CN, C₁₋₆alkyl, C₁₋₆haloalkyl, and C₁₋₆alkoxy;
L¹ is absent or is *—O—CH₂—**, *—CH₂—O—**, or —O—, wherein ** denotes the point of attachment to the R² moiety and * denotes the point of attachment to the remainder of the molecule;
R² is C₂₋₁₂alkyl, C₂₋₁₂alkenyl, or C₆₋₁₀aryl, wherein the C₂₋₁₂alkyl, C₂₋₁₂alkenyl, and C₆₋₁₀aryl of R² are independently optionally substituted with one or more substituents selected from the group consisting of halo, —OH, —CN, C₁₋₆alkyl, C₁₋₆haloalkyl, C₁₋₆alkoxy, C₆₋₁₀aryl, and C₃₋₁₀cycloalkyl, wherein the C₁₋₆alkyl, C₁₋₆alkoxy, and C₃₋₁₀cycloalkyl are independently optionally substituted with one or more halo, C₁₋₆haloalkyl, C₆₋₁₀aryl, or C₃₋₁₀cycloalkyl; and
R³ and R⁴ are each independently H or C₁₋₆alkyl, wherein the C₁₋₆alkyl of R³ or R⁴ is optionally substituted with one or more substituents selected from the group consisting of halo, —OH, —CN, C₁₋₆alkyl, C₁₋₆haloalkyl, and C₁₋₆alkoxy,
or R³ and R⁴, together with the atoms to which they are attached, form a 3-10 membered heterocyclyl, wherein the 3-10 membered heterocyclyl is optionally substituted with one or more substituents selected from the group consisting of halo, —OH, —CN, and C₁₋₆alkyl, wherein the C₁₋₆alkyl is optionally substituted with one or more substituents selected from the group consisting of halo, —OH, —CN, C₁₋₆alkyl, C₁₋₆haloalkyl, and C₁₋₆alkoxy;
(b) a compound of formula (II):

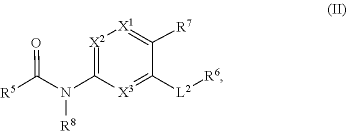

or a stereoisomer or tautomer thereof, or a pharmaceutically acceptable salt of any of the foregoing, wherein:
X¹ is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h}), or
the R⁹ of X¹ is taken together with R⁷, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH;
X² is N or C—R⁹, wherein each R⁹ is independently selected from the group consisting of H, —CN, halo, —C(O)—NH₂, —N(R^{g})(R^{h}), C₃₋₁₀cycloalkyl, C₁₋₆alkoxy, C₆₋₂₀aryl, and C₁₋₆alkyl, wherein the C₁₋₆alkyl of R⁹ is optionally substituted with one or more —OH or —N(R^{g})(R^{h});
X³ is N or C—H,
   provided that, when X³ is N, and R⁵ is

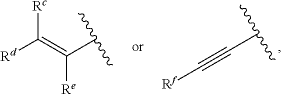

then at least one of X¹ and X² is N;
R⁵ is:
(i) oxiranyl or oxetanyl, wherein the oxiranyl or oxetanyl of R⁵ is optionally substituted with one or more C₁₋₆alkyl, wherein the C₁₋₆alkyl is independently optionally substituted with one or more —C(O)—NH₂, and
   L² is absent or is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule, or
(ii) —N(R^{g})(CN), and
   L² is absent or is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule, or
(iii)

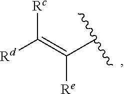

wherein R^{c}, R^{d}, and R^{e} are each independently selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{c}, R^{d}, or R^{e} is optionally substituted with one or more —OH, provided that at least two of R^{c}, R^{d}, and R^{e} are H, and
   L² is absent or is selected from the group consisting of *—CH₂—O—**,
   *—O—CH₂—**, —CH═CH—, and —C≡C—, wherein ** indicates the attachment point to the R⁶ moiety and * indicates the attachment point to the remainder of the molecule, or
(iv)

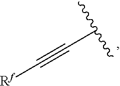

wherein R^{f} is selected from the group consisting of H, halo, —CN, —OH, C₁₋₆alkyl, C₆₋₂₀aryl, 3-10 membered heterocyclyl, and 5-20 membered heteroaryl, wherein the C₁₋₆alkyl of R^{f} is optionally substituted with —OH, and
   L² is selected from the group consisting of —O—, *—CH₂—O—**, *—O—CH₂—**, —CH═CH—, and —C≡C—, wherein ** indicates the attachment point to the R⁶ moiety and * indicates the attachment point to the remainder of the molecule; R⁶ is C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl, wherein
   the C₁₋₁₂alkyl, C₃₋₁₀cycloalkyl, 3-10 membered saturated heterocyclyl, C₆₋₂₀aryl, C₅₋₁₃spirocyclyl, or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂,
   —N(R^{g})(R^{h}), —O(R^{g}), and —SF₅,
   provided that, when R⁶ is C₁₋₁₂alkyl, wherein the C₁₋₁₂alkyl is independently optionally substituted with one or two substituents selected from the group consisting of —CN, halo, C₁₋₆alkyl, C₁₋₆haloalkyl, C₃₋₁₀cycloalkyl, —NO₂, —N(R^{g})(R^{h}), and —O(R^{g}), then L² is —CH═CH— or —C≡C—;
R⁷ is —CN, C₁₋₆alkyl, C₁₋₄alkoxy, or C₂₋₄alkenyl, wherein the C₂₋₄alkenyl of R⁷ is optionally substituted with —N(R^{g})(R^{h}), or
R⁷ is taken together with R⁹ of X¹, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, provided that X³ is CH, or
R⁷ is taken together with the carbon atom of *—CH₂—O—** of L², and the atoms to which they are attached, to form a C₆aryl or a 6-membered heteroaryl,
   provided that:
(i) when R⁷ is —CN, C₁₋₆alkyl, C₁₋₄alkoxy, or C₂₋₄alkenyl, wherein the C₂₋₄alkenyl is optionally substituted with —N(R^{g})(R^{h}), and
   R⁵ is

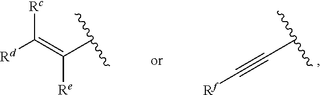

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is *—CH₂—O—**, —CH═CH—, or —C≡C—, wherein ** indicates the attachment point to the R⁶ moiety and * indicates the attachment point to the remainder of the molecule, and
(ii) when R⁷ is taken together with R⁹ of X¹, and the atoms to which they are attached, to form a 5-membered heterocyclyl or a 5-membered heteroaryl, provided that X³ is CH, and
   R⁵ is

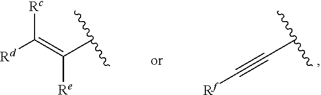

and
R⁶ is 3-10 membered saturated heterocyclyl or 5-20 membered heteroaryl, wherein the 3-10 membered saturated heterocyclyl or 5-20 membered heteroaryl of R⁶ is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and —O(R^{g}),
then L² is absent or is *—CH₂—O—**, —CH═CH—, or —C≡C—, wherein ** indicates the attachment point to the R⁶ moiety and * indicates the attachment point to the remainder of the molecule, and
(iii) when R⁷ is taken together with the carbon atom of *—CH₂—O—** of L², and the atoms to which they are attached, to form a C₆aryl or a 6-membered heteroaryl, and
   R⁵ is

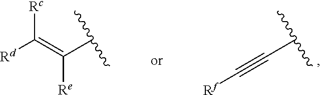

then R⁶ is 3-10 membered saturated heterocyclyl or 5-20 membered heteroaryl, wherein the 3-10 membered saturated heterocyclyl or 5-20 membered heteroaryl is independently optionally substituted with one or two substituents independently selected from the group consisting of —CN, halo, C₁₋₆alkyl, C₁₋₆haloalkyl, C₃₋₁₀cycloalkyl, —NO₂, —N(R^{g})(R^{h}), and O(R⁹);
R⁸ is H or C₁₋₆alkyl, wherein the C₁₋₆alkyl is optionally substituted with one or more —OH; and
R^{g} and R^{h} are, independently of each other and independently at each occurrence, selected from the group consisting of H, —CN, —OH, C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₆₋₂₀aryl, and 3-20 membered heteroaryl of R^{g} and R^{h} are each independently optionally substituted with one or more substituents independently selected from the group consisting of C₁₋₆alkyl, C₁₋₆haloalkyl, C₁₋₆alkoxy, oxo, —CN, halo, —NO₂, and —OH; or
(c) a compound of formula (III):

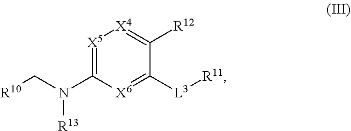

or a stereoisomer or tautomer thereof, or a pharmaceutically acceptable salt of any of the foregoing, wherein:
X⁴ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ), or
the R¹⁴ of X⁴ is taken together with R¹², and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl;
X⁵ is N or C—R¹⁴, wherein each R¹⁴ is independently selected from the group consisting of H, —CN, halo, —C(O)—NH₂, —N(R^{m})(Rⁿ), C₃₋₁₀cycloalkyl, C₁₋₆alkoxy, C₆₋₂₀aryl, and C₁₋₆alkyl, wherein the C₁₋₆alkyl of R¹⁴ is optionally substituted with one or more —OH or —N(R^{m})(Rⁿ);
X₆ is N or C—H;
R¹⁰ is:
   (i) a 3-5 membered saturated heterocyclyl comprising at least one annular oxygen atom, wherein the 3-5 membered saturated heterocyclyl is optionally substituted with one or more C₁₋₆alkyl, or
   (ii) —N(R^{m})(Rⁿ), or
   (iii)

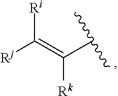

wherein Rⁱ, R^{j}, and R^{k} are each independently selected from the group consisting of H, halo, —CN, —OH, —B(OH)₂, —C(O)—OH,
—C(O)—N(R^{m})(Rⁿ), —C(O)—C₁₋₆alkoxy, —C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆alkyl of Rⁱ, R^{j}, or R^{k} is optionally substituted with one or more —OH, or
(iv)

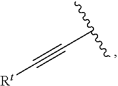

wherein R^{t} is selected from the group consisting of H, halo, —CN, —OH, —B(OH)₂, —C(O)—OH, —C(O)—N(R^{m})(Rⁿ), —C(O)—C₁₋₆alkoxy,
—C(O)—C₁₋₆alkyl, C₁₋₆alkyl, C₃₋₁₀cycloalkyl, C₆₋₂₀aryl, 3-10 membered heterocyclyl, C₅₋₁₃spirocyclyl, and 5-20 membered heteroaryl, wherein the C₁₋₆ alkyl is optionally substituted with one or more —OH;
L³ is absent or is selected from the group consisting of —O—, *—CH₂—O—**, *—O—CH₂—**, —CH═CH—, and —C≡C—, wherein ** indicates the attachment point to the R¹¹ moiety and * indicates the attachment point to the remainder of the molecule;
R¹¹ is C₁₋₁₂alkyl, C₃₋₁₀cycloalkyl, 3-10 membered heterocyclyl, C₅₋₁₃spirocyclyl, C₆₋₂₀aryl, or 5-20 membered heteroaryl,
   wherein the C₁₋₁₂alkyl, C₃₋₁₀cycloalkyl, 3-10 membered saturated heterocyclyl, C₅₋₁₃spirocyclyl, C₆₋₂₀aryl, or 5-20 membered heteroaryl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}),
   provided that, when R¹¹ is C₁₋₁₂alkyl, wherein the C₁₋₁₂alkyl of R¹¹ is independently optionally substituted with one or more substituents selected from the group consisting of —CN, halo, C₁₋₆alkyl, C₁₋₆haloalkyl, C₃₋₁₀cycloalkyl, —NO₂, —N(R^{m})(Rⁿ), and —O(R^{m}), then L³ is —CH═CH— or —C≡C—;
R¹² is —CN, C₁₋₆alkyl, C₁₋₄alkoxy, or C₂₋₄alkenyl, wherein the C₂₋₄alkenyl is optionally substituted with —N(R^{m})(Rⁿ), or
R¹² is taken together with R¹⁴ of X⁴, and the atoms to which they are attached, to form a 5-membered heterocyclyl or a 5-membered heteroaryl, wherein the 5-membered heterocyclyl or 5-membered heteroaryl is optionally substituted with one or more C₁₋₆alkyl, or
R¹² is taken together with the carbon atom of *—CH₂—O—** of L³, and the atoms to which they are attached, to form a C₆aryl or a 6-membered heteroaryl; R¹³ is H or C₁₋₆alkyl, wherein the C₁₋₆alkyl of R¹³ is optionally substituted with one or more —OH; and
R^{m} and Rⁿ are, independently of each other and independently at each occurrence, selected from the group consisting of H, —CN, —OH, C₁₋₆alkyl, C₂-6alkenyl, C₂₋₆alkynyl, C₃₋₁₀cycloalkyl, —C₁₋₆alkyl-C₃₋₁₀cycloalkyl, 3-10 membered heterocyclyl, C₅₋₁₃spirocyclyl, C₆₋₂₀aryl, and 5-20 membered heteroaryl, wherein the C₁₋₆alkyl, C₂₋₆alkenyl, C₂₋₆alkynyl, C₃₋₁₀cycloalkyl, C₁₋₆alkyl-C₃₋₁₀cycloalkyl, 3-10 membered heterocyclyl, C₅₋₁₃spirocyclyl, C₆₋₂₀aryl, and 5-20 membered heteroaryl of R^{m} and Rⁿ are each independently optionally substituted with one or more substituents independently selected from the group consisting of C₁₋₆alkyl, C₁₋₆haloalkyl, C₁₋₆alkoxy, oxo, —CN, halo, —NO₂, and —OH,
or any combination thereof.
Embodiment 43. The kit of embodiment 42, wherein the one or more YAP/TAZ-TEAD inhibitors comprise a compound of formula (I), or a stereoisomer or tautomer thereof, or a pharmaceutically acceptable salt of any of the foregoing.
Embodiment 44. The kit of embodiment 43, wherein the one or more YAP/TAZ-TEAD inhibitors comprise

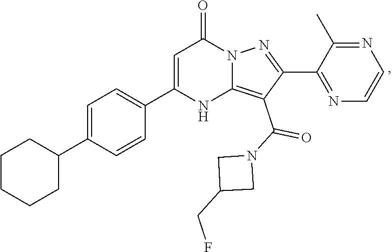 [SMILES: Cc1nccnc1-c1nn2c(=O)cc(-c3ccc(C4CCCCC4)cc3)[nH]c2c1C(=O)N1CC(CF)C1]

or a stereoisomer or tautomer thereof, or a pharmaceutically acceptable salt of any of the foregoing.
Embodiment 45. The kit of any one of embodiments 42-44, wherein the one or more YAP/TAZ-TEAD inhibitors comprise a compound of formula (II), or a stereoisomer or tautomer thereof, or a pharmaceutically acceptable salt of any of the foregoing.
Embodiment 46. The kit of embodiment 45, wherein, for the compound of formula (II), R⁵ is oxiranyl, wherein the oxiranyl of R⁵ is optionally substituted with one or more C₁₋₆alkyl, wherein the C₁₋₆alkyl is optionally substituted with one or more —C(O)—NH₂.
Embodiment 47. The kit of embodiment 45 or embodiment 46, wherein the one or more YAP/TAZ-TEAD inhibitors comprise

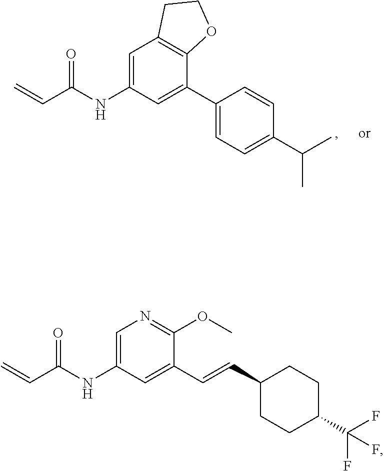

or both, or a stereoisomer or tautomer thereof, or a pharmaceutically acceptable salt of any of the foregoing.
Embodiment 48. The kit of any one of embodiments 42-47, wherein the one or more YAP/TAZ-TEAD inhibitors comprise a compound of formula (III), or a stereoisomer or tautomer thereof, or a pharmaceutically acceptable salt of any of the foregoing.
Embodiment 49. The kit of embodiment 48, wherein, for the compound of formula (III), R¹⁰ is oxiranyl, wherein the oxiranyl of R¹⁰ is optionally substituted with one or more C₁₋₆alkyl.
Embodiment 50. The kit of embodiment 48 or embodiment 49, wherein the one or more YAP/TAZ-TEAD inhibitors comprise

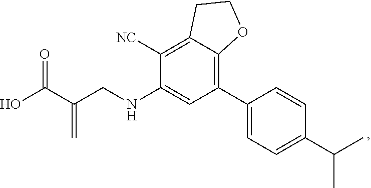 [SMILES: C=C(CNc1cc(-c2ccc(C(C)C)cc2)c2c(c1C#N)CCO2)C(=O)O]

or a stereoisomer or tautomer thereof, or a pharmaceutically acceptable salt of any of the foregoing.
Embodiment 51. The kit of any one of embodiments 38-50, wherein the one or more KRAS inhibitors comprise one or more inhibitors of the KRAS G12C mutant.
Embodiment 52. The kit of any one of embodiments 38-51, wherein the one or more KRAS inhibitors comprise

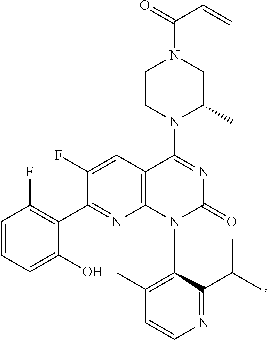

or a stereoisomer or tautomer thereof, or a pharmaceutically acceptable salt of any of the foregoing.
Embodiment 53. The kit of any one of embodiments 38-52, wherein the one or more KRAS inhibitors comprise

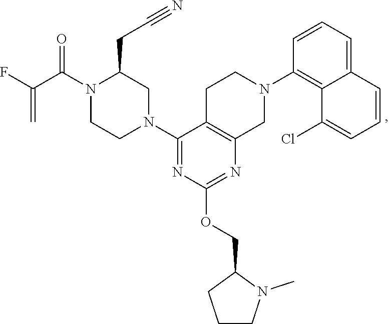 [SMILES: C=C(F)C(=O)N1CCN(c2nc(OC[C@@H]3CCCN3C)nc3c2CCN(c2cccc4cccc(Cl)c24)C3)C[C@@H]1CC#N]

or a stereoisomer or tautomer thereof, or a pharmaceutically acceptable salt of any of the foregoing.
Embodiment 54. The kit of any one of embodiments 38-53, wherein the one or more KRAS inhibitors comprise

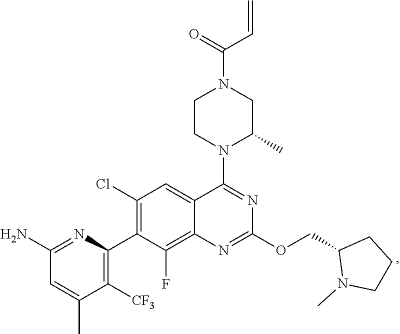

or a stereoisomer or tautomer thereof, or a pharmaceutically acceptable salt of any of the foregoing.
Embodiment 55. The kit of any one of embodiments 38-54, wherein the combination further comprises one or more pharmaceutically acceptable excipients.
Embodiment 56. The kit of any one of embodiments 38-55, wherein the one or more YAP/TAZ-TEAD inhibitors and the one or more KRAS inhibitors are administered simultaneously.
Embodiment 57. The kit of embodiment 56, wherein the one or more YAP/TAZ-TEAD inhibitors and the one or more KRAS inhibitors are formulated together in a single composition.
Embodiment 58. The kit of embodiment 56, wherein the one or more YAP/TAZ-TEAD inhibitors and the one or more KRAS inhibitors are formulated in separate compositions.
Embodiment 59. The kit of any one of embodiments 38-55, wherein the one or more YAP/TAZ-TEAD inhibitors and the one or more KRAS inhibitors are administered sequentially.
Embodiment 60. Use of a composition of any one of embodiments 23-37 in the manufacture of a medicament for use in the treatment of cancer.
Embodiment 61. The use of embodiment 60, wherein the cancer is selected from the group consisting of hematological cancer, pancreatic cancer, MYH-associated polyposis, colorectal cancer, and lung cancer.
Embodiment 62. The use of embodiment 60, wherein the cancer is lung cancer.
Embodiment 63. The use of embodiment 62, wherein the lung cancer is non-small-cell lung cancer.
Embodiment 64. A composition of any one of embodiments 23-37 for use in the treatment of cancer.
Embodiment 65. The composition of embodiment 64, wherein the cancer is selected from the group consisting of hematological cancer, pancreatic cancer, MYH-associated polyposis, colorectal cancer, and lung cancer.
Embodiment 66. The composition of embodiment 64, wherein the cancer is lung cancer.
Embodiment 67. The composition of embodiment 66, wherein the lung cancer is non-small-cell lung cancer.
Embodiment 68. A process for preparing a composition, comprising: (i) one or more YAP/TAZ-TEAD inhibitors; and (ii) one or more KRAS inhibitors.
Embodiment 69. A composition prepared by the process of embodiment 68.
Embodiment 70. A method of treating cancer in a subject in need thereof, comprising administering to the subject an effective amount of a combination, comprising:
   (i) one or more YAP-TAZ-TEAD inhibitors selected from the group consisting of

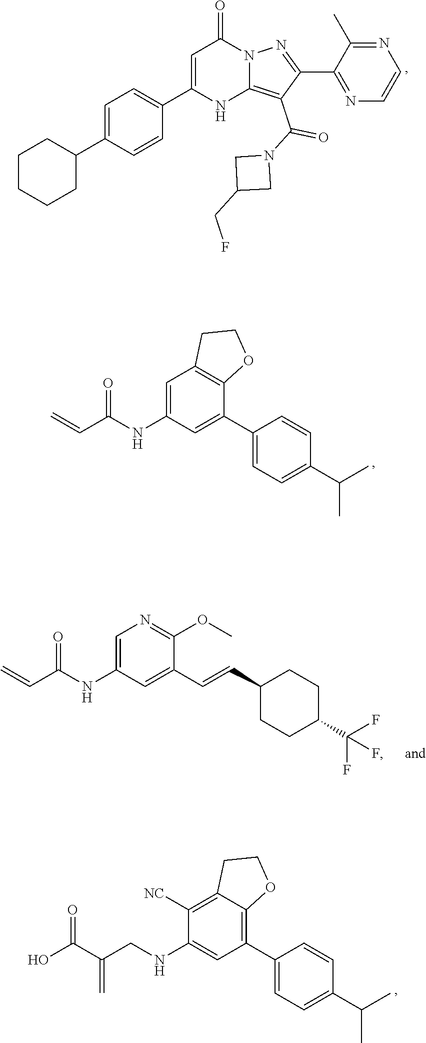

or a stereoisomer or tautomer thereof, or a pharmaceutically acceptable salt of any of the foregoing; and
(ii) one or more KRAS inhibitors selected from the group consisting of

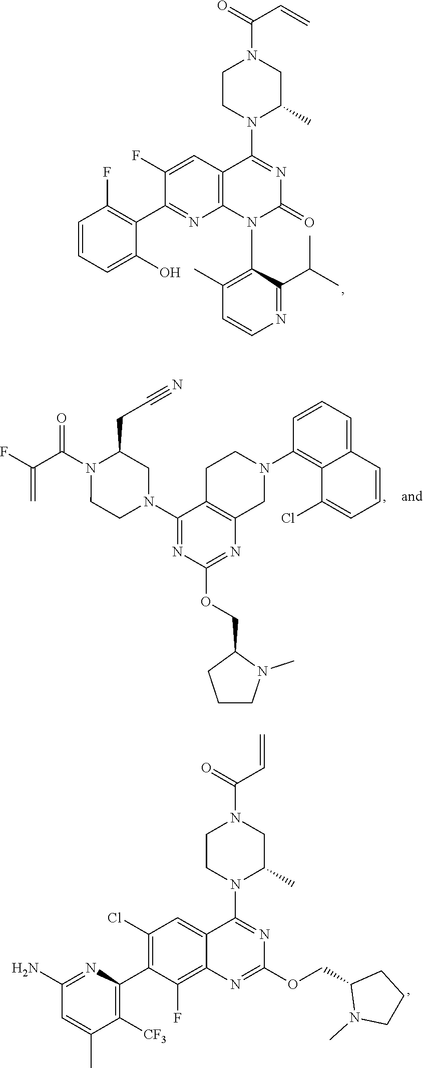

or a stereoisomer or tautomer thereof, or a pharmaceutically acceptable salt of any of the foregoing.
Embodiment 71. A composition, comprising:
   (i) one or more YAP-TAZ-TEAD inhibitors selected from the group consisting of

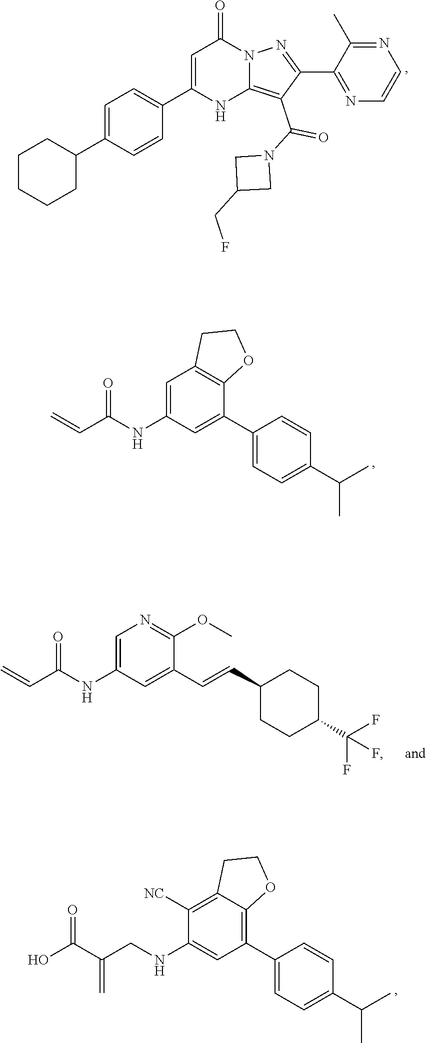

or a stereoisomer or tautomer thereof, or a pharmaceutically acceptable salt of any of the foregoing; and
(ii) one or more KRAS inhibitors selected from the group consisting of

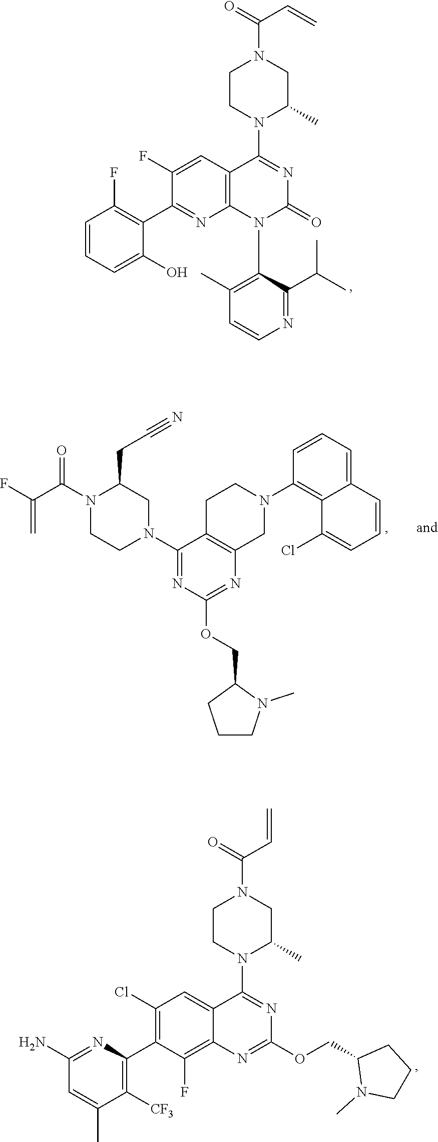

or a stereoisomer or tautomer thereof, or a pharmaceutically acceptable salt of any of the foregoing.
Embodiment 72. A kit, comprising:
   (i) one or more YAP-TAZ-TEAD inhibitors selected from the group consisting of

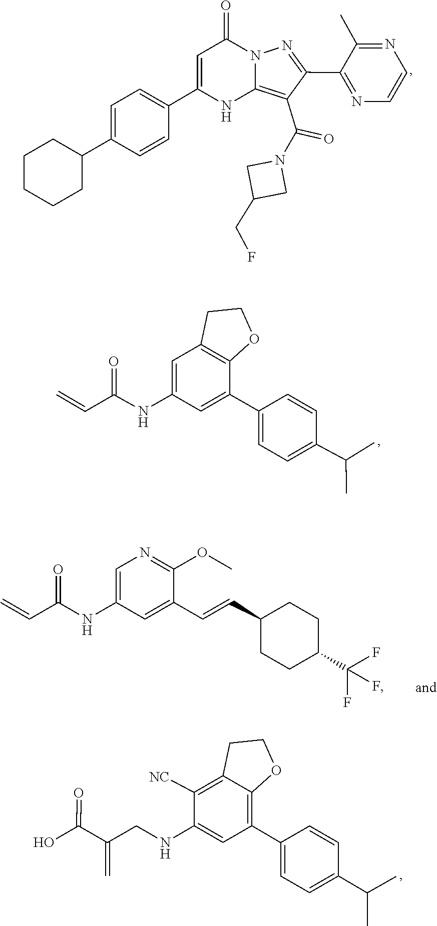

stereoisomer or tautomer thereof, or a pharmaceutically acceptable salt of any of the foregoing;
(ii) one or more KRAS inhibitors selected from the group consisting of

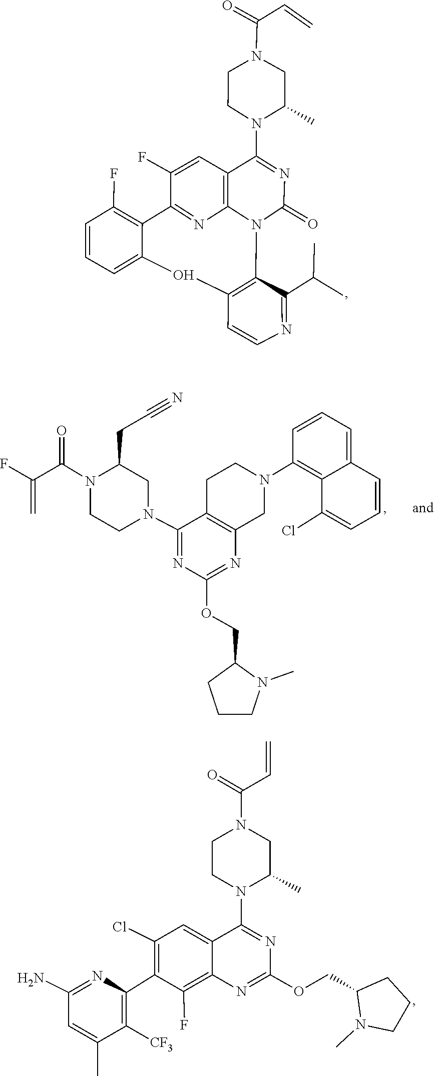

or a stereoisomer or tautomer thereof, or a pharmaceutically acceptable salt of any of the foregoing; and
(iii) instructions for administering the combination to treat cancer in a subject in need thereof.
Embodiment 73. The disclosure as described hereinbefore.

1. A method of modulating YAP/TAZ-TEAD activity, or KRAS activity, or both, in a cell, comprising administering to the cell an effective amount of a combination, comprising: (i) one or more YAP/TAZ-TEAD inhibitors; and (ii) one or more KRAS inhibitors.

2. A method of inhibiting YAP/TAZ-TEAD activity, or KRAS activity, or both, in a cell, comprising administering to the cell an effective amount of a combination, comprising: (i) one or more YAP/TAZ-TEAD inhibitors; and (ii) one or more KRAS inhibitors.

3. A method of treating cancer in a subject in need thereof, comprising administering to the subject an effective amount of a combination, comprising: (i) one or more YAP/TAZ-TEAD inhibitors; and (ii) one or more KRAS inhibitors.

4. The method of claim 3, wherein the cancer is selected from the group consisting of hematological cancer, pancreatic cancer, MYH-associated polyposis, colorectal cancer, and lung cancer.

5. The method of claim 3, wherein the cancer is lung cancer.

6. The method of claim 5, wherein the lung cancer is non-small-cell lung cancer.

7. The method of any one of claims 1-6, wherein the one or more YAP/TAZ-TEAD inhibitors comprise
a compound of formula (I), or a stereoisomer or tautomer thereof, or a pharmaceutically acceptable salt of any of the foregoing;
a compound of formula (II), or a stereoisomer or tautomer thereof, or a pharmaceutically acceptable salt of any of the foregoing;
a compound of formula (III), or a stereoisomer or tautomer thereof, or a pharmaceutically acceptable salt of any of the foregoing;
a compound of formula (IV), or a stereoisomer or tautomer thereof, or a pharmaceutically acceptable salt of any of the foregoing;
a compound of formula (V), or a stereoisomer or tautomer thereof, or a pharmaceutically acceptable salt of any of the foregoing;
a compound of formula (VI), or a stereoisomer or tautomer thereof, or a pharmaceutically acceptable salt of any of the foregoing;
a compound of formula (VII), or a stereoisomer or tautomer thereof, or a pharmaceutically acceptable salt of any of the foregoing; or
a compound of formula (VIII), or a stereoisomer or tautomer thereof, or a pharmaceutically acceptable salt of any of the foregoing,
or any combination thereof.

8. The method of claim 7, wherein the one or more YAP/TAZ-TEAD inhibitors comprise a compound of formula (I), or a stereoisomer or tautomer thereof, or a pharmaceutically acceptable salt of any of the foregoing.

9. The method of claim 8, wherein the one or more YAP/TAZ-TEAD inhibitors comprise 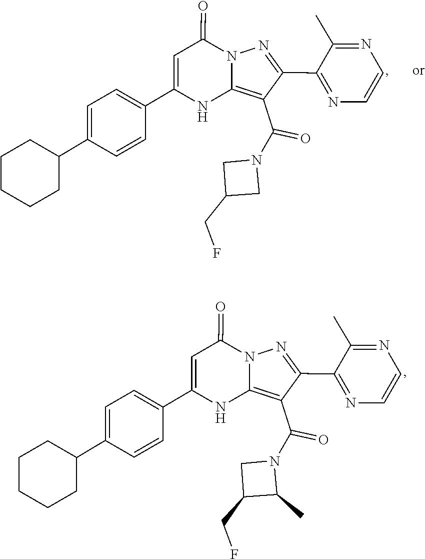 [SMILES: Cc1nccnc1-c1nn2c(=O)cc(-c3ccc(C4CCCCC4)cc3)[nH]c2c1C(=O)N1CC(CF)C1 ; Cc1nccnc1-c1nn2c(=O)cc(-c3ccc(C4CCCCC4)cc3)[nH]c2c1C(=O)N1C[C@H](CF)[C@@H]1C]
or a stereoisomer or tautomer thereof, or a pharmaceutically acceptable salt of any of the foregoing.

10. The method of claim 7, wherein the one or more YAP/TAZ-TEAD inhibitors comprise a compound of formula (II), or a stereoisomer or tautomer thereof, or a pharmaceutically acceptable salt of any of the foregoing.

11. The method of claim 10, wherein the one or more YAP/TAZ-TEAD inhibitors comprise 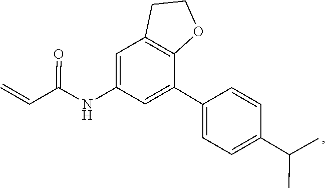 [SMILES: C=CC(=O)Nc1cc2c(c(-c3ccc(C(C)C)cc3)c1)OCC2]
or a stereoisomer or tautomer thereof, or a pharmaceutically acceptable salt of any of the foregoing.

12. The method of claim 10, wherein the one or more YAP/TAZ-TEAD inhibitors comprise 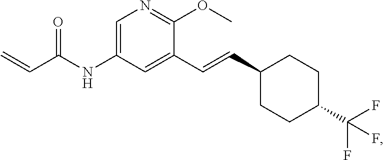 [SMILES: C=CC(=O)Nc1cnc(OC)c(/C=C/[C@H]2CC[C@H](C(F)(F)F)CC2)c1]
or a stereoisomer or tautomer thereof, or a pharmaceutically acceptable salt of any of the foregoing.

13. The method of claim 7, wherein the one or more YAP/TAZ-TEAD inhibitors comprise a compound of formula (III), or a stereoisomer or tautomer thereof, or a pharmaceutically acceptable salt of any of the foregoing.

14. The method of claim 13, wherein the one or more YAP/TAZ-TEAD inhibitors comprise 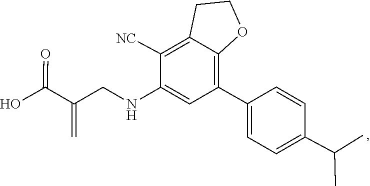 [SMILES: C=C(CNc1cc(-c2ccc(C(C)C)cc2)c2c(c1C#N)CCO2)C(=O)O]
or a stereoisomer or tautomer thereof, or a pharmaceutically acceptable salt of any of the foregoing.

15. The method of claim 7, wherein the one or more YAP/TAZ-TEAD inhibitors comprise a compound of formula (IV), or a stereoisomer or tautomer thereof, or a pharmaceutically acceptable salt of any of the foregoing.

16. The method of claim 15, wherein the one or more YAP/TAZ-TEAD inhibitors comprise 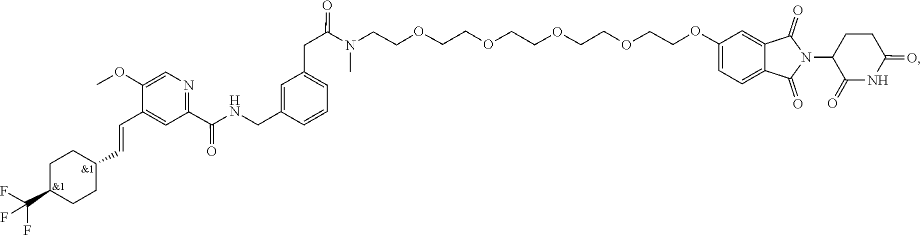 [SMILES: COc1cnc(C(=O)NCc2cccc(CC(=O)N(C)CCOCCOCCOCCOCCOc3ccc4c(c3)C(=O)N(C3CCC(=O)NC3=O)C4=O)c2)cc1/C=C/[C@H]1CC[C@H](C(F)(F)F)CC1]
or a stereoisomer or tautomer thereof, or a pharmaceutically acceptable salt of any of the foregoing.

17. The method of claim 7, wherein the one or more YAP/TAZ-TEAD inhibitors comprise a compound of formula (V), or a stereoisomer or tautomer thereof, or a pharmaceutically acceptable salt of any of the foregoing.

18. The method of claim 17, wherein the one or more YAP/TAZ-TEAD inhibitors comprise 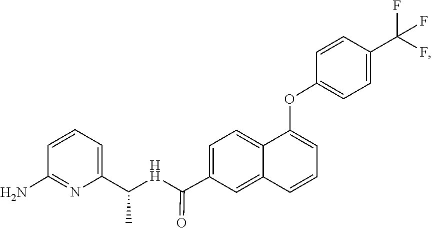
or a stereoisomer or tautomer thereof, or a pharmaceutically acceptable salt of any of the foregoing.

19. The method of claim 7, wherein the one or more YAP/TAZ-TEAD inhibitors comprise a compound of formula (VI), or a stereoisomer or tautomer thereof, or a pharmaceutically acceptable salt of any of the foregoing.

20. The method of claim 19, wherein the one or more YAP/TAZ-TEAD inhibitors comprise 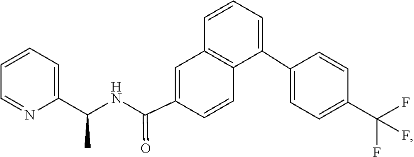 [SMILES: C[C@H](NC(=O)c1ccc2c(-c3ccc(C(F)(F)F)cc3)cccc2c1)c1ccccn1]
or a stereoisomer or tautomer thereof, or a pharmaceutically acceptable salt of any of the foregoing.

21. The method of claim 7, wherein the one or more YAP/TAZ-TEAD inhibitors comprise a compound of formula (VII), or a stereoisomer or tautomer thereof, or a pharmaceutically acceptable salt of any of the foregoing.

22. The method of claim 21, wherein the one or more YAP/TAZ-TEAD inhibitors comprise 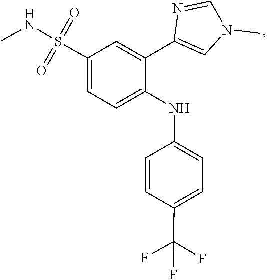
or a stereoisomer or tautomer thereof, or a pharmaceutically acceptable salt of any of the foregoing.

23. The method of claim 7, wherein the one or more YAP/TAZ-TEAD inhibitors comprise a compound of formula (VIII), or a stereoisomer or tautomer thereof, or a pharmaceutically acceptable salt of any of the foregoing.

24. The method of claim 23, wherein the one or more YAP/TAZ-TEAD inhibitors comprise 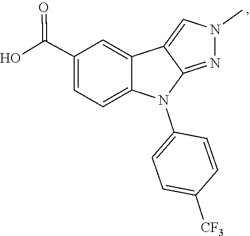 [SMILES: Cn1cc2c3cc(C(=O)O)ccc3n(-c3ccc(C(F)(F)F)cc3)c2n1]
or a stereoisomer or tautomer thereof, or a pharmaceutically acceptable salt of any of the foregoing.

25. The method of any one of claims 1-24, wherein the one or more KRAS inhibitors comprise one or more inhibitors of the KRAS G12C mutant.

26. The method of any one of claims 1-25, wherein the one or more KRAS inhibitors comprise:
a compound of formula (K-I), or a stereoisomer or tautomer thereof, or a pharmaceutically acceptable salt of any of the foregoing;
a compound of formula (K-II), or a stereoisomer or tautomer thereof, or a pharmaceutically acceptable salt of any of the foregoing;
a compound of formula (K-III), or a stereoisomer or tautomer thereof, or a pharmaceutically acceptable salt of any of the foregoing; or
a compound of formula (KIV), or a stereoisomer or tautomer thereof, or a pharmaceutically acceptable salt of any of the foregoing,
or any combination thereof.

27. The method of claim 26, wherein the one or more KRAS inhibitors comprise a compound of formula (K-I), or a stereoisomer or tautomer thereof, or a pharmaceutically acceptable salt of any of the foregoing.

28. The method of claim 27, wherein the one or more KRAS inhibitors comprise 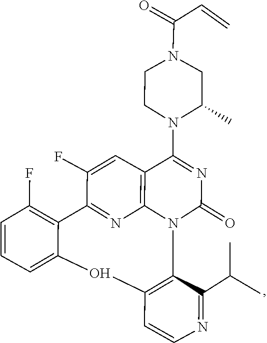
or a stereoisomer or tautomer thereof, or a pharmaceutically acceptable salt of any of the foregoing.

29. The method of claim 26, wherein the one or more KRAS inhibitors comprise a compound of formula (K-II), or a stereoisomer or tautomer thereof, or a pharmaceutically acceptable salt of any of the foregoing.

30. The method of claim 29, wherein the one or more KRAS inhibitors comprise 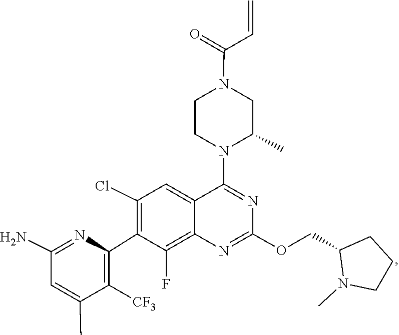
or a stereoisomer or tautomer thereof, or a pharmaceutically acceptable salt of any of the foregoing.

31. The method of claim 26, wherein the one or more KRAS inhibitors comprise a compound of formula (K-III), or a stereoisomer or tautomer thereof, or a pharmaceutically acceptable salt of any of the foregoing.

32. The method of claim 31, wherein the one or more KRAS inhibitors comprise 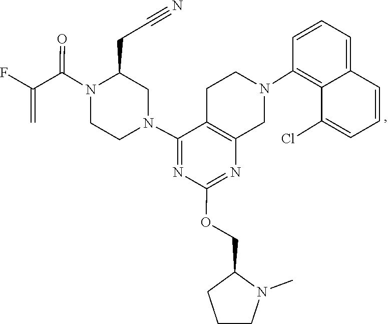 [SMILES: C=C(F)C(=O)N1CCN(c2nc(OC[C@@H]3CCCN3C)nc3c2CCN(c2cccc4cccc(Cl)c24)C3)C[C@@H]1CC#N]
or a stereoisomer or tautomer thereof, or a pharmaceutically acceptable salt of any of the foregoing.

33. The method of claim 26, wherein the one or more KRAS inhibitors comprise a compound of formula (K-IV), or a stereoisomer or tautomer thereof, or a pharmaceutically acceptable salt of any of the foregoing.

34. The method of claim 33, wherein the one or more KRAS inhibitors comprise 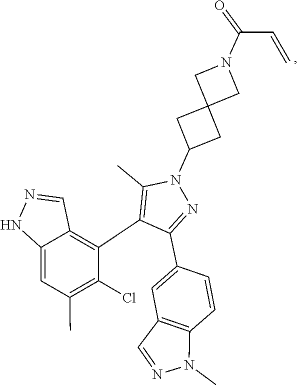 [SMILES: C=CC(=O)N1CC2(CC(n3nc(-c4ccc5c(cnn5C)c4)c(-c4c(Cl)c(C)cc5[nH]ncc45)c3C)C2)C1]
or a stereoisomer or tautomer thereof, or a pharmaceutically acceptable salt of any of the foregoing.

35. The method of any one of claims 1-34, wherein the combination further comprises one or more pharmaceutically acceptable excipients.

36. The method of any one of claims 1-35, wherein the one or more YAP/TAZ-TEAD inhibitors and the one or more KRAS inhibitors are administered simultaneously.

37. The method of any one of claims 1-35, wherein the one or more YAP/TAZ-TEAD inhibitors and the one or more KRAS inhibitors are administered sequentially.

38. A composition, comprising: (i) one or more YAP/TAZ-TEAD inhibitors; and (ii) one or more KRAS inhibitors.

39. The composition of claim 38, wherein the one or more YAP/TAZ-TEAD inhibitors comprise a compound of formulae (I), (II), (III), (IV), (V), (VI), (VII), or (VIII), or a stereoisomer or tautomer thereof, or a pharmaceutically acceptable salt of any of the foregoing, or any combination thereof.

40. The composition of claims 38 or 39, wherein the one or more KRAS inhibitors comprise one or more inhibitors of the KRAS G12C mutant.

41. The composition of any one of claims 38-40, wherein the one or more KRAS inhibitors comprise a compound of formulae (K-I), (K-II), (K-III), or (K-IV), or a stereoisomer or tautomer thereof, or a pharmaceutically acceptable salt of any of the foregoing, or any combination thereof.

42. The composition of any one of claims 38-41, wherein the composition further comprises one or more pharmaceutically acceptable excipients.

43. A kit, comprising (i) an effective amount of a combination comprising one or more YAP/TAZ-TEAD inhibitors and one or more KRAS inhibitors; and (ii) instructions for administering the combination to treat cancer in a subject in need thereof.

44. The kit of claim 43, wherein the cancer is selected from the group consisting of hematological cancer, pancreatic cancer, MYH-associated polyposis, colorectal cancer, and lung cancer.

45. The kit of claim 43, wherein the cancer is lung cancer.

46. The kit of claim 45, wherein the lung cancer is non-small-cell lung cancer.

47. The kit of any one of claims 43-46, wherein one or more YAP/TAZ-TEAD inhibitors comprise a compound of formulae (I), (II), (III), (IV), (V), (VI), (VII), or (VIII), or a stereoisomer or tautomer thereof, or a pharmaceutically acceptable salt of any of the foregoing, or any combination thereof.

48. The kit of any one of claims 43-47, wherein the one or more KRAS inhibitors comprise one or more inhibitors of the KRAS G12C mutant.

49. The kit of any one of claims 43-48, wherein the one or more KRAS inhibitors comprise a compound of formulae (K-I), (K-II), (K-III), or (K-IV), or a stereoisomer or tautomer thereof, or a pharmaceutically acceptable salt of any of the foregoing, or any combination thereof.

50. The kit of any one of claims 43-49, wherein the combination further comprises one or more pharmaceutically acceptable excipients.

51. The kit of any one of claims 43-49, wherein the one or more YAP/TAZ-TEAD inhibitors and the one or more KRAS inhibitors are administered simultaneously.

52. The kit of claim 51, wherein the one or more YAP/TAZ-TEAD inhibitors and the one or more KRAS inhibitors are formulated together in a single composition.

53. The kit of claim 51, wherein the one or more YAP/TAZ-TEAD inhibitors and the one or more KRAS inhibitors are formulated in separate compositions.

54. The kit of any one of claims 43-49, wherein the one or more YAP/TAZ-TEAD inhibitors and the one or more KRAS inhibitors are administered sequentially.

55. Use of a composition of any one of claims 38-42 in the manufacture of a medicament for use in the treatment of cancer.

56. The use of claim 55, wherein the cancer is selected from the group consisting of hematological cancer, pancreatic cancer, MYH-associated polyposis, colorectal cancer, and lung cancer.

57. The use of claim 55, wherein the cancer is lung cancer.

58. The use of claim 57, wherein the lung cancer is non-small-cell lung cancer.

59. A composition of any one of claims 38-42 for use in the treatment of cancer.

60. The composition of claim 59, wherein the cancer is selected from the group consisting of hematological cancer, pancreatic cancer, MYH-associated polyposis, colorectal cancer, and lung cancer.

61. The composition of claim 60, wherein the cancer is lung cancer.

62. The composition of claim 61, wherein the lung cancer is non-small-cell lung cancer.

63. A process for preparing a composition, comprising: (i) one or more YAP/TAZ-TEAD inhibitors; and (ii) one or more KRAS inhibitors.

64. A composition prepared by the process of claim 63.

65. A method of treating cancer in a subject in need thereof, comprising administering to the subject an effective amount of a combination, comprising:
(i) one or more YAP-TAZ-TEAD inhibitors selected from the group consisting of 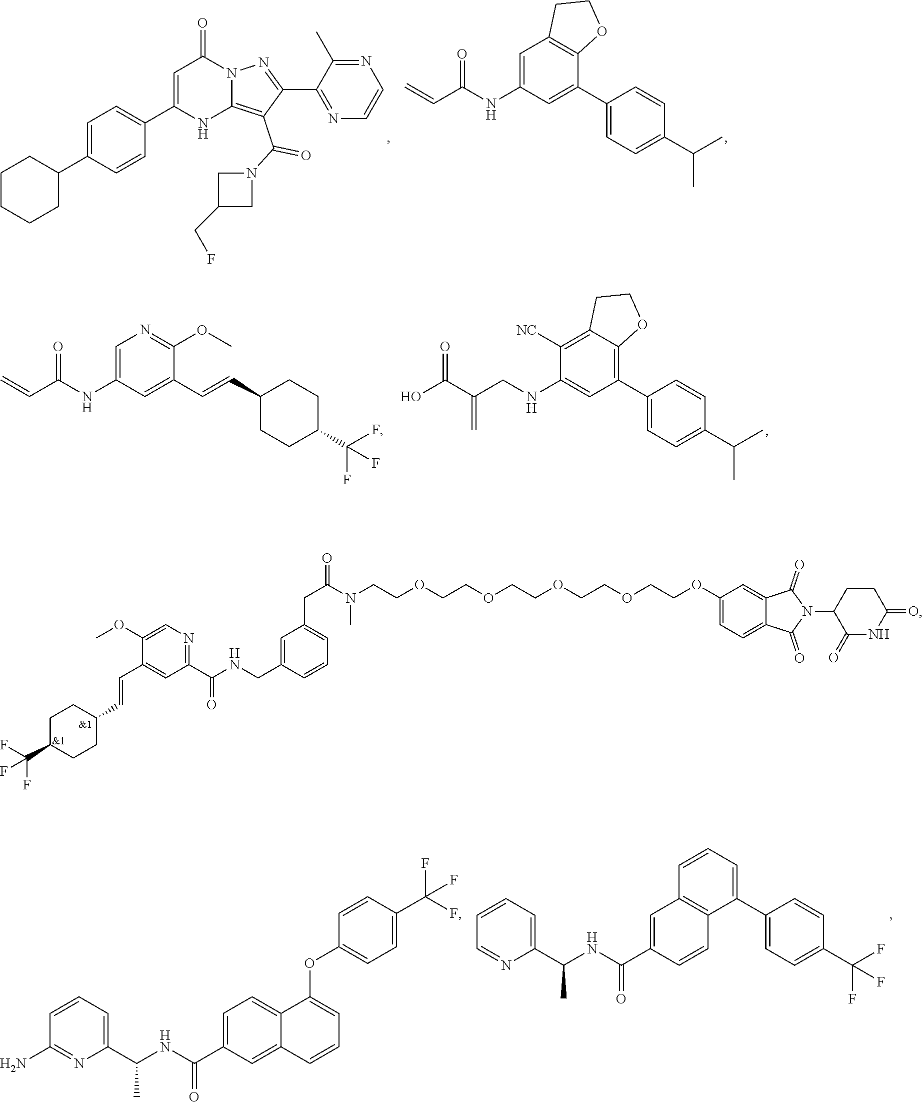 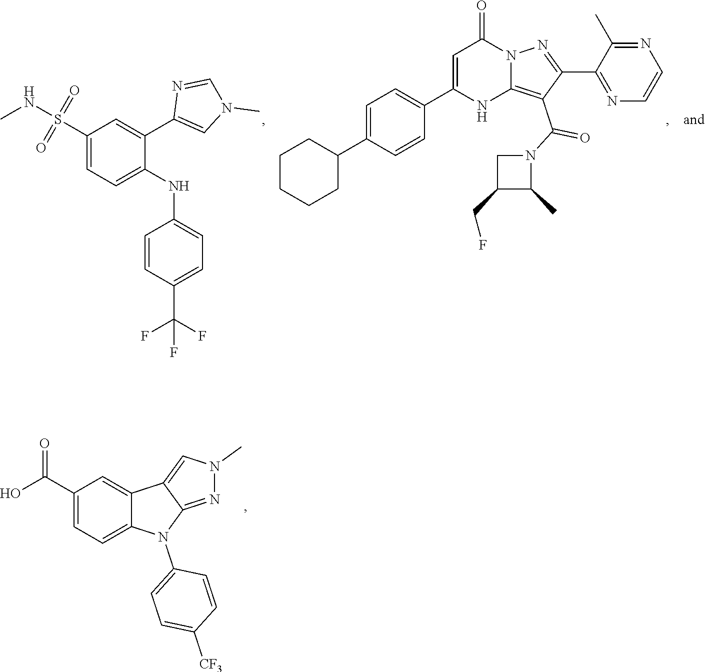
or a stereoisomer or tautomer thereof, or a pharmaceutically acceptable salt of any of the foregoing; and
(ii) one or more KRAS inhibitors selected from the group consisting of 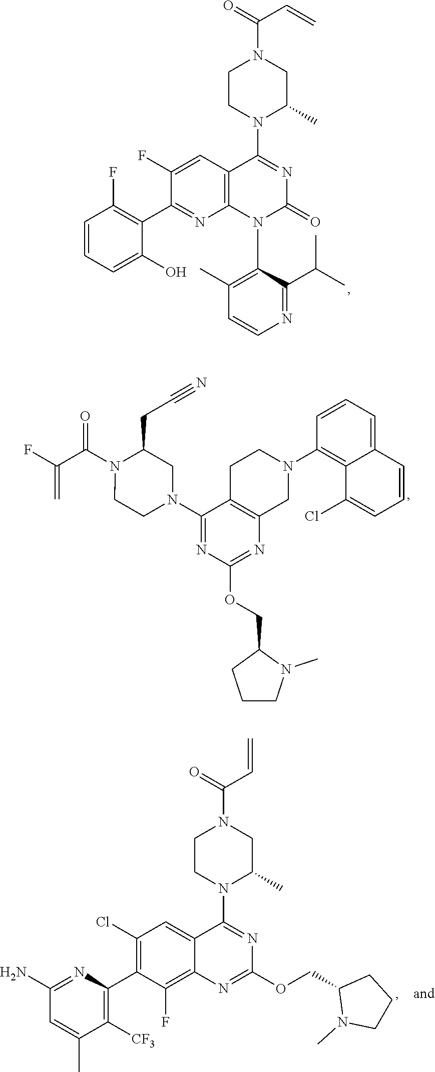 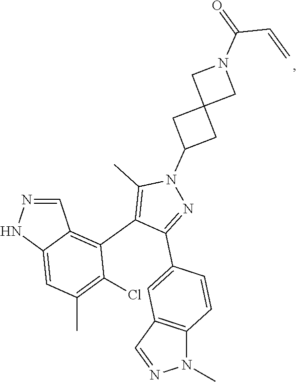 [SMILES: C=CC(=O)N1CC2(CC(n3nc(-c4ccc5c(cnn5C)c4)c(-c4c(Cl)c(C)cc5[nH]ncc45)c3C)C2)C1]
or a stereoisomer or tautomer thereof, or a pharmaceutically acceptable salt of any of the foregoing.

66. A composition, comprising:
(i) one or more YAP-TAZ-TEAD inhibitors selected from the group consisting of 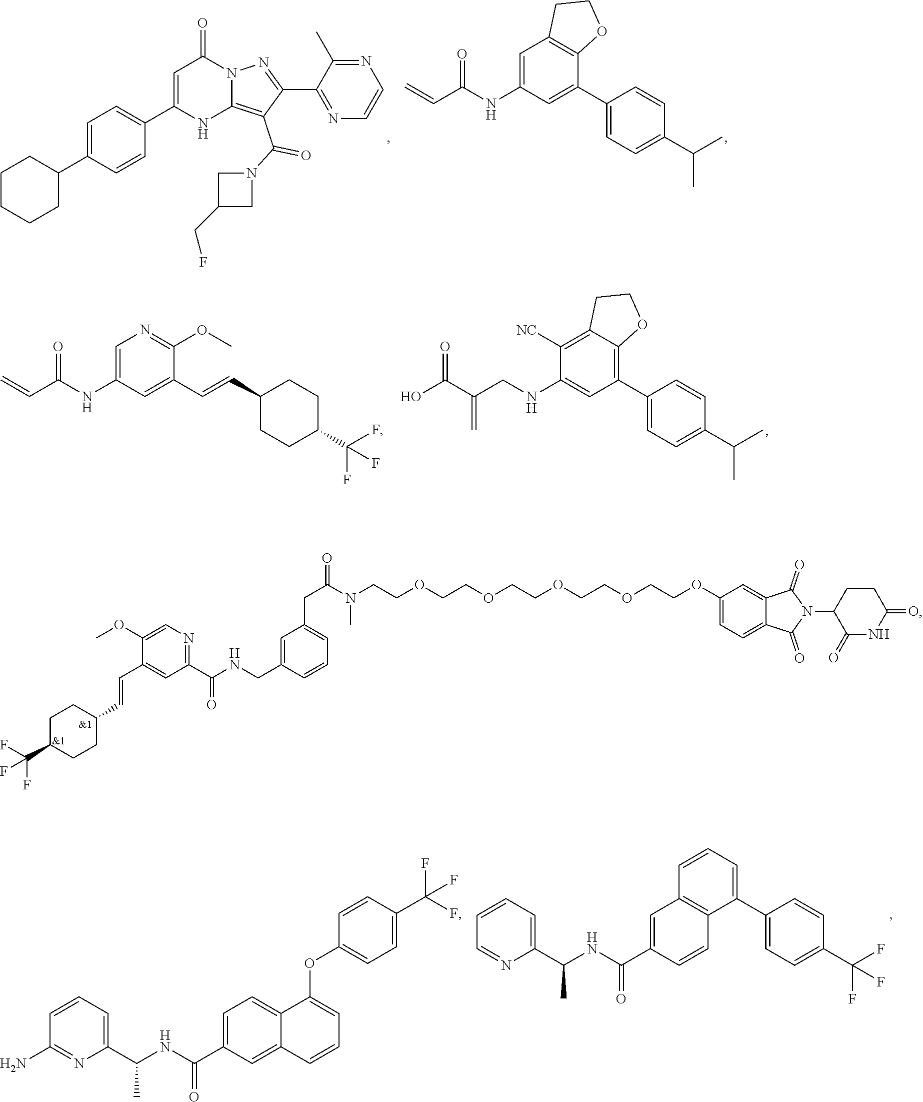 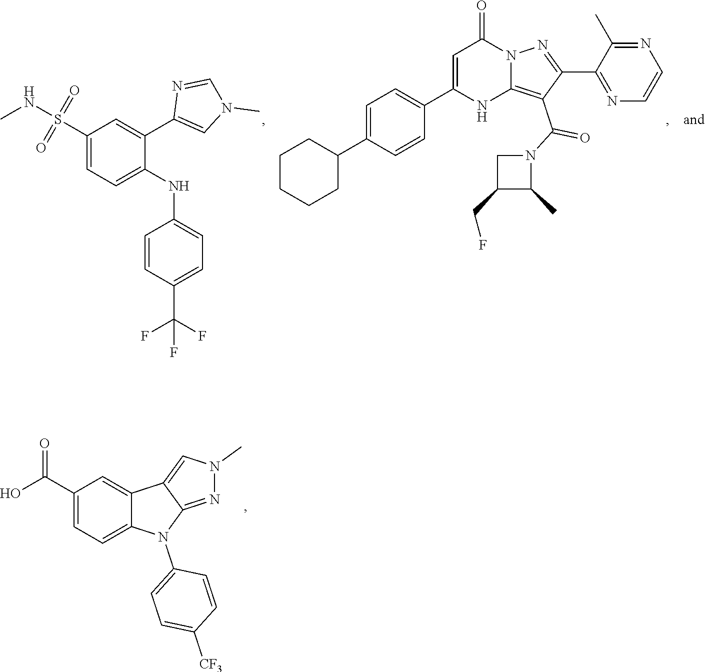
or a stereoisomer or tautomer thereof, or a pharmaceutically acceptable salt of any of the foregoing; and
(ii) one or more KRAS inhibitors selected from the group consisting of 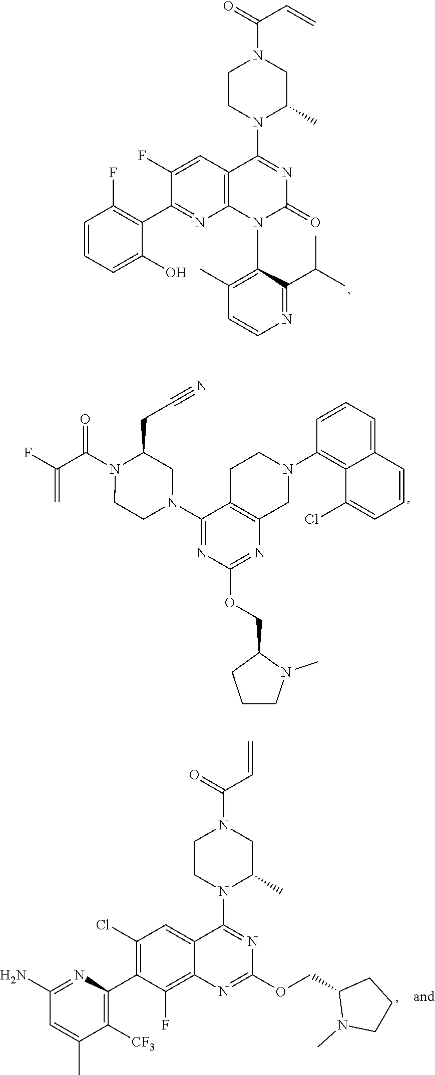 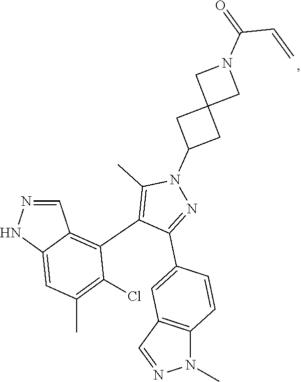 [SMILES: C=CC(=O)N1CC2(CC(n3nc(-c4ccc5c(cnn5C)c4)c(-c4c(Cl)c(C)cc5[nH]ncc45)c3C)C2)C1]
or a stereoisomer or tautomer thereof, or a pharmaceutically acceptable salt of any of the foregoing.

67. A kit, comprising:
(i) one or more YAP-TAZ-TEAD inhibitors selected from the group consisting of 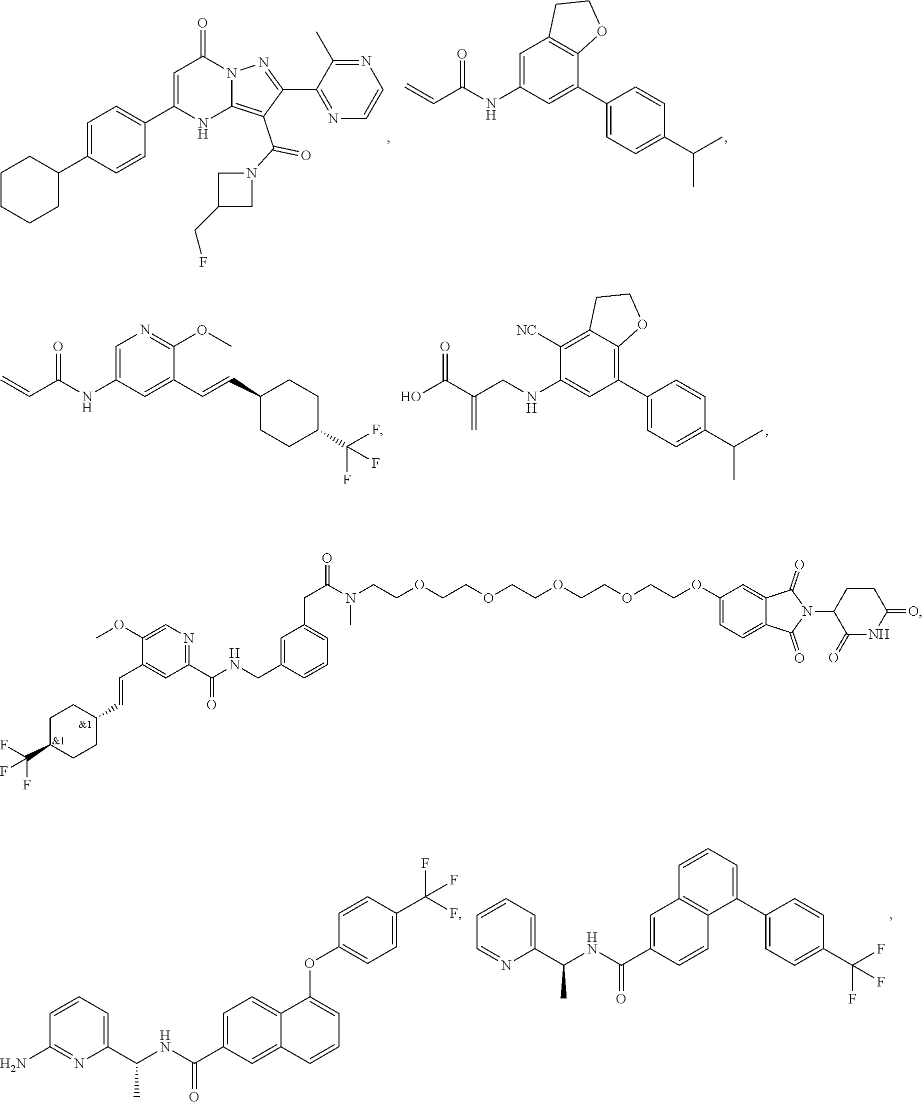 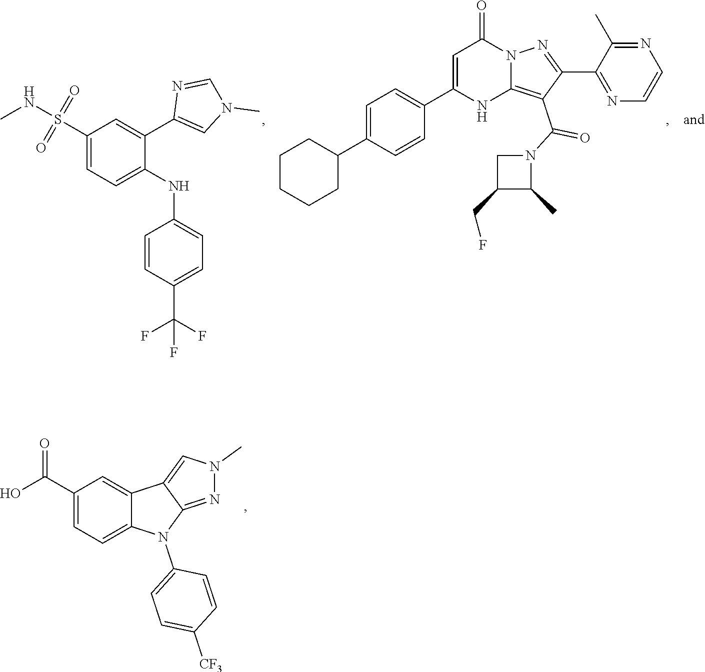
or a stereoisomer or tautomer thereof, or a pharmaceutically acceptable salt of any of the foregoing;
(ii) one or more KRAS inhibitors selected from the group consisting of 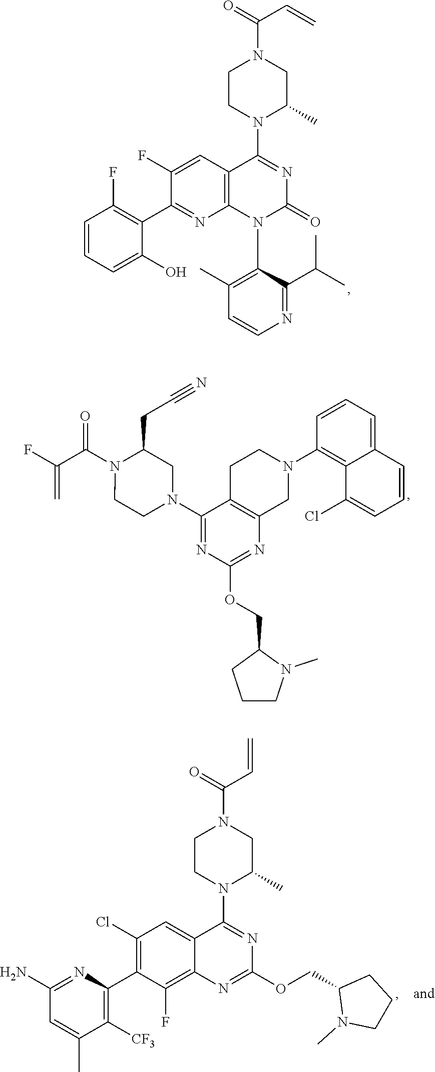 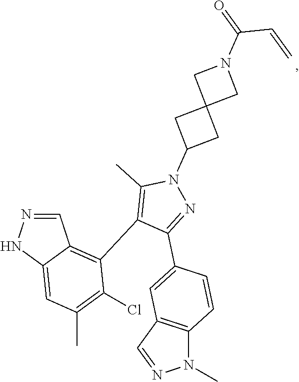 [SMILES: C=CC(=O)N1CC2(CC(n3nc(-c4ccc5c(cnn5C)c4)c(-c4c(Cl)c(C)cc5[nH]ncc45)c3C)C2)C1]
or a stereoisomer or tautomer thereof, or a pharmaceutically acceptable salt of any of the foregoing; and
(iii) instructions for administering the combination to treat cancer in a subject in need thereof.

68. A method of treating cancer in a subject in need thereof, comprising administering to the subject an effective amount of a TEAD inhibitor and an effective amount of a KRAS inhibitor,
wherein the TEAD inhibitor is a TEAD palmitate pocket binding inhibitor, or a covalent TEAD inhibitor; and
wherein the KRAS inhibitors is a G12C KRAS inhibitor.
